SEQUENCE LISTING (2 sequences)

SEQ ID NO: 1 - RNA, 24 nt, synthetic construct
  modified_base 1: mod_base = OTHER; note = inverted abasic deoxyribonucleotide, 5' end (NH2-C6) modification with phosphorothioate linkage
  modified_base 1^2: mod_base = OTHER; note = phosphorothioate linkage
  modified_base 2: mod_base = gm
  modified_base 3: mod_base = gm
  modified_base 4: mod_base = cm
  modified_base 5: mod_base = cm
  modified_base 6: mod_base = OTHER; note = 2'-O-methyladenosine
  modified_base 7: mod_base = um
  modified_base 8: mod_base = gm
  modified_base 9: mod_base = OTHER; note = 2'-O-methyladenosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-fluorouridine
  modified_base 13: mod_base = um
  modified_base 14: mod_base = gm
  modified_base 15: mod_base = cm
  modified_base 16: mod_base = um
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = cm
  modified_base 22: mod_base = OTHER; note = 2'-O-methyladenosine
  modified_base 22^23: mod_base = OTHER; note = phosphorothioate linkage
  modified_base 23: mod_base = OTHER; note = inverted abasic deoxyribonucleotide
  modified_base 23^24: mod_base = OTHER; note = C6-SS-C6 linkage
  modified_base 24: mod_base = OTHER; note = 2 -deoxythymidine
  source 1..24: mol_type = other RNA
nggccatgatcttgctgtaacant

SEQ ID NO: 2 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-fluoroguanosine
  modified_base 19: mod_base = gm
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tgttacagcaagatcatggcc

(19) United States
(12) Patent Application Publication
(10) Pub. No.: US 2023/0407313 A1
(43) Pub. Date: Dec. 21, 2023

(54) INTEGRIN TARGETING LIGANDS AND USES THEREOF

(71) Applicant: Arrowhead Pharmaceuticals, Inc., Pasadena, CA (US)

(72) Inventors: Xiaokai Li, Middleton, WI (US); Tao Pei, Middleton, WI (US); Susan Phan, Fitchburg, WI (US); Andrei V. Blokhin, Fitchburg, WI (US)

(21) Appl. No.: 18/181,340

(22) Filed: Mar. 9, 2023

Related U.S. Application Data
(63) Continuation of application No. PCT/US2021/049905, filed on Sep. 10, 2021.
(60) Provisional application No. 63/077,245, filed on Sep. 11, 2020.

(51) Int. Cl. C12N 15/113; A61P 21/00; C07H 19/00

(52) U.S. Cl. CPC C12N 15/1138; A61P 21/00; C07H 19/00; C12N 2310/14

(57) ABSTRACT

Synthetic αvβ6 integrin ligands of Formula I having serum stability and affinity for integrin αvβ6, which is a receptor expressed in a variety of cell types, are described. The described ligands are useful for delivering cargo molecules, such as RNAi agents or other oligonucleotide-based compounds, to cells that express integrin αvβ6, and thereby facilitating the uptake of the cargo molecules into these cells. Compositions that include αvβ6 integrin ligands and methods of use are also described.

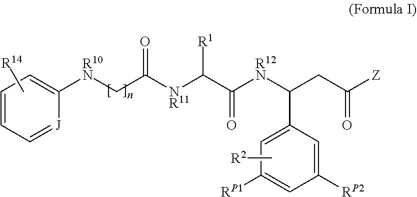

### CROSS REFERENCE TO RELATED APPLICATIONS

This application is a continuation application under 35 U.S.C. 111(a) of PCT application PCT/US2021/049905, filed Sep. 10, 2021, which claims the benefit of U.S. provisional application No. 63/077,245, filed on Sep. 11, 2020. These documents are hereby incorporated by reference in their entirety.

### SEQUENCE LISTING

This application contains a Sequence Listing which has been submitted in XML format and is hereby incorporated by reference in its entirety. The XML copy is named 30695-US1_ST26_SeqListing.xml, created Mar. 8, 2023, and is 27 kb in size.

### FIELD OF THE INVENTION

The present disclosure relates to targeting ligands that bind to integrin receptors for the delivery of oligonucleotide-based compounds, e.g., double-stranded RNAi agents, to certain cell types in vivo, for inhibition of genes expressed in those cells.

### BACKGROUND

It has been shown that αvβ6 integrin can promote cell invasion and migration in metastasis, and inhibit apoptosis. αvβ6 integrin may also regulate expression of matrix metalloproteases (MMPs) and activate TGF-β1. There is increasing evidence, primarily from in vitro studies, that suggest that αvβ6 integrin may promote carcinoma progression. Thus, integrin αvβ6 is attractive as a tumor biomarker and potential therapeutic target in view of, among other things, its role in expression of matrix metalloproteases (MMPs) and activation of TGF-β1.

The in vivo delivery of therapeutically effective compounds, such as drug compounds, to the desired cells and/or tissues, continues to be a general challenge for the development of drug products. There continues to exist a need for stable and effective targeting ligands that are able to selectively target cells or tissues, which can be employed to facilitate the targeted delivery of cargo molecules (e.g., a therapeutically active compound or ingredient) to specific cells or tissues. Indeed, there is a general need for targeting ligands that can be conjugated to one or more cargo molecules of choice, such as one or more drug products or other payloads, to facilitate the delivery of the cargo molecules to desired cells or tissues in vivo. Moreover, there exists a need for compounds that target integrin alpha-v beta-6, which are suitable to be conjugated to cargo molecules, to deliver the cargo molecules to cells expressing integrin alpha-v beta-6, in vivo. With respect to specific cargo molecules, such as therapeutic oligonucleotide-based compounds (e.g., an antisense oligonucleotides or an RNAi agents), there exists a need for targeting ligands that are able to target integrin alpha-v beta-6 that can be conjugated to oligonucleotide-based compounds to deliver the therapeutic to cells and/or tissues expressing integrin alpha-v beta-6, and facilitate the entry of the therapeutic into the cell through receptor-mediated endocytosis, pinocytosis, or by other means.

### SUMMARY

Described herein are novel, synthetic αvβ6 integrin ligands (also referred to herein as αvβ6 ligands). The αvβ6 integrin ligands disclosed herein are stable in serum and have affinity for, and can bind with specificity to, αvβ6 integrins. The αvβ6 integrin ligands can be conjugated to cargo molecules to facilitate the delivery of the cargo molecule to desired cells or tissues that express αvβ6 integrin, such as to skeletal muscle cells.

Also disclosed herein are methods of delivery of a cargo molecule to a tissue and/or cell expressing αvβ6 integrin in vivo, wherein the methods including administering to a subject one or more αvβ6 integrin ligands disclosed herein that have been conjugated to one or more cargo molecules. Further disclosed are methods of treatment of a subject having a disease, symptom, or disorder for which the delivery of a therapeutic cargo molecule (e.g., an active pharmaceutical ingredient) to a cell expressing αvβ6 integrin is capable of treating the subject, wherein the methods include administering to a subject one or more αvβ6 integrin ligands disclosed herein that have been conjugated to one or more therapeutic cargo molecules.

In some embodiments, described herein are methods of inhibiting expression of a target gene in a cell, wherein the methods include administering to the cell an effective amount of one or more αvβ6 integrin ligands that have been conjugated to one or more oligonucleotide-based compounds (e.g., an oligonucleotide-based therapeutic) capable of inhibiting expression of a target gene in a cell, such as an RNAi agent. In some embodiments, described herein are methods of inhibiting expression of a target gene in a cell of a subject, wherein the subject is administered an effective amount of one or more αvβ6 integrin ligands that have been conjugated to one or more oligonucleotide-based compounds capable of inhibiting expression of a target gene in a cell, such as an RNAi agent.

Further described herein are compositions that include αvβ6 integrin ligands. The compositions described herein can be pharmaceutical compositions that include one or more αvβ6 integrin ligands disclosed herein conjugated to one or more therapeutic substances, such as an RNAi agent or other cargo molecule.

In some embodiments, described herein are methods of treatment of a subject having a disease or disorder mediated at least in part by expression of a target gene, wherein the methods including administering to a subject in need thereof an effective amount of a pharmaceutical composition, wherein the pharmaceutical composition includes one or more αvβ6 integrin ligands disclosed herein conjugated to one or more oligonucleotide-based compounds, such as an RNAi agent.

In a first aspect, this disclosure provides synthetic αvβ6 integrin ligands.

In some embodiments, an αvβ6 integrin ligand disclosed herein includes the structure of the Formula I:

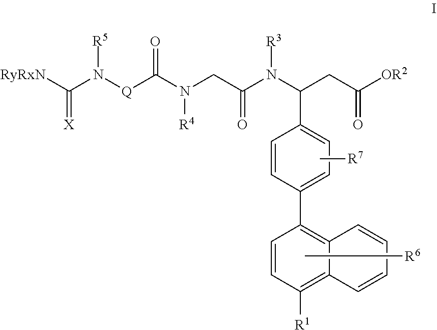

or a pharmaceutically acceptable salt thereof, wherein
R¹ is optionally substituted alkyl, optionally substituted alkoxy, or

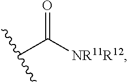

wherein R¹¹ and R¹² are each independently optionally substituted alkyl or a cargo molecule, or R¹ is a cargo molecule;
R² is H, optionally substituted alkyl, or a cargo molecule;
R³ is H or optionally substituted alkyl;
R⁴ is H or optionally substituted alkyl;
R⁵ is H or optionally substituted alkyl;
R⁶ is selected from the group consisting of H, optionally substituted alkyl, optionally substituted alkoxy, halo, optionally substituted amino, or a cargo molecule;
Q is optionally substituted aryl or optionally substituted alkylene;
X is O, CR⁸R⁹, NR⁸;
wherein R⁸ is selected from H, optionally substituted alkyl, or R⁸ is taken together with Rx or Ry to form a 4-, 5-, 6-, 7-, 8- or 9-membered ring, and R⁹ is H or optionally substituted alkyl;
Rx and Ry are each independently H, optionally substituted alkyl, a cargo molecule or Rx and Ry may be taken together to form a double bond with R¹⁰, wherein R¹⁰ is H, optionally substituted alkyl, or R¹⁰ may be taken together with X and the atoms to which it is attached to form a 4-, 5-, 6-, 7-, 8, or 9-membered ring;
wherein at least one of R¹, R², R⁶, R¹¹, R¹², Rx and Ry comprise a cargo molecule; and
wherein when Q is optionally substituted alkylene and the length of the optionally substituted alkylene chain represented by Q is 3 carbons, then R¹ is

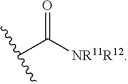

In some embodiments, an αvβ6 integrin ligand disclosed herein can be conjugated to one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30; or 10 to 30, 1 to 25, 1 to 20, 1 to 15, 1 to 10, 1 to 5, 5 to 30, 5 to 25, 5 to 20, 5 to 15, 5 to 10, 10 to 30, 10 to 25, 10 to 20, 10 to 15, 15 to 30, 15 to 25, 15 to 20, 20 to 30, 20 to 25, or 25 to 30) cargo molecules (e.g., any of the cargo molecules described herein or known in the art).

In some embodiments, more than one αvβ6 integrin ligand disclosed herein (e.g., 2, 3, 4, 5, 6, 7, 8, or 10 to 8, 1 to 7, 1 to 6, 1 to 5, 1 to 4, 1 to 3, 1 to 2, 2 to 8, 2 to 7, 2 to 6, 2 to 5, 2 to 4, 2 to 3, 3 to 8, 3 to 7, 3 to 6, 3 to 5, 3 to 4, 4 to 8, 4 to 7, 4 to 6, or 4 to 5 αvβ6 integrin ligands) can be conjugated to one cargo molecule (e.g., any of the cargo molecules described herein or known in the art).

In another aspect, this disclosure provides compositions that include one or more of the αvβ6 integrin ligands described herein. For example, in some embodiments, compositions comprising one or more αvβ6 integrin ligands disclosed herein include one or more oligonucleotide-based compound(s), such as one or more RNAi agent(s), to be delivered to a cell in vivo. In some embodiments, described herein are compositions for delivering an RNAi agent to a cell in vivo, wherein the RNAi agent is linked to one or more αvβ6 integrin ligands.

Compositions that include one or more αvβ6 integrin ligands are described. In some embodiments, a composition comprises a pharmaceutically acceptable excipient. In some embodiments, a composition that includes one or more αvβ6 integrin ligands comprises one or more other pharmaceutical substances or pharmaceutically active ingredients or compounds. In some embodiments, medicaments that include one or more αvβ6 integrin ligands are described herein.

Compositions that include one or more αvβ6 integrin ligands disclosed herein conjugated to one or more cargo molecules can facilitate the delivery of the cargo molecule in vivo or in vitro to cells that express integrin αvβ6. For example, compositions that include one or more αvβ6 integrin ligands disclosed herein can deliver cargo molecules, such as oligonucleotide-based compounds, in vivo or in vitro, to skeletal muscle cells, type I and II alveolar epithelial cells, goblet cells, secretory epithelial cells, ciliated epithelial cells, corneal and conjunctival epithelial cells, dermal epithelial cells, cholangiocytes, enterocytes, ductal epithelial cells, glandular epithelial cells, and epithelial tumors (carcinomas).

In another aspect, the present disclosure provides methods comprising the use of one or more αvβ6 integrin ligands and/or compositions as described herein and, if desired, bringing the disclosed αvβ6 integrin ligands and/or compositions into a form suitable for administration as a pharmaceutical product. In other embodiments, the disclosure provides methods for the manufacture of the ligands and compositions, e.g., medicaments, described herein.

Compositions that include one or more αvβ6 integrin ligands can be administered to subjects in vivo using routes of administration known in the art to be suitable for such administration in view of the cargo molecule sought to be administered, including, for example, subcutaneous, intravenous, intraperitoneal, intradermal, transdermal, oral, sublingual, topical, or intratumoral administration. In some embodiments, the compositions that include one or more αvβ6 integrin ligands may be administered for systemic delivery, for example, by intravenous or subcutaneous administration. In some embodiments, the compositions that include one or more αvβ6 integrin ligands may be administered for localized delivery, for example, by inhaled delivery via dry powder inhaler or nebulizer. In some embodiments, the compositions that include one or more αvβ6 integrin ligands may be administered for localized delivery by topical administration.

In some embodiments, disclosed herein are methods for delivering one or more desired cargo molecule(s) to a skeletal muscle cell in vivo, wherein the methods include administering to the subject one or more αvβ6 integrin ligands conjugated to the one or more cargo molecule.

In some embodiments, disclosed herein are methods for delivering one or more desired cargo molecule(s) to a type II alveolar epithelial cell in vivo, wherein the methods include administering to the subject one or more αvβ6 integrin ligands conjugated to the one or more cargo molecule.

In some embodiments, disclosed herein are methods for delivering one or more desired cargo molecule(s) to a goblet cell in vivo, wherein the methods include administering to the subject one or more αvβ6 integrin ligands conjugated to the one or more cargo molecule.

In some embodiments, disclosed herein are methods for delivering one or more desired cargo molecule(s) to a secretory epithelial cell in vivo, wherein the methods include administering to the subject one or more αvβ6 integrin ligands conjugated to the one or more cargo molecule.

In some embodiments, disclosed herein are methods for delivering one or more desired cargo molecule(s) to a ciliated epithelial cell in vivo, wherein the methods include administering to the subject one or more αvβ6 integrin ligands conjugated to the one or more cargo molecule.

In some embodiments, disclosed herein are methods for delivering one or more desired cargo molecule(s) to a corneal epithelial cell in vivo, wherein the methods include administering to the subject one or more αvβ6 integrin ligands conjugated to the one or more cargo molecule.

In some embodiments, disclosed herein are methods for delivering one or more desired cargo molecule(s) to a conjunctival epithelial cell in vivo, wherein the methods include administering to the subject one or more αvβ6 integrin ligands conjugated to the one or more cargo molecule.

In some embodiments, disclosed herein are methods for delivering one or more desired cargo molecule(s) to a dermal epithelial cell in vivo, wherein the methods include administering to the subject one or more αvβ6 integrin ligands conjugated to the one or more cargo molecule.

In some embodiments, disclosed herein are methods for delivering one or more desired cargo molecule(s) to a cholangiocyte in vivo, wherein the methods include administering to the subject one or more αvβ6 integrin ligands conjugated to the one or more cargo molecule.

In some embodiments, disclosed herein are methods for delivering one or more desired cargo molecule(s) to an enterocyte in vivo, wherein the methods include administering to the subject one or more αvβ6 integrin ligands conjugated to the one or more cargo molecule.

In some embodiments, disclosed herein are methods for delivering one or more desired cargo molecule(s) to a ductal epithelial cell in vivo, wherein the methods include administering to the subject one or more αvβ6 integrin ligands conjugated to the one or more cargo molecule.

In some embodiments, disclosed herein are methods for delivering one or more desired cargo molecule(s) to a glandular epithelial cell in vivo, wherein the methods include administering to the subject one or more αvβ6 integrin ligands conjugated to the one or more cargo molecule.

In some embodiments, disclosed herein are methods for delivering one or more desired cargo molecule(s) to an epithelial tumor (carcinoma) in vivo, wherein the methods include administering to the subject one or more αvβ6 integrin ligands conjugated to the one or more cargo molecules.

In some embodiments, disclosed herein are methods of delivering an oligonucleotide-based compound to a type I alveolar epithelial cell in vivo, wherein the methods include administering to the subject one or more αvβ6 integrin ligands conjugated to the one or more oligonucleotide-based compounds. In some embodiments, disclosed herein are methods of delivering an RNAi agent to a type I alveolar epithelial cell in vivo, wherein the methods include administering to the subject one or more αvβ6 integrin ligands conjugated to the one or more RNAi agents. In some embodiments, disclosed herein are methods of inhibiting the expression of a target gene in a type I alveolar epithelial cell in vivo, wherein the methods include administering to the subject an RNAi agent conjugated to one or more ligands having affinity for αvβ6 integrin.

In some embodiments, disclosed herein are methods of delivering an oligonucleotide-based compound to a type II alveolar epithelial cell in vivo, wherein the methods include administering to the subject one or more αvβ6 integrin ligands conjugated to the one or more oligonucleotide-based compounds. In some embodiments, disclosed herein are methods of delivering an RNAi agent to a type II alveolar epithelial cell in vivo, wherein the methods include administering to the subject one or more αvβ6 integrin ligands conjugated to the one or more RNAi agents. In some embodiments, disclosed herein are methods of inhibiting the expression of a target gene in a type II alveolar epithelial cell in vivo, wherein the methods include administering to the subject an RNAi agent conjugated to one or more ligands having affinity for αvβ6 integrin.

In some embodiments, disclosed herein are methods of delivering an oligonucleotide-based compound to a goblet cell in vivo, wherein the methods include administering to the subject one or more αvβ6 integrin ligands conjugated to the one or more oligonucleotide-based compounds. In some embodiments, disclosed herein are methods of delivering an RNAi agent to a goblet cell in vivo, wherein the methods include administering to the subject one or more αvβ6 integrin ligands conjugated to the one or more RNAi agents. In some embodiments, disclosed herein are methods of inhibiting the expression of a target gene in a goblet cell in vivo, wherein the methods include administering to the subject an RNAi agent conjugated to one or more ligands having affinity for αvβ6 integrin.

In some embodiments, disclosed herein are methods of delivering an oligonucleotide-based compound to a secretory epithelial cell in vivo, wherein the methods include administering to the subject one or more αvβ6 integrin ligands conjugated to the one or more oligonucleotide-based compounds. In some embodiments, disclosed herein are methods of delivering an RNAi agent to a secretory epithelial cell in vivo, wherein the methods include administering to the subject one or more αvβ6 integrin ligands conjugated to the one or more RNAi agents. In some embodiments, disclosed herein are methods of inhibiting the expression of a target gene in a secretory epithelial cell in vivo, wherein the methods include administering to the subject an RNAi agent conjugated to one or more ligands having affinity for αvβ6 integrin.

In some embodiments, disclosed herein are methods of delivering an oligonucleotide-based compound to a ciliated epithelial cell in vivo, wherein the methods include administering to the subject one or more αvβ6 integrin ligands conjugated to the one or more oligonucleotide-based compounds. In some embodiments, disclosed herein are methods of delivering an RNAi agent to a ciliated epithelial cell in vivo, wherein the methods include administering to the subject one or more αvβ6 integrin ligands conjugated to the one or more RNAi agents. In some embodiments, disclosed herein are methods of inhibiting the expression of a target gene in a ciliated epithelial cell in vivo, wherein the methods include administering to the subject an RNAi agent conjugated to one or more ligands having affinity for αvβ6 integrin.

In some embodiments, disclosed herein are methods of delivering an oligonucleotide-based compound to a corneal epithelial cell in vivo, wherein the methods include administering to the subject one or more αvβ6 integrin ligands conjugated to the one or more oligonucleotide-based compounds. In some embodiments, disclosed herein are methods of delivering an RNAi agent to a corneal epithelial cell in vivo, wherein the methods include administering to the subject one or more αvβ6 integrin ligands conjugated to the one or more RNAi agents. In some embodiments, disclosed herein are methods of inhibiting the expression of a target gene in a corneal epithelial cell in vivo, wherein the methods include administering to the subject an RNAi agent conjugated to one or more ligands having affinity for αvβ6 integrin.

In some embodiments, disclosed herein are methods of delivering an oligonucleotide-based compound to a conjunctival epithelial cell in vivo, wherein the methods include administering to the subject one or more αvβ6 integrin ligands conjugated to the one or more oligonucleotide-based compounds. In some embodiments, disclosed herein are methods of delivering an RNAi agent to a conjunctival epithelial cell in vivo, wherein the methods include administering to the subject one or more αvβ6 integrin ligands conjugated to the one or more RNAi agents. In some embodiments, disclosed herein are methods of inhibiting the expression of a target gene in a conjunctival epithelial cell in vivo, wherein the methods include administering to the subject an RNAi agent conjugated to one or more ligands having affinity for αvβ6 integrin.

In some embodiments, disclosed herein are methods of delivering an oligonucleotide-based compound to a dermal epithelial cell in vivo, wherein the methods include administering to the subject one or more αvβ6 integrin ligands conjugated to the one or more oligonucleotide-based compounds. In some embodiments, disclosed herein are methods of delivering an RNAi agent to a dermal epithelial cell in vivo, wherein the methods include administering to the subject one or more αvβ6 integrin ligands conjugated to the one or more RNAi agents. In some embodiments, disclosed herein are methods of inhibiting the expression of a target gene in a dermal epithelial cell in vivo, wherein the methods include administering to the subject an RNAi agent conjugated to one or more ligands having affinity for αvβ6 integrin.

In some embodiments, disclosed herein are methods of delivering an oligonucleotide-based compound to a cholangiocyte in vivo, wherein the methods include administering to the subject one or more αvβ6 integrin ligands conjugated to the one or more oligonucleotide-based compounds. In some embodiments, disclosed herein are methods of delivering an RNAi agent to a cholangiocyte in vivo, wherein the methods include administering to the subject one or more αvβ6 integrin ligands conjugated to the one or more RNAi agents. In some embodiments, disclosed herein are methods of inhibiting the expression of a target gene in a cholangiocyte in vivo, wherein the methods include administering to the subject an RNAi agent conjugated to one or more ligands having affinity for αvβ6 integrin.

In some embodiments, disclosed herein are methods of delivering an oligonucleotide-based compound to an enterocyte in vivo, wherein the methods include administering to the subject one or more αvβ6 integrin ligands conjugated to the one or more oligonucleotide-based compounds. In some embodiments, disclosed herein are methods of delivering an RNAi agent to an enterocyte in vivo, wherein the methods include administering to the subject one or more αvβ6 integrin ligands conjugated to the one or more RNAi agents. In some embodiments, disclosed herein are methods of inhibiting the expression of a target gene in an enterocyte in vivo, wherein the methods include administering to the subject an RNAi agent conjugated to one or more ligands having affinity for αvβ6 integrin.

In some embodiments, disclosed herein are methods of delivering an oligonucleotide-based compound to a ductal epithelial cell in vivo, wherein the methods include administering to the subject one or more αvβ6 integrin ligands conjugated to the one or more oligonucleotide-based compounds. In some embodiments, disclosed herein are methods of delivering an RNAi agent to a ductal epithelial cell in vivo, wherein the methods include administering to the subject one or more αvβ6 integrin ligands conjugated to the one or more RNAi agents. In some embodiments, disclosed herein are methods of inhibiting the expression of a target gene in a ductal epithelial cell in vivo, wherein the methods include administering to the subject an RNAi agent conjugated to one or more ligands having affinity for αvβ6 integrin.

In some embodiments, disclosed herein are methods of delivering an oligonucleotide-based compound to a glandular epithelial cell in vivo, wherein the methods include administering to the subject one or more αvβ6 integrin ligands conjugated to the one or more oligonucleotide-based compounds. In some embodiments, disclosed herein are methods of delivering an RNAi agent to a glandular epithelial cell in vivo, wherein the methods include administering to the subject one or more αvβ6 integrin ligands conjugated to the one or more RNAi agents. In some embodiments, disclosed herein are methods of inhibiting the expression of a target gene in a glandular epithelial cell in vivo, wherein the methods include administering to the subject an RNAi agent conjugated to one or more ligands having affinity for αvβ6 integrin.

In some embodiments, disclosed herein are methods of delivering an oligonucleotide-based compound to an epithelial tumor (carcinoma) in vivo, wherein the methods include administering to the subject one or more αvβ6 integrin ligands conjugated to the one or more oligonucleotide-based compounds. In some embodiments, disclosed herein are methods of delivering an RNAi agent to an epithelial tumor (carcinoma) in vivo, wherein the methods include administering to the subject one or more αvβ6 integrin ligands conjugated to the one or more RNAi agents. In some embodiments, disclosed herein are methods of inhibiting the expression of a target gene in an epithelial tumor (carcinoma) in vivo, wherein the methods include administering to the subject an RNAi agent conjugated to one or more ligands having affinity for αvβ6 integrin.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description and from the claims.

### DETAILED DESCRIPTION

αvρ6 Integrin Ligands.

Described herein are synthetic αvβ6 integrin ligands having serum stability and affinity for integrin αvβ6. The αvβ6 integrin ligands can be used to target cells that express integrin αvβ6 in vitro, in situ, ex vivo, and/or in vivo. In some embodiments, the αvβ6 integrin ligands disclosed herein can be conjugated to one or more cargo molecules to preferentially direct and target the cargo molecules to cells that express integrin αvβ6 in vitro, in situ, ex vivo, and/or in vivo. In some embodiments, the cargo molecules include or consist of pharmaceutically active compounds. In some embodiments, the cargo molecules include or consist of oligonucleotide-based compounds, such as RNAi agents. In some embodiments, the αvβ6 integrin ligands disclosed herein are conjugated to cargo molecules to direct the cargo molecules to epithelial cells in vivo.

In a first aspect, this disclosure provides synthetic αvβ6 integrin ligands.

In some embodiments, an αvβ6 integrin ligand disclosed herein includes the structure of Formula I.

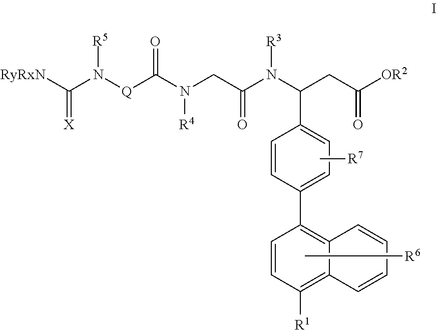

or a pharmaceutically acceptable salt thereof, wherein
R¹ is optionally substituted alkyl, optionally substituted alkoxy, or

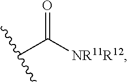

wherein R¹¹ and R¹² are each independently optionally substituted alkyl or a cargo molecule, or R¹ is a cargo molecule;
R² is H, optionally substituted alkyl, or a cargo molecule;
R³ is H or optionally substituted alkyl;
R⁴ is H or optionally substituted alkyl;
R⁵ is H or optionally substituted alkyl;
R⁶ is selected from the group consisting of H, optionally substituted alkyl, optionally substituted alkoxy, halo, optionally substituted amino, or a cargo molecule;
Q is optionally substituted aryl or optionally substituted alkylene;
X is O, CR⁸R⁹, NR⁸;
wherein R⁸ is selected from H, optionally substituted alkyl, or R⁸ is taken together with Rx or Ry to form a 4-, 5-, 6-, 7-, 8- or 9-membered ring, and R⁹ is H or optionally substituted alkyl;
Rx and Ry are each independently H, optionally substituted alkyl, a cargo molecule or Rx and Ry may be taken together to form a double bond with R¹⁰ wherein R¹⁰ is H, optionally substituted alkyl, or R¹⁰ may be taken together with X and the atoms to which it is attached to form a 4-, 5-, 6-, 7-, 8, or 9-membered ring;
wherein at least one of R¹, R², R⁶, R¹¹, R¹², Rx and Ry comprise a cargo molecule; and
wherein when Q is optionally substituted alkylene and the length of the optionally substituted alkylene chain represented by Q is 3 carbons, then R¹ is

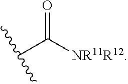

In some embodiments, the compound of Formula I is a compound of Formula Ia:

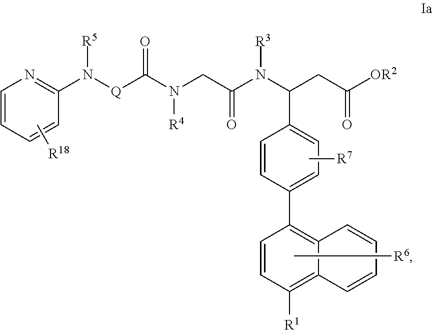

wherein R¹⁸ is selected from the group consisting of H, optionally substituted alkyl, optionally substituted alkoxy, halo, —NR¹⁹R²⁰, wherein R¹⁹ and R²⁰ are each independently H or optionally substituted alkyl.

In some embodiments, the compound of Formula I is a compound of Formula Ib:

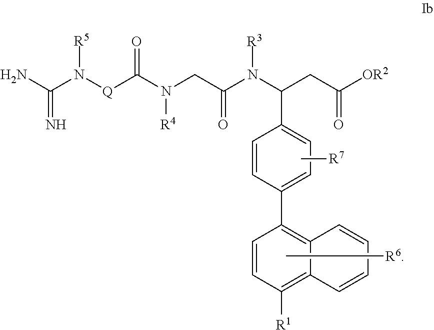

In some embodiments, the compound of Formula I is a compound of Formula Ic:

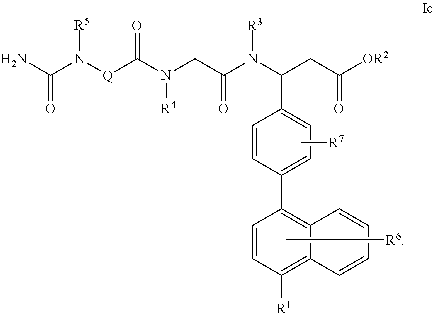

In some embodiments, the compound of Formula I is a compound of Formula Id:

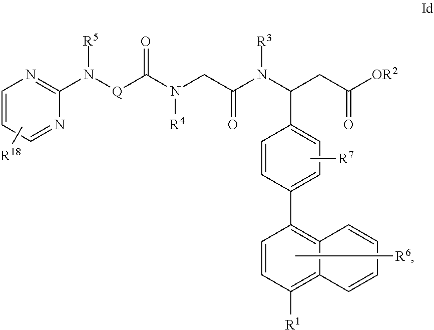

wherein R¹⁸ is selected from the group consisting of H, optionally substituted alkyl, optionally substituted alkoxy, halo, —NR¹⁹R²⁰, wherein R¹⁹ and R²⁰ are each independently H or optionally substituted alkyl.

In some embodiments of Formula I, Q is

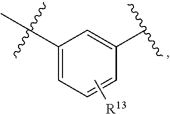

wherein R¹³ is selected from the group consisting of H, OH, optionally substituted alkyl, optionally substituted alkoxy, halo, and optionally substituted amino. In further embodiments of Formula I, Q is

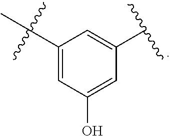 [SMILES: *c1cc(*)cc(O)c1]

In other embodiments, Q is

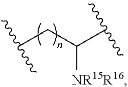

wherein R¹⁵ and R¹⁶ are each independently H,

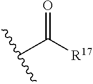

wherein R¹⁷ is optionally substituted alkyl, or optionally substituted alkyl; and n is an integer from 1 to 10. In some embodiments, n is 4. In further embodiments of Formula I, Q is

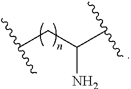

In further embodiments of Formula I, Q is

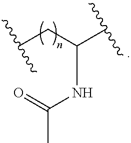

In some embodiments, n is 4. In other embodiments of Formula I, Q is C₁-C₁₀ alkylene. In further embodiments, Q is —(CH₂)₄—.

In some embodiments of Formula I, R¹ comprises a cargo molecule. In further embodiments, of Formula I, R¹ comprises at least one polyethylene glycol (PEG) unit and a cargo molecule. In some embodiments of Formula I, R¹ comprises between 1 and 10 PEG units. In further embodiments, R¹ comprises 5 PEG units.

In some embodiments of Formula I, R⁶ and R⁷ are both H. In some embodiments of Formula I, R³, R⁴, and R⁵ are all H.

In some embodiments, an αvβ6 integrin ligand disclosed herein can be conjugated to one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30; or 10 to 30, 1 to 25, 1 to 20, 1 to 15, 1 to 10, 1 to 5, 5 to 30, 5 to 25, 5 to 20, 5 to 15, 5 to 10, 10 to 30, 10 to 25, 10 to 20, 10 to 15, 15 to 30, 15 to 25, 15 to 20, 20 to 30, 20 to 25, or 25 to 30) cargo molecules (e.g., any of the cargo molecules described herein or known in the art).

In some embodiments, more than one αvβ6 integrin ligand disclosed herein (e.g., 2, 3, 4, 5, 6, 7, 8, or 10 to 8, 1 to 7, 1 to 6, 1 to 5, 1 to 4, 1 to 3, 1 to 2, 2 to 8, 2 to 7, 2 to 6, 2 to 5, 2 to 4, 2 to 3, 3 to 8, 3 to 7, 3 to 6, 3 to 5, 3 to 4, 4 to 8, 4 to 7, 4 to 6, or 4 to 5 αvβ6 integrin ligands) can be conjugated to one cargo molecule (e.g., any of the cargo molecules described herein or known in the art).

In some embodiments, the αvβ6 integrin ligands disclosed herein are optionally conjugated to one or more cargo molecules via a linking group, such as, for example, a polyethylene glycol (PEG) group.

In some embodiments, the αvβ6 integrin ligands disclosed herein are optionally conjugated to one or more cargo molecules via a scaffold that includes at least one attachment point for each ligand and at least one attachment point for each cargo molecule. In some embodiments, the αvβ6 integrin ligands comprise, consist of, or consist essentially of, one cargo molecule. In some embodiments, the αvβ6 integrin ligands comprise, consist of, or consist essentially of, more than one cargo molecule.

In some embodiments, the αvβ6 integrin ligand comprises Compound 41a, 41b, 42a, 42b, 43a, 43b, 44a, 44b, 45a, 45b, 46a, 46b, 47a, 47b, 48a, 48b, 49a, 49b, 50a, 50b, 51a, 51b, 52a, 52b, 53a, 53b, 54a, 54b, 55a, 55b, 56a, 56b, 57a, 57b, 58a, 58b, 59a, 59b, 60a, or 60b.

In one aspect, the invention provides a targeting ligand having the structure:

| Compound | |
|---|---|
| Number | Formula |
| 40a | 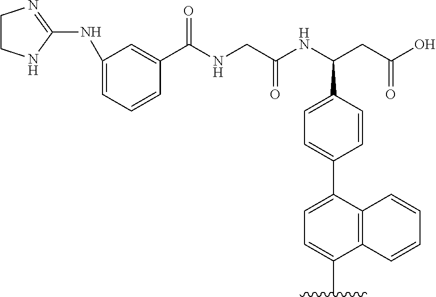 [SMILES: *c1ccc(-c2ccc([C@H](CC(=O)O)NC(=O)CNC(=O)c3cccc(NC4=NCCN4)c3)cc2)c2ccccc12] |
| 41a | 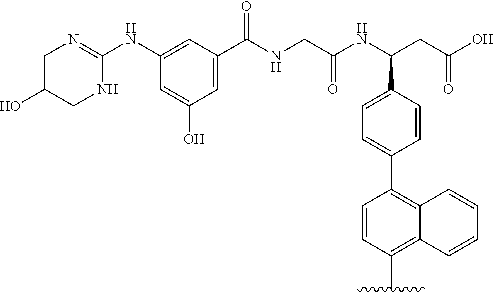 [SMILES: *c1ccc(-c2ccc([C@H](CC(=O)O)NC(=O)CNC(=O)c3cc(O)cc(NC4=NCC(O)CN4)c3)cc2)c2ccccc12] |
| 42a | 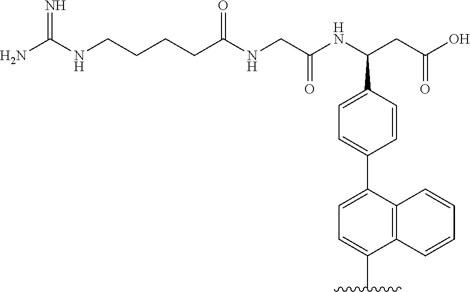 [SMILES: *c1ccc(-c2ccc([C@H](CC(=O)O)NC(=O)CNC(=O)CCCCNC(=N)N)cc2)c2ccccc12] |
| 43a | 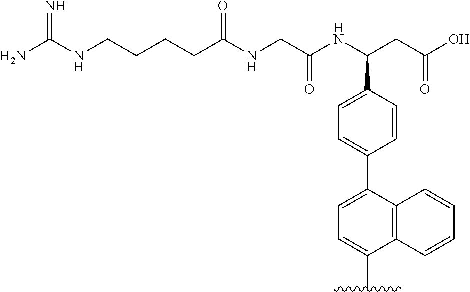 [SMILES: *c1ccc(-c2ccc([C@H](CC(=O)O)NC(=O)CNC(=O)CCCCNC(=N)N)cc2)c2ccccc12] |
| 44a | 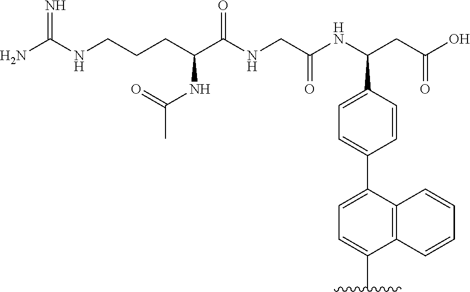 [SMILES: *c1ccc(-c2ccc([C@H](CC(=O)O)NC(=O)CNC(=O)[C@H](CCCNC(=N)N)NC(C)=O)cc2)c2ccccc12] |
| 45a | 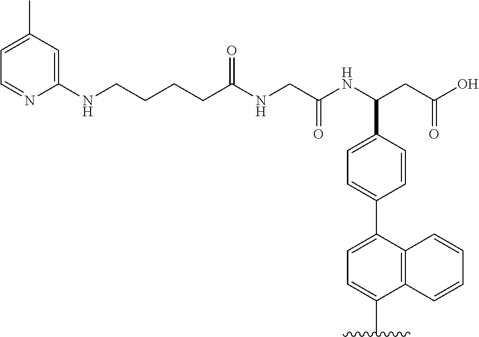 [SMILES: *c1ccc(-c2ccc([C@H](CC(=O)O)NC(=O)CNC(=O)CCCCNc3cc(C)ccn3)cc2)c2ccccc12] |
| 46a | 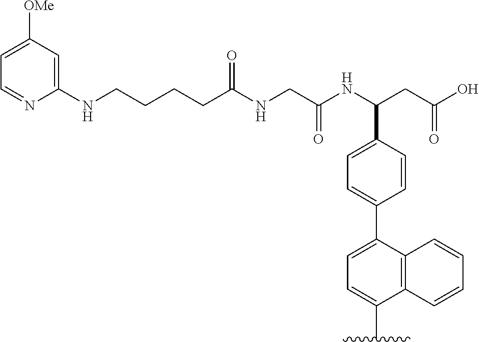 [SMILES: *c1ccc(-c2ccc([C@H](CC(=O)O)NC(=O)CNC(=O)CCCCNc3cc(OC)ccn3)cc2)c2ccccc12] |
| 47a | 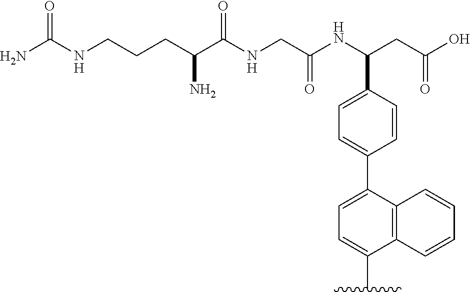 [SMILES: *c1ccc(-c2ccc([C@H](CC(=O)O)NC(=O)CNC(=O)[C@@H](N)CCCNC(N)=O)cc2)c2ccccc12] |
| 48a | 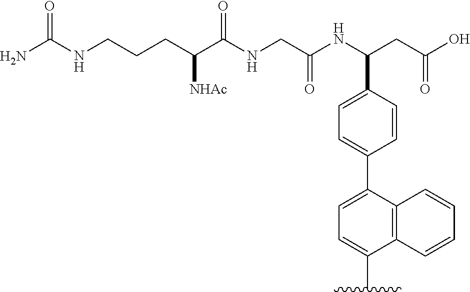 [SMILES: *c1ccc(-c2ccc([C@H](CC(=O)O)NC(=O)CNC(=O)[C@H](CCCNC(N)=O)NC(C)=O)cc2)c2ccccc12] |
| 49a | 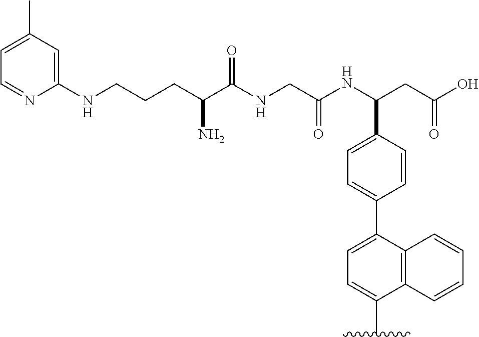 [SMILES: *c1ccc(-c2ccc([C@H](CC(=O)O)NC(=O)CNC(=O)[C@@H](N)CCCNc3cc(C)ccn3)cc2)c2ccccc12] |
| 50a | 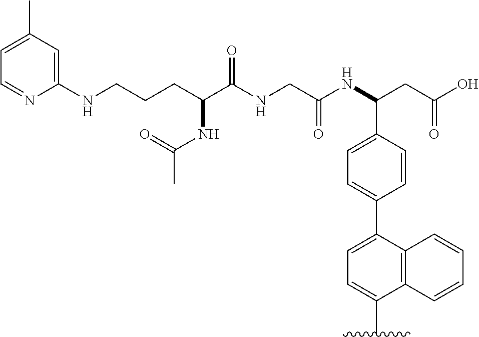 [SMILES: *c1ccc(-c2ccc([C@H](CC(=O)O)NC(=O)CNC(=O)[C@H](CCCNc3cc(C)ccn3)NC(C)=O)cc2)c2ccccc12] |
| 51a | 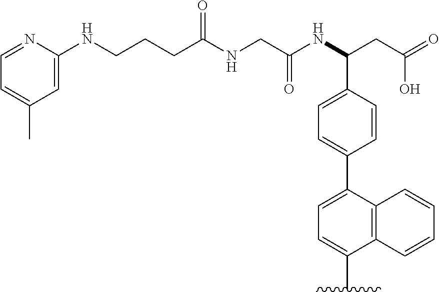 [SMILES: *c1ccc(-c2ccc([C@H](CC(=O)O)NC(=O)CNC(=O)CCCNc3cc(C)ccn3)cc2)c2ccccc12] |
| 52a | 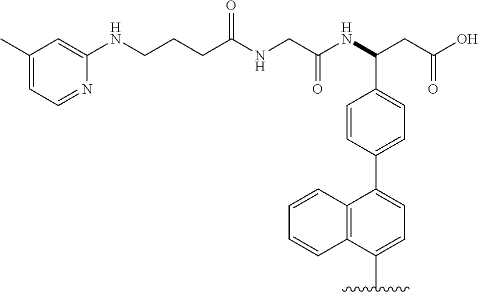 [SMILES: *c1ccc(-c2ccc([C@H](CC(=O)O)NC(=O)CNC(=O)CCCNc3cc(C)ccn3)cc2)c2ccccc12] |
| 53a | 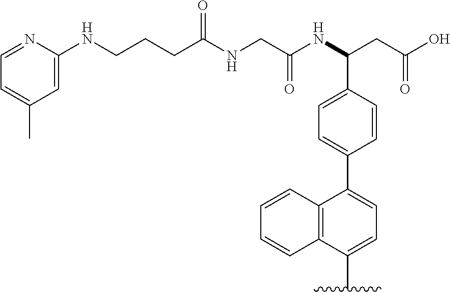 [SMILES: *c1ccc(-c2ccc([C@H](CC(=O)O)NC(=O)CNC(=O)CCCNc3cc(C)ccn3)cc2)c2ccccc12] |
| 54a | 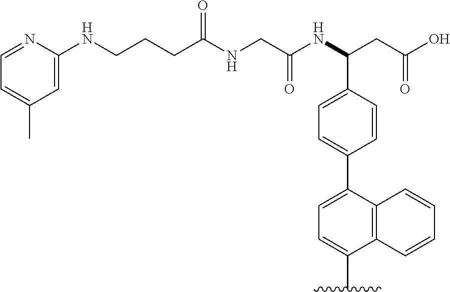 [SMILES: *c1ccc(-c2ccc([C@H](CC(=O)O)NC(=O)CNC(=O)CCCNc3cc(C)ccn3)cc2)c2ccccc12] |
| 55a | 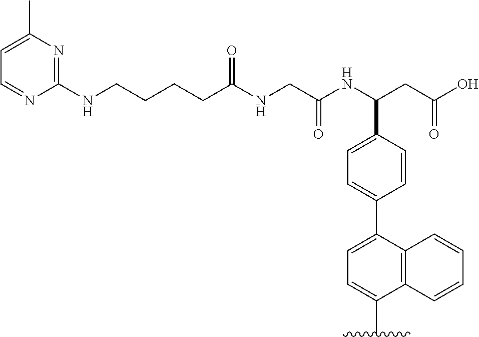 [SMILES: *c1ccc(-c2ccc([C@H](CC(=O)O)NC(=O)CNC(=O)CCCCNc3nccc(C)n3)cc2)c2ccccc12] |
| 56a | 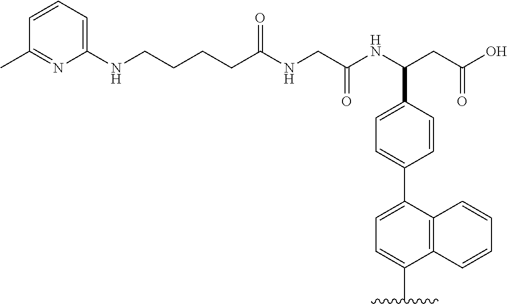 [SMILES: *c1ccc(-c2ccc([C@H](CC(=O)O)NC(=O)CNC(=O)CCCCNc3cccc(C)n3)cc2)c2ccccc12] |
| 57a | 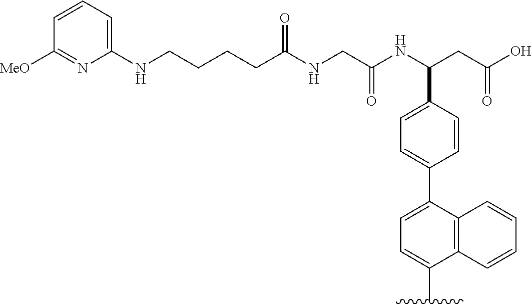 [SMILES: *c1ccc(-c2ccc([C@H](CC(=O)O)NC(=O)CNC(=O)CCCCNc3cccc(OC)n3)cc2)c2ccccc12] |
| 58a | 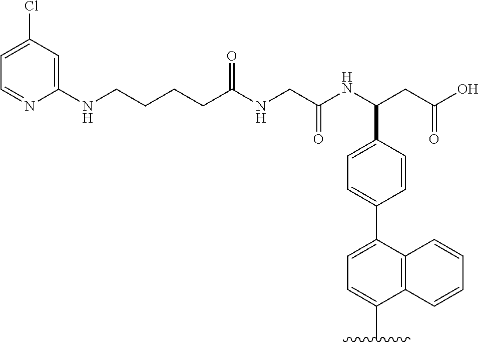 [SMILES: *c1ccc(-c2ccc([C@H](CC(=O)O)NC(=O)CNC(=O)CCCCNc3cc(Cl)ccn3)cc2)c2ccccc12] |
| 59a | 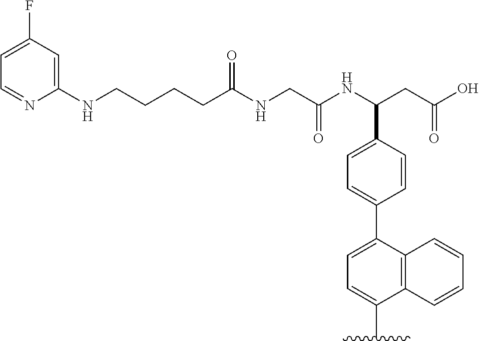 [SMILES: *c1ccc(-c2ccc([C@H](CC(=O)O)NC(=O)CNC(=O)CCCCNc3cc(F)ccn3)cc2)c2ccccc12] |
| 60a | 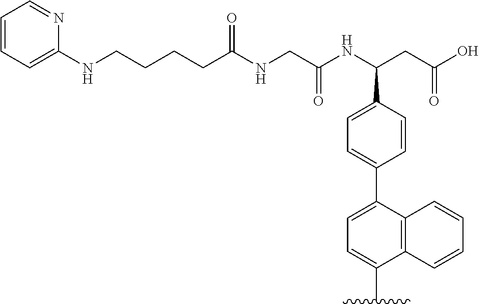 [SMILES: *c1ccc(-c2ccc([C@H](CC(=O)O)NC(=O)CNC(=O)CCCCNc3ccccn3)cc2)c2ccccc12] |

or a pharmaceutically acceptable salt thereof, wherein [symbol] indicates the point of connection to a cargo molecule.

Another aspect of the invention provides a compound having the formula:

| Compound | |
|---|---|
| Number | Formula |
| 40b | 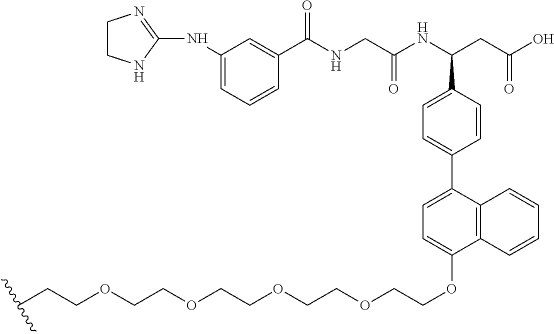 [SMILES: *CCOCCOCCOCCOCCOc1ccc(-c2ccc([C@H](CC(=O)O)NC(=O)CNC(=O)c3cccc(NC4=NCCN4)c3)cc2)c2ccccc12] |
| 41b | 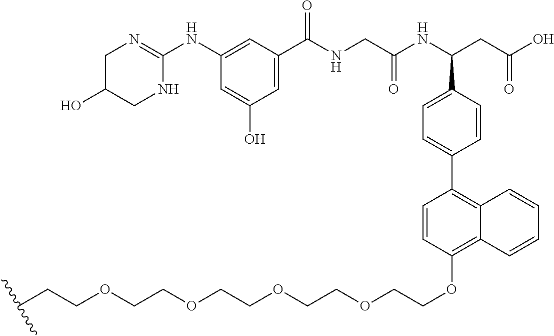 [SMILES: *CCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12] |
| 42b | 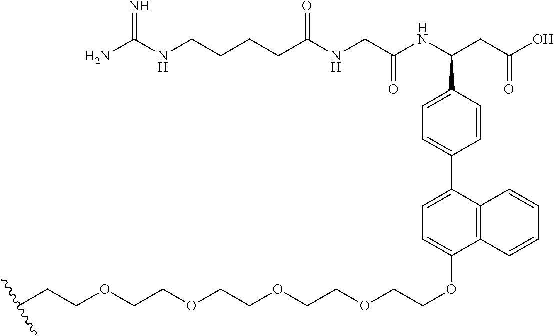 [SMILES: *CCOCCOCCOCCOCCOc1ccc(-c2ccc([C@H](CC(=O)O)NC(=O)CNC(=O)CCCCNC(=N)N)cc2)c2ccccc12] |
| 43b | 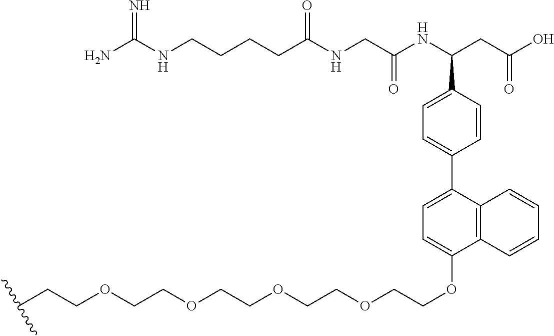 [SMILES: *CCOCCOCCOCCOCCOc1ccc(-c2ccc([C@H](CC(=O)O)NC(=O)CNC(=O)CCCCNC(=N)N)cc2)c2ccccc12] |
| 44b | 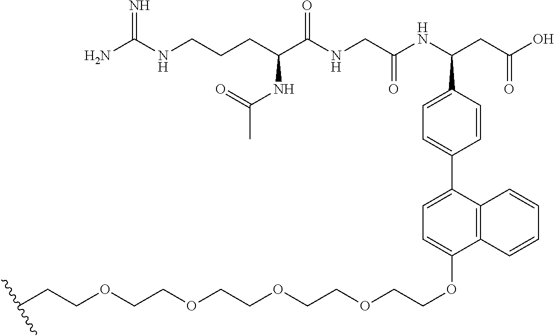 [SMILES: *CCOCCOCCOCCOCCOc1ccc(-c2ccc([C@H](CC(=O)O)NC(=O)CNC(=O)[C@H](CCCNC(=N)N)NC(C)=O)cc2)c2ccccc12] |
| 45b | 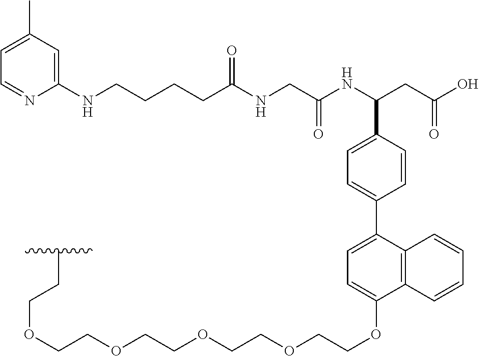 [SMILES: *CCOCCOCCOCCOCCOc1ccc(-c2ccc([C@H](CC(=O)O)NC(=O)CNC(=O)CCCCNc3cc(C)ccn3)cc2)c2ccccc12] |
| 46b | 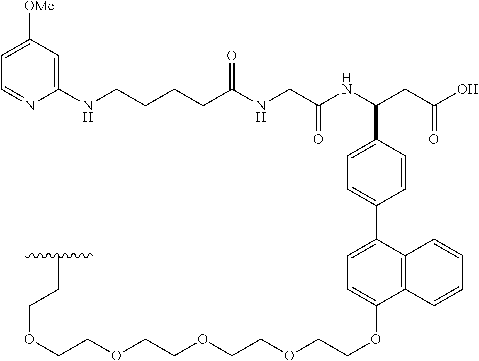 [SMILES: *CCOCCOCCOCCOCCOc1ccc(-c2ccc([C@H](CC(=O)O)NC(=O)CNC(=O)CCCCNc3cc(OC)ccn3)cc2)c2ccccc12] |
| 47b | 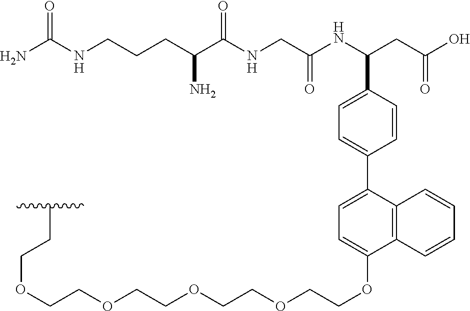 [SMILES: *CCOCCOCCOCCOCCOc1ccc(-c2ccc([C@H](CC(=O)O)NC(=O)CNC(=O)[C@@H](N)CCCNC(N)=O)cc2)c2ccccc12] |
| 48b | 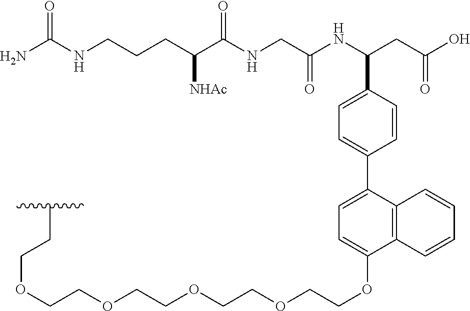 [SMILES: *CCOCCOCCOCCOCCOc1ccc(-c2ccc([C@H](CC(=O)O)NC(=O)CNC(=O)[C@H](CCCNC(N)=O)NC(C)=O)cc2)c2ccccc12] |
| 49b | 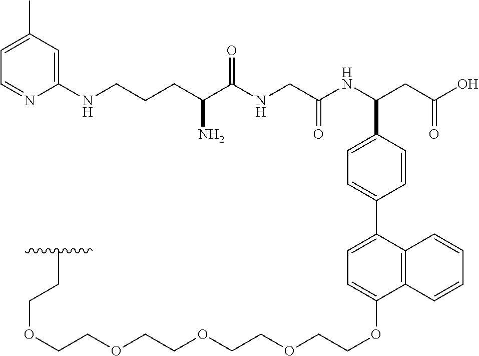 [SMILES: *CCOCCOCCOCCOCCOc1ccc(-c2ccc([C@H](CC(=O)O)NC(=O)CNC(=O)[C@@H](N)CCCNc3cc(C)ccn3)cc2)c2ccccc12] |
| 50b | 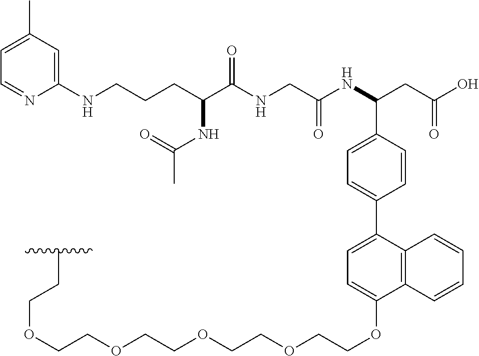 [SMILES: *CCOCCOCCOCCOCCOc1ccc(-c2ccc([C@H](CC(=O)O)NC(=O)CNC(=O)[C@H](CCCNc3cc(C)ccn3)NC(C)=O)cc2)c2ccccc12] |
| 51b | 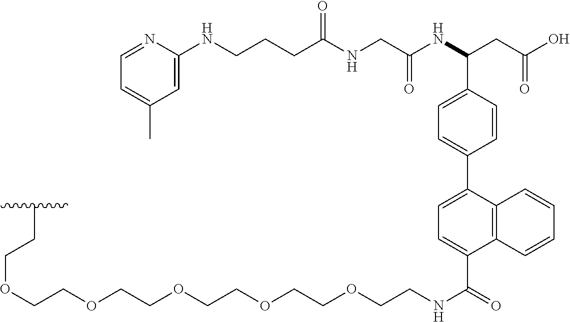 [SMILES: *CCOCCOCCOCCOCCOCCNC(=O)c1ccc(-c2ccc([C@H](CC(=O)O)NC(=O)CNC(=O)CCCNc3cc(C)ccn3)cc2)c2ccccc12] |
| 52b | 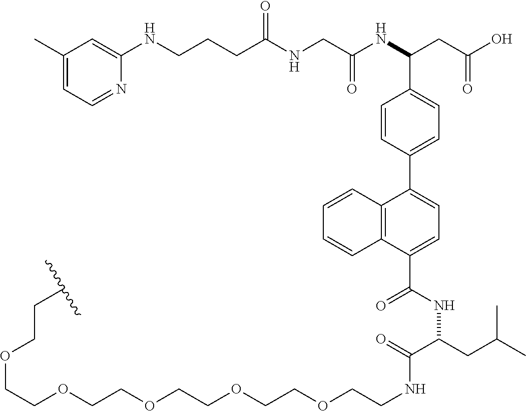 [SMILES: *CCOCCOCCOCCOCCOCCNC(=O)[C@@H](CC(C)C)NC(=O)c1ccc(-c2ccc([C@H](CC(=O)O)NC(=O)CNC(=O)CCCNc3cc(C)ccn3)cc2)c2ccccc12] |
| 53b | 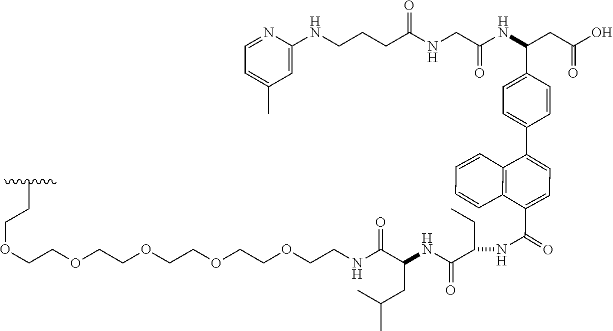 |
| 54b | 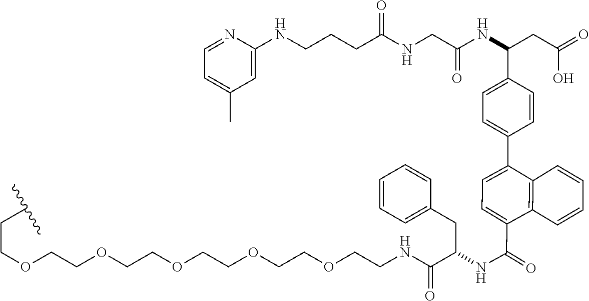 |
| 55b | 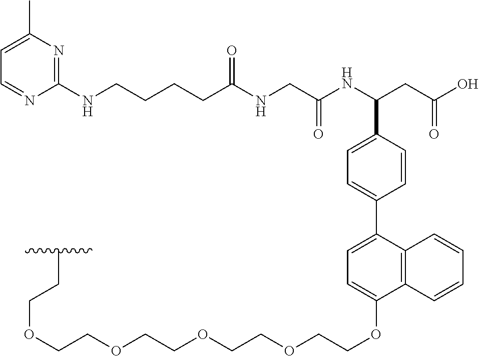 [SMILES: *CCOCCOCCOCCOCCOc1ccc(-c2ccc([C@H](CC(=O)O)NC(=O)CNC(=O)CCCCNc3nccc(C)n3)cc2)c2ccccc12] |
| 56b | 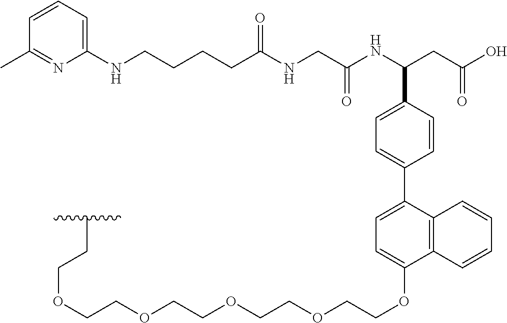 [SMILES: *CCOCCOCCOCCOCCOc1ccc(-c2ccc([C@H](CC(=O)O)NC(=O)CNC(=O)CCCCNc3cccc(C)n3)cc2)c2ccccc12] |
| 57b | 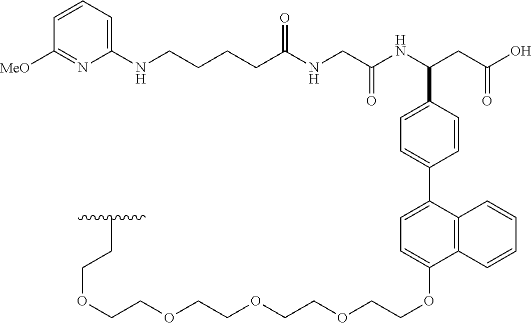 [SMILES: *CCOCCOCCOCCOCCOc1ccc(-c2ccc([C@H](CC(=O)O)NC(=O)CNC(=O)CCCCNc3cccc(OC)n3)cc2)c2ccccc12] |
| 58b | 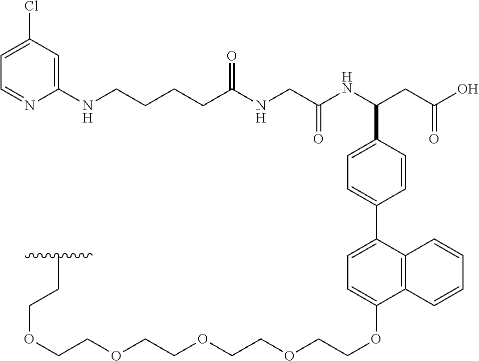 [SMILES: *CCOCCOCCOCCOCCOc1ccc(-c2ccc([C@H](CC(=O)O)NC(=O)CNC(=O)CCCCNc3cc(Cl)ccn3)cc2)c2ccccc12] |
| 59b | 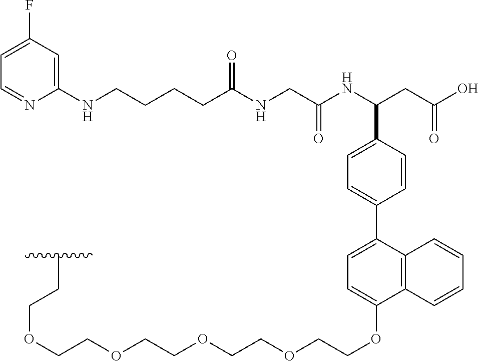 [SMILES: *CCOCCOCCOCCOCCOc1ccc(-c2ccc([C@H](CC(=O)O)NC(=O)CNC(=O)CCCCNc3cc(F)ccn3)cc2)c2ccccc12] |
| 60b | 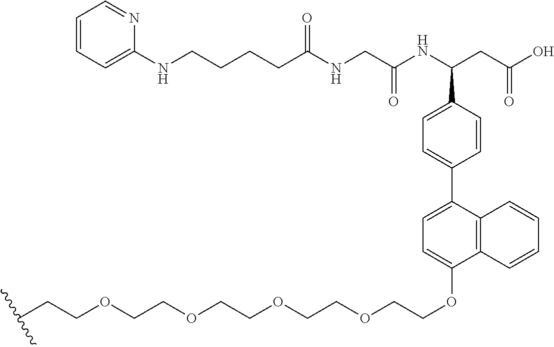 [SMILES: *CCOCCOCCOCCOCCOc1ccc(-c2ccc([C@H](CC(=O)O)NC(=O)CNC(=O)CCCCNc3ccccn3)cc2)c2ccccc12] |

or a pharmaceutically acceptable salt thereof, and wherein [symbol] indicates the point of connection to a cargo molecule.

Another aspect of the invention provides for a compound of Formula Ip:

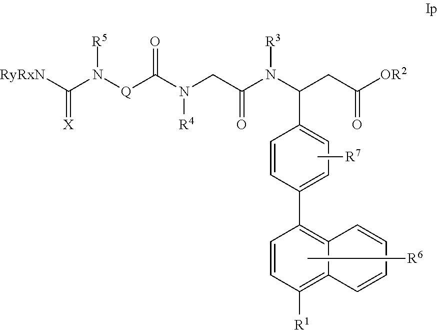

or a pharmaceutically acceptable salt thereof, wherein
R¹ is optionally substituted alkyl, optionally substituted alkoxy, or

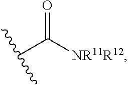

wherein R¹¹ and R¹² are each independently optionally substituted alkyl or a linking moiety, or R¹ is a linking moiety;
R² is H, optionally substituted alkyl, or a linking moiety;
R³ is H or optionally substituted alkyl;
R⁴ is H or optionally substituted alkyl;
R⁵ is H or optionally substituted alkyl;
R⁶ is selected from the group consisting of H, optionally substituted alkyl, optionally substituted alkoxy, halo, optionally substituted amino, or a linking moiety;
Q is optionally substituted aryl or optionally substituted alkylene;
X is O, CR⁸R⁹, NR⁸;
wherein R⁸ is selected from H, optionally substituted alkyl, or R⁸ is taken together with Rx or Ry to form a 4-, 5-, 6-, 7-, 8- or 9-membered ring, and R⁹ is H or optionally substituted alkyl;
Rx and Ry are each independently H, optionally substituted alkyl, a linking moiety or Rx and Ry may be taken together to form a double bond with R¹⁰, wherein R¹⁰ is H, optionally substituted alkyl, or R¹⁰ may be taken together with X and the atoms to which it is attached to form a 4-, 5-, 6-, 7-, 8, or 9-membered ring;
wherein at least one of R¹, R², R⁶, R¹¹, R¹², Rx and Ry comprise a linking moiety; and
wherein when Q is optionally substituted alkyl and the length of the optionally substituted alkyl chain represented by Q is 3 carbons, then R¹ is

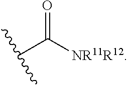

In some embodiments of Formula Ip, the linking moiety comprises a functional group selected from the group consisting of: azide, ester, carbamate, alkene, alcohol, amine, amide, carbonate, and alkyne. In some embodiments of Formula Ip, the linking moiety comprises an azide.

Another aspect of the invention provides compounds that may be precursors for compounds of Formula I. Example compounds of these precursors have the formula:

| Compound | |
|---|---|
| Number | Formula |
| 40p | 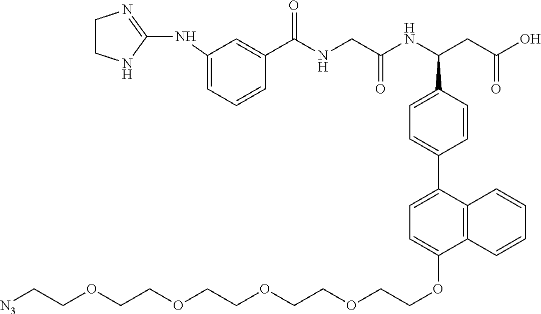 [SMILES: [N-]=[N+]=NCCOCCOCCOCCOCCOc1ccc(-c2ccc([C@H](CC(=O)O)NC(=O)CNC(=O)c3cccc(NC4=NCCN4)c3)cc2)c2ccccc12] |
| 41p | 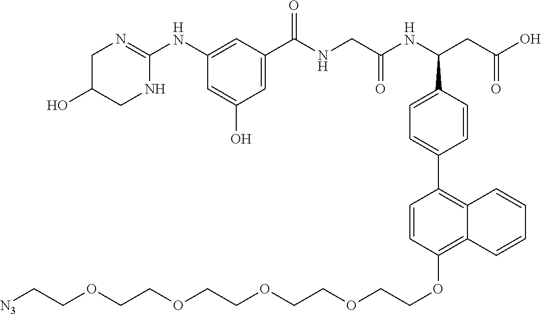 [SMILES: [N-]=[N+]=NCCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12] |
| 42p | 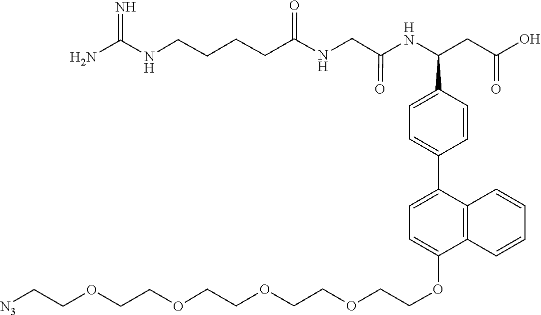 [SMILES: [N-]=[N+]=NCCOCCOCCOCCOCCOc1ccc(-c2ccc([C@H](CC(=O)O)NC(=O)CNC(=O)CCCCNC(=N)N)cc2)c2ccccc12] |
| 43p | 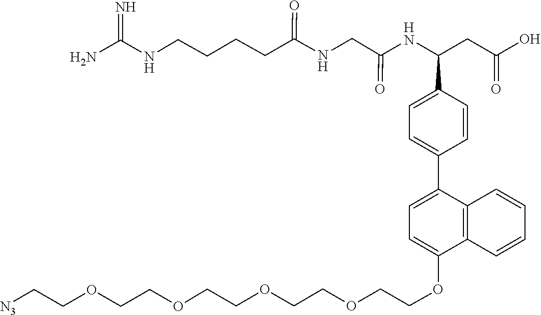 [SMILES: [N-]=[N+]=NCCOCCOCCOCCOCCOc1ccc(-c2ccc([C@H](CC(=O)O)NC(=O)CNC(=O)CCCCNC(=N)N)cc2)c2ccccc12] |
| 44p | 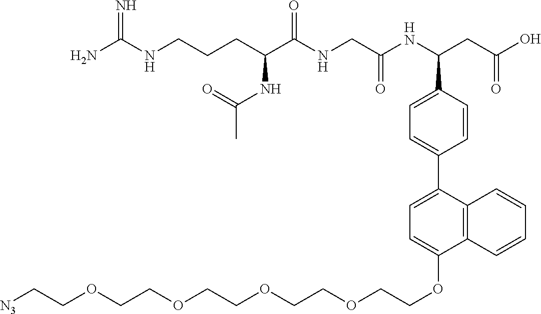 [SMILES: CC(=O)N[C@@H](CCCNC(=N)N)C(=O)NCC(=O)N[C@@H](CC(=O)O)c1ccc(-c2ccc(OCCOCCOCCOCCOCCN=[N+]=[N-])c3ccccc23)cc1] |
| 45p | 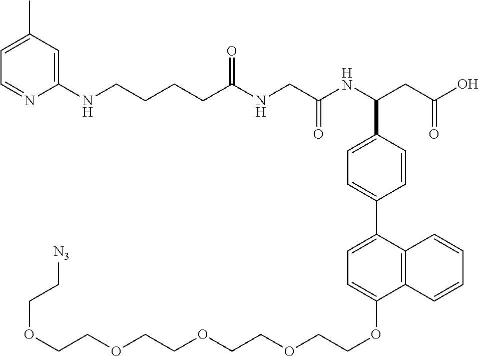 [SMILES: Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1] |
| 46p | 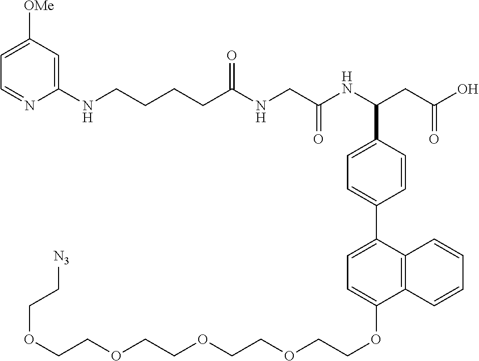 [SMILES: COc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1] |
| 47p | 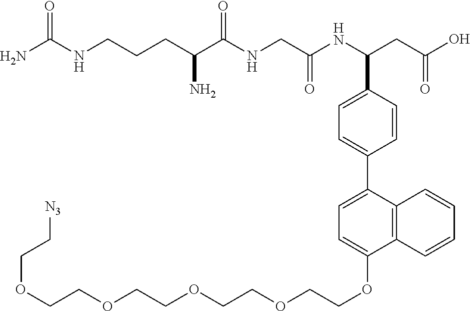 [SMILES: [N-]=[N+]=NCCOCCOCCOCCOCCOc1ccc(-c2ccc([C@H](CC(=O)O)NC(=O)CNC(=O)[C@@H](N)CCCNC(N)=O)cc2)c2ccccc12] |
| 48p | 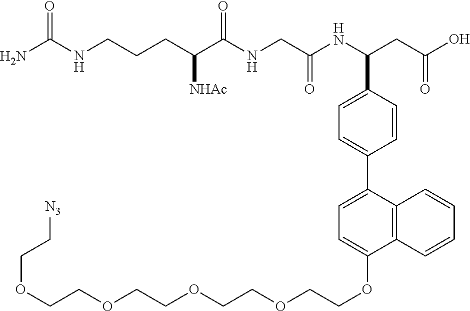 [SMILES: CC(=O)N[C@@H](CCCNC(N)=O)C(=O)NCC(=O)N[C@@H](CC(=O)O)c1ccc(-c2ccc(OCCOCCOCCOCCOCCN=[N+]=[N-])c3ccccc23)cc1] |
| 49p | 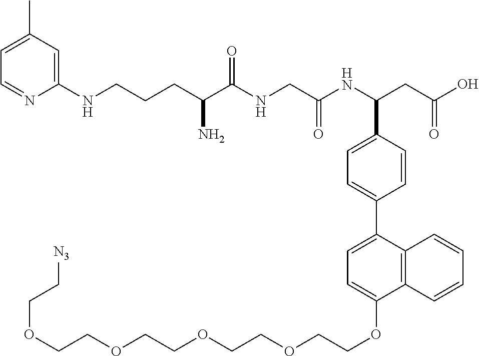 [SMILES: Cc1ccnc(NCCC[C@H](N)C(=O)NCC(=O)N[C@@H](CC(=O)O)c2ccc(-c3ccc(OCCOCCOCCOCCOCCN=[N+]=[N-])c4ccccc34)cc2)c1] |
| 50p | 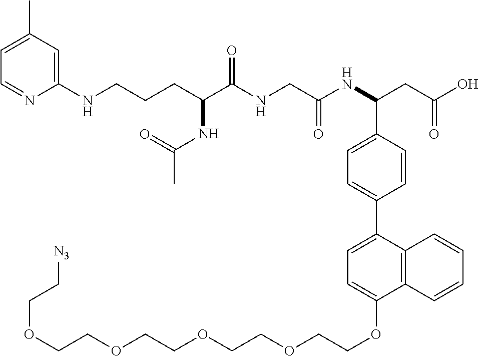 [SMILES: CC(=O)N[C@@H](CCCNc1cc(C)ccn1)C(=O)NCC(=O)N[C@@H](CC(=O)O)c1ccc(-c2ccc(OCCOCCOCCOCCOCCN=[N+]=[N-])c3ccccc23)cc1] |
| 51p | 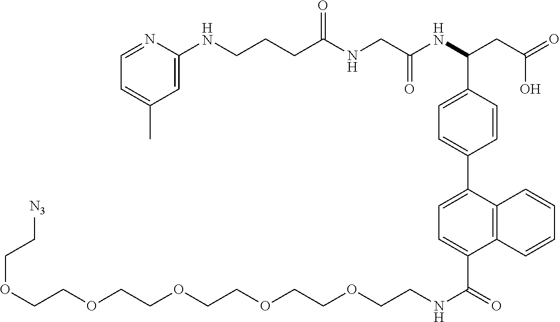 [SMILES: Cc1ccnc(NCCCC(=O)NCC(=O)N[C@@H](CC(=O)O)c2ccc(-c3ccc(C(=O)NCCOCCOCCOCCOCCOCCN=[N+]=[N-])c4ccccc34)cc2)c1] |
| 52p | 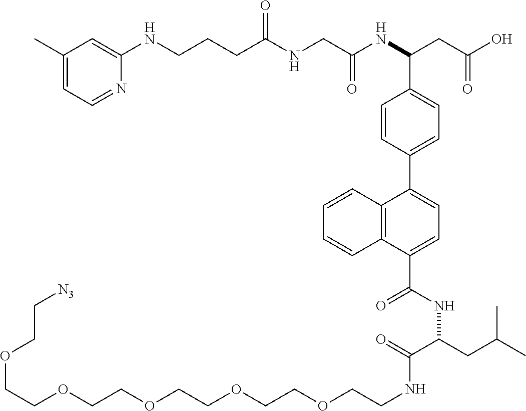 [SMILES: Cc1ccnc(NCCCC(=O)NCC(=O)N[C@@H](CC(=O)O)c2ccc(-c3ccc(C(=O)N[C@H](CC(C)C)C(=O)NCCOCCOCCOCCOCCOCCN=[N+]=[N-])c4ccccc34)cc2)c1] |
| 53p | 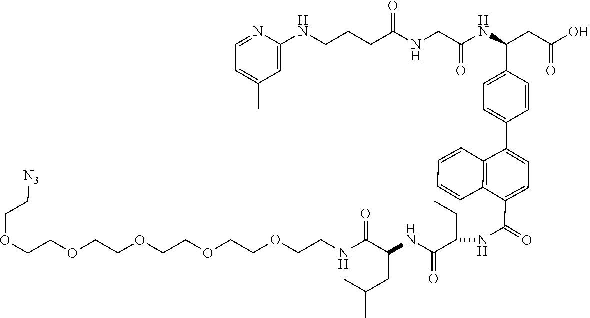 [SMILES: CC[C@H](NC(=O)c1ccc(-c2ccc([C@H](CC(=O)O)NC(=O)CNC(=O)CCCNc3cc(C)ccn3)cc2)c2ccccc12)C(=O)N[C@@H](CC(C)C)C(=O)NCCOCCOCCOCCOCCOCCN=[N+]=[N-]] |
| 54p | 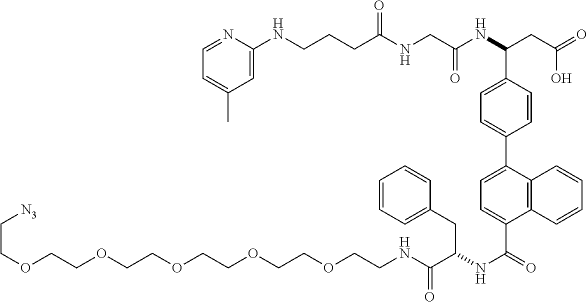 [SMILES: Cc1ccnc(NCCCC(=O)NCC(=O)N[C@@H](CC(=O)O)c2ccc(-c3ccc(C(=O)N[C@@H](Cc4ccccc4)C(=O)NCCOCCOCCOCCOCCOCCN=[N+]=[N-])c4ccccc34)cc2)c1] |
| 55p | 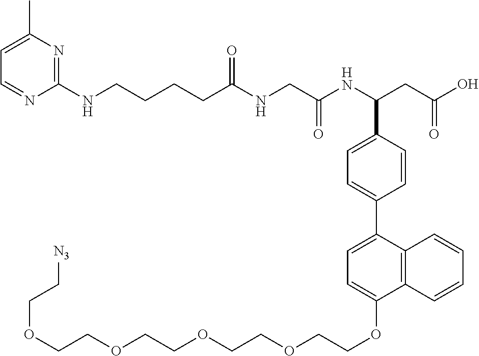 [SMILES: Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)n1] |
| 56p | 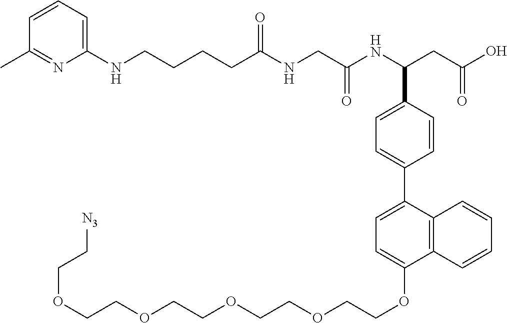 [SMILES: Cc1cccc(NCCCCC(=O)NCC(=O)N[C@@H](CC(=O)O)c2ccc(-c3ccc(OCCOCCOCCOCCOCCN=[N+]=[N-])c4ccccc34)cc2)n1] |
| 57p | 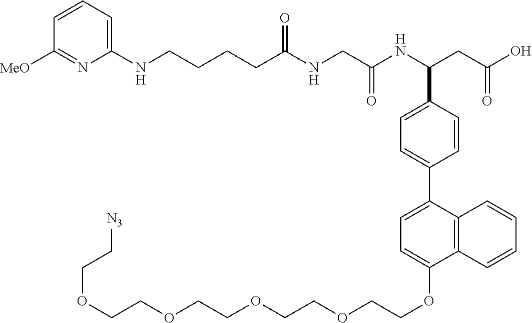 [SMILES: COc1cccc(NCCCCC(=O)NCC(=O)N[C@@H](CC(=O)O)c2ccc(-c3ccc(OCCOCCOCCOCCOCCN=[N+]=[N-])c4ccccc34)cc2)n1] |
| 58p | 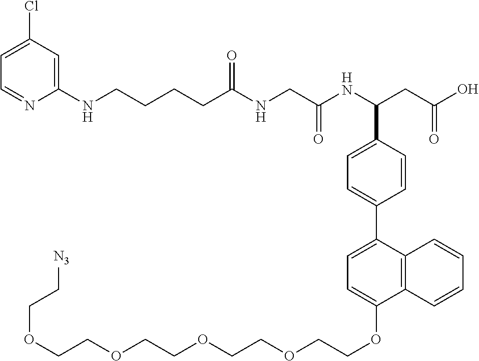 [SMILES: [N-]=[N+]=NCCOCCOCCOCCOCCOc1ccc(-c2ccc([C@H](CC(=O)O)NC(=O)CNC(=O)CCCCNc3cc(Cl)ccn3)cc2)c2ccccc12] |
| 59p | 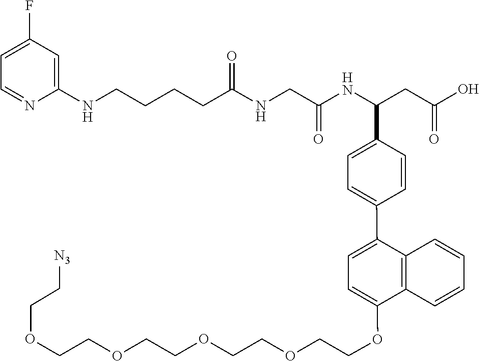 [SMILES: [N-]=[N+]=NCCOCCOCCOCCOCCOc1ccc(-c2ccc([C@H](CC(=O)O)NC(=O)CNC(=O)CCCCNc3cc(F)ccn3)cc2)c2ccccc12] |
| 60p | 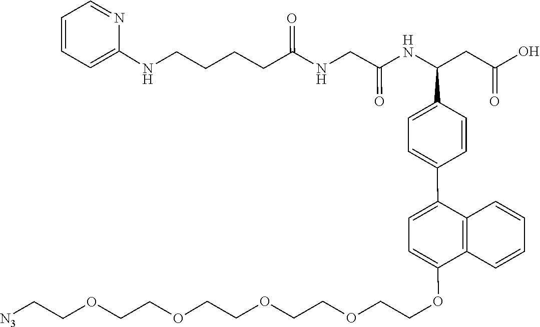 [SMILES: [N-]=[N+]=NCCOCCOCCOCCOCCOc1ccc(-c2ccc([C@H](CC(=O)O)NC(=O)CNC(=O)CCCCNc3ccccn3)cc2)c2ccccc12] |

or an acceptable salt thereof.

Any of the αvβ6 integrin ligands disclosed herein can be linked to a cargo molecule, a linking moiety, and/or a protected linking moiety. A linking moiety can be used to facilitate conjugation of the αvβ6 integrin ligand to a cargo molecule. The αvβ6 integrin ligands disclosed herein can increase targeting of a cargo molecule to an αvβ6 integrin or to a cell expressing an αvβ6 integrin. A cargo molecule can be, but is not limited to, a pharmaceutically active ingredient or compound, a prodrug, or another substance with known therapeutic or diagnostic benefit. In some embodiments, a cargo molecule can be, but is not limited to, a small molecule, an antibody, an antibody fragment, an immunoglobulin, a monoclonal antibody, a label or marker, a lipid, a natural or modified oligonucleotide-based compound (e.g., an antisense oligonucleotide or an RNAi agent), a natural or modified nucleic acid, a peptide, an aptamer, a polymer, a polyamine, a protein, a toxin, a vitamin, a polyethylene glycol, a hapten, a digoxigenin, a biotin, a radioactive atom or molecule, or a fluorophore. In some embodiments, a cargo molecule includes a pharmaceutically active ingredient or a prodrug. In some embodiments, a cargo molecule includes an oligonucleotide-based compound as a pharmaceutically active ingredient. In some embodiments, a cargo molecule includes an RNAi agent as a pharmaceutically active ingredient.

As used herein, the term “alkyl” refers to a saturated aliphatic hydrocarbon group, straight chain or branched, having from 1 to 10 carbon atoms unless otherwise specified. For example, “C₁-C₆ alkyl” includes alkyl groups having 1, 2, 3, 4, 5, or 6 carbons in a linear or branched arrangement. Non-limiting examples of alkyl groups include methyl, ethyl, iso-propyl, tert-butyl, n-hexyl. As used herein, the term “aminoalkyl” refers to an alkyl group as defined above, substituted at any position with one or more amino groups as permitted by normal valency. The amino groups may be unsubstituted, monosubstituted, or di-substituted. Non-limiting examples of aminoalkyl groups include aminomethyl, dimethylaminomethyl, and 2-aminoprop-1-yl.

As used herein, the term “cycloalkyl” means a saturated or unsaturated nonaromatic hydrocarbon ring group having from 3 to 14 carbon atoms, unless otherwise specified. Non-limiting examples of cycloalkyl groups include, but are not limited to, cyclopropyl, methyl-cyclopropyl, 2,2-dimethyl-cyclobutyl, 2-ethyl-cyclopentyl, and cyclohexyl. Cycloalkyls may include multiple spiro- or fused rings. Cycloalkyl groups are optionally mono-, di-, tri-, tetra-, or penta-substituted on any position as permitted by normal valency.

As used herein, the term “cycloalkylene” refers to a divalent radical of a cycloalkyl group as described herein. Cycloalkylene is a subset of cycloalkyl, referring to the same residues as cycloalkyl, but having two points of substitution. Examples of cycloalkylene include cyclopropylene,

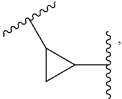

1,4-cyclohexylene,

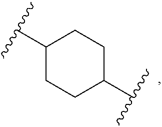 [SMILES: *C1CCC(*)CC1]

and 1,5-cyclooxylene

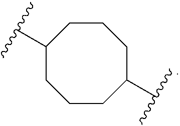 [SMILES: *C1CCCC(*)CCC1]

Cycloalkylene groups are optionally mono-, di-, tri-, tetra-, or penta-substituted on any position as permitted by normal valency. Cycloalkylene groups may mono-, di-, or tri-cyclic.

As used herein, the term “alkenyl” refers to a non-aromatic hydrocarbon radical, straight, or branched, containing at least one carbon-carbon double bond, and having from 2 to 10 carbon atoms unless otherwise specified. Up to five carbon-carbon double bonds may be present in such groups. For example, “C₂-C₆” alkenyl is defined as an alkenyl radical having from 2 to 6 carbon atoms. Examples of alkenyl groups include, but are not limited to, ethenyl, propenyl, butenyl, and cyclohexenyl. The straight, branched, or cyclic portion of the alkenyl group may contain double bonds and is optionally mono-, di-, tri-, tetra-, or penta-substituted on any position as permitted by normal valency. The term “cycloalkenyl” means a monocyclic hydrocarbon group having the specified number of carbon atoms and at least one carbon-carbon double bond.

As used herein, the term “alkynyl” refers to a hydrocarbon radical, straight or branched, containing from 2 to 10 carbon atoms, unless otherwise specified, and containing at least one carbon-carbon triple bond. Up to 5 carbon-carbon triple bonds may be present. Thus, “C₂-C₆ alkynyl” means an alkynyl radical having from 2 to 6 carbon atoms. Examples of alkynyl groups include, but are not limited to, ethynyl, 2-propynyl, and 2-butynyl. The straight or branched portion of the alkynyl group may be optionally mono-, di-, tri-, tetra-, or penta-substituted on any position as permitted by normal valency.

As used herein, “alkoxyl” or “alkoxy” refers to —O-alkyl radical having the indicated number of carbon atoms. For example, C₁₋₆ alkoxy is intended to include C₁, C₂, C₃, C₄, C₅, and C₆ alkoxy groups. For example, C₁₋₈ alkoxy, is intended to include C₁, C₂, C₃, C₄, C₅, C₆, C₇, and C₈ alkoxy groups. Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy.

As used herein, “keto” refers to any alkyl, alkenyl, alkynyl, cycloalkyl, cycloalkenyl, heterocyclyl, heteroaryl, or aryl group as defined herein attached through a carbonyl bridge. Examples of keto groups include, but are not limited to, alkanoyl (e.g., acetyl, propionyl, butanoyl, pentanoyl, or hexanoyl), alkenoyl (e.g., acryloyl) alkynoyl (e.g., ethynoyl, propynoyl, butynoyl, pentynoyl, or hexynoyl), aryloyl (e.g., benzoyl), heteroaryloyl (e.g., pyrroloyl, imidazoloyl, quinolinoyl, or pyridinoyl).

As used herein, “alkoxycarbonyl” refers to any alkoxy group as defined above attached through a carbonyl bridge (i.e., —C(O)O-alkyl). Examples of alkoxycarbonyl groups include, but are not limited to, methoxycarbonyl, ethoxycarbonyl, iso-propoxycarbonyl, n-propoxycarbonyl, t-butoxycarbonyl, benzyloxvcarbonyl, or n-pentoxycarbonyl.

As used herein, “aryloxycarbonyl” refers to any aryl group as defined herein attached through an oxycarbonyl bridge (i.e., —C(O)O-aryl). Examples of aryloxycarbonyl groups include, but are not limited to, phenoxycarbonyl and naphthyloxvcarbonyl.

As used herein, “heteroaryloxycarbonyl” refers to any heteroaryl group as defined herein attached through an oxycarbonyl bridge (i.e., —C(O)O-heteroaryl). Examples of heteroaryloxycarbonyl groups include, but are not limited to, 2-pyridyloxycarbonyl, 2-oxazolyloxvcarbonvl, 4-thiazolyloxycarbonyl, or pyrimidinyloxycarbonyl.

As used herein, “aryl” or “aromatic” means any stable monocyclic or polycyclic carbon ring of up to 6 atoms in each ring, wherein at least one ring is aromatic. Examples of aryl groups include, but are not limited to, phenyl, naphthyl, anthracenyl, tetrahydronaphthyl, indanyl, and biphenyl. In cases where the aryl substituent is bicyclic and one ring is non-aromatic, it is understood that attachment is via the aromatic ring. Aryl groups are optionally mono-, di-, tri-, tetra-, or penta-substituted on any position as permitted by normal valency.

As used herein, the term “arylene” refers to a divalent radical of an aryl group as described herein. Arylene is a subset of aryl, referring to the same residues as aryl, but having two points of substitution. Examples of arylene include phenylene, which refers to a divalent phenyl group. Arylene groups are optionally mono-, di-, tri-, tetra-, or penta-substituted on any position as permitted by normal valency.

As used herein, the term “halo” refers to a halogen radical. For instance, “halo” may refer to a fluorine (F), chlorine (Cl), bromine (Br), or an iodine (I) radical.

As used herein, the term “heteroaryl” represents a stable monocyclic or polycyclic ring of up to 7 atoms in each ring, wherein at least one ring is aromatic and contains from 1 to 4 heteroatoms selected from the group consisting of O, N, and S. Examples of heteroaryl groups include, but are not limited to, acridinyl, carbazolyl, cinnolinyl, quinoxalinyl, pyrrazolyl, indolyl, benzotriazolyl, furanyl, thienyl, benzothienyl, benzofuranyl, benzimidazolonyl, benzoxazolonyl, quinolinyl, isoquinolinyl, dihydroisoindolonyl, imidazopyridinyl, isoindolonyl, indazolyl, oxazolyl, oxadiazolyl, isoxazolyl, indolyl, pyrazinyl, pyridazinyl, pyridinyl, pyrimidinyl, pyrrolyl, and tetrahydroquinoline. “Heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring. Heteroaryl groups are optionally mono-, di-, tri-, tetra-, or penta-substituted on any position as permitted by normal valency.

As used herein, the term “heteroarylene” refers to a divalent radical of a heteroaryl group as described herein. Heteroarylene is a subset of heteroaryl, referring to the same residues as heteroaryl, but having two points of substitution. Examples of heteroaryl include pyridinylene, pyrimidinylene, and pyrrolylene. Heteroarylene groups are optionally mono-, di-tri-, tetra-, or penta-substituted on any position as permitted by normal valency.

As used herein, the term “heterocycle,” “heterocyclic,” or “heterocyclyl” means a 3- to 14-membered aromatic or nonaromatic heterocycle containing from 1 to 4 heteroatoms selected from the group consisting of O, N, and S, including polycyclic groups. As used herein, the term “heterocyclic” is also considered to be synonymous with the terms “heterocycle” and “heterocyclyl” and is understood as also having the same definitions set forth herein. “Heterocyclyl” includes the above mentioned heteroaryls, as well as dihydro and tetrahydro analogs thereof. Examples of heterocyclyl groups include, but are not limited to, azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxooxazolidinyl, oxazolyl, oxazoline, oxopiperazinyl, oxopyrrolidinyl, oxomorpholinyl, isoxazoline, oxetanyl, pyranyl, pyrazinyl, pyrazolyl, pyridazinyl, pyridopyridinyl, pyridazinyl, pyridyl, pyridinonyl, pyrimidyl, pyrimidinonyl, pyrrolyl, quinazolinyl, quinolyl, quinoxalinyl, tetrahydropyranyl, tetrahydrofuranyl, tetrahydrothiopyranyl, tetrahydroisoquinolinyl, tetrazolyl, tetrazolopyridyl, thiadiazolyl, thiazolyl, thienyl, triazolyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxidothiomorpholinyl, methylenedioxybenzoyl, tetrahydrofuranyl, and tetrahydrothienyl, and N-oxides thereof. Attachment of a heterocyclyl substituent can occur via a carbon atom or via a heteroatom. Heterocyclyl groups are optionally mono-, di-, tri-, tetra-, or penta-substituted on any position as permitted by normal valency.

As used herein, the term “heterocycloalkvl” means a 3- to 14-membered nonaromatic heterocycle containing from 1 to 4 heteroatoms selected from the group consisting of O, N, and S, including polycyclic groups. Examples of heterocyclyl groups include, but are not limited to, azetidinyl, oxopiperazinyl, oxopyrrolidinyl, oxomorpholinyl, oxetanyl, pyranyl, pyridinonyl, pyrimidinonyl, tetrahydropyranyl, tetrahydrofuranyl, tetrahydrothiopyranyl, tetrahydroisoquinolinyl, 1,4-dioxanyl, hexahydroazepinyl, piperazinyl, piperidinyl, pyrrolidinyl, morpholinyl, thiomorpholinyl, dihydrofuranyl, dihydroimidazolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydrotetrazolyl, dihydrothiadiazolyl, dihydrothiazolyl, dihydrothienyl, dihydrotriazolyl, dioxidothiomorpholinyl, and tetrahydrothienyl, and N-oxides thereof. Attachment of a heterocycloalkyl substituent can occur via a carbon atom or via a heteroatom. Heterocyclyl groups are optionally mono-, di-, tri-, tetra-, or penta-substituted on any position as permitted by normal valency.

As used herein, the term “heterocycloalkylene” refers to a divalent radical of a heterocycloalkyl group as described herein. Heteroycloalkylene is a subset of heterocycloalkyl, referring to the same residues as heterocycloalkyl, but having two points of substitution. Examples of heterocycloalkylene include piperidinylene, azetidinylene, and tetrahydrofuranylene. Heterocycloalkylene groups are optionally mono-, di-, tri-, tetra-, or penta-substituted on any position as permitted by normal valency.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means that delivering the RNAi agent to the cell in a manner that enables the RNAi agent to have the expected biological activity, e.g., sequence-specific inhibition of gene expression.

Unless stated otherwise, use of the symbol

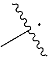

as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.” When the compounds described herein contain olefinic double bonds or other centers of geometric asymmetry for which the isomeric structure is not specifically defined, it is intended that the compounds can include both E and Z geometric isomers individually or in a mixture. The compounds of Formula I or their pharmaceutically acceptable salts, for example, are meant to include all possible isomers, as well as their racmeic and optically pure forms. Likewise, unless expressly stated otherwise, all tautomeric forms are also intended to be included.

As used herein, a linking group is one or more atoms that connects one molecule or portion of a molecule to another to second molecule or second portion of a molecule. In the art, the terms linking group and spacers are sometimes used interchangeably. Similarly, as used in the art, the term scaffold is sometimes used interchangeably with a linking group. In some embodiments, a linking group can include a peptide-cleavable linking group. In some embodiments, a linking group can include or consist of the peptide phenylalanine-citrulline-phenylalanine-proline. In some embodiments, a linking group can include or consist of a PEG group.

As used herein, the term “linked” when referring to the connection between two molecules means that two molecules are joined by a covalent bond or that two molecules are associated via noncovalent bonds (e.g., hydrogen bonds or ionic bonds). In some examples, where the term “linked” refers to the association between two molecules via noncovalent bonds, the association between the two different molecules has a K_{D} of less than 1×10⁻⁴ M (e.g., less than 1×10⁻⁵ M, less than 1×10⁻⁶ M, or less than 1×10⁻⁷ M) in physiologically acceptable buffer (e.g., phosphate buffered saline). Unless stated, the term linked as used herein may refer to the connection between a first compound and a second compound either with or without any intervening atoms or groups of atoms.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the pH of the environment, as would be readily understood by the person of ordinary skill in the art.

Structures may be depicted as having a bond “floating” over a ring structure to indicate binding to any carbon or heteroatom on the ring as permitted by valency. For example, the structure

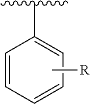

indicates that R may replace any hydrogen atom at any of the five available positions on the ring. “Floating” bonds may also be used in bicyclic structures to indicate a bond to any position on either ring of the bicycle as permitted by valency. In the case of bicycles, the bond will be shown “floating” over both rings, for example,

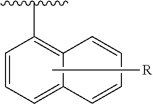

indicates that R may replace any hydrogen atom at any of the seven available positions on the ring.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

Described herein is the use of the described αvβ6 integrin ligands to target and deliver a cargo molecule to a cell that expresses αvβ6 integrin. The cargo molecule can be delivered to a cell in vitro, in situ, ex vivo, or in vivo.

In some embodiments, an αvβ6 integrin ligand disclosed herein can be conjugated to one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30; or 10 to 30, 1 to 25, 1 to 20, 1 to 15, 1 to 10, 1 to 5, 5 to 30, 5 to 25, 5 to 20, 5 to 15, 5 to 10, 10 to 30, 10 to 25, 10 to 20, 10 to 15, 15 to 30, 15 to 25, 15 to 20, 20 to 30, 20 to 25, or 25 to 30) cargo molecules (e.g., any of the cargo molecules described herein or known in the art).

In some embodiments, more than one αvβ6 integrin ligand disclosed herein (e.g., 2, 3, 4, 5, 6, 7, 8, or 10 to 8, 1 to 7, 1 to 6, 1 to 5, 1 to 4, 1 to 3, 1 to 2, 2 to 8, 2 to 7, 2 to 6, 2 to 5, 2 to 4, 2 to 3, 3 to 8, 3 to 7, 3 to 6, 3 to 5, 3 to 4, 4 to 8, 4 to 7, 4 to 6, or 4 to 5 αvβ6 integrin ligands) can be conjugated to one cargo molecule (e.g., any of the cargo molecules described herein or known in the art).

In some embodiments, the αvβ6 integrin ligands disclosed herein are optionally conjugated to one or more cargo molecules via a linking group, such as, for example, a polyethylene glycol (PEG) group.

In some embodiments, the αvβ6 integrin ligands disclosed herein are optionally conjugated to one or more cargo molecules via a scaffold that includes at least one attachment point for each ligand and at least one attachment point for each cargo molecule. In some embodiments, the αvβ6 integrin ligands comprise, consist of, or consist essentially of, one cargo molecule. In some embodiments, the αvβ6 integrin ligands comprise, consist of, or consist essentially of, more than one cargo molecule.

Any of the αvβ6 integrin ligands disclosed herein can be linked to a cargo molecule, a linking moiety, and/or a protected linking moiety. A linking moiety can be used to facilitate conjugation of the αvβ6 integrin ligand to a cargo molecule. The αvβ6 integrin ligands disclosed herein can increase targeting of a cargo molecule to an αvβ6 integrin or to a cell expressing an αvβ6 integrin. A cargo molecule can be, but is not limited to, a pharmaceutically active ingredient or compound, a prodrug, or another substance with known therapeutic benefit. In some embodiments, a cargo molecule can be, but is not limited to, a small molecule, an antibody, an antibody fragment, an immunoglobulin, a monoclonal antibody, a label or marker, a lipid, a natural or modified oligonucleotide-based compound (e.g., an antisense oligonucleotide or an RNAi agent), a natural or modified nucleic acid, a peptide, an aptamer, a polymer, a polyamine, a protein, a toxin, a vitamin, a polyethylene glycol, a hapten, a digoxigenin, a biotin, a radioactive atom or molecule, or a fluorophore. In some embodiments, a cargo molecule includes a pharmaceutically active ingredient or a prodrug. In some embodiments, a cargo molecule includes an oligonucleotide-based compound as a pharmaceutically active ingredient. In some embodiments, a cargo molecule includes an RNAi agent as a pharmaceutically active ingredient.

In one aspect, the invention provides for a structure comprising an αvβ6 integrin ligand as described herein, a linking group, and a scaffold, wherein the scaffold is bound to a cargo molecule. In some embodiments, the structure may comprise the ligand in monodentate form. In some embodiments, the structure may comprise the ligand in bidentate form. In some embodiments, the structure may comprise the ligand in tridentate form. In some embodiments, the structure may comprise the ligand in tetradentate form.

Multidentate αvβ6 Integrin Ligands and Scaffolds

As disclosed herein, in some embodiments, one or more αvβ6 integrin ligands may be linked to one or more cargo molecules. In some embodiments, only one αvβ6 integrin ligand is conjugated to a cargo molecule (referred to herein as a “monodentate” or “monovalent” ligand). In some embodiments, two αvβ6 integrin ligands are conjugated to a cargo molecule (referred to herein as a “bidentate” or “divalent” ligand). In some embodiments, three αvβ6 integrin ligands are conjugated to a cargo molecule (referred to herein as a “tridentate” or “trivalent” ligand). In some embodiments, four αvβ6 integrin ligands are conjugated to a cargo molecule (referred to herein as a “tetradentate” or “tetravalent” ligand). In some embodiments, more than four αvβ6 integrin ligands are conjugated to a cargo molecule.

In some embodiments, where only one αvβ6 integrin ligand is conjugated to a cargo molecule (referred to herein as a “monodentate” ligand), the αvβ6 integrin ligand may be conjugated directly to the cargo molecule. In some embodiments, an αvβ6 integrin ligand disclosed herein can be conjugated to a cargo molecule via a scaffold or other linker structure.

In some embodiments, the αvβ6 integrin ligands disclosed herein include one or more scaffolds. Scaffolds, also sometimes referred to in the art as linking groups or linkers, can be used to facilitate the linkage of one or more cargo molecules to one or more αvβ6 integrin ligands disclosed herein. Useful scaffolds compatible with the ligands disclosed herein are generally known in the art. Non-limiting examples of scaffolds that can be used with the αvβ6 integrin ligands disclosed herein include, but are not limited to polymers and polyamino acids (e.g., bis-glutamic acid, poly-L-lysine, etc.). In some embodiments, scaffolds may include cysteine linkers or groups, DBCO-PEG₁₋₂₄-NHS, Propargyl-PEG₁₋₂₄-NHS, and/or multidentate DBCO and/or propargyl moieties.

### Linking Moieties and Protected Linking Moieties.

Linking moieties are well known in the art and provide for formation of covalent linkages between two molecules or reactants. Suitable linking moieties for use in the scope of the inventions herein include, but are not limited to: amino groups, amide groups, carboxylic acid groups, azides, alkynes, propargyl groups, BCN(biclclo[6.1.0]nonyne, DBCO(dibenzocyclooctyne) thiols, maleimide groups, aminooxy groups, N-hydroxysuccinimide (NHS) or other activated ester (for example, PNP, TFP, PFP), bromo groups, aldehydes, carbonates, tosylates, tetrazines, trans-cyclooctene (TCO), hydrazides, hydroxyl groups, disulfides, and orthopyridyl disulfide groups.

Incorporation of linking moieties can facilitate conjugation of an αvβ6 integrin ligand disclosed herein to a cargo molecule. Conjugation reactions are well known in the art and provide for formation of covalent linkages between two molecules or reactants. Suitable conjugation reactions for use in the scope of the inventions herein include, but are not limited to, amide coupling reaction, Michael addition reaction, hydrazone formation reaction and click chemistry cycloaddition reaction.

In some embodiments, the αvβ6 integrin targeting ligands disclosed herein are synthesized as a tetrafluorophenyl (TFP) ester, which can be displaced by a reactive amino group to attach a cargo molecule. In some embodiments, the integrin targeting ligands disclosed herein are synthesized as an azide, which can be conjugated to a propargyl or DBCO group, for example, via click chemistry cycloaddition reaction, to attach a cargo molecule.

Protected linking moieties are also commonly used in the art. A protecting group provides temporary chemical transformation of a linking moiety into a group that does not react under conditions where the non-protected group reacts, e.g, to provide chemo-selectivity in a subsequent chemical reaction. Suitable protected linking moieties for use in the scope of the inventions herein include, but are not limited to, BOC groups (t-butoxycarbonyl), Fmoc (9-fluorenylmethoxycarbonyl), carboxybenzyl (CBZ) groups, benzyl esters, and PBF (2,2,4,6,7-pentamethyldihydrobenzofuran-5-sulfonyl).

### Cargo Molecules (Including RNAi Agents)

A cargo molecule is any molecule which, when detached from the αvβ6 integrin ligands described herein, would have a desirable effect on a cell comprising an αvβ6 integrin receptor. A cargo molecule can be, but is not limited to, a pharmaceutical ingredient, a drug product, a prodrug, a substance with a known therapeutic benefit, a small molecule, an antibody, an antibody fragment, an immunoglobulin, a monoclonal antibody, a label or marker, a lipid, a natural or modified nucleic acid or polynucleotide, a peptide, a polymer, a polyamine, a protein, an aptamer, a toxin, a vitamin, a PEG, a hapten, a digoxigenin, a biotin, a radioactive atom or molecule, or a fluorophore. In some embodiments, one or more cargo molecules (e.g., the same or different cargo molecules) are linked to one or more αvβ6 integrin ligands to target the cargo molecules to a cell expressing an αvβ6 integrin.

In some embodiments, the one or more cargo molecules is a pharmaceutical ingredient or pharmaceutical composition. In some embodiments, the one or more cargo molecules is an oligonucleotide-based compound. As used herein, an “oligonucleotide-based compound” is a nucleotide sequence containing about 10-50 (e.g., 10 to 48, 10 to 46, 10 to 44, 10 to 42, 10 to 40, 10 to 38, 10 to 36, 10 to 34, 10 to 32, 10 to 30, 10 to 28, 10 to 26, 10 to 24, 10 to 22, 10 to 20, 10 to 18, 10 to 16, 10 to 14, 10 to 12, 12 to 50, 12 to 48, 12 to 46, 12 to 44, 12 to 42, 12 to 40, 12 to 38, 12 to 36, 12 to 34, 12 to 32, 12 to 30, 12 to 28, 12 to 26, 12 to 24, 12 to 22, 12 to 20, 12 to 18, 12 to 16, 12 to 14, 14 to 50, 14 to 48, 14 to 46, 14 to 44, 14 to 42, 14 to 40, 14 to 38, 14 to 36, 14 to 34, 14 to 32, 14 to 30, 14 to 28, 14 to 26, 14 to 24, 14 to 22, 14 to 20, 14 to 18, 14 to 16, 16 to 50, 16 to 48, 16 to 46, 16 to 44, 16 to 42, 16 to 40, 16 to 38, 16 to 36, 16 to 34, 16 to 32, 16 to 30, 16 to 28, 16 to 26, 16 to 24, 16 to 22, 16 to 20, 16 to 18, 18 to 50, 18 to 48, 18 to 46, 18 to 44, 18 to 42, 18 to 40, 18 to 38, 18 to 36, 18 to 34, 18 to 32, 18 to 30, 18 to 28, 18 to 26, 18 to 24, 18 to 22, 18 to 20, 20 to 50, 20 to 48, 20 to 46, 20 to 44, 20 to 42, 20 to 40, 20 to 38, 20 to 36, 20 to 34, 20 to 32, 20 to 30, 20 to 28, 20 to 26, 20 to 24, 20 to 22, 22 to 50, 22 to 48, 22 to 46, 22 to 44, 22 to 42, 22 to 40, 22 to 38, 22 to 36, 22 to 34, 22 to 32, 22 to 30, 22 to 28, 22 to 26, 22 to 24, 24 to 50, 24 to 48, 24 to 46, 24 to 44, 24 to 42, 24 to 40, 24 to 38, 24 to 36, 24 to 34, 24 to 32, 24 to 30, 24 to 28, 24 to 26, 26 to 50, 26 to 48, 26 to 46, 26 to 44, 26 to 42, 26 to 40, 26 to 38, 26 to 36, 26 to 34, 26 to 32, 26 to 30, 26 to 28, 28 to 50, 28 to 48, 28 to 46, 28 to 44, 28 to 42, 28 to 40, 28 to 38, 28 to 36, 28 to 34, 28 to 32, to 28 to 30, 30 to 50, 30 to 48, 30 to 46, 30 to 44, 30 to 42, 30 to 40, 30 to 38, 30 to 36, 30 to 34, 30 to 32, 32 to 50, 32 to 48, 32 to 46, 32 to 44, 32 to 42, 32 to 40, 32 to 38, 32 to 36, 32 to 34, 34 to 50, 34 to 48, 34 to 46, 34 to 44, 34 to 42, 34 to 40, 34 to 38, 34 to 36, 36 to 50, 36 to 48, 36 to 46, 36 to 44, 36 to 42, 36 to 40, 36 to 38, 38 to 50, 38 to 48, 38 to 46, 38 to 44, 38 to 42, 38 to 40, 40 to 50, 40 to 48, 40 to 46, 40 to 44, 40 to 42, 42 to 50, 42 to 48, 42 to 46, 42 to 44, 44 to 50, 44 to 48, 44 to 46, 46 to 50, 46 to 48, or 48 to 50) nucleotides or nucleotide base pairs. In some embodiments, an oligonucleotide-based compound has a nucleobase sequence that is at least partially complementary to a coding sequence in an expressed target nucleic acid or target gene within a cell. In some embodiments, the oligonucleotide-based compounds, upon delivery to a cell expressing a gene, are able to inhibit the expression of the underlying gene, and are referred to herein as “expression-inhibiting oligonucleotide-based compounds.” The gene expression can be inhibited in vitro or in vivo.

“Oligonucleotide-based compounds” include, but are not limited to: single-stranded oligonucleotides, single-stranded antisense oligonucleotides, short interfering RNAs (siRNAs), double-strand RNAs (dsRNA), micro RNAs (miRNAs), short hairpin RNAs (shRNA), ribozymes, interfering RNA molecules, and dicer substrates. In some embodiments, an oligonucleotide-based compound is a single-stranded oligonucleotide, such as an antisense oligonucleotide. In some embodiments, an oligonucleotide-based compound is a double-stranded oligonucleotide. In some embodiments, an oligonucleotide-based compound is a double-stranded oligonucleotide that is an RNAi agent.

In some embodiments, the one or more cargo molecules is/are an “RNAi agent,” which as defined herein is a composition that contains an RNA or RNA-like (e.g., chemically modified RNA) oligonucleotide molecule that is capable of degrading or inhibiting translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double-strand RNAs (dsRNA), micro RNAs (miRNAs), short hairpin RNAs (shRNA), and dicer substrates. The antisense strand of the RNAi agents described herein is at least partially complementary to the mRNA being targeted. RNAi agents can include one or more modified nucleotides and/or one or more non-phosphodiester linkages.

Typically, RNAi agents can be comprised of at least a sense strand (also referred to as a passenger strand) that includes a first sequence, and an antisense strand (also referred to as a guide strand) that includes a second sequence. The length of an RNAi agent sense and antisense strands can each be 16 to 49 nucleotides in length. In some embodiments, the sense and antisense strands of an RNAi agent are independently 17 to 26 nucleotides in length. In some embodiments, the sense and antisense strands are independently 19 to 26 nucleotides in length. In some embodiments, the sense and antisense strands are independently 21 to 26 nucleotides in length. In some embodiments, the sense and antisense strands are independently 21 to 24 nucleotides in length. The sense and antisense strands can be either the same length or different lengths. The RNAi agents include an antisense strand sequence that is at least partially complementary to a sequence in the target gene, and upon delivery to a cell expressing the target, an RNAi agent may inhibit the expression of one or more target genes in vivo or in vitro.

Oligonucleotide-based compounds generally, and RNAi agents specifically, may be comprised of modified nucleotides and/or one or more non-phosphodiester linkages. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides, 2′-modified nucleotides, 3′ to 3′ linkages (inverted) nucleotides, non-natural base-comprising nucleotides, bridged nucleotides, peptide nucleic acids, 2′,3′-seco nucleotide mimics (unlocked nucleobase analogues, locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides. 2′-modified nucleotides (i.e. a nucleotide with a group other than a hydroxyl group at the 2′ position of the five-membered sugar ring) include, but are not limited to, 2′-O-methyl nucleotides, 2′-deoxy-2′-fluoro nucleotides, 2′-deoxy nucleotides, 2′-methoxyethyl (2′-O-2-methoxylethyl) nucleotides, 2′-amino nucleotides, and 2′-alkyl nucleotides.

Moreover, one or more nucleotides of an oligonucleotide-based compound, such as an RNAi agent, may be linked by non-standard linkages or backbones (i.e., modified internucleoside linkages or modified backbones). A modified internucleoside linkage may be a non-phosphate-containing covalent internucleoside linkage. Modified internucleoside linkages or backbones include, but are not limited to, 5′-phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′.

It is not necessary for all positions in a given compound to be uniformly modified. Conversely, more than one modification may be incorporated in a single oligonucleotide-based compound or even in a single nucleotide thereof.

In some embodiments, the cargo molecule is an RNAi agent for inhibiting myostatin gene expression.

The RNAi agent sense strands and antisense strands may be synthesized and/or modified by methods known in the art. Additional disclosures related to RNAi agents may be found, for example, in the disclosure of modifications may be found, for example, in International Patent Application No. PCT/US2017/045446 to Arrowhead Pharmaceuticals, Inc., which also is incorporated by reference herein in its entirety.

In some embodiments, the one or more cargo molecule(s) can include or consist of a PEG moiety that can acts as a pharmacokinetic and/or pharmacodynamic (PK/PD) modulator. In some embodiments, the one or more cargo molecules can include a PEG moiety having about 20-900 ethylene oxide (CH₂—CH₂—O) units (e.g., 20 to 850, 20 to 800, 20 to 750, 20 to 700, 20 to 650, 20 to 600, 20 to 550, 20 to 500, 20 to 450, 20 to 400, 20 to 350, 20 to 300, 20 to 250, 20 to 200, 20 to 150, 20 to 100, 20 to 75, 20 to 50, 100 to 850, 100 to 800, 100 to 750, 100 to 700, 100 to 650, 100 to 600, 100 to 550, 100 to 500, 100 to 450, 100 to 400, 100 to 350, 100 to 300, 100 to 250, 100 to 200, 100 to 150, 200 to 850, 200 to 800, 200 to 750, 200 to 700, 200 to 650, 200 to 600, 200 to 550, 200 to 500, 200 to 450, 200 to 400, 200 to 350, 200 to 300, 200 to 250, 250 to 900, 250 to 850, 250 to 800, 250 to 750, 250 to 700, 250 to 650, 250 to 600, 250 to 550, 250 to 500, 250 to 450, 250 to 400, 250 to 350, 250 to 300, 300 to 900, 300 to 850, 300 to 800, 300 to 750, 300 to 700, 300 to 650, 300 to 600, 300 to 550, 300 to 500, 300 to 450, 300 to 400, 300 to 350, 350 to 900, 350 to 850, 350 to 800, 350 to 750, 350 to 700, 350 to 650, 350 to 600, 350 to 550, 350 to 500, 350 to 450, 350 to 400, 400 to 900, 400 to 850, 400 to 800, 400 to 750, 400 to 700, 400 to 650, 400 to 600, 400 to 550, 400 to 500, 400 to 450, 450 to 900, 450 to 850, 450 to 800, 450 to 750, 450 to 700, 450 to 650, 450 to 600, 450 to 550, 450 to 500, 500 to 900, 500 to 850, 500 to 800, 500 to 750, 500 to 700, 500 to 650, 500 to 600, 500 to 550, 550 to 900, 550 to 850, 550 to 800, 550 to 750, 550 to 700, 550 to 650, 550 to 600, 600 to 900, 600 to 850, 600 to 800, 600 to 750, 600 to 700, 600 to 650, 650 to 900, 650 to 850, 650 to 800, 650 to 750, 650 to 700, 700 to 900, 700 to 850, 700 to 800, 700 to 750, 750 to 900, 750 to 850, 750 to 800, 800 to 900, 850 to 900, or 850 to 900 ethylene oxide units). In some embodiments, the one or more cargo molecule(s) consist of a PEG moiety having approximately 455 ethylene oxide units (about 20 kilodalton (kDa) molecular weight). In some embodiments, a PEG moiety has a molecular weight of about 2 kilodaltons. In some embodiments, a PEG moiety has a molecular weight of about 20 kilodaltons. In some embodiments, a PEG moiety has a molecular weight of about 40 kilodaltons. The PEG moieties described herein may be linear or branched. The PEG moieties may be discrete (monodispersed) or non-discrete (polydispersed). PEG moieties for use as a PK enhancing cargo molecule may be purchase commercially. In some embodiments, the one or more cargo molecule(s) include a PEG moiety that can act as a PK/PD modulator or enhancer, as well as a different cargo molecule, such as a pharmaceutically active ingredient or compound.

The described αvβ6 integrin ligands include salts or solvates thereof. Solvates of an αvβ6 integrin ligand is taken to mean adductions of inert solvent molecules onto the αvβ6 integrin ligand which form owing to their mutual attractive force. Solvates are, for example, mono- or dihydrates or addition compounds with alcohols, such as, for example, with methanol or ethanol.

Free amino groups or free hydroxyl groups can be provided as substituents of αvβ6 integrin ligands with corresponding protecting groups.

The αvβ6 integrin ligands also include, e.g., derivatives, i.e., αvβ6 integrin ligands modified with, for example, alkyl or acyl groups, sugars or oligopeptides, which are cleaved either in vitro or in an organism.

In some embodiments, an αvβ6 integrin ligand disclosed herein facilitates the delivery of a cargo molecule into the cytosol of a cell presenting an αvβ6 integrin on its surface, either through ligand-mediated endocytosis, pinocytosis, or by other means. In some embodiments, an αvβ6 integrin ligand disclosed herein facilitates the delivery of a cargo molecule to the plasma membrane of a cell presenting an αvβ6 integrin.

### Pharmaceutical Compositions

In some embodiments, the present disclosure provides pharmaceutical compositions that include, consist of, or consist essentially of, one or more of the αvβ6 integrin ligands disclosed herein.

As used herein, a “pharmaceutical composition” comprises a pharmacologically effective amount of an Active Pharmaceutical Ingredient (API), and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions described herein can contain other additional components commonly found in pharmaceutical compositions. In some embodiments, the additional component is a pharmaceutically-active material. Pharmaceutically-active materials include, but are not limited to: anti-pruritics, astringents, local anesthetics, or anti-inflammatory agents (e.g., antihistamine, diphenhydramine, etc.), small molecule drug, antibody, antibody fragment, aptamers, and/or vaccine.

The pharmaceutical compositions may also contain preserving agents, solubilizing agents, stabilizing agents, wetting agents, emulsifiers, sweeteners, colorants, odorants, salts for the variation of osmotic pressure, buffers, coating agents, or antioxidants. They may also contain other agent with a known therapeutic benefit.

The pharmaceutical compositions can be administered in a number of ways depending upon whether local or systemic treatment is desired and upon the area to be treated. Administration can be made by any way commonly known in the art, such as, but not limited to, topical (e.g., by a transdermal patch), pulmonary (e.g., by inhalation or insufflation of powders or aerosols, including by nebulizer, intratracheal, intranasal), epidermal, transdermal, oral or parenteral. Parenteral administration includes, but is not limited to, intravenous, intraarterial, subcutaneous, intraperitoneal or intramuscular injection or infusion; subdermal (e.g., via an implanted device), intracranial, intraparenchymal, intrathecal, and intraventricular, administration. In some embodiments, the pharmaceutical compositions described herein are administered by subcutaneous injection. The pharmaceutical compositions may be administered orally, for example in the form of tablets, coated tablets, dragees, hard or soft gelatine capsules, solutions, emulsions or suspensions. Administration can also be carried out rectally, for example using suppositories; locally or percutaneously, for example using ointments, creams, gels, or solutions; or parenterally, for example using injectable solutions.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of any of the ligands described herein that can be in microcrystalline form, for example, in the form of an aqueous microcrystalline suspension. Liposomal formulations or biodegradable polymer systems can also be used to present any of the ligands described herein for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). As used herein, “pharmacologically effective amount,” “therapeutically effective amount,” or simply “effective amount” refers to that amount of an the pharmaceutically active agent to produce a pharmacological, therapeutic or preventive result.

Medicaments containing an αvβ6 integrin ligand are also an object of the present invention, as are processes for the manufacture of such medicaments, which processes comprise bringing one or more compounds containing a αvβ6 integrin ligand, and, if desired, one or more other substances with a known therapeutic benefit, into a pharmaceutically acceptable form.

The described αvβ6 integrin ligands and pharmaceutical compositions comprising αvβ6 integrin ligands disclosed herein may be packaged or included in a kit, container, pack, or dispenser. The αvβ6 integrin ligands and pharmaceutical compositions comprising the αvβ6 integrin ligands may be packaged in pre-filled syringes or vials.

### Cells, Tissues, and Non-Human Organisms

Cells, tissues, and non-human organisms that include at least one of the αvβ6 integrin ligands described herein is contemplated. The cell, tissue, or non-human organism is made by delivering the αvβ6 integrin ligand to the cell, tissue, or non-human organism by any means available in the art. In some embodiments, the cell is a mammalian cell, including, but not limited to, a human cell.

Linking Groups, Pharmacokinetic and/or Pharmacodynamic (PK/PD) Modulators, and Delivery Vehicles

In some embodiments, an αvβ6 ligand is conjugated to one or more non-nucleotide groups including, but not limited to, a linking group, a pharmacokinetic and/or pharmacodynamic (PK/PD) modulator, a delivery polymer, or a delivery vehicle. The non-nucleotide group can enhance targeting, delivery, or attachment of the cargo molecule. Examples of targeting groups and linking groups are provided in Table 6. The non-nucleotide group can be covalently linked to the 3′ and/or 5′ end of either the sense strand and/or the antisense strand. In embodiments where the cargo molecule is an RNAi agent, the RNAi agent contains a non-nucleotide group linked to the 3′ and/or 5′ end of the sense strand. In some embodiments, a non-nucleotide group is linked to the 5′ end of an RNAi agent sense strand. An αvβ6 ligand can be linked directly or indirectly to the cargo molecule via a linker/linking group. In some embodiments, a αvβ6 ligand is linked to the cargo molecule via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting moieties enhance the pharmacokinetic or biodistribution properties of a cargo molecule to which they are attached to improve cell-specific (including, in some cases, organ specific) distribution and cell-specific (or organ specific) uptake of the cargo molecule. In some embodiments, a targeting group may comprise an αvβ6 ligand as described herein. In some embodiments, a targeting group comprises a linker. In some embodiments, a targeting group comprises a PK/PD modulator. In some embodiments, an αvβ6 ligand is linked to a cargo molecule using a linker, such as a PEG linker or one, two, or three abasic and/or ribitol (abasic ribose) residues, which in some instances can serve as linkers.

Cargo molecules can be synthesized having a linking moiety, such as an amino group (also referred to herein as an amine). In embodiments where the cargo molecule is an RNAi agent, the linking moiety may be linked at the 5′-terminus and/or the 3′-terminus. The linking moiety can be used subsequently to attach an αvβ6 ligand using methods typical in the art.

For example, in some embodiments, an RNAi agent is synthesized having an NH₂-C₆ group at the 5′-terminus of the sense strand of the RNAi agent. The terminal amino group subsequently can be reacted to form a conjugate with, for example, a group that includes an αvβ6 integrin targeting ligand. In some embodiments, an RNAi agent is synthesized having one or more alkyne groups at the 5′-terminus of the sense strand of the RNAi agent. The terminal alkyne group(s) can subsequently be reacted to form a conjugate with, for example, a group that includes an αvβ6 integrin targeting ligand.

In some embodiments, a linking group is conjugated to the αvβ6 ligand. The linking group facilitates covalent linkage of the αvβ6 ligand to a cargo molecule, PK/PD modulator, delivery polymer, or delivery vehicle. Examples of linking groups, include, but are not limited to: Alk-SMPT-C6, Alk-SS-C6, DBCO-TEG, Me-Alk-SS-C6, and C6-SS-Alk-Me, linking moieties such a primary amines and alkynes, alkyl groups, abasic residues/nucleotides, amino acids, tri-alkyne functionalized groups, ribitol, and/or PEG groups.

A linker or linking group is a connection between two atoms that links one chemical group (such as an RNAi agent) or segment of interest to another chemical group (such as an αvβ6 ligand, PK/PD modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

In some embodiments, αvβ6 ligands are linked to cargo molecules without the use of an additional linker. In some embodiments, the αvβ6 ligand is designed having a linker readily present to facilitate the linkage to a cargo molecule. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

Examples of certain linking groups are provided in Table A.

TABLE A
Structures Representing Various Linking Groups

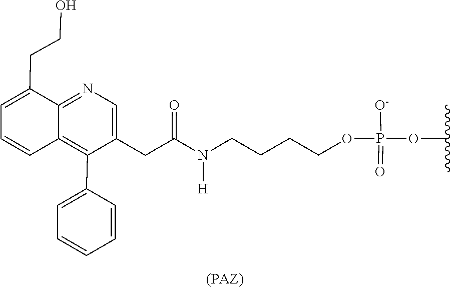 [SMILES: *OP(=O)([O-])OCCCCNC(=O)Cc1cnc2c(CCO)cccc2c1-c1ccccc1]

When positioned at the 3′ terminal end of oligonucleotide:

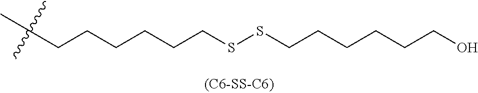 [SMILES: *CCCCCCSSCCCCCCO]

When positioned internally in oligonucleotide:

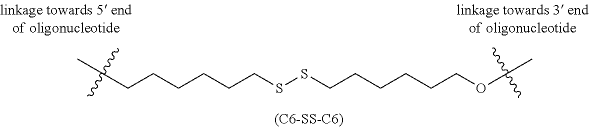 [SMILES: *CCCCCCSSCCCCCCO*]

When positioned at the 3′ terminal end of oligonucleotide:

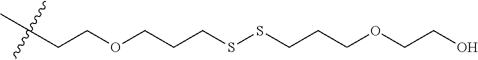 [SMILES: *CCOCCCSSCCCOCCO]

When positioned internally in oligonucleotide:

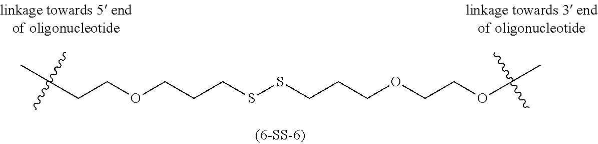 [SMILES: *CCOCCCSSCCCOCCO*]

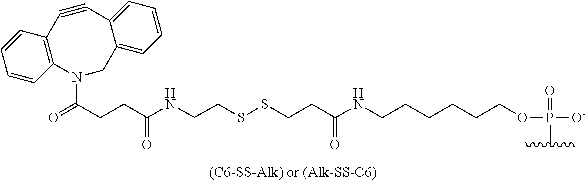 [SMILES: *P(=O)([O-])OCCCCCCNC(=O)CCSSCCNC(=O)CCC(=O)N1Cc2ccccc2C#Cc2ccccc21]

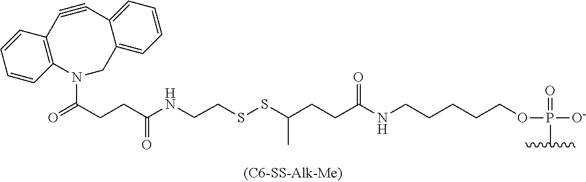 [SMILES: *P(=O)([O-])OCCCCCNC(=O)CCC(C)SSCCNC(=O)CCC(=O)N1Cc2ccccc2C#Cc2ccccc21]

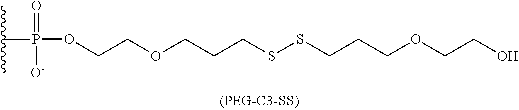 [SMILES: *P(=O)([O-])OCCOCCCSSCCCOCCO]

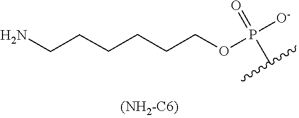 [SMILES: *P(=O)([O-])OCCCCCCN]

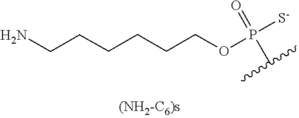 [SMILES: *P(=O)([S-])OCCCCCCN]

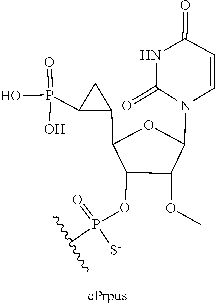 [SMILES: *P(=O)([S-])OC1C(C2CC2P(=O)(O)O)OC(n2ccc(=O)[nH]c2=O)C1OC]

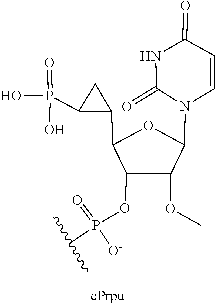 [SMILES: *P(=O)([O-])OC1C(C2CC2P(=O)(O)O)OC(n2ccc(=O)[nH]c2=O)C1OC]

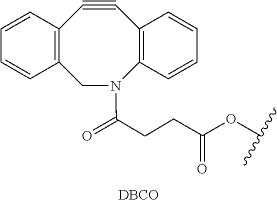

wherein [symbol] indicates the point of attachment to a cargo molecule.

Alternatively, other linking groups known in the art may be used.

The above provided embodiments and items are now illustrated with the following, non-limiting examples.

### EXAMPLES

The following examples are not limiting and are intended to illustrate certain embodiments disclosed herein.

### Example 1. Synthesis of αvβ6 Integrin Ligands

Some of the abbreviations used in the following experimental details of the synthesis of the examples are defined as follows: h or hr=hour(s); min=minute(s); mol=mole(s); mmol=millimole(s); M=molar; M=micromolar; g=gram(s); μg=microgram(s); rt or RT=room temperature; L=liter(s); mL=milliliter(s); wt=weight; Et₂O=diethyl ether; THF=tetrahydrofuran; DMSO=dimethyl sulfoxide; EtOAc=ethyl acetate; Et₃N or TEA=triethylamine; i-Pr₂NEt or DIPEA or DIEA=diisopropylethylamine; CH₂Cl₂ or DCM=methylene chloride; CHCl₃=chloroform; CDCl₃=deuterated chloroform; CCl₄=carbon tetrachloride; MeOH=methanol; EtOH=ethanol; DMF=dimethylformamide; BOC=t-butoxycarbonyl; CBZ=benzyloxycarbonyl; TBS=t-butyldimethylsilyl; TBSCl or TBDMSCl=t-butyldimethylsilyl chloride; TFA=trifluoroacetic acid; DMAP=4-dimethylaminopyridine; NaN₃=sodium azide; Na₂SO₄=sodium sulfate; NaHCO₃=sodium bicarbonate; NaOH=sodium hydroxide; MgSO₄=magnesium sulfate; K₂CO₃=potassium carbonate; KOH=potassium hydroxide; NH₄OH=ammonium hydroxide; NH₄Cl=ammonium chloride; SiO₂=silica; Pd—C=palladium on carbon; HCl=hydrogen chloride or hydrochloric acid; NMM=N-methylmorpholine; H₂=hydrogen gas; KF=potassium fluoride; EDC-HCl=N-(3-Dimethylaminopropyl)-N′-ethylcarbodiimide hydrochloride; MTBE=methyl-tert-butyl ether; Ar=argon; N₂=nitrogen; RT=retention time.

Chemical names for structures 40p-60p were automatically generated using ChemDraw® software.

### Synthesis of Compound 40p, (S)-3-(4-(4-((14-azido-3,6,9,12-tetraoxatetradecyl)oxy)naphthalen-1-yl)phenyl)-3-(2-(3-((4,5-dihydro-1H-imidazol-2-yl)amino)benzamido)acetamido)propanoic acid

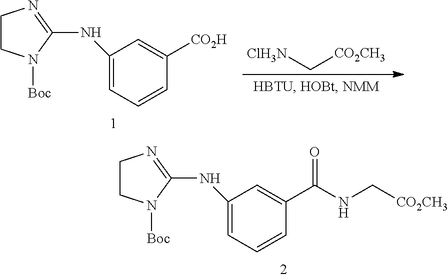

HBTU (239 mg, 0.629 mmol) was added to the ice-cold solution of acid 1 (160 mg, 0.523 mmol), glycine methyl ester hydrochloride (79 mg, 0.639 mmol), HOBt 948 mg, 0.312 mmol), and 4-methylmorpholine (338 uL, 3 mmol) in DMF (10 mL). The cooling bath was removed, and the reaction mixture was stirred for 2 h at RT. Water (1 mL) was added and the reaction mixture was concentrated to dryness in high vacuo. The residue was partitioned between EtOAc and water (1:1, 50 mL). EtOAc layer was washed twice with water. The aqueous washes were back-extracted once with EtOAc, organic phases were combined, dried with Na₂SO₄ and concentrated in vacuo, and product was purified on combiflash using the system DCM: 20% MeOH in DCM, gradient 5-30%, 20 min. Yield 192 mg (97%). NMR (DMSO-d₆): 1.5 s (9H); 3.65 s (3H); 3.7 m (4H); 4.0 d (2H), 7.38 t (1H); 7.45 m (1H); 7.96 bs (1H); 8.3 s (1H); 8.88 t (1H); 9.44 bs (1H). Molecular mass calculated: 376.17 Found: MS (ES, pos): 377.30 [M+1]⁺, 277.33 [M+1−Boc]⁺.

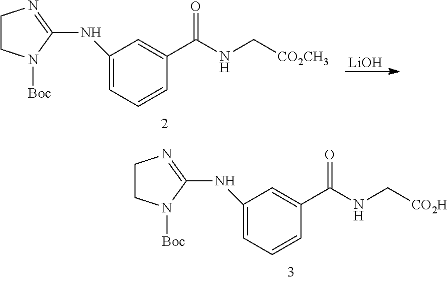 [SMILES: COC(=O)CNC(=O)c1cccc(NC2=NCCN2C(=O)OC(C)(C)C)c1 ; "LiOH" ; CC(C)(C)OC(=O)N1CCN=C1Nc1cccc(C(=O)NCC(=O)O)c1]

A solution of LiOH (36 mg, 1.515 mmol) in water (3 mL) was added dropwise to a stirred solution of the ester 2 in THF (5 mL). Following 2 h of stirring the reaction mixture was cooled in an ice bath and acidified to pH=4.5 with 1N HCl. About ½ of the solvent volume was removed in vacuo and the product was 5 times extracted with EtOAc. Product was dried (Na₂SO₄), filtered, and concentrated and dried in vacuo. Yield 114 mg (63%). The product was used directly in the next step. NMR (DMSO-d₆): 1.51 s (9H); 3.59 m (2H); 3.95 d (2H); 4.05 m (2H); 7.09 m (2H); 7.9 m (2H); 8.92 t (1H); 9.35 bs (1H); 10.52 bs(1H), 12.6 bs (1H).

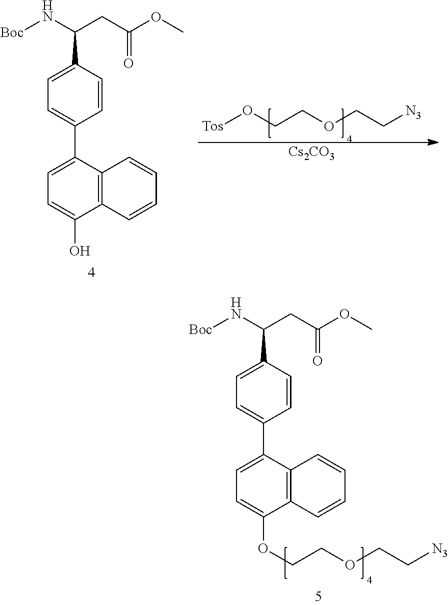

Cesium carbonate (2.556 g, 7.845 mmol) was added into a solution of methyl ester of 3-(N-Boc-amino)-3-[4-[4-hydroxynaphthyl]phenyl]-propionic acid 4 (3 g, 7.132 mmol) and Tos-Peg5-N3 (3.275 g, 7.845 mmol) in DMF (100 mL). The reaction mixture was stirred at 40° C. for 3 h followed by 14 h at RT, cooled to 0° C. and poured into cold saturated solution of NaHCO₃. The product was extracted with 4×200 mL of EtOAc, dried (Na₂SO₄), and concentrated in vacuo. The residual DMF was removed by 2 co-evaporation of toluene from the product on a rotavapor. Combiflash purification using system DCM: 20% MeOH in DCM, gradient=0-20%. Yield 4.757 g (88%). NMR (DMSO-d₆): 1.39 s (9H); 2.80 m(2H); 3.36 t (2H); 3.456 m (12H), 3.69 m (2H); 3.92 m (2H); 4.32 m (2H); 5.03 q (1H); 7.06 d (1H); 7.33 d (1H); 7.40 d (2H); 7.44 d (2H); 7.54 m (3H); 7.77 m (1H); 8.27 m (1H). Molecular mass calculated: 666.33, 684.33 [M+NH₄]⁺ Found MS (ES, pos): 684.54 [M+NH₄]⁺; 567.43 [M+1−Boc]⁺.

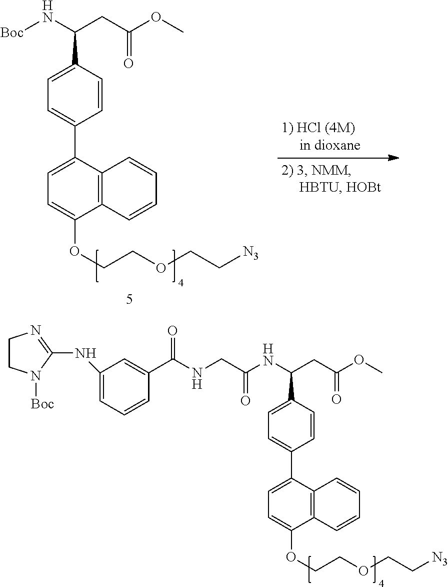

Compound 5 (200 mg, 0.3 mmol) was treated with ice-cold solution of 4M HCl in dioxane, the cooling bath was removed, and the reaction mixture was stirred for 30 min at RT. The product was concentrated and dried in vacuo. The residual HCl was removed by co-evaporation of dioxane. MS (ES, pos): 567 [M+1]⁺. The obtained free amine was dissolved in DMF (10 mL), compound 3 (108 mg, 0.3 mmol), HOBt (28 mg, 0.18 mmol), 4-methylmorpholine (200 uL, 1.8 mmol) were added and the mixture was cooled on an ice bath. HBTU (137 mg, 0.36 mmol) was added, the cooling bath was removed, and the mixture was stirred at RT for 14 h. Water (0.5 mL) was added, DMF was evaporated in high vacuo. The residue was partitioned between EtOAc and water (1:1, 50 mL), basified to pH=8 with NaHCO₃, and the product was extracted with EtOAc 3 times. The EtOAc solution was dried (Na₂SO₄), filtered, and concentrated to dryness. Product was purified on Combiflash® using the system DCM: 20% MeOH in DCM, gradient 0-40%, 20 min. Yield 132 mg (48%). NMR (DMSO-d₆): 1.51 s (9H); 2.60 m (2H); 3.38 t (2H); 3.59 m (2H); 3.95 m 6H); 4.32 m (2H); 5.27 q (1H); 7.06 d (1H); 7.33 d (1H); 7.42 d (2H); 7.48 d (2H); 7.53 m (2H); 7.58 m (2H); 7.77 m (1H); 7.89 m (2H); 8.28 m (1H); 8.62 d (1H); 8.79 t (1H); 9.2 bs (1H). Molecular mass calculated: 910.422 Found MS (ES, pos): 911.58 [M+1]⁺; 811.48 [M+1−Boc]⁺.

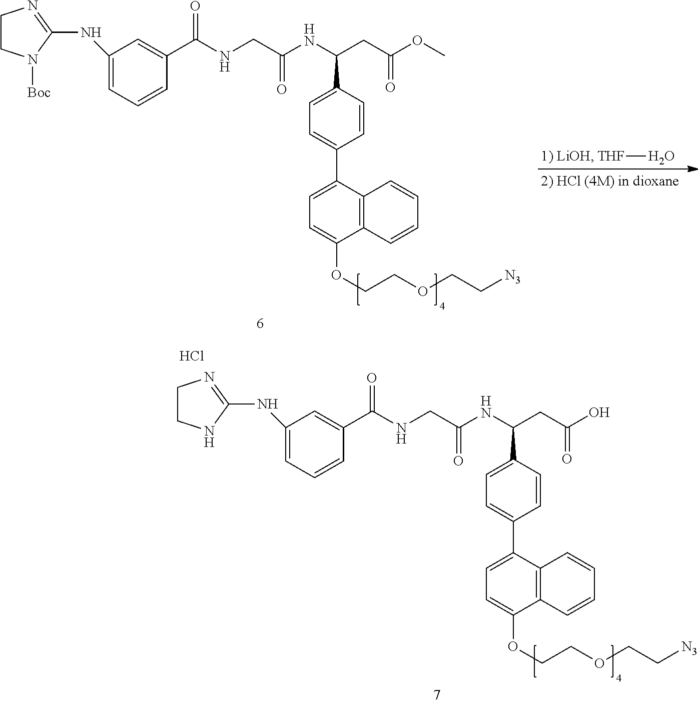

Compound 6 (68.4 mg, 0.075 mmol) was stirred with a solution of LiOH (11 mg, 0.224 mmol) in THF:water=1:1 (2 ml) for 2 h at RT. THF was evaporated in vacuo, the aqueous residue was diluted with water to 10 mL, acidified to pH=4 with 1N HCl, brine (3 mL) was added, and the product was extracted 3 times with EtOAc. MS (ES, pos): 897.90 [M+1]⁺; 797.61 [M+1−Boc]⁺. The crude product was treated with ice-cold 4M HCl solution of HCl in dioxane, the cooling bath was removed and the mixture was stirred for 90 min at RT. All volatiles were removed in vacuo, the residual HCl was removed by 2 co-evaporations of dioxane. Yield 59 mg (94%). Molecular mass calculated: 796.35 Found MS (ES, pos): 797.43 [M+1]⁺.

### Synthesis of Compound 41p, (3S)-3-(4-(4-((14-azido-3,6,9,12-tetraoxatetradecyl)oxy)naphthalen-1-yl)phenyl)-3-(2-(3-hydroxy-5-((5-hydroxy-1,4,5,6-tetrahydropyrimidin-2-yl)amino)benzamido)acetamido)propanoic acid

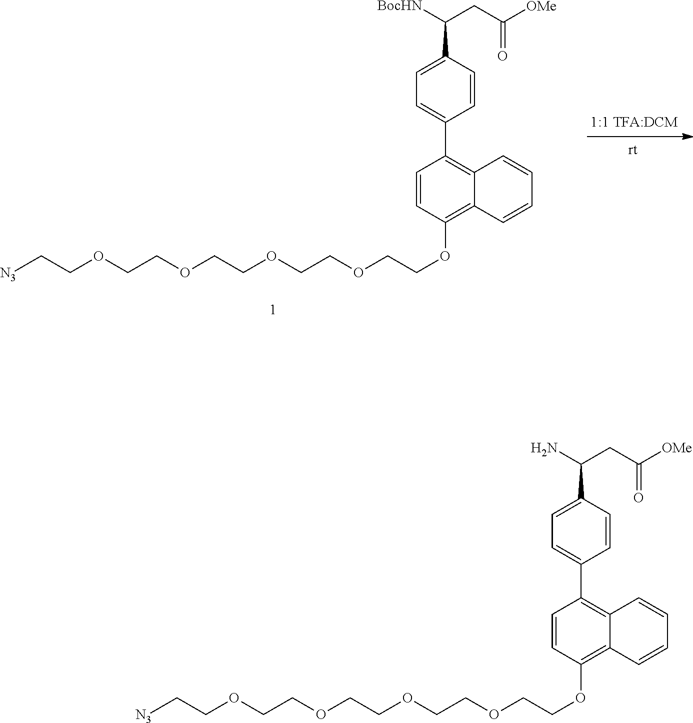 [SMILES: COC(=O)C[C@H](NC(=O)OC(C)(C)C)c1ccc(-c2ccc(OCCOCCOCCOCCOCCN=[N+]=[N-])c3ccccc23)cc1 ; COC(=O)C[C@H](N)c1ccc(-c2ccc(OCCOCCOCCOCCOCCN=[N+]=[N-])c3ccccc23)cc1]

Into a 50-mL round bottom flask with stir bar was added 1.5 g of compound 1, 4 mL of DCM, and 4 mL of TFA. The reaction was allowed to stir under ambient atmosphere at rt at 500 rpm.

After 2 h, the reaction showed full conversion by LC-MS. Reaction was azeotroped with toluene and concentrated under vacuum. The product was subjected to a base extraction with NaHCO₃ and EtOAc to obtain the free amine. LC-MS: calculated [M+H]+ 567.27 m/z, observed 567.52 m/z.

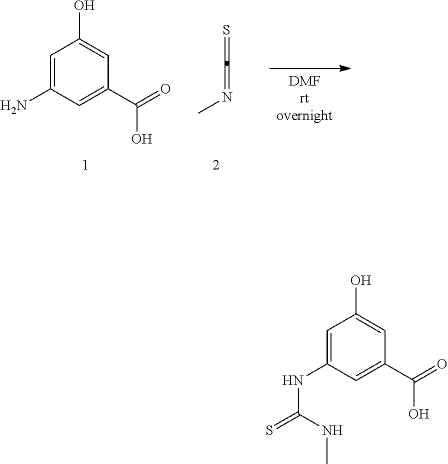

To a solution of compound (4.80 g) 1 in DMF, 2 (2.29 g) was added under a strong flow of N₂(g) via solid-phase transfer; due to 2 sticking to the weight boat, reaction mixture was used to rinse and transfer contents of 2 into reaction flask. The reaction was stirred under ambient conditions for 1-3 h. Upon confirmation of full reaction conversion by LC-MS, crude reaction mixture was carried through to the next step. LC-MS: calculated [M+H]+ 227.04 m/z, observed 227.05 m/z.

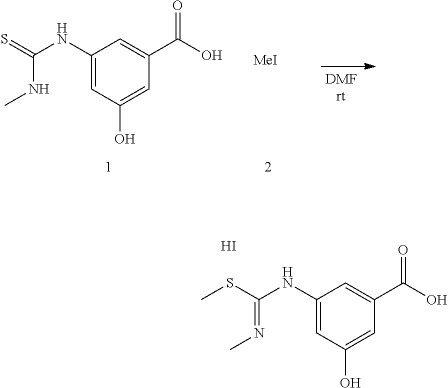

To a solution of compound 1 (0.28 g) in DMF, compound 2 (2.967 mL) was added via syringe and hypodermic needle under ambient conditions (1:15 pm). Reaction was stirred under ambient conditions overnight. Upon confirmation of full reaction conversion by LC-MS, crude reaction mixture was carried through to the next step. LC-MS: calculated [M+H]+241.06 m/z, observed 241.00 m/z.

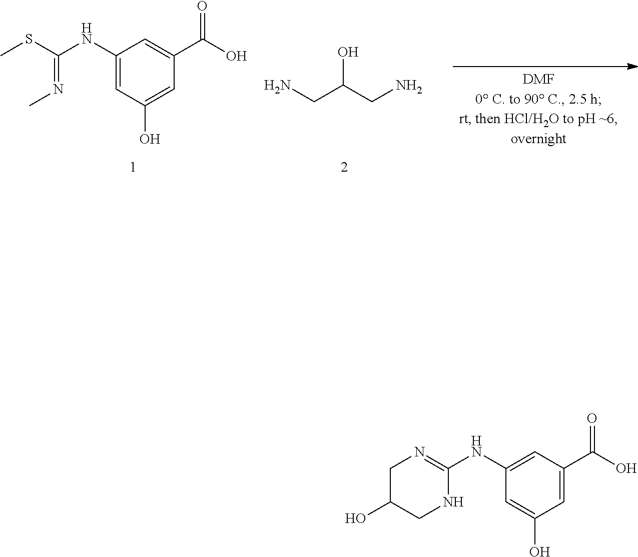 [SMILES: C/N=C(/Nc1cc(O)cc(C(=O)O)c1)SC ; NCC(O)CN ; O=C(O)c1cc(O)cc(NC2=NCC(O)CN2)c1]

To a solution of compound 1 (0.28 g) in DMF, cooled to 0° C., compound 2 (4.60 g) was added under ambient conditions. The reaction was heated to 90° C. and allowed to stir for 3 h. Then reaction mixture was cooled to rt, and water (10 mL) and concentrated HCl were added to adjust reaction pH to 5-6. The reaction was stirred at rt overnight. Upon confirmation of full reaction conversion by LC-MS, reaction mixture was filtered and rinsed with EtOAc to recover a taupe solid product in the filter cake. No product was observed in the filtrate. LC-MS: calculated [M+H]+ 252.09 m/z, observed 252.08 m/z. The isolated product weighed 0.4287 g. Yield over 4 steps: 5.0%.

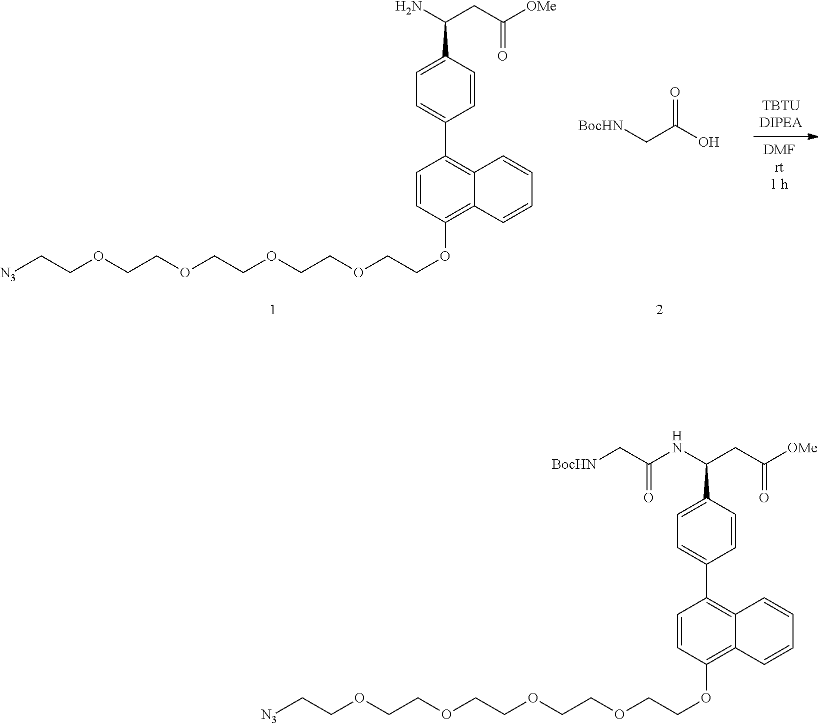 [SMILES: COC(=O)C[C@H](N)c1ccc(-c2ccc(OCCOCCOCCOCCOCCN=[N+]=[N-])c3ccccc23)cc1 ; CC(C)(C)OC(=O)NCC(=O)O ; COC(=O)C[C@H](NC(=O)CNC(=O)OC(C)(C)C)c1ccc(-c2ccc(OCCOCCOCCOCCOCCN=[N+]=[N-])c3ccccc23)cc1]

To a solution of compounds 1 (0.54 g) and 2 (0.25 g) in DMF was added TBTU (0.37 g) and then DIPEA (0.50 mL) under ambient conditions. Reaction was stirred for 3 h. Then reaction mixture was quenched with NaHCO₃(10 mL) and brine (15 mL). The product was extracted with EtOAc (3×15 mL). The combined organic phase was dried over Na₂SO₄, filtered, and concentrated. The residue was purified by CombiFlash® using silica gel as the stationary phase with a gradient of DCM to 20% MeOH in DCM (0-40%), in which product eluted at 13% B. Recovery of product: 0.50 g (71.9% yield). LC-MS: calculated [M+H]+ 724.35 m/z, observed 724.69 m/z.

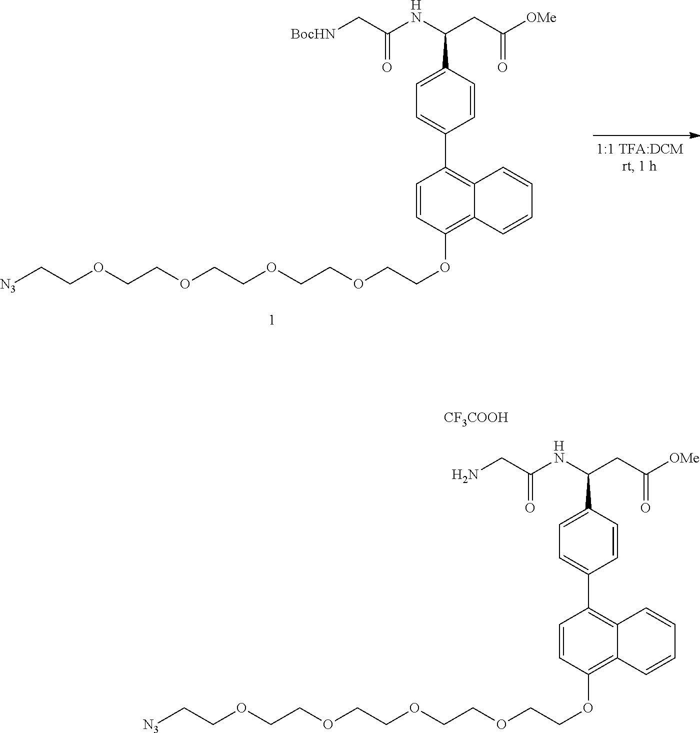 [SMILES: COC(=O)C[C@H](NC(=O)CNC(=O)OC(C)(C)C)c1ccc(-c2ccc(OCCOCCOCCOCCOCCN=[N+]=[N-])c3ccccc23)cc1 ; COC(=O)C[C@H](NC(=O)CN)c1ccc(-c2ccc(OCCOCCOCCOCCOCCN=[N+]=[N-])c3ccccc23)cc1 ; "CF3COOH"]

To a solution of compound 1 (0.50 g) in DCM was added TFA (1.59 mL) at rt. The reaction was stirred under ambient conditions. After 1 h, full conversion was confirmed via LC-MS. The reaction mixture was quenched with NaHCO₃(10 mL), extracted with EtOAc (3×10 mL), and concentrated under vacuum. No isolation was necessary. Concentration provided a yellow oil (0.28 g, 54.8%.) LC-MS: calculated [M+H]+ 624.30 m/z, observed 624.50 m/z.

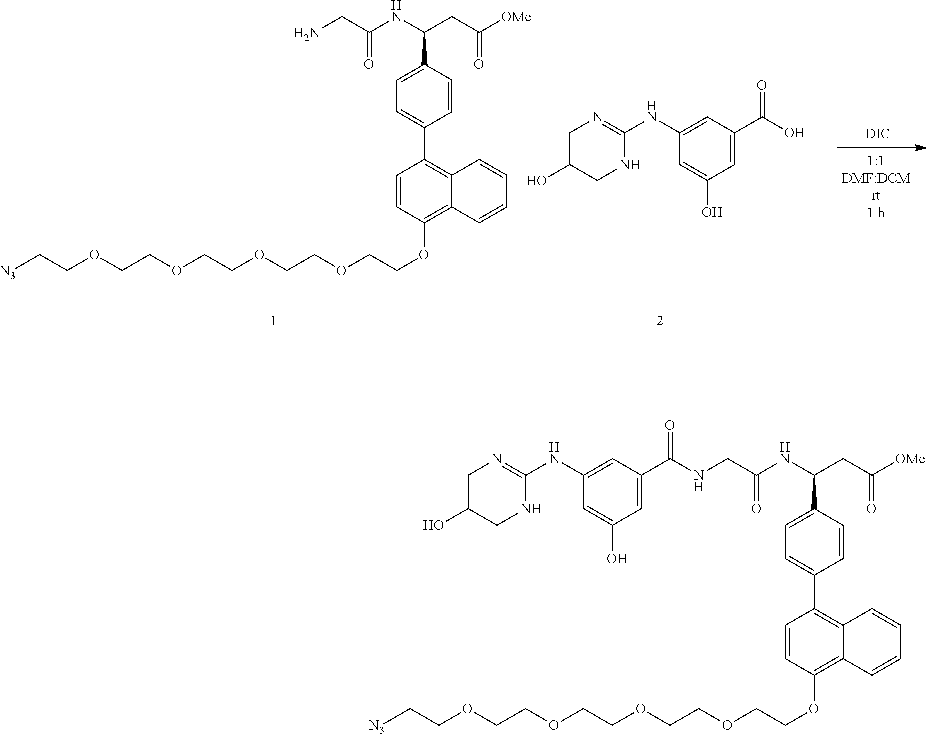

To a solution of compounds 1 (0.050 g) and 2 (0.0211 g) in 1:1 DMF:DCM under N₂(g) was added DIC (0.015 mL) at rt. Reaction was stirred under N₂(g) at rt over the weekend. By LC-MS, the observed mixture consisted of unreacted starting materials and some urea intermediate. Two equivalents of DIPEA (0.028 mL) were then added. After 40 min., the observed mixture also included undesired side product. After 5 h, no desired product was observed, so reaction was heated to 40° C., and reaction was stirred overnight. With no observation of desired product, DIC (0.1 mL) and HOBt (˜10-20 mg) were added, and reaction was allowed to continue stirring at 40° C. for 1.5 h until full conversion to product was observed. The crude reaction mixture was then employed for the next step in-situ. LC-MS: calculated [M+H]+ 857.38 m/z, observed 857.84 m/z.

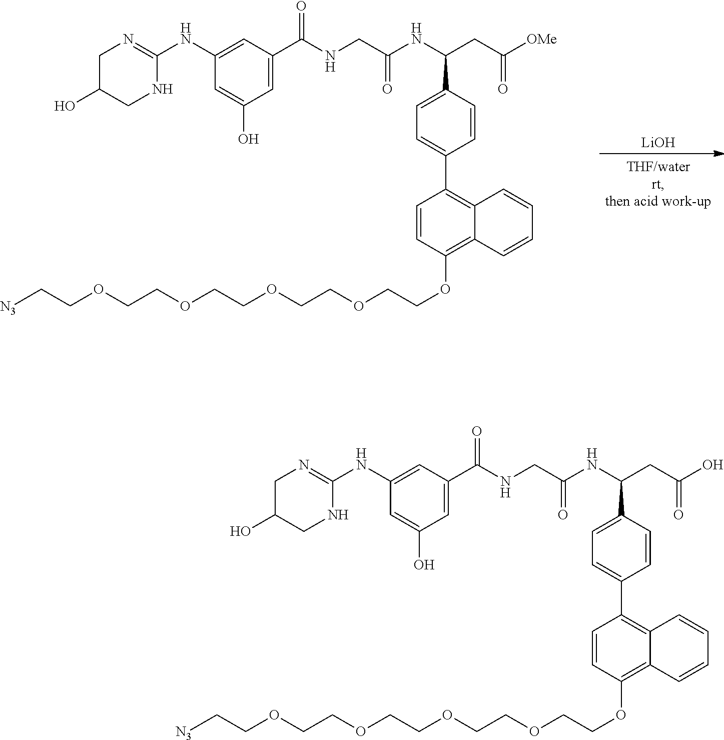 [SMILES: COC(=O)C[C@H](NC(=O)CNC(=O)c1cc(O)cc(NC2=NCC(O)CN2)c1)c1ccc(-c2ccc(OCCOCCOCCOCCOCCN=[N+]=[N-])c3ccccc23)cc1 ; "LiOH" ; [N-]=[N+]=NCCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12]

Saponification was performed in-situ of ester (0.069 g). To the crude reaction mixture were added ˜2 mL of water and then ˜10 mg of LiOH at rt under normal atmosphere. The reaction was stirred at rt until full conversion was observed by LC-MS. Mixture was then concentrated under vacuum and azeotroped with PhMe. The mixture was resuspended in 1 mL of DMF and 1 mL of water and isolated via reverse-phase HPLC. Recovery of compound 41p: 0.029 g (43.0% over two steps). LC-MS: calculated [M+H]+ 843.36 m/z, observed 843.35 m/z.

### Synthesis of Compound 42p, (S)-3-(4-(4-((14-azido-3,6,9,12-tetraoxatetradecyl)oxy)naphthalen-1-yl)phenyl)-3-(2-(5-guanidinopentanamido)acetamido)propanoic acid

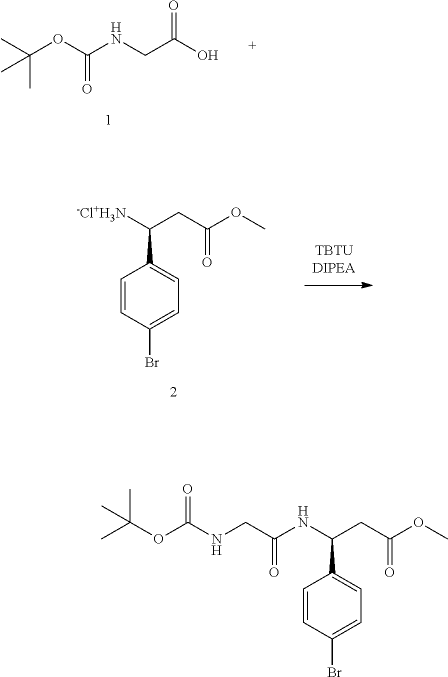

To a solution of compound 1 (1300 mg, 7.42 mmol, 1.0 equiv.), compound 2 (2295 mg, 7.792 mmol, 1.05 equiv.) and diisopropylethylamine (3.878 mL, 22.262 mmol, 3.0 equiv.) in anhydrous DMF (10 mL) was added TBTU (2859 mg, 8.905 mmol, 1.2 equiv.) at room temperature. The reaction was kept at room temperature for 2 hrs. The reaction was quenched with saturated NaHCO₃ aqueous solution (5 mL) and the aqueous was extracted with ethyl acetate (3×5 mL). The organic phase was combined, dried over anhydrous Na₂SO₄, and concentrated. The product was purified by CombiFlash and was eluted with 2-4% methanol in dichloromethane. LC-MS: calculated [M+H]+ 415.08, found 415.29. Yield: 0.19 g, 6.04%.

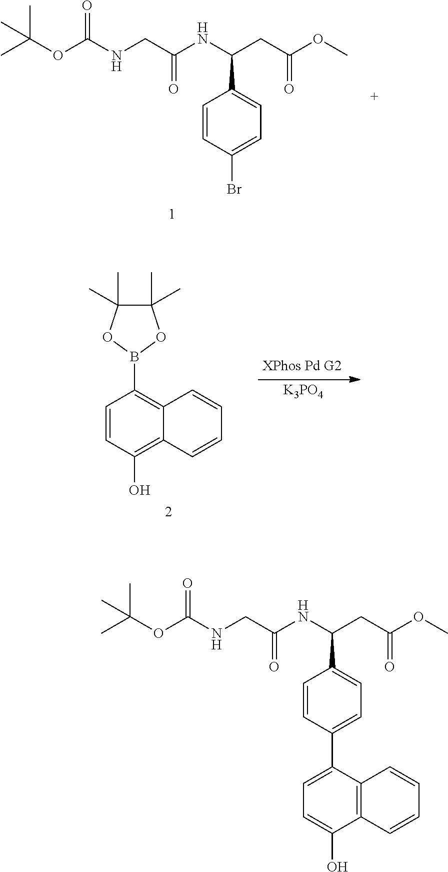

Compound 1 (3.20 g, 7.705 mmol, 1.0 equiv.), compound 2 (3.12 g, 11.558 mmol, 1.5 equiv.), XPhos Pd G2 (121 mg, 0.154 mmol, 0.02 equiv.), and K3PO4 (3.27 g, 15.411 mmol, 2.0 equiv.) were mixed in a round-bottom flask. The flask was sealed with a screw-cap septum, and then evacuated and backfilled with nitrogen (this process was repeated a total of 3 times). Then, THF (20 mL) and water (4 mL) were added via syringe. The mixture was bubbled with nitrogen for 10 min and the reaction was kept at 40° C. for 3 hrs. The reaction was quenched with saturated NaHCO₃ aqueous solution (20 mL), and the aqueous phase was extracted with ethyl acetate (3×20 mL). The organic phase was combined, dried over Na₂SO₄, and concentrated. The compound was separated by CombiFlash®, and was eluted with 2-4% methanol in DCM.

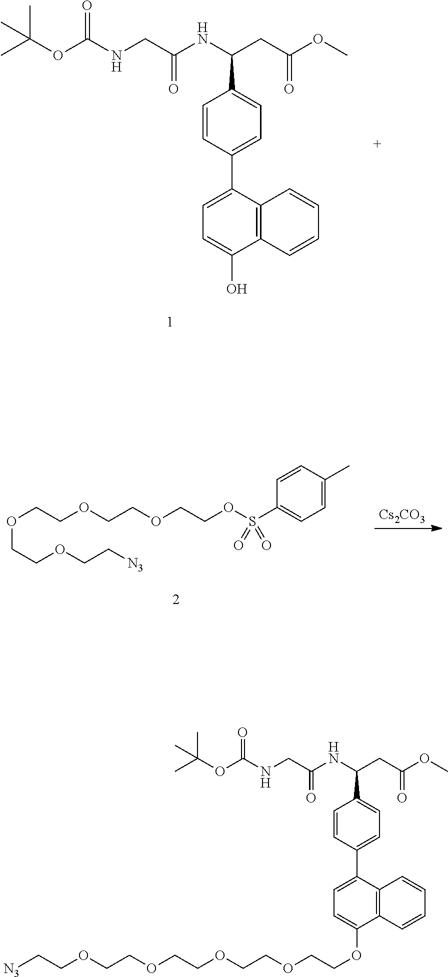

To a solution of compound 1 (1.61 g, 3.364 mmol, 1.0 equiv.) and compound 2 (1.75 g, 4.205 mmol, 1.25 equiv.) in anhydrous DMF (10 mL) was added cesium carbonate (2.19 g, 6.728 mmol, 2.0 equiv.) at room temperature. The reaction was kept at 50° C. for 2 hrs. The reaction was quenched with water (20 mL) and was extracted with ethyl acetate (3×10 mL). The organic phase was combined, dried over anhydrous Na₂SO₄, and concentrated. The product was purified by CombiFlash, and was eluted with 2-4% methanol in dichloromethane. LC-MS: calculated [M+H]+ 724.35, found 724.60.

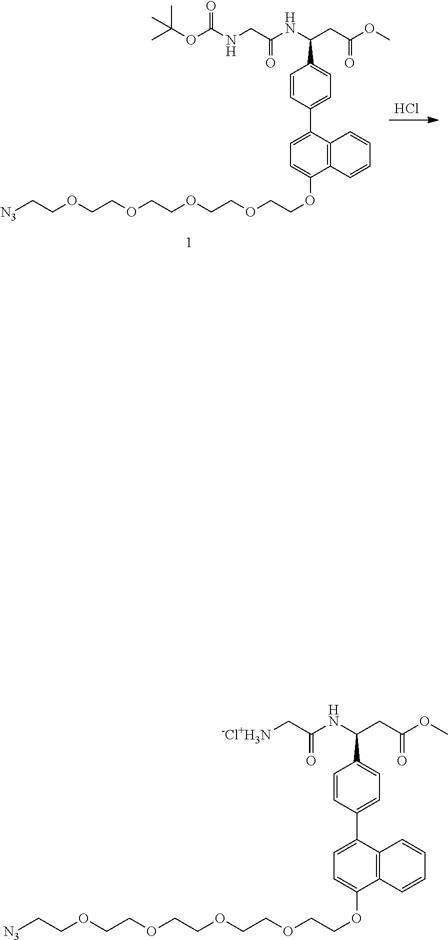

To a solution of compound 1 (1880 mg, 2.597 mmol, 1.0 equiv.) in anhydrous dioxane (3 mL) was added HCl in dioxane (3.25 mL, 12.986 mmol, 5.0 equiv.) at room temperature. The reaction was kept at room temperature for 2 hrs. The solvent was removed and the product was used directly without purification. LC-MS: calculated [M+H]+ 624.30,

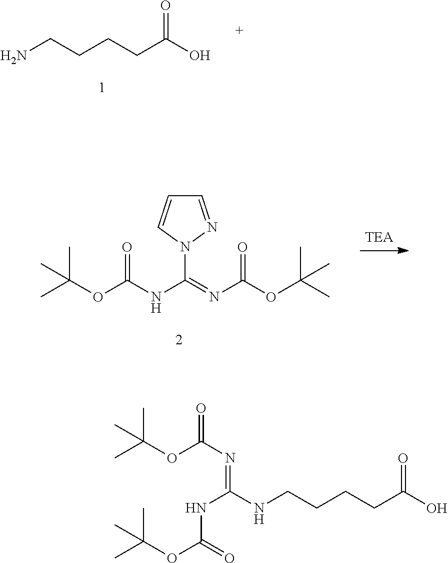

To a solution of compound 1 (500 mg, 4.268 mmol, 1.0 equiv.) and compound 2 (1.607 g, 5.335 mmol, 1.25 equiv.) in anhydrous methanol (10 mL) was added triethylamine (1.786 mL, 12.804 mmol, 3.0 equiv.) at room temperature. The reaction was kept at room temperature overnight. The reaction mixture was concentrated and the product was separated by CombiFlash. The product was eluted with 2-3% methanol in dichloromethane. LC-MS: calculated [M+H]+ 360.21, found 360.46.

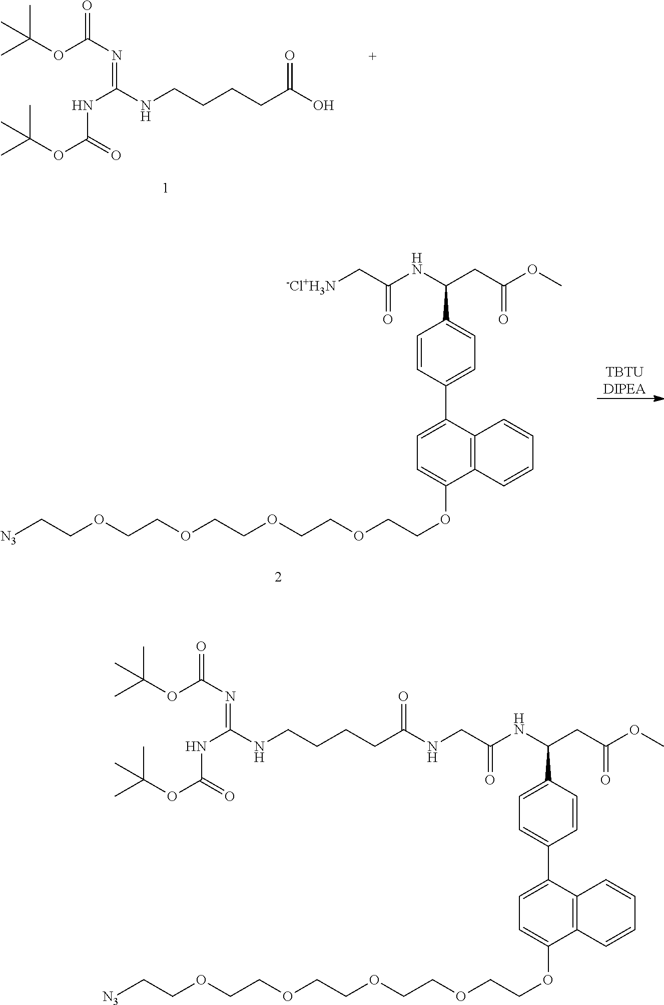

To a solution of compound 1 (66 mg, 0.183 mmol, 1.0 equiv.), compound 2 (127 mg, 0.192 mmol, 1.05 equiv.) and diisopropylethylamine (0.096 mL, 0.550 mmol, 3.0 equiv.) in anhydrous DMF (1 mL) was added TBTU (70 mg, 0.220 mmol, 1.2 equiv.) at room temperature. The reaction was kept at room temperature for 2 hrs. The reaction was quenched with saturated NaHCO₃ aqueous solution (5 mL) and the aqueous was extracted with ethyl acetate (3×5 mL). The organic phase was combined, dried over anhydrous Na₂SO₄, and concentrated. The product was purified by CombiFlash and was eluted with 2-4% methanol in dichloromethane. LC-MS: calculated [M+H]+ 965.49, found 965.69.

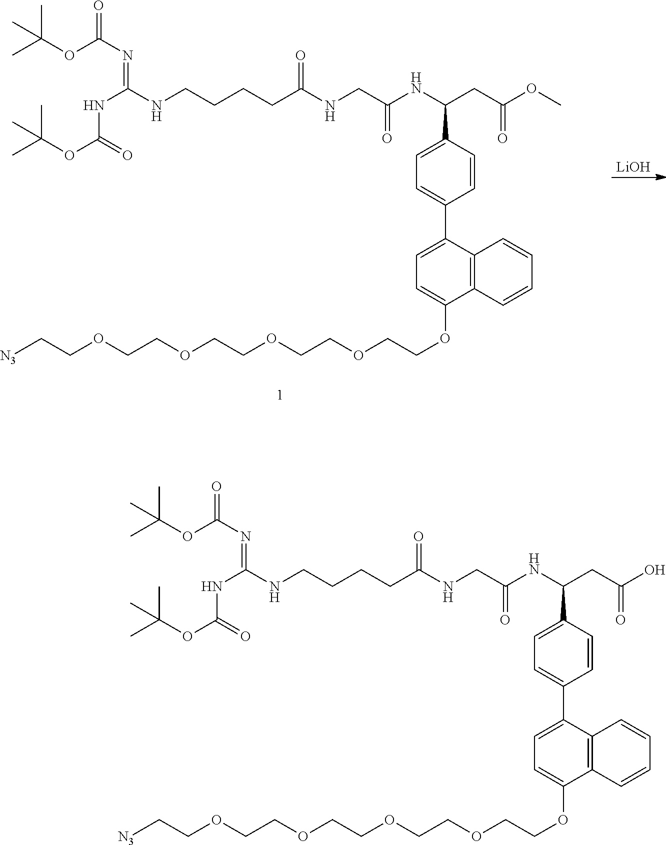

To a solution of compound 1 (120 mg, 0.124 mmol, 1.0 equiv.) in THF (2 mL) and water (2 mL) was added lithium hydroxide (9 mg, 0.373 mmol, 3.0 equiv.) at room temperature. The reaction was kept at room temperature for 2 hrs. The reaction was quenched with HCl (6.0N) and the pH was adjusted to 4.0. The mixture was extracted with ethyl acetate (3×5 mL). The organic phase was combined, dried over anhydrous Na₂SO₄, and concentrated. The product was used directly without further purification. LC-MS: calculated [M+H]+ 951.47, found 951.47.

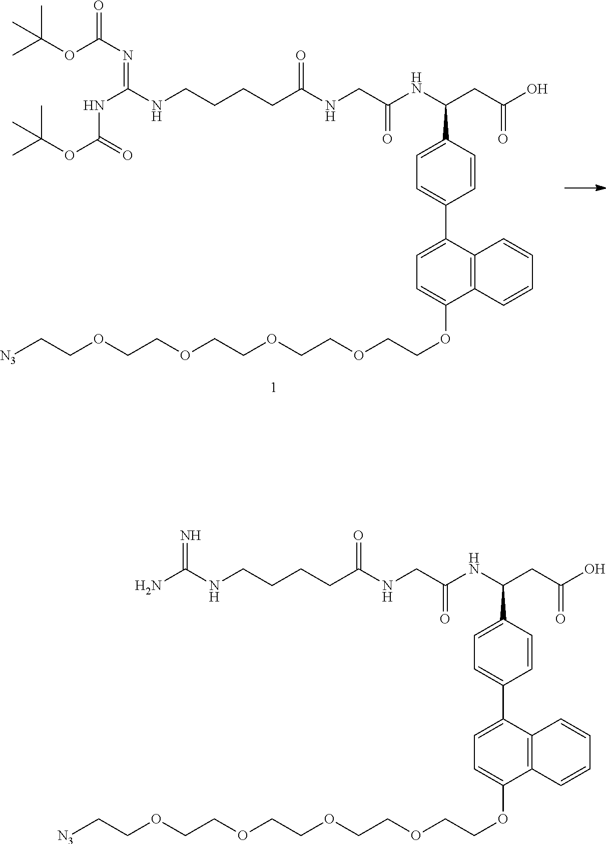 [SMILES: CC(C)(C)OC(=O)/N=C(/NCCCCC(=O)NCC(=O)N[C@@H](CC(=O)O)c1ccc(-c2ccc(OCCOCCOCCOCCOCCN=[N+]=[N-])c3ccccc23)cc1)NC(=O)OC(C)(C)C ; [N-]=[N+]=NCCOCCOCCOCCOCCOc1ccc(-c2ccc([C@H](CC(=O)O)NC(=O)CNC(=O)CCCCNC(=N)N)cc2)c2ccccc12]

To a solution of compound 1 (115 mg, 0.135 mmol, 1.0 equiv.) in dichloromethane (1 mL) was added trifluoroacetic acid (1 mL) at room temperature. The reaction was kept at room temperature for 3 hrs. The solvent was concentrated and the product was used directly without further purification. LC-MS: calculated [M+H]+ 751.37, found 751.43.

### Synthesis of Compound 43p, (S)-3-(2-((S)-2-amino-5-guanidinopentanamido)acetamido)-3-(4-(4-((14-azido-3,6,9,12-tetraoxatetradecyl)oxy)naphthalen-1-yl)phenyl)propanoic acid

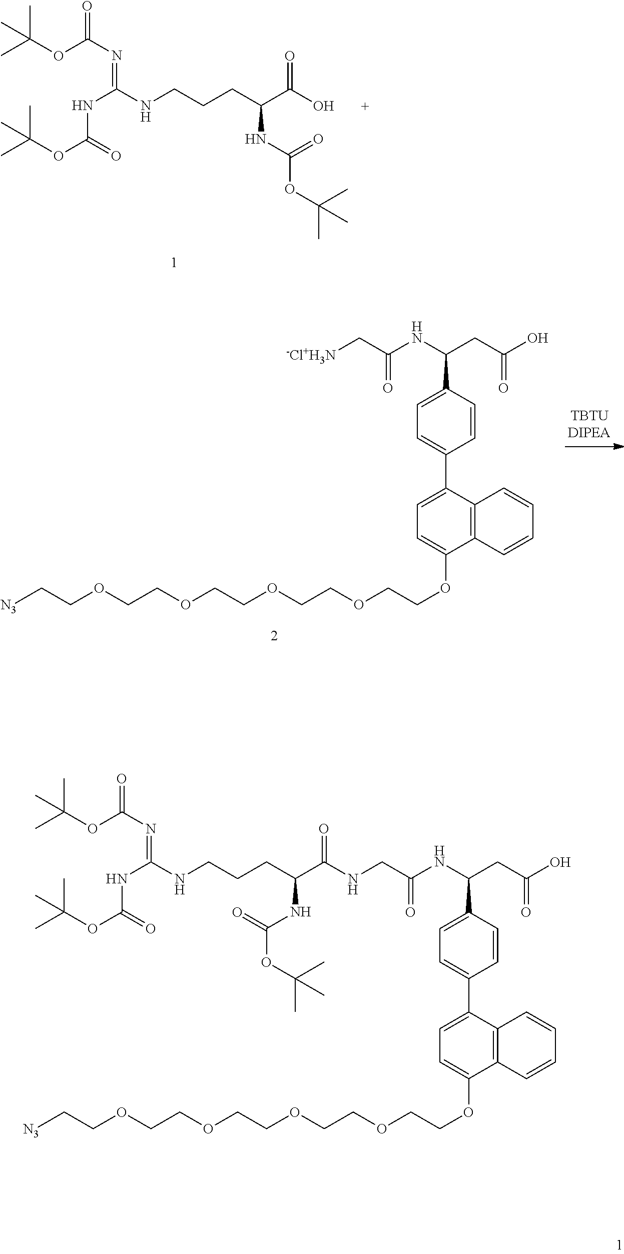

To a solution of compound 1 (72 mg, 0.151 mmol, 1.0 equiv.), compound 2 (105 mg, 0.159 mmol, 1.05 equiv.) and diisopropylethylamine (0.079 mL, 0.588 mmol, 3.0 equiv.) in anhydrous DMF (1 mL) was added TBTU (58 mg, 0.182 mmol, 1.2 equiv.) at room temperature. The reaction was kept at room temperature for 2 hrs. The reaction was quenched with saturated NaHCO₃ aqueous solution (5 mL) and the aqueous was extracted with ethyl acetate (3×5 mL). The organic phase was combined, dried over anhydrous Na₂SO₄, and concentrated. The product was purified by CombiFlash and was eluted with 2-4% methanol in dichloromethane. LC-MS: calculated [M+H]+ 1080.55, found 1080.57.

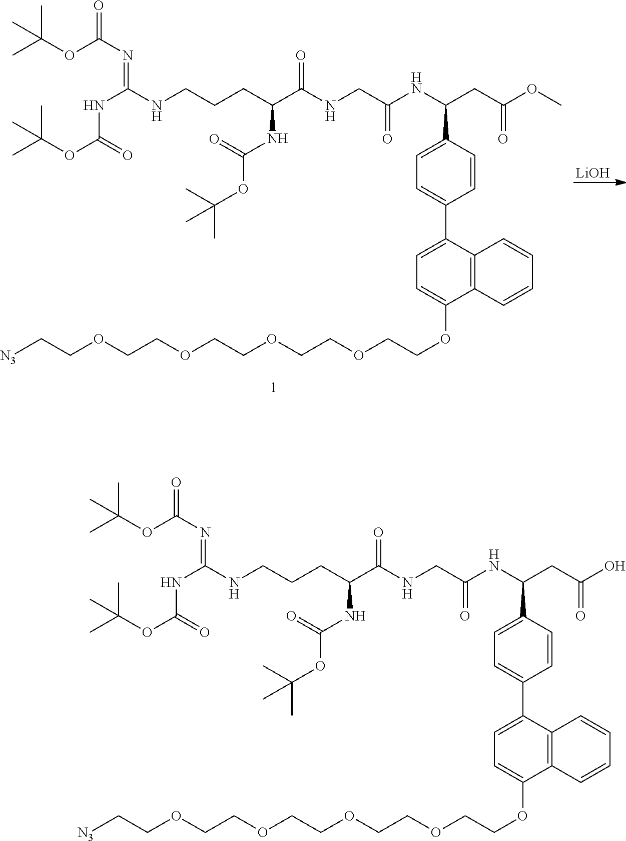

To a solution of compound 1 (100 mg, 0.926 mmol, 1.0 equiv.) in THF (2 mL) and water (2 mL) was added lithium hydroxide (7 mg, 0.277 mmol, 3.0 equiv.) at room temperature. The reaction was kept at room temperature for 2 hrs. The reaction was quenched with HCl (6.0N) and the pH was adjusted to 4.0. The mixture was extracted with ethyl acetate (3×5 mL). The organic phase was combined, dried over anhydrous Na₂SO₄, and concentrated. The product was used directly without further purification. LC-MS: calculated [M+H]+ 1066.54, found 1067.01.

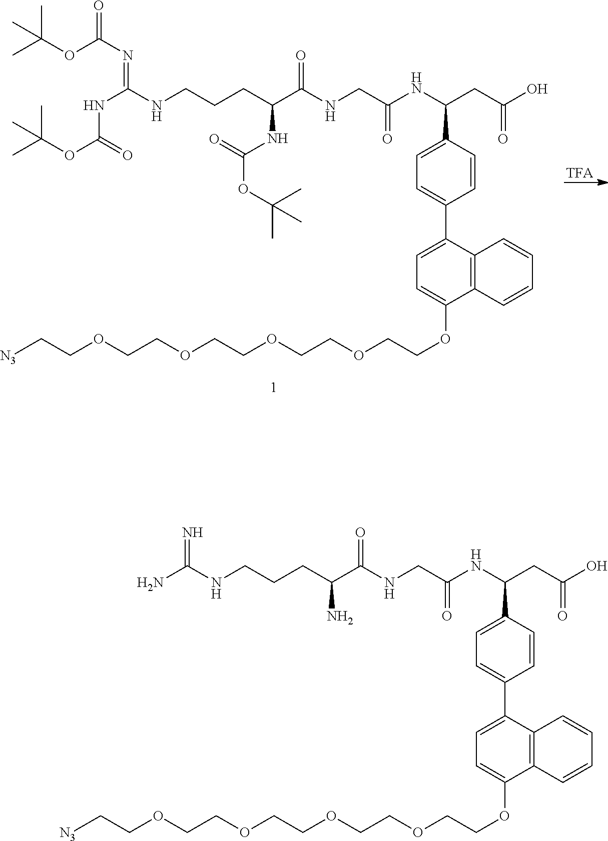

To a solution of compound 1 (100 mg, 0.0938 mmol, 1.0 equiv.) in dichloromethane (1 mL) was added trifluoroacetic acid (1 mL) at room temperature. The reaction was kept at room temperature for 3 hrs. The solvent was concentrated and the product was used directly without further purification. LC-MS: calculated [M+H]+ 766.38, found 766.55.

### Synthesis of Compound 44p, (S)-3-(2-((S)-2-acetamido-5-guanidinopentanamido)acetamido)-3-(4-(4-((14-azido-3,6,9,12-tetraoxatetradecyl)oxy)naphthalen-1-yl)phenyl)propanoic acid

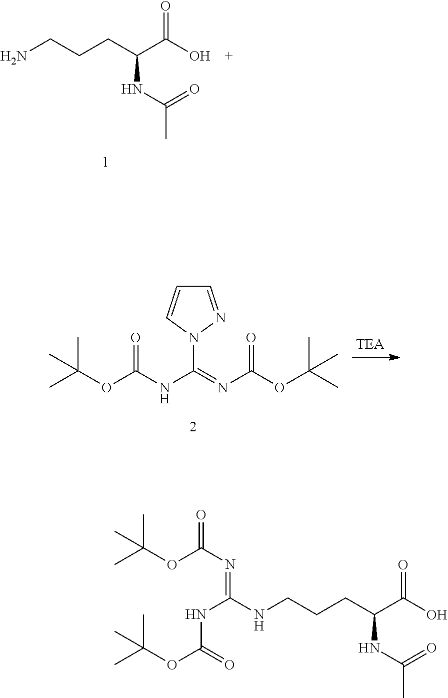

To a solution of compound 1 (500 mg, 2.870 mmol, 1.0 equiv.) and compound 2 (1.081 g, 3.587 mmol, 1.25 equiv.) in anhydrous methanol (10 mL) was added triethylamine (1.20 mL, 8.610 mmol, 3.0 equiv.) at room temperature. The reaction was kept at 40° C. for 2 hrs. The reaction mixture was concentrated and the product was separated by CombiFlash. The product was eluted with 4-6% methanol in dichloromethane. LC-MS: calculated [M+H]+ 417.23, found 417.45.

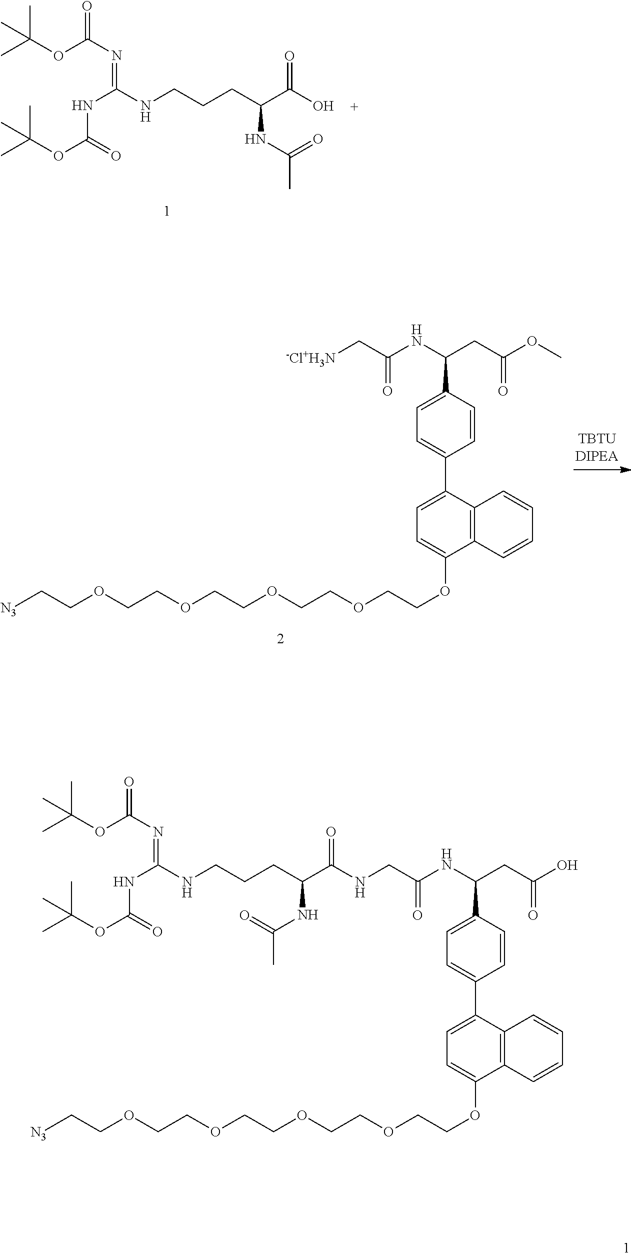

To a solution of compound 1 (66 mg, 0.158 mmol, 1.0 equiv.), compound 2 (109 mg, 0.166 mmol, 1.05 equiv.) and diisopropylethylamine (0.083 mL, 0.475 mmol, 3.0 equiv.) in anhydrous DMF (1 mL) was added TBTU (61 mg, 0.190 mmol, 1.2 equiv.) at room temperature. The reaction was kept at room temperature for 2 hrs. The reaction was quenched with saturated NaHCO₃ aqueous solution (5 mL) and the aqueous was extracted with ethyl acetate (3×5 mL). The organic phase was combined, dried over anhydrous Na₂SO₄, and concentrated. The product was purified by CombiFlash and was eluted with 2-4% methanol in dichloromethane. LC-MS: calculated [M+H]+ 1022.51, found 1022.36.

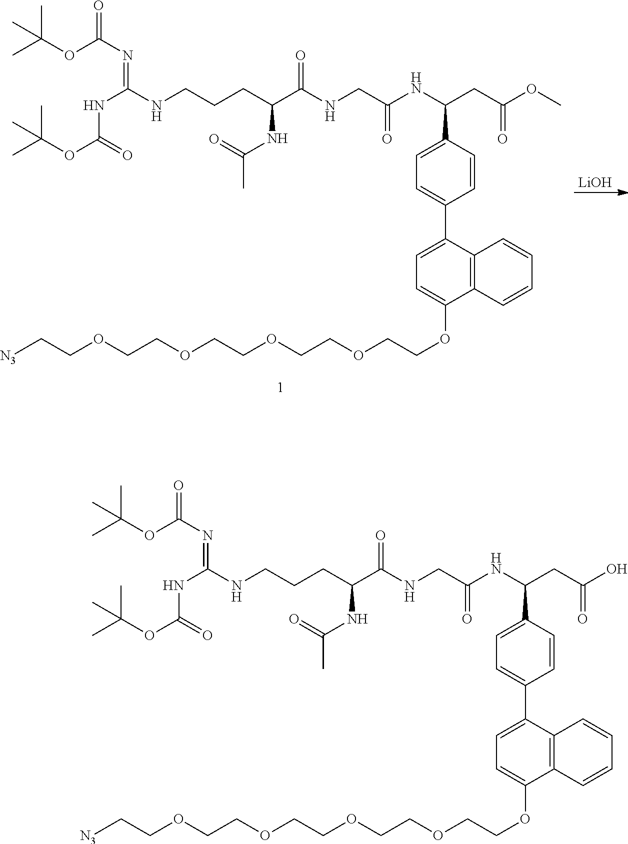

To a solution of compound 1 (125 mg, 0.122 mmol, 1.0 equiv.) in THF (2 mL) and water (2 mL) was added lithium hydroxide (9 mg, 0.366 mmol, 3.0 equiv.) at room temperature. The reaction was kept at room temperature for 2 hrs. The reaction was quenched with HCl (6.0N) and the pH was adjusted to 4.0. The mixture was extracted with ethyl acetate (3×5 mL). The organic phase was combined, dried over anhydrous Na₂SO₄, and concentrated. The product was used directly without further purification. LC-MS: calculated [M+H]+ 1008.50, found 1008.79.

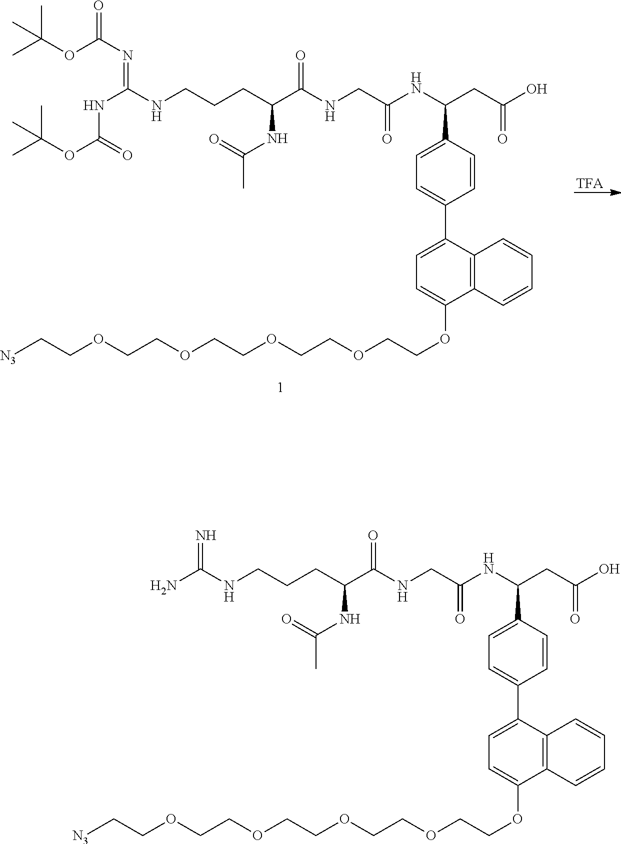

To a solution of compound 1 (120 mg, 0.119 mmol, 1.0 equiv.) in dichloromethane (1 mL) was added trifluoroacetic acid (1 mL) at room temperature. The reaction was kept at room temperature for 3 hrs. The solvent was concentrated and the product was used directly without further purification. LC-MS: calculated [M+H]+ 808.39, found 808.33.

### Synthesis of Compound 45p, (S)-3-(4-(4-((14-azido-3,6,9,12-tetraoxatetradecyl)oxy)naphthalen-1-yl)phenyl)-3-(2-(5-((4-methylpyridin-2-yl)amino)pentanamido)acetamido)propanoic acid

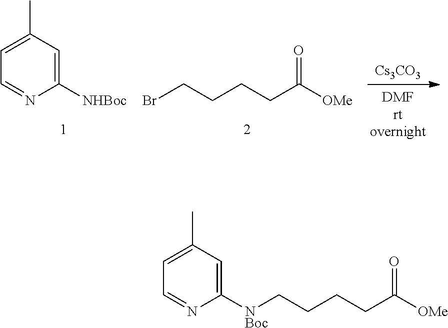

To a solution of compound 1 (0.50 g) in DMF under N₂ (g) at rt was added Cs₂CO₃ (0.94 g). Compound 2 (0.49 g) was then added slowly dropwise. The reaction was stirred overnight. Approx. 50% conversion to desired product by LC-MS was then confirmed. The reaction mixture was quenched with NaHCO₃(10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash using silica gel as the stationary phase with a gradient of hex to EtOAc (0-70%), in which product eluted at 16% B. The product was concentrated under vacuum to provide a clear oil (0.35 g, 45.0% yield). LC-MS: calculated [M+H]+ 323.19 m/z, observed 328.38 m/z.

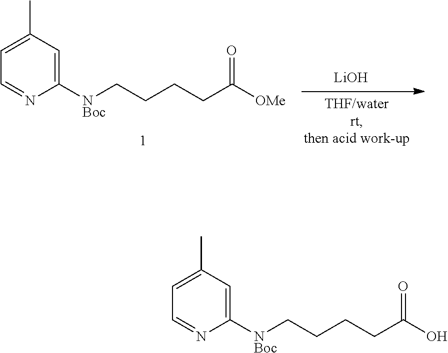 [SMILES: COC(=O)CCCCN(C(=O)OC(C)(C)C)c1cc(C)ccn1 ; "LiOH" ; Cc1ccnc(N(CCCCC(=O)O)C(=O)OC(C)(C)C)c1]

To a solution of compound 1 (0.35 g) in 1:1 THF/water was added LiOH (0.078 g) at rt under normal atmosphere. The reaction was stirred at rt until full conversion was observed by LC-MS. After 1 h, the reaction mixture was acidifed with 6 N HCl to a pH of ˜3. The product was extracted with EtOAc (3×15 mL). The combined organic phase was dried over Na₂SO₄, filtered, and concentrated, providing a clear, colorless oil (0.32 g, 94.9% yield). No isolation was necessary. LC-MS: calculated [M+H]+ 309.17 m/z, observed 309.24 m/z.

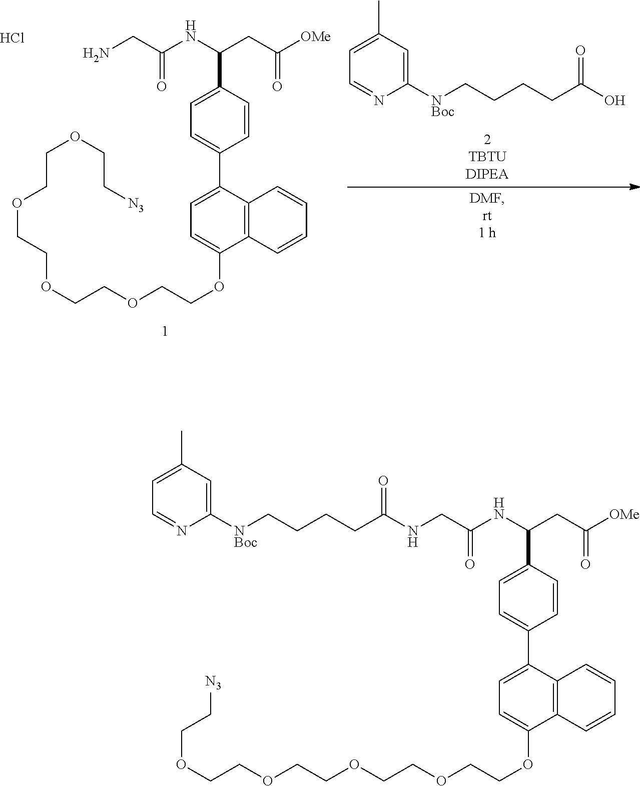

To a solution of compounds 1 (0.10 g) and 2 (0.049 g) in DMF was added TBTU (0.058 g) and then DIPEA (0.079 mL) under ambient conditions. Reaction was stirred for 1 h until full conversion was observed by LC-MS. The reaction mixture was then quenched with NaHCO₃(10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of DCM to 20% MeOH in DCM (0-70%), in which product eluted at 23% B. The product was concentrated under vacuum to provide a clear colorless oil (0.088 g, yield 63.6%.)

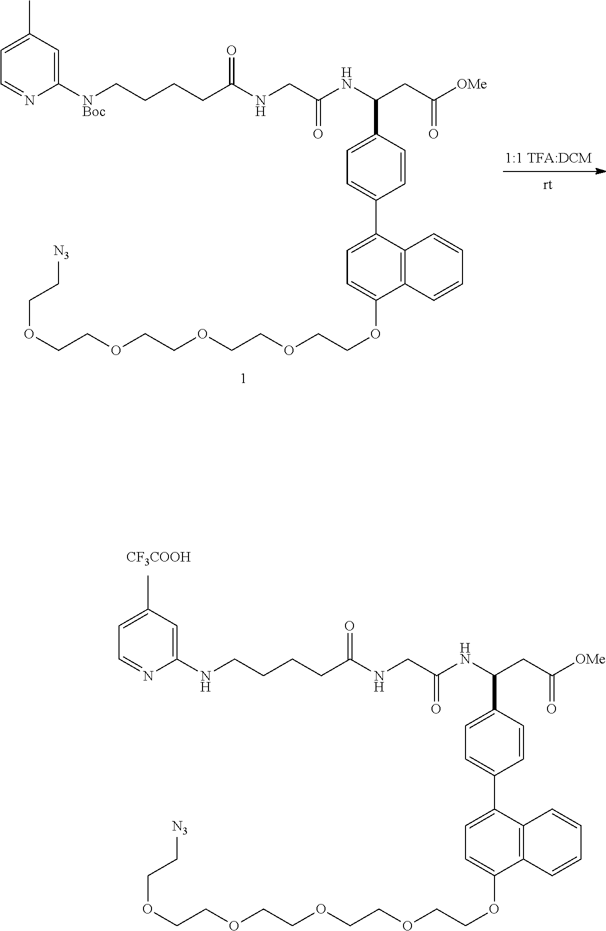 [SMILES: COC(=O)C[C@H](NC(=O)CNC(=O)CCCCN(C(=O)OC(C)(C)C)c1cc(C)ccn1)c1ccc(-c2ccc(OCCOCCOCCOCCOCCN=[N+]=[N-])c3ccccc23)cc1 ; "CF3COOH" ; COC(=O)C[C@H](NC(=O)CNC(=O)CCCCNc1cc(C)ccn1)c1ccc(-c2ccc(OCCOCCOCCOCCOCCN=[N+]=[N-])c3ccccc23)cc1]

To a solution of compound 1 (0.088 g) in DCM was added TFA (0.22 mL) at rt. The reaction was stirred under ambient conditions. Reaction was stirred for 5 h until full conversion was confirmed via LC-MS. The reaction mixture was azeotroped with PhMe and concentrated under vacuum. No isolation was necessary. Concentration provided a clear colorless oil (0.10 g, yield 113%.) LC-MS: calculated [M+H]+ 814.41 m/z, observed 814.63 m/z.

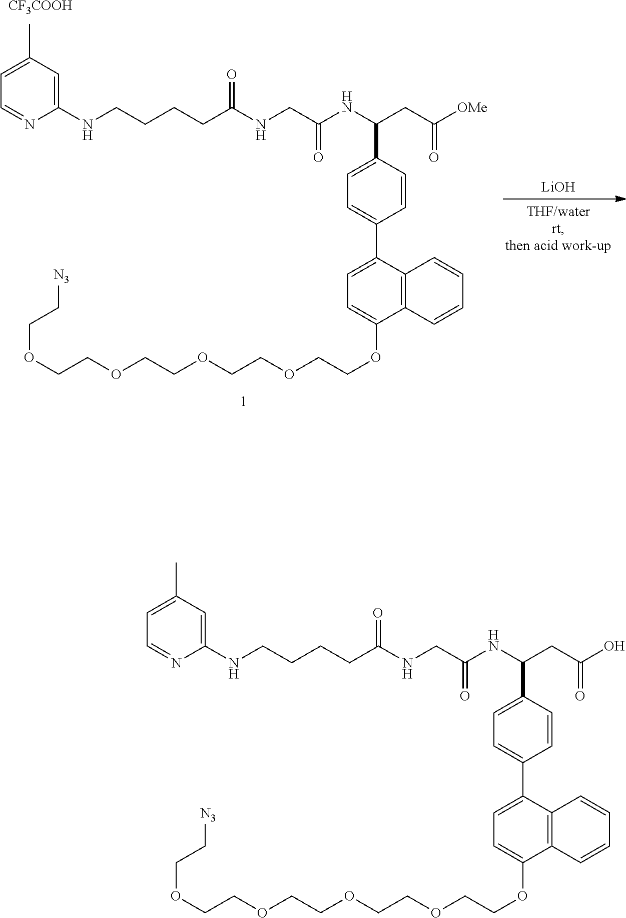 [SMILES: "CF3COOH" ; COC(=O)C[C@H](NC(=O)CNC(=O)CCCCNc1cc(C)ccn1)c1ccc(-c2ccc(OCCOCCOCCOCCOCCN=[N+]=[N-])c3ccccc23)cc1 ; "LiOH" ; Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 1 (0.10 g) in 1:1 THF/water was added LiOH (0.0078 g) at rt under normal atmosphere. The reaction was stirred at rt until full conversion was observed by LC-MS. After 4 h, the reaction mixture was acidifed with 6 N HCl to a pH of ˜3. The product was extracted with 20% CF₃CH₂OH/DCM (3×15 mL). The combined organic phase was dried over Na₂SO₄, filtered, and concentrated, providing a light yellow solid (0.104 g, yield 119%.) LC-MS: calculated [M+H]+ 800.39 m/z, observed 800.76 m/z.

### Synthesis of Compound 46p, (S)-3-(4-(4-((14-azido-3,6,9,12-tetraoxatetradecyl)oxy)naphthalen-1-yl)phenyl)-3-(2-(5-((4-methoxypyridin-2-yl)amino)pentanamido)acetamido)propanoic acid

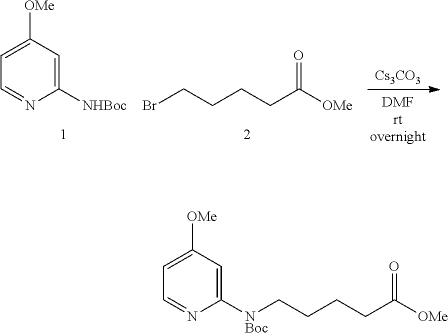

To a solution of compound 1 (0.500 g) in DMF under N₂ (g) at rt was added Cs₂CO₃ (0.872 g). Compound 2 (0.457 g) was then added slowly dropwise. The reaction was stirred overnight. Approx. 50% conversion to desired product by LC-MS was then confirmed. The reaction mixture was quenched with NaHCO₃ (10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of hex to EtOAc (0-70%), in which product eluted at 21% B. The product was concentrated under vacuum to provide a clear oil. Yield 0.191 g (25.3%.) LC-MS: calculated [M+H]+ 339.18 m/z, observed 339.31 m/z.

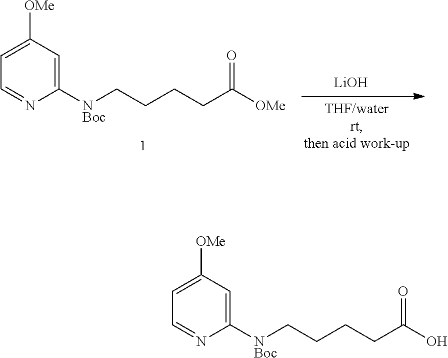 [SMILES: COC(=O)CCCCN(C(=O)OC(C)(C)C)c1cc(OC)ccn1 ; "LiOH" ; COc1ccnc(N(CCCCC(=O)O)C(=O)OC(C)(C)C)c1]

To a solution of compound 1 (0.191 g) in 1:1 THF/water was added LiOH (0.0406 g) at room temperature under normal atmosphere. The reaction was stirred at room temperature until full conversion was observed by LC-MS. After 3 h, the reaction mixture was acidifed with 6 N HCl to a pH of ˜3. The product was extracted with EtOAc (3×15 mL). The combined organic phase was dried over Na₂SO₄, filtered, and concentrated, providing a clear, colorless oil. Yield: 0.176 g (96.1%). LC-MS: calculated [M+H]+ 325.17 m/z, observed 325.27 m/z.

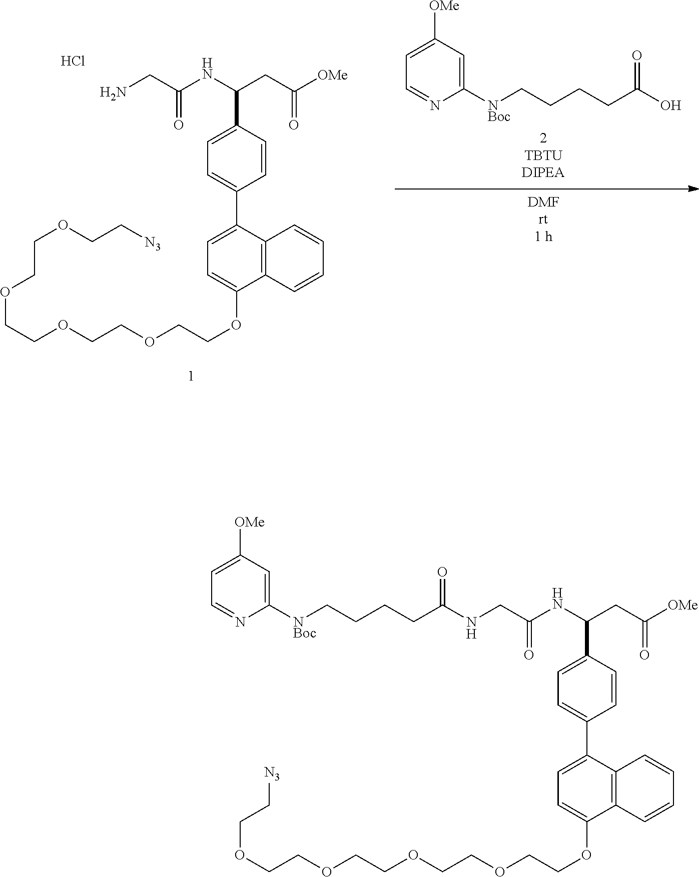 [SMILES: COC(=O)C[C@H](NC(=O)CN)c1ccc(-c2ccc(OCCOCCOCCOCCOCCN=[N+]=[N-])c3ccccc23)cc1 ; Cl ; COc1ccnc(N(CCCCC(=O)O)C(=O)OC(C)(C)C)c1 ; COC(=O)C[C@H](NC(=O)CNC(=O)CCCCN(C(=O)OC(C)(C)C)c1cc(OC)ccn1)c1ccc(-c2ccc(OCCOCCOCCOCCOCCN=[N+]=[N-])c3ccccc23)cc1]

To a solution of compounds 1 (0.100 g) and 2 (0.0516 g) in DMF was added TBTU (0.0584 g) and then DIPEA (0.0587 g) under ambient conditions. The reaction was stirred for 1 h until full conversion was observed by LC-MS. The reaction mixture was then quenched with NaHCO₃ (10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of DCM to 20% MeOH in DCM (0-75%), in which product eluted at 25% B. The product was concentrated under vacuum to provide a clear colorless oil. Yield: 0.108 g (76.7%.) LC-MS: calculated [M+H]+ 930.45 m/z, observed 930.94 m/z.

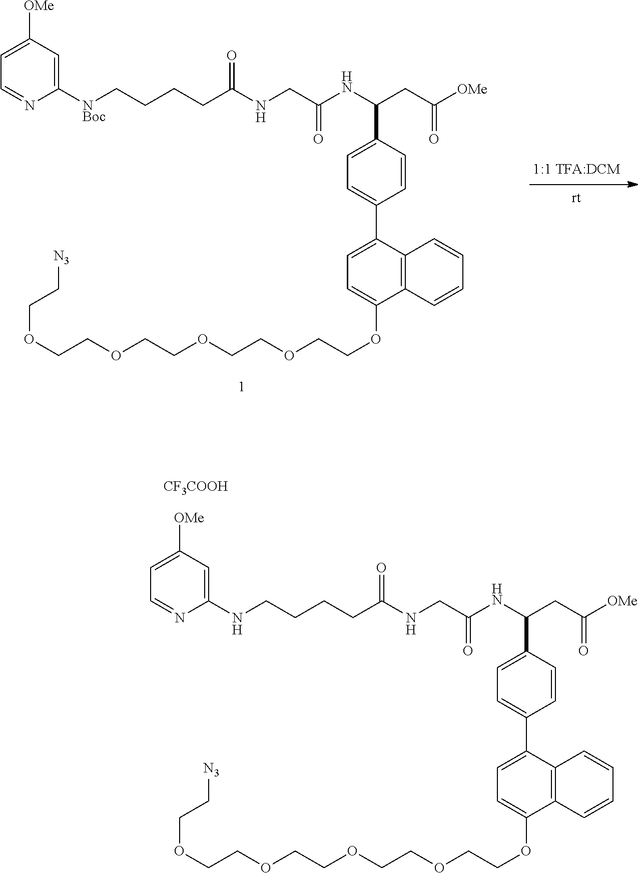 [SMILES: COC(=O)C[C@H](NC(=O)CNC(=O)CCCCN(C(=O)OC(C)(C)C)c1cc(OC)ccn1)c1ccc(-c2ccc(OCCOCCOCCOCCOCCN=[N+]=[N-])c3ccccc23)cc1 ; "CF3COOH" ; COC(=O)C[C@H](NC(=O)CNC(=O)CCCCNc1cc(OC)ccn1)c1ccc(-c2ccc(OCCOCCOCCOCCOCCN=[N+]=[N-])c3ccccc23)cc1]

To a solution of compound 1 (0.180 g) in DCM was added TFA (0.3972 g) at room temperature. The reaction was stirred under ambient conditions. Reaction was stirred for 5 h until full conversion was confirmed via LC-MS. The reaction mixture was azeotroped with PhMe and concentrated under vacuum. No isolation was necessary. Concentration provided a clear colorless oil. Yield 0.121 g (110%). LC-MS: calculated [M+H]+ 830.40 m/z, observed 830.65 m/z.

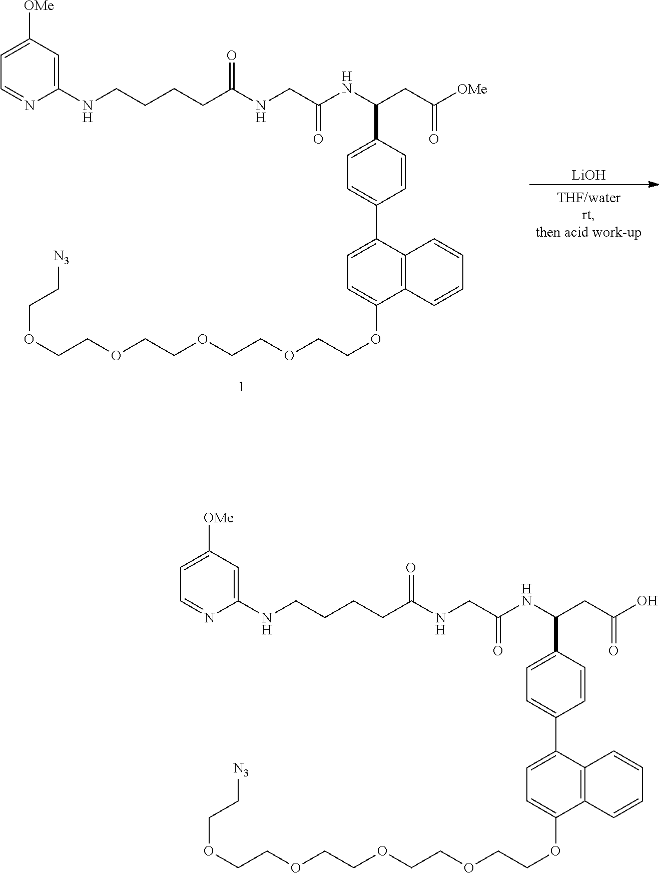 [SMILES: COC(=O)C[C@H](NC(=O)CNC(=O)CCCCNc1cc(OC)ccn1)c1ccc(-c2ccc(OCCOCCOCCOCCOCCN=[N+]=[N-])c3ccccc23)cc1 ; "LiOH" ; COc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 1 (0.121 g) in 1:1 THF/water was added LiOH (0.0092 g) at rt under normal atmosphere. The reaction was stirred at room temperature until full conversion was observed by LC-MS. After 4 h, the reaction mixture was acidifed with 6 N HCl to a pH of ˜3. The product was extracted with 20% CF₃CH₂OH/DCM (3×15 mL). The combined organic phase was dried over Na₂SO₄, filtered, and concentrated, providing a cream white solid. Yield 0.122 g (117%.) LC-MS: calculated [M+H]+ 816.39 m/z, observed 816.52 m/z.

### Synthesis of Compound 47p, (S)-3-(2-((S)-2-amino-5-ureidopentanamido)acetamido)-3-(4-(4-((14-azido-3,6,9,12-tetraoxatetradecyl)oxy)naphthalen-1-yl)phenyl)propanoic acid

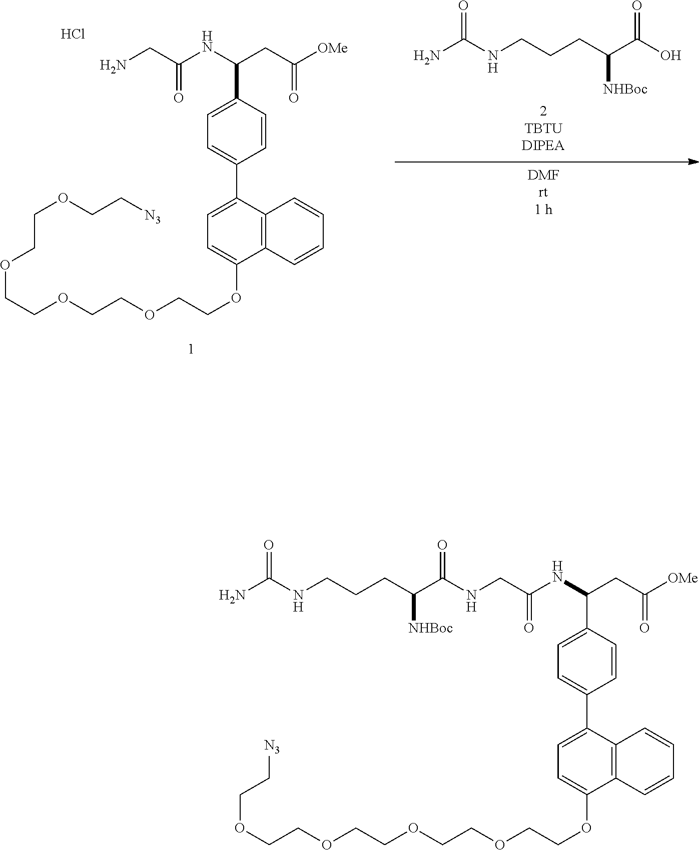 [SMILES: COC(=O)C[C@H](NC(=O)CN)c1ccc(-c2ccc(OCCOCCOCCOCCOCCN=[N+]=[N-])c3ccccc23)cc1 ; Cl ; CC(C)(C)OC(=O)N[C@@H](CCCNC(N)=O)C(=O)O ; COC(=O)C[C@H](NC(=O)CNC(=O)[C@H](CCCNC(N)=O)NC(=O)OC(C)(C)C)c1ccc(-c2ccc(OCCOCCOCCOCCOCCN=[N+]=[N-])c3ccccc23)cc1]

To a solution of compounds 1 (0.144 g) and 2 (0.0601 g) in DMF was added TBTU (0.0840 g) and then DIPEA (0.114 mL) under ambient conditions. The reaction was stirred for 1 h until full conversion was observed by LC-MS. The reaction mixture was then quenched with NaHCO₃ (10 mL). The product was extracted with 20% CF₃CH₂OH/DCM (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of DCM to 20% MeOH in DCM (0-100%), in which product eluted at 47% B. The product was concentrated under vacuum to provide a clear colorless oil. Yield 0.149 g (77.7%.) LC-MS: calculated [M+H]+881.43 m/z, observed 881.61 m/z.

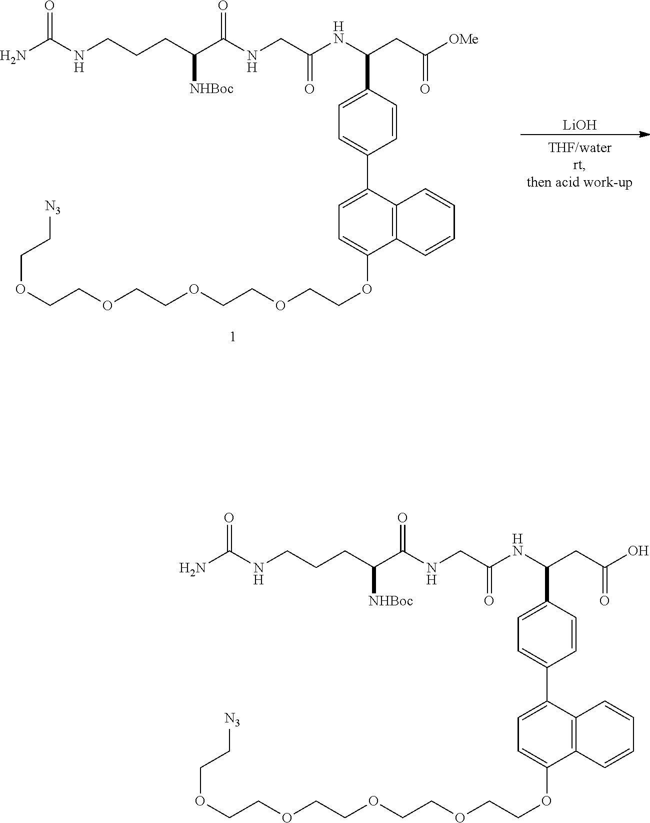 [SMILES: COC(=O)C[C@H](NC(=O)CNC(=O)[C@H](CCCNC(N)=O)NC(=O)OC(C)(C)C)c1ccc(-c2ccc(OCCOCCOCCOCCOCCN=[N+]=[N-])c3ccccc23)cc1 ; "LiOH" ; CC(C)(C)OC(=O)N[C@@H](CCCNC(N)=O)C(=O)NCC(=O)N[C@@H](CC(=O)O)c1ccc(-c2ccc(OCCOCCOCCOCCOCCN=[N+]=[N-])c3ccccc23)cc1]

To a solution of compound 1 (0.149 g) in 1:1 THF/water was added LiOH (0.0122 g) at rt under normal atmosphere. The reaction was stirred at room temperature until full conversion was observed by LC-MS. After 1 h, the reaction mixture was acidifed with 6 N HCl to a pH of ˜3. The product was extracted with 20% CF₃CH₂OH/DCM (5×10 mL). The combined organic phase was dried over Na₂SO₄, filtered, and concentrated, providing a white solid. Yield: 0.148 g (100%.) LC-MS: calculated [M+H]+ 867.42 m/z, observed 867.83 m/z.

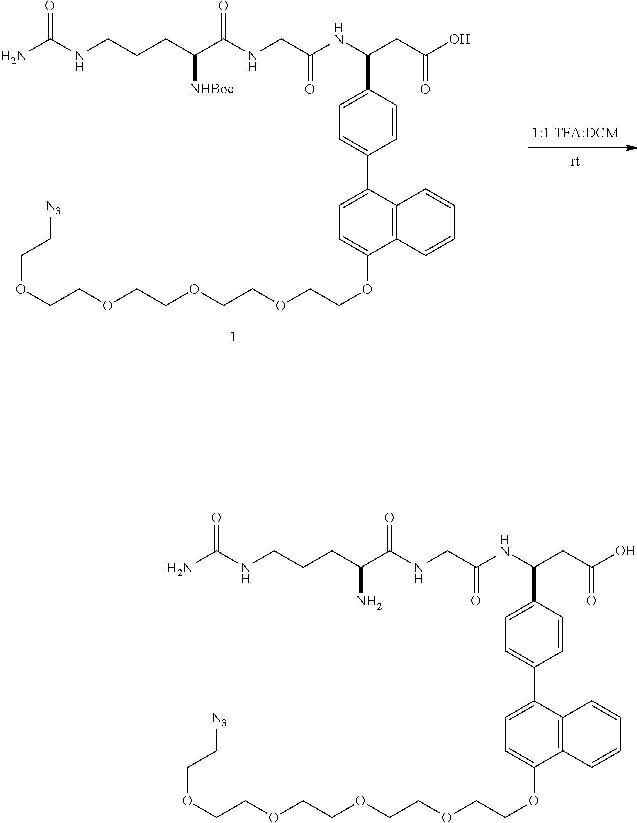 [SMILES: CC(C)(C)OC(=O)N[C@@H](CCCNC(N)=O)C(=O)NCC(=O)N[C@@H](CC(=O)O)c1ccc(-c2ccc(OCCOCCOCCOCCOCCN=[N+]=[N-])c3ccccc23)cc1 ; [N-]=[N+]=NCCOCCOCCOCCOCCOc1ccc(-c2ccc([C@H](CC(=O)O)NC(=O)CNC(=O)[C@@H](N)CCCNC(N)=O)cc2)c2ccccc12]

To a solution of compound 1 (0.148 g) in DCM was added TFA (0.392 mL) at room temperature. The reaction was stirred under ambient conditions. The reaction was stirred for 1 h until full conversion was confirmed via LC-MS. The reaction mixture was azeotroped with PhMe and concentrated under vacuum.

The mixture was found to be messy due to incomplete saponification in previous step, so the mixture was resubjected to basic conditions (LiOH, THF/water, rt) for 1 h. Upon confirmation of full conversion, mixture was acidified with 6 N HCl to a pH of ˜3, and product was extracted with EtOAc and then 20% CF₃CH₂OH/DCM and then dried over Na₂SO₄, filtered, and concentrated. Concentration provided a white solid. Yield 0.162 g (124%.) LC-MS: calculated [M+H]+ 767.36 m/z, observed 767.55 m/z.

### Synthesis of Compound 48p, (S)-3-(2-((S)-2-acetamido-5-ureidopentanamido)acetamido)-3-(4-(4-((14-azido-3,6,9,12-tetraoxatetradecyl)oxy)naphthalen-1-yl)phenyl)propanoic acid

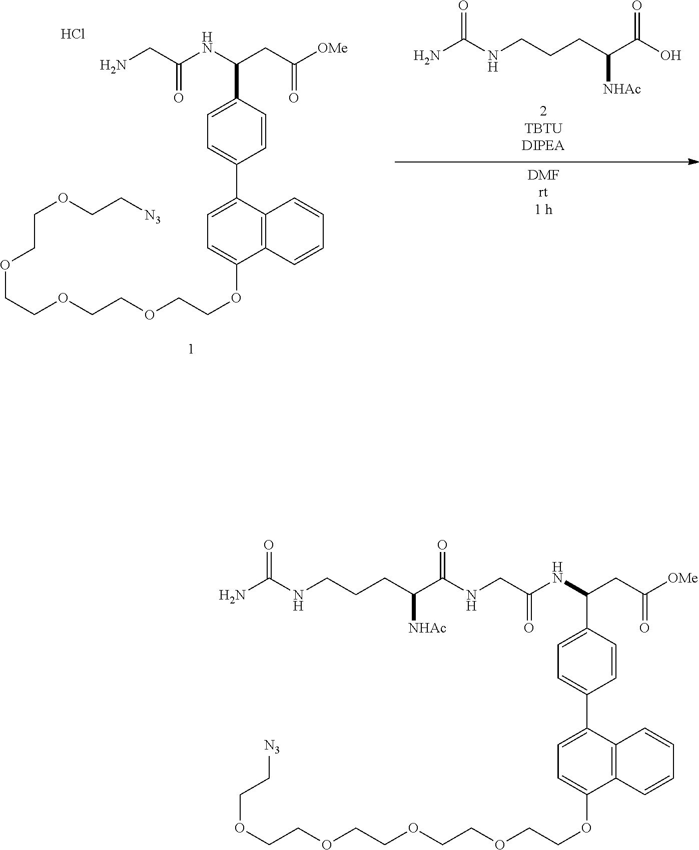 [SMILES: COC(=O)C[C@H](NC(=O)CN)c1ccc(-c2ccc(OCCOCCOCCOCCOCCN=[N+]=[N-])c3ccccc23)cc1 ; Cl ; CC(=O)N[C@@H](CCCNC(N)=O)C(=O)O ; COC(=O)C[C@H](NC(=O)CNC(=O)[C@H](CCCNC(N)=O)NC(C)=O)c1ccc(-c2ccc(OCCOCCOCCOCCOCCN=[N+]=[N-])c3ccccc23)cc1]

To a solution of compounds 1 (0.183 g) and 2 (0.0602 g) in DMF was added TBTU (0.107 g) and then DIPEA (0.145 mL) under ambient conditions. The reaction was stirred for 1 h until full conversion was observed by LC-MS. The reaction mixture was then quenched with NaHCO₃ (10 mL). The product was extracted with EtOAc and then 20% CF₃CH₂OH/DCM (3×15 mL). The combined organic phase was dried over Na₂SO₄, filtered, and concentrated. The mixture was then azeotroped with PhMe. The residue was purified by CombiFlash® using silica gel as the stationary phase with a gradient of DCM to 20% MeOH in DCM (0-100%), in which product eluted at 65% B. An impurity had eluted with product, so the residue was reisolated via a gradient of DCM to 20% MeOH in DCM (0-80%), in which product eluted from 0-70% B; however, the impurity was not able to be isolated. The product was concentrated under vacuum to provide a clear colorless oil. Yield: 0.0378 g (16.6%.) LC-MS: calculated [M+H]+ 823.39 m/z, observed 823.27 m/z.

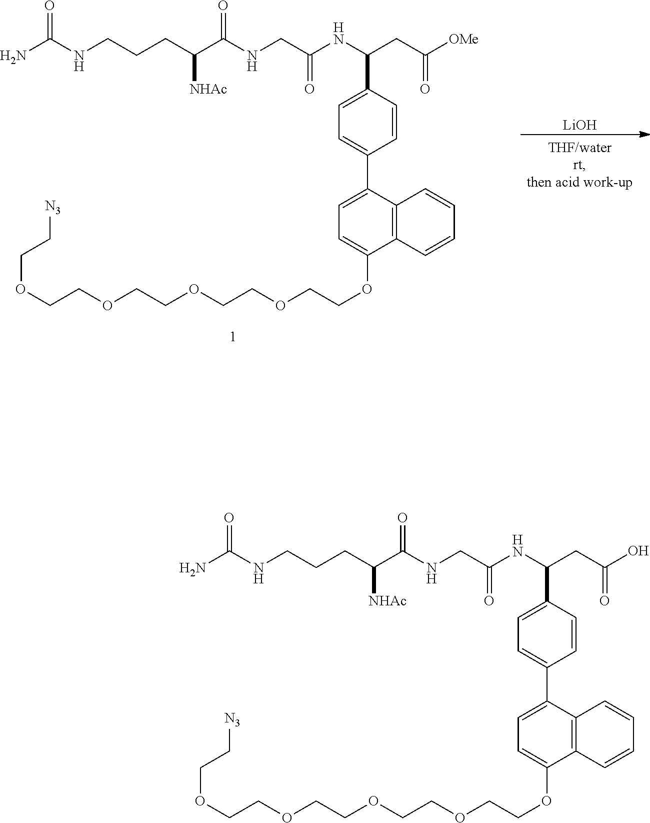 [SMILES: COC(=O)C[C@H](NC(=O)CNC(=O)[C@H](CCCNC(N)=O)NC(C)=O)c1ccc(-c2ccc(OCCOCCOCCOCCOCCN=[N+]=[N-])c3ccccc23)cc1 ; "LiOH" ; CC(=O)N[C@@H](CCCNC(N)=O)C(=O)NCC(=O)N[C@@H](CC(=O)O)c1ccc(-c2ccc(OCCOCCOCCOCCOCCN=[N+]=[N-])c3ccccc23)cc1]

To a solution of compound 1 (0.0378 g) in 1:1 THF/water was added LiOH (0.0033) at room temperature under normal atmosphere. The reaction was stirred at room temperature until full conversion was observed by LC-MS. After 1 h, the reaction mixture was acidifed with 6 N HCl to a pH of ˜3. The product was extracted with 20% CF₃CH₂OH/DCM (5×10 mL). The combined organic phase was dried over Na₂SO₄, filtered, and concentrated, providing a yellow solid. Isolation was found to be necessary. The mixture was solvated in 1 mL of DMF, and product was isolated via reverse-phase HPLC to provide a clear and colorless residue. Yield: 0.088 g (237%.) LC-MS: calculated [M+H]+ 809.38 m/z, observed 809.68 m/z.

### Synthesis of Compound 49p, (S)-3-(2-((S)-2-amino-5-((4-methylpyridin-2-yl)amino)pentanamido)acetamido)-3-(4-(4-((14-azido-3,6,9,12-tetraoxatetradecyl)oxy)naphthalen-1-yl)phenyl)propanoic acid

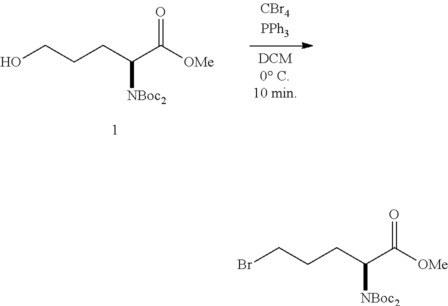

To a solution of compound 1 (0.620 g) in DCM, under N₂(g) at 0° C. in ice-water bath, was added CBr₄ (0.680 g); the mixture was stirred on ice for 15 min. Then PPh₃ (0.538 g) was added, and reaction was stirred for 10 min., after which full conversion to the desired product was observed by LC-MS; a clean mixture of desired pdt, O═PPh3, and other PPh3-based by-product was observed. The reaction mixture was then quenched with NaHCO₃ (10 mL). The product was extracted with DCM (3×10 mL) and then washed with brine (10 mL). The combined organic phase was dried over Na₂SO₄, filtered, and concentrated. The residue was purified by CombiFlash® using silica gel as the stationary phase with a gradient of hex to EtOAc (0-30%), in which product eluted at 8.5% B. The product was concentrated under vacuum, providing a clear colorless oil. Yield: 0.597 g (81.6%.) LC-MS: calculated [M+H]+ 410.11 m/z, observed 410.43 m/z.

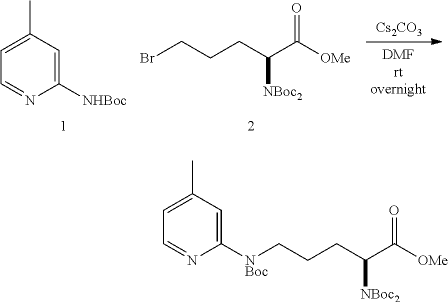

To a solution of compounds 1 (0.134 g) and 2 (0.238 g) in DMF at room temperature was added Cs₂CO₃ (0.315 g). The reaction was stirred overnight. Approx. 50% conversion to desired product by LC-MS was then confirmed. The reaction mixture was quenched with NaHCO₃ (10 mL). The product was extracted with DCM (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of hexanes to EtOAc (0-70%), in which product eluted at 15% B. The product was concentrated under vacuum to provide a clear oil. Yield: 0.196 g (56.6%.) LC-MS: calculated [M+H]+ 538.31 m/z, observed 538.44 m/z.

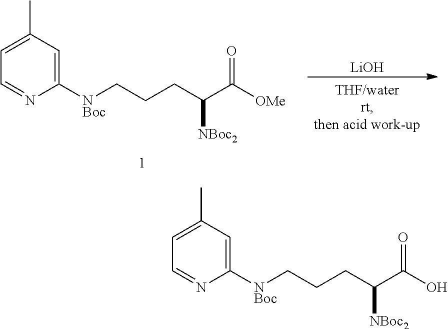

To a solution of compound 1 (0.196 g) in 1:1 THF/water was added LiOH (0.262 g) at room temperature under normal atmosphere. The reaction was stirred at room temperature until full conversion was observed by LC-MS. After 7 h with low conversion, the reaction mixture was heated to 30° C. and stirred overnight. Once full conversion was confirmed by LC-MS, the reaction mixture was slowly acidifed with 6 N HCl to a pH of ˜5. The product was extracted with EtOAc (3×15 mL). The combined organic phase was dried over Na₂SO₄, filtered, and concentrated, providing a clear, colorless oil. Yield: 0.186 g (97.1%.) LC-MS: calculated [M+H]+ 524.29 m/z, observed 524.67 m/z.

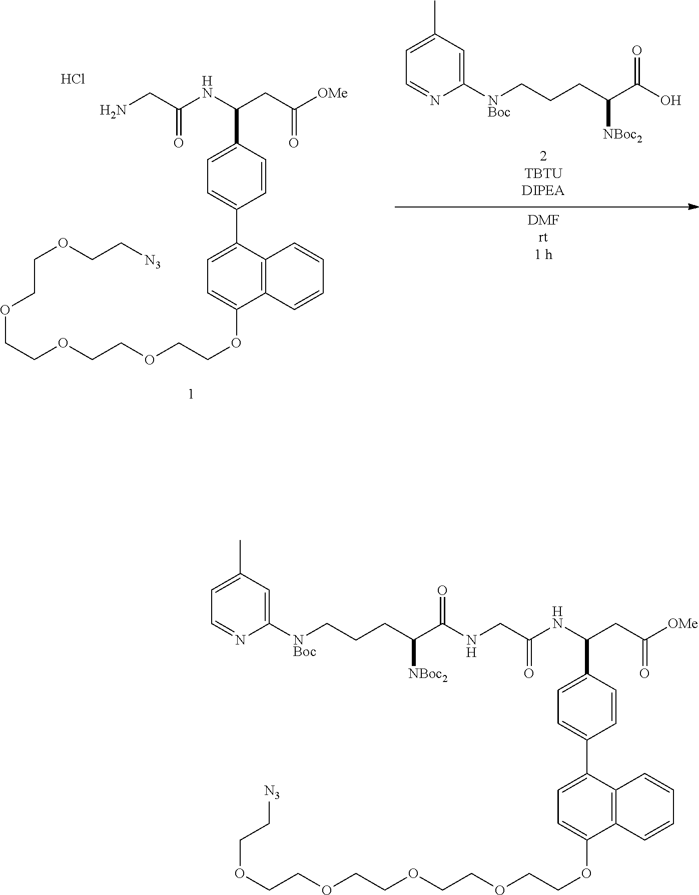

To a solution of compounds 1 (0.246 g) and 2 (0.185 g) in DMF was added TBTU (0.1436 g) and then DIPEA (0.195 mL) under ambient conditions. The reaction was stirred for 1 h until full conversion was observed by LC-MS. The reaction mixture was then quenched with NaHCO₃ (10 mL). The product was extracted with EtOAc and then 20% CF₃CH₂OH/DCM (3×15 mL), and then washed with water (3×10 mL). The combined organic phase was dried over Na₂SO₄, filtered, and concentrated. The residue was purified by CombiFlash® using silica gel as the stationary phase with a gradient of DCM to 20% MeOH in DCM (0-60%), in which product eluted at 13-26% B. The product was concentrated under vacuum to provide a clear colorless oil. Product appears to contain a mixture of desired product and mono-Boc-deprotected product. Yield: 0.212 g (50.3%.) LC-MS: calculated [M+H]+ 1129.57 m/z, observed 1130.02 m/z.

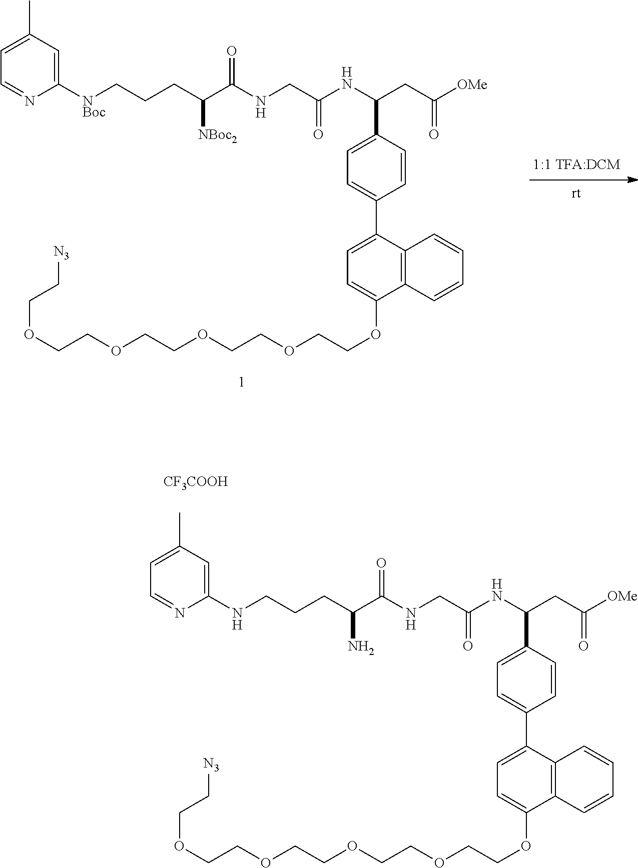

To a solution of compound 1 (0.0636 g) in DCM was added TFA (0.129 mL) at room temperature. The reaction was stirred under ambient conditions. After 6 h, full conversion was confirmed via LC-MS. The reaction mixture was azeotroped with PhMe and concentrated under vacuum. No isolation was necessary. Concentration provided a sticky yellow residue. Yield: 0.0686 g (129%.) LC-MS: calculated [M+H]+ 829.42 m/z, observed 829.57 m/z.

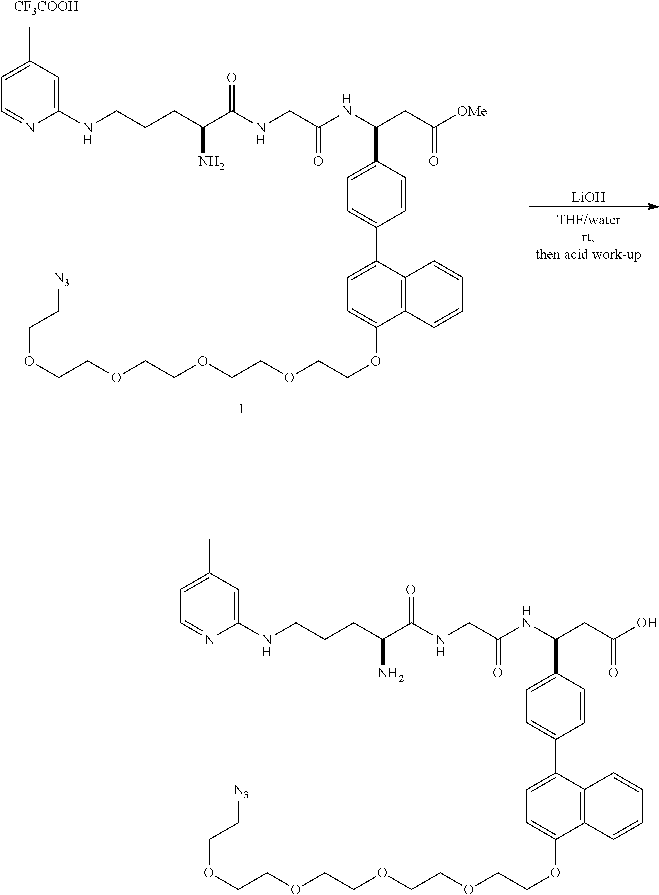 [SMILES: "CF3COOH" ; COC(=O)C[C@H](NC(=O)CNC(=O)[C@@H](N)CCCNc1cc(C)ccn1)c1ccc(-c2ccc(OCCOCCOCCOCCOCCN=[N+]=[N-])c3ccccc23)cc1 ; "LiOH" ; Cc1ccnc(NCCC[C@H](N)C(=O)NCC(=O)N[C@@H](CC(=O)O)c2ccc(-c3ccc(OCCOCCOCCOCCOCCN=[N+]=[N-])c4ccccc34)cc2)c1]

To a solution of compound 1 (0.0250 g) in 1:1 DMF/water was added LiOH (0.0019 g) at room temperature under normal atmosphere. The reaction was stirred at room temperature for 3 h, 40° C. for 3-4 h, and then room temperature overnight. The following day, the reaction was stirred at 40° C. until full conversion was observed by LC-MS. The reaction mixture was then acidifed with 6 N HCl to a pH of ˜7. The mixture was concentrated to 2 mL of solution and isolated via reverse-phase HPLC. The product was then concentrated, providing a clear and colorless residue. Yield 0.0111 g (51.4%.) LC-MS: calculated [M+H]+ 815.40 m/z, observed 815.98 m/z.

### Synthesis of Compound 50p, (S)-3-(2-((S)-2-acetamido-5-((4-methylpyridin-2-yl)amino)pentanamido)acetamido)-3-(4-(4-((14-azido-3,6,9,12-tetraoxatetradecyl)oxy)naphthalen-1-yl)phenyl)propanoic acid

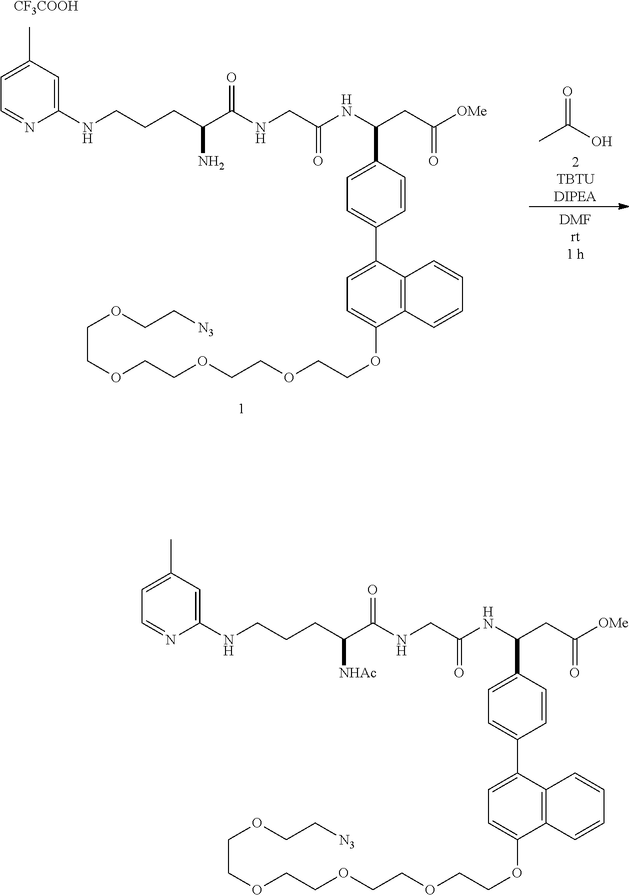

To a solution of compounds 1 (0.0350 g) and 2 (0.0022 g) in DMF was added TBTU (0.0143 g) and then DIPEA (0.019 mL) under ambient conditions. The reaction was stirred for 2 h until full conversion was observed by LC-MS. The reaction mixture was then quenched with NaHCO₃ (10 mL). The product was extracted with EtOAc and then 20% CF₃CH₂OH/DCM (3×15 mL) and then washed with water (3×10 mL). The combined organic phase was dried over Na₂SO₄, filtered, and concentrated. The residue was purified by CombiFlash® using silica gel as the stationary phase with a gradient of DCM to 20% MeOH in DCM (0-80%), in which product eluted at 47% B. The product was concentrated under vacuum to provide a clear colorless residue. Yield: 0.0126 g (39.0%.) LC-MS: calculated [M+H]+ 871.43 m/z, observed 872.33 m/z.

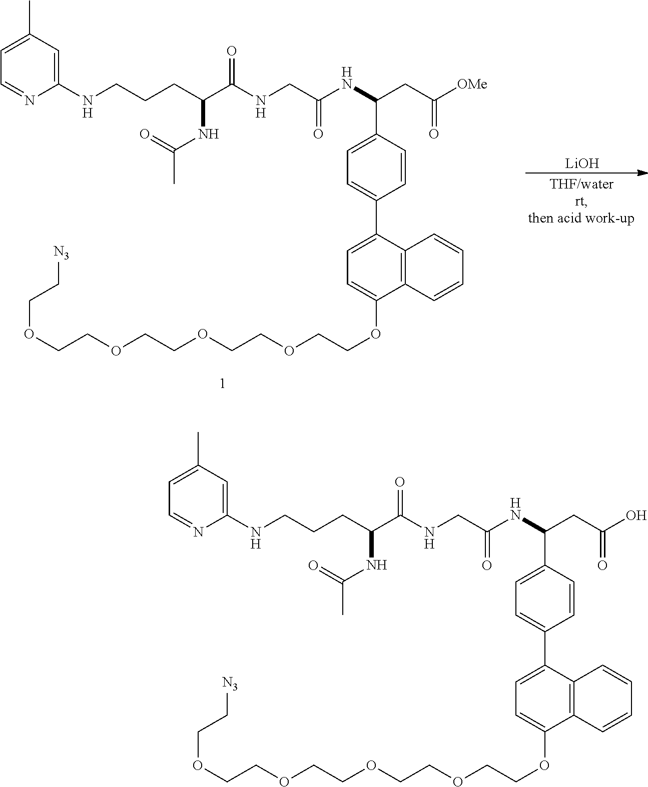 [SMILES: COC(=O)C[C@H](NC(=O)CNC(=O)[C@H](CCCNc1cc(C)ccn1)NC(C)=O)c1ccc(-c2ccc(OCCOCCOCCOCCOCCN=[N+]=[N-])c3ccccc23)cc1 ; "LiOH" ; CC(=O)N[C@@H](CCCNc1cc(C)ccn1)C(=O)NCC(=O)N[C@@H](CC(=O)O)c1ccc(-c2ccc(OCCOCCOCCOCCOCCN=[N+]=[N-])c3ccccc23)cc1]

To a solution of compound 1 (0.0126 g) in 1:1 THF/water was added LiOH (0.0010 g) at room temperature under normal atmosphere. The reaction was stirred at room temperature until full conversion was observed by LC-MS. After 1 h, the reaction mixture was acidifed with 6 N HCl to a pH of ˜4. The product was extracted with EtOAc and then 20% CF₃CH₂OH/DCM (3×10 mL) and washed with water (3×5 mL) and brine (1×5 mL). The combined organic phase was dried over Na₂SO₄, filtered, and concentrated, providing a honey-colored residue. No isolation was necessary. Yield: 0.166 g (134%.) LC-MS: calculated [M+H]+ 857.41 m/z, observed 857.21 m/z.

### Synthesis of Compound 51p, (S)-3-(4-(4-((17-azido-3,6,9,12,15-pentaoxaheptadecyl)carbamoyl)naphthalen-1-yl)phenyl)-3-(2-(4-((4-methylpyridin-2-yl)amino)butanamido)acetamido)propanoic acid

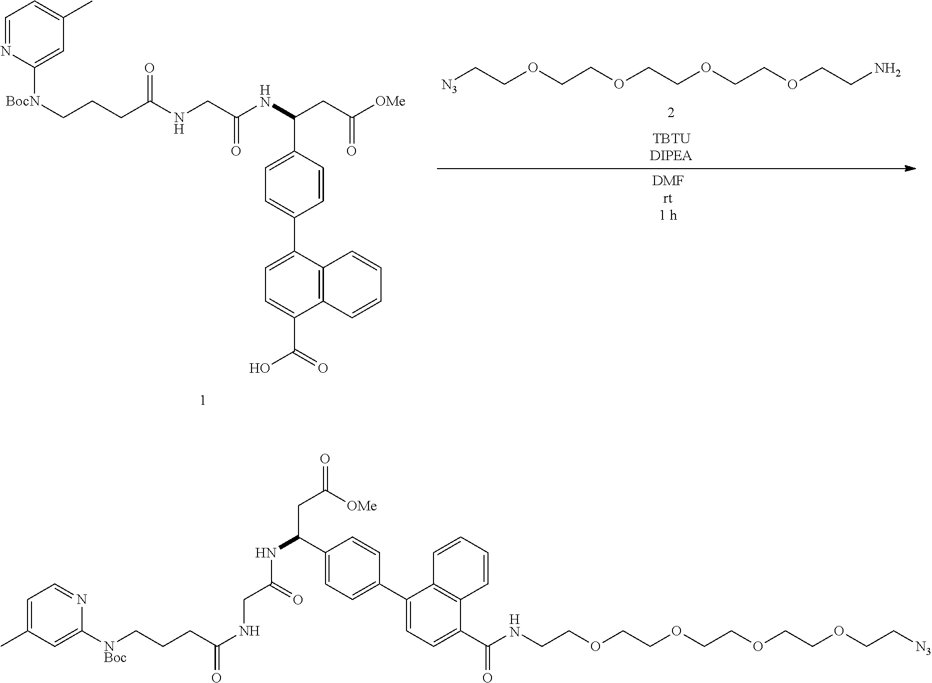 [SMILES: COC(=O)C[C@H](NC(=O)CNC(=O)CCCN(C(=O)OC(C)(C)C)c1cc(C)ccn1)c1ccc(-c2ccc(C(=O)O)c3ccccc23)cc1 ; [N-]=[N+]=NCCOCCOCCOCCOCCN ; COC(=O)C[C@H](NC(=O)CNC(=O)CCCN(C(=O)OC(C)(C)C)c1cc(C)ccn1)c1ccc(-c2ccc(C(=O)NCCOCCOCCOCCOCCN=[N+]=[N-])c3ccccc23)cc1]

To a solution of compounds 1 (0.0250 g) and 2 (0.0118 g) in DMF was added TBTU (0.0141 g) and then DIPEA (0.019 mL) under ambient conditions. The reaction was stirred for 3 h until full conversion was observed by LC-MS. The reaction mixture was then quenched with NaHCO₃ (10 mL). The product was extracted with EtOAc and then 20% CF₃CH₂OH/DCM (3×15 mL) and then washed with water (3×10 mL). The combined organic phase was dried over Na₂SO₄, filtered, and concentrated. The residue was purified by CombiFlash® using silica gel as the stationary phase with a gradient of DCM to 20% MeOH in DCM (0-80%), in which product eluted at 36% B. The product was concentrated under vacuum to provide a clear colorless oil. Yield: 0.0233 g (65.5%.) LC-MS: calculated [M+H]+ 971.48 m/z, observed 971.99 m/z.

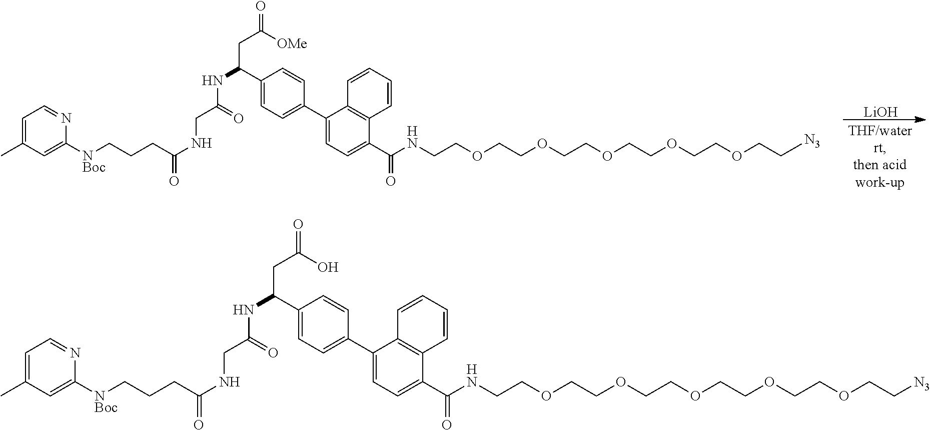 [SMILES: COC(=O)C[C@H](NC(=O)CNC(=O)CCCN(C(=O)OC(C)(C)C)c1cc(C)ccn1)c1ccc(-c2ccc(C(=O)NCCOCCOCCOCCOCCOCCN=[N+]=[N-])c3ccccc23)cc1 ; "LiOH" ; Cc1ccnc(N(CCCC(=O)NCC(=O)N[C@@H](CC(=O)O)c2ccc(-c3ccc(C(=O)NCCOCCOCCOCCOCCOCCN=[N+]=[N-])c4ccccc34)cc2)C(=O)OC(C)(C)C)c1]

To a solution of compound 1 (0.0233 g) in 1:1 DMF/water was added LiOH (0.0017 g) at rt under normal atmosphere. The reaction was stirred at room temperature for 1 h until full conversion was observed by LC-MS. The reaction mixture was then acidifed with 6 N HCl to a pH of ˜4, extracted with EtOAc and then 20% CF₃CH₂OH/DCM (5×8 mL), and then washed with water and brine (3×8 mL). The product was then concentrated, providing a white solid. Yield: 0.0281 g (123%.) LC-MS: calculated [M+H]+ 957.46 m/z, observed 957.86 m/z.

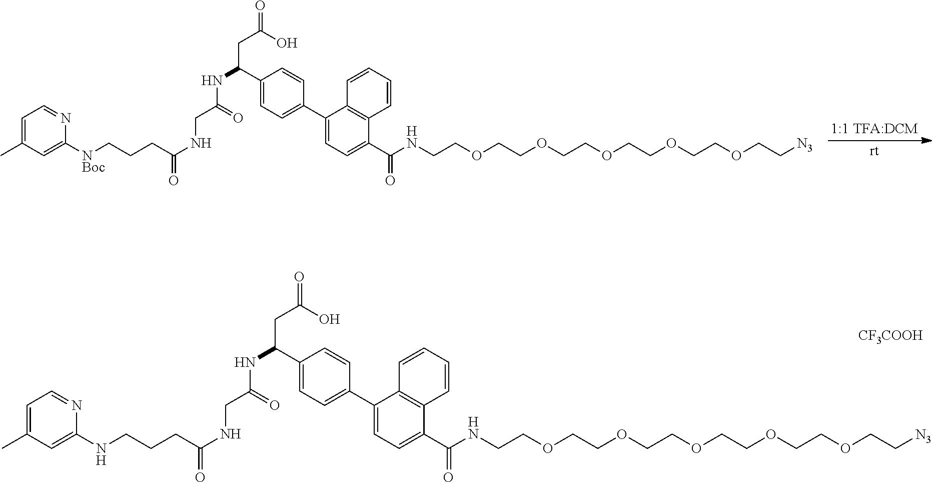 [SMILES: Cc1ccnc(N(CCCC(=O)NCC(=O)N[C@@H](CC(=O)O)c2ccc(-c3ccc(C(=O)NCCOCCOCCOCCOCCOCCN=[N+]=[N-])c4ccccc34)cc2)C(=O)OC(C)(C)C)c1 ; Cc1ccnc(NCCCC(=O)NCC(=O)N[C@@H](CC(=O)O)c2ccc(-c3ccc(C(=O)NCCOCCOCCOCCOCCOCCN=[N+]=[N-])c4ccccc34)cc2)c1 ; "CF3COOH"]

To a solution of compound 1 (0.0281 g) in DCM was added TFA (0.067 mL) at rt. The reaction was stirred under ambient conditions. After 2 h, full conversion was confirmed via LC-MS. The reaction mixture was azeotroped with PhMe and concentrated under vacuum. No isolation was necessary. Concentration provided a clear and colorless residue. Yield: 0.0415 (146%.) LC-MS: calculated [M+H]+ 857.41 m/z, observed 857.39 m/z.

### Synthesis of Compound 52p, (S)-3-(4-(4-(((S)-1-azido-22-methyl-19-oxo-3,6,9,12,15-pentaoxa-18-azatricosan-20-yl)carbamoyl)naphthalen-1-yl)phenyl)-3-(2-(4-((4-methylpyridin-2-yl)amino)butanamido)acetamido)propanoic acid

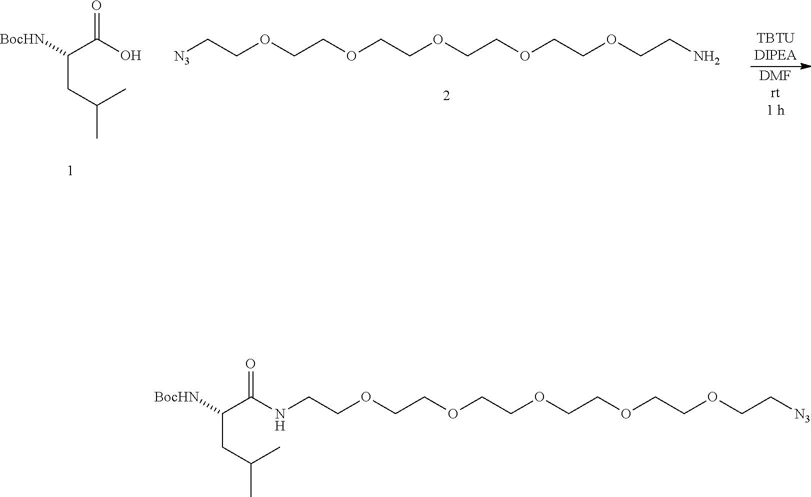 [SMILES: CC(C)C[C@H](NC(=O)OC(C)(C)C)C(=O)O ; [N-]=[N+]=NCCOCCOCCOCCOCCOCCN ; CC(C)C[C@H](NC(=O)OC(C)(C)C)C(=O)NCCOCCOCCOCCOCCOCCN=[N+]=[N-]]

To a solution of compounds 1 (0.162 g) and 2 (0.225 g) in DMF was added TBTU (0.270 g) and then DIPEA (0.366 mL) under ambient conditions. The reaction was stirred for 1 h until full conversion was observed by LC-MS. The reaction mixture was then quenched with NaHCO₃ (10 mL). The product was extracted with EtOAc (3×15 mL) and then washed with water (3×10 mL). The combined organic phase was dried over Na₂SO₄, filtered, and concentrated. The residue was purified by CombiFlash® using silica gel as the stationary phase with a gradient of hex to EtOAc (0-100%), in which product eluted at 100% B. The product was concentrated under vacuum to provide a clear colorless oil. Yield: 0.245 g (67.4%.) LC-MS: calculated [M+H]+ 520.33 m/z, observed 520.61 m/z.

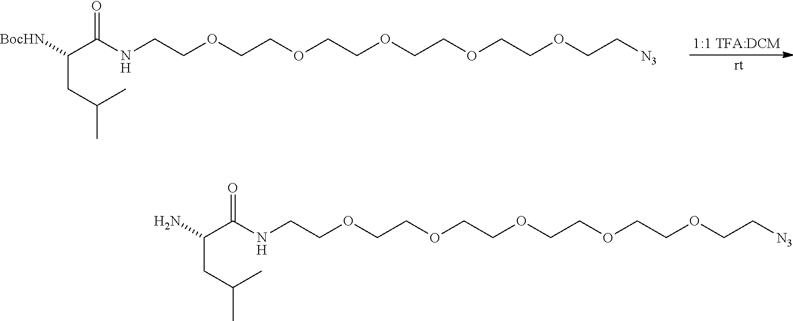 [SMILES: CC(C)C[C@H](NC(=O)OC(C)(C)C)C(=O)NCCOCCOCCOCCOCCOCCN=[N+]=[N-] ; CC(C)C[C@H](N)C(=O)NCCOCCOCCOCCOCCOCCN=[N+]=[N-]]

To a solution of compound 1 (0.245 g) in DCM was added TFA (1.08 mL) at room temperature. The reaction was stirred under ambient conditions. After 1 h, full conversion was confirmed via LC-MS. The reaction mixture was azeotroped with PhMe and subjected to a base extraction with NaHC03. Product was extracted with EtOAc and then 20% CF₃CH₂OH/DCM and then washed with water and brine. Mixture was then concentrated under vacuum. No isolation was necessary. Concentration provided a white solid. Yield: 0.224 (113%.) LC-MS: calculated [M+H]+ 420.27 m/z, observed 420.51 m/z.

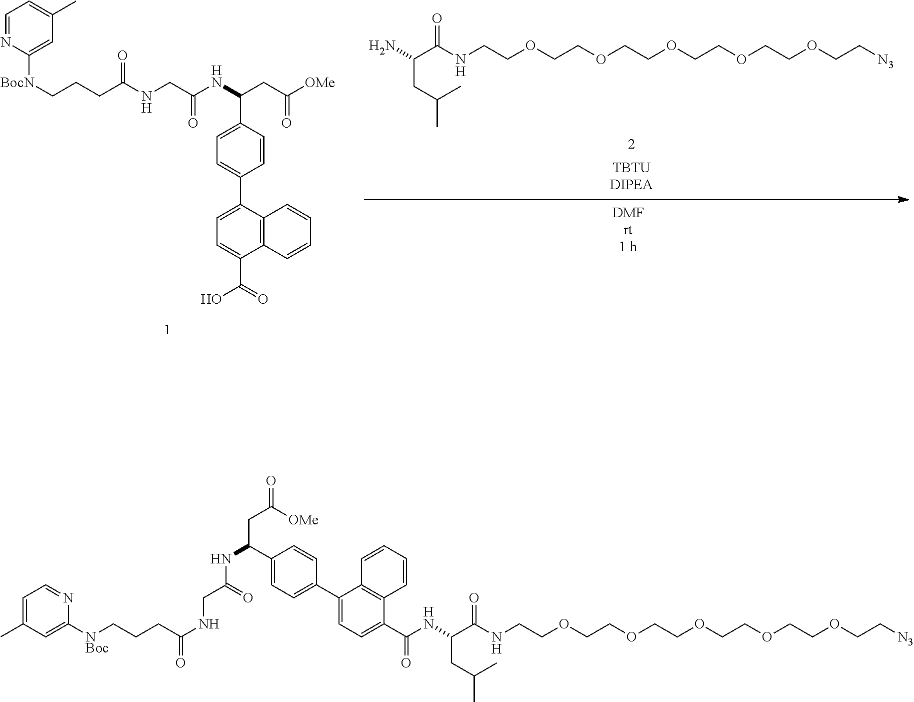 [SMILES: COC(=O)C[C@H](NC(=O)CNC(=O)CCCN(C(=O)OC(C)(C)C)c1cc(C)ccn1)c1ccc(-c2ccc(C(=O)O)c3ccccc23)cc1 ; CC(C)C[C@H](N)C(=O)NCCOCCOCCOCCOCCOCCN=[N+]=[N-] ; COC(=O)C[C@H](NC(=O)CNC(=O)CCCN(C(=O)OC(C)(C)C)c1cc(C)ccn1)c1ccc(-c2ccc(C(=O)N[C@@H](CC(C)C)C(=O)NCCOCCOCCOCCOCCOCCN=[N+]=[N-])c3ccccc23)cc1]

To a solution of compounds 1 (0.440 g) and 2 (0.270 g) in DMF was added TBTU (0.0248 g) and then DIPEA (0.034 mL) under ambient conditions. The reaction was stirred for 3 h until full conversion was observed by TLC. Due to scale, reaction mixture was concentrated and then resolvated in EtOAc and concentrated over silica for isolation. The residue was purified by CombiFlash® using silica gel as the stationary phase with a gradient of DCM to 20% MeOH in DCM (0-50%), in which product eluted at 18% B. The product was concentrated under vacuum to provide a clear colorless oil. Yield: 0.0475 g (68.0%.) LC-MS: calculated [M+H]+ 1084.56 m/z, observed 1085.17 m/z.

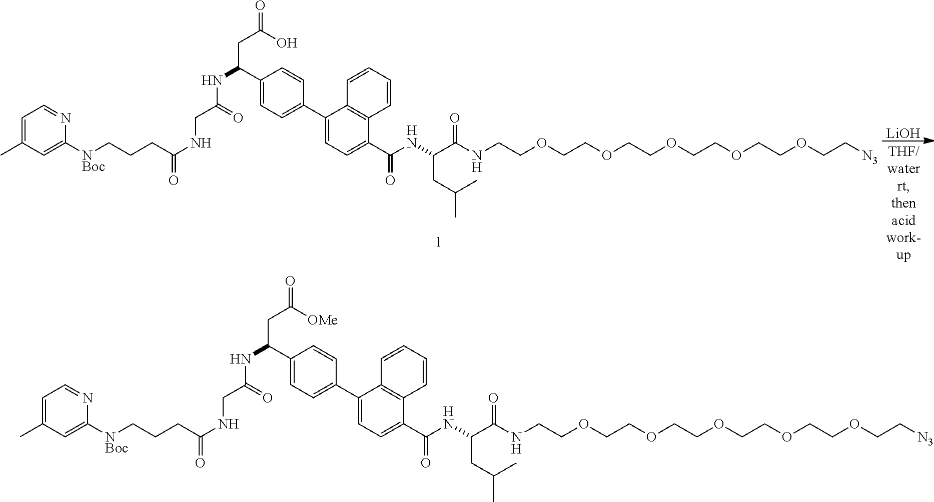

To a solution of compound 1 (0.0475 g) in 1:1 DMF/water was added LiOH (0.0031 g) at room temperature under normal atmosphere. The reaction was stirred at room temperature for 3 h until full conversion was observed by LC-MS. The reaction mixture was then acidifed with 6 N HCl to a pH of ˜4, extracted with EtOAc and then 20% CF₃CH₂OH/DCM (5×8 mL), and then washed with water and brine (3×8 mL). The product was then concentrated, providing a white solid. Yield: 0.0312 g (66.5%.) LC-MS: calculated [M+H]+ 1070.55 m/z, observed 1071.12 m/z.

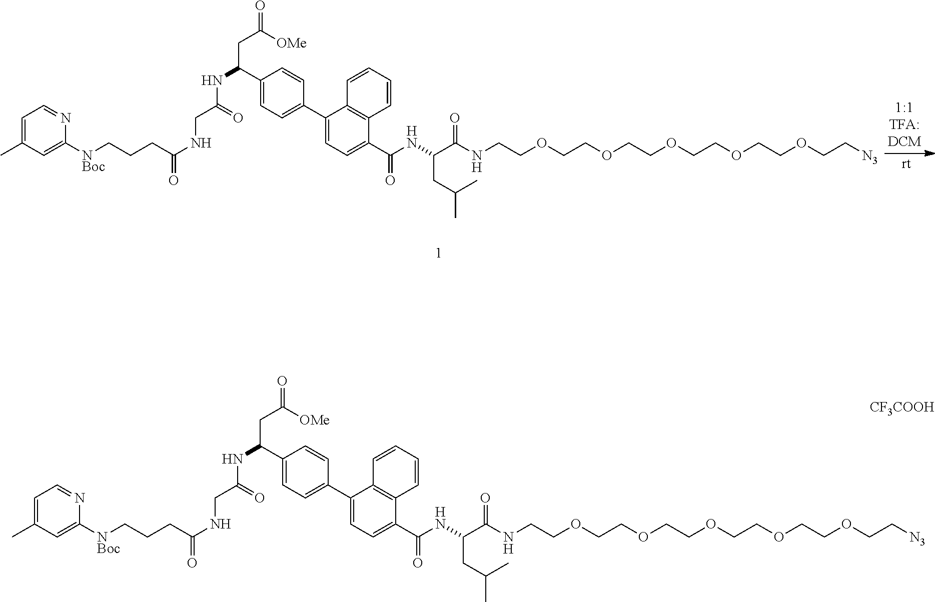 [SMILES: COC(=O)C[C@H](NC(=O)CNC(=O)CCCN(C(=O)OC(C)(C)C)c1cc(C)ccn1)c1ccc(-c2ccc(C(=O)N[C@@H](CC(C)C)C(=O)NCCOCCOCCOCCOCCOCCN=[N+]=[N-])c3ccccc23)cc1 ; COC(=O)C[C@H](NC(=O)CNC(=O)CCCN(C(=O)OC(C)(C)C)c1cc(C)ccn1)c1ccc(-c2ccc(C(=O)N[C@@H](CC(C)C)C(=O)NCCOCCOCCOCCOCCOCCN=[N+]=[N-])c3ccccc23)cc1 ; "CF3COOH"]

To a solution of compound 1 (0.0312 g) in DCM was added TFA (0.067 mL) at room temperature. The reaction was stirred under ambient conditions overnight. The following day, full conversion was confirmed via LC-MS. The reaction mixture was azeotroped with PhMe and concentrated under vacuum. No isolation was necessary. Concentration provided a clear and colorless residue. Yield: 0.0545 g (172%.) LC-MS: calculated [M+H]+ 970.50 m/z, observed 970.38 m/z.

### Synthesis of Compound 53p, (S)-3-(4-(4-(((20S,23S)-1-azido-20-isobutyl-19,22-dioxo-3,6,9,12,15-pentaoxa-18,21-diazapentacosan-23-yl)carbamoyl)naphthalen-1-yl)phenyl)-3-(2-(4-((4-methylpyridin-2-yl)amino)butanamido)acetamido)propanoic acid

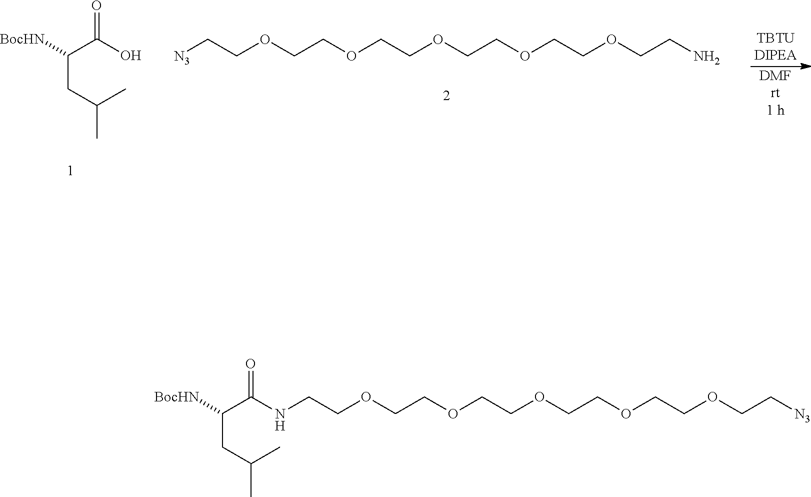 [SMILES: CC(C)C[C@H](NC(=O)OC(C)(C)C)C(=O)O ; [N-]=[N+]=NCCOCCOCCOCCOCCOCCN ; CC(C)C[C@H](NC(=O)OC(C)(C)C)C(=O)NCCOCCOCCOCCOCCOCCN=[N+]=[N-]]

To a solution of compounds 1 (0.162 g) and 2 (0.225 g) in DMF was added TBTU (0.270 g) and then DIPEA (0.366 mL) under ambient conditions. The reaction was stirred for 1 h until full conversion was observed by LC-MS. The reaction mixture was then quenched with NaHCO₃ (10 mL). The product was extracted with EtOAc (3×15 mL) and then washed with water (3×10 mL). The combined organic phase was dried over Na₂SO₄, filtered, and concentrated. The residue was purified by CombiFlash® using silica gel as the stationary phase with a gradient of hexanes to EtOAc (0-100%), in which product eluted at 100% B. The product was concentrated under vacuum to provide a clear colorless oil. Yield: 0.245 g (67.3%.) LC-MS: calculated [M+H]+ 520.33 m/z, observed 520.61 m/z.

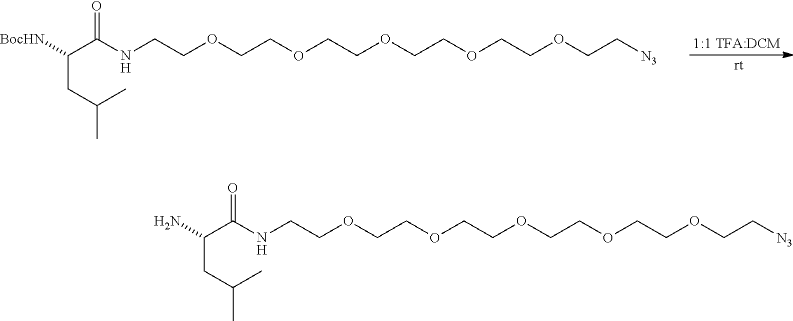 [SMILES: CC(C)C[C@H](NC(=O)OC(C)(C)C)C(=O)NCCOCCOCCOCCOCCOCCN=[N+]=[N-] ; CC(C)C[C@H](N)C(=O)NCCOCCOCCOCCOCCOCCN=[N+]=[N-]]

To a solution of compound 1 (0.245 g) in DCM was added TFA (1.61 g) at room temperature. The reaction was stirred under ambient conditions. After 1 h, full conversion was confirmed via LC-MS. The reaction mixture was azeotroped with PhMe and subjected to a base extraction with NaHCO₃. Product was extracted with EtOAc and then 20% CF₃CH₂OH/DCM and then washed with water and brine. The mixture was then concentrated under vacuum. No isolation was necessary. Concentration provided a white solid. Yield: 0.224 g (113%.) LC-MS: calculated [M+H]+ 420.27 m/z, observed 420.51 m/z.

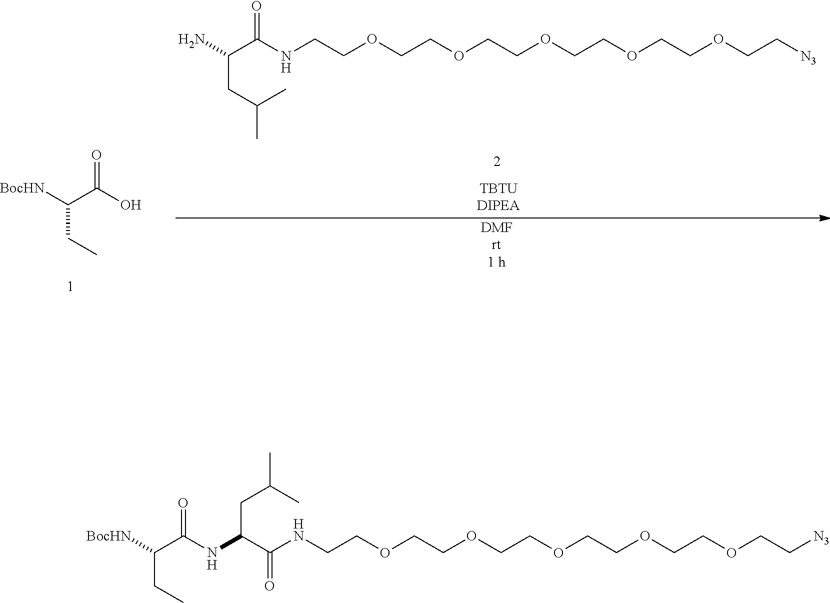

To a solution of compounds 1 (0.610 g) and 2 (0.126 g) in DMF was added TBTU (0.116 g) and then DIPEA (0.157 mL) under ambient conditions. The reaction was stirred for 1 h until full conversion was observed by LC-MS. The reaction mixture was quenched with NaHCO₃ (8 mL), extracted with EtOAc and then 20% CF₃CH₂OH/DCM (3×8 mL), and then washed with water and brine (3×8 mL). Mixture was then dried over Na₂SO₄, filtered, and concentrated. The residue was purified by CombiFlash® using silica gel as the stationary phase with a gradient of DCM to 20% MeOH in DCM (0-45%), in which product eluted at 17% B. The product was concentrated under vacuum to provide a clear colorless oil. Yield: 0.110 g (60.6%.) LC-MS: calculated [M+H]+ 605.38 m/z, observed 605.52 m/z.

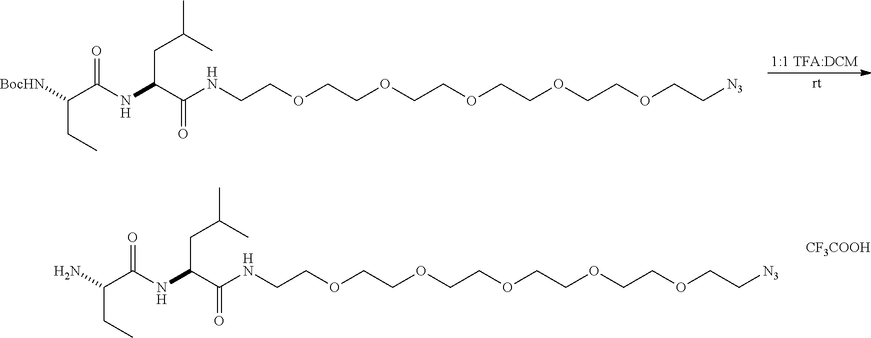 [SMILES: CC[C@H](NC(=O)OC(C)(C)C)C(=O)N[C@@H](CC(C)C)C(=O)NCCOCCOCCOCCOCCOCCN=[N+]=[N-] ; CC[C@H](N)C(=O)N[C@@H](CC(C)C)C(=O)NCCOCCOCCOCCOCCOCCN=[N+]=[N-] ; "CF3COOH"]

To a solution of compound 1 (0.110 g) in DCM was added TFA (0.418 mL) at room temperature. The reaction was stirred under ambient conditions. After 2 h, full conversion was confirmed via LC-MS. The reaction mixture was azeotroped with PhMe and concentrated under vacuum. No isolation was necessary. Concentration provided a clear colorless oil. Yield: 0.148 g (132%.) LC-MS: calculated [M+H]+ 505.33 m/z, observed 505.67 m/z.

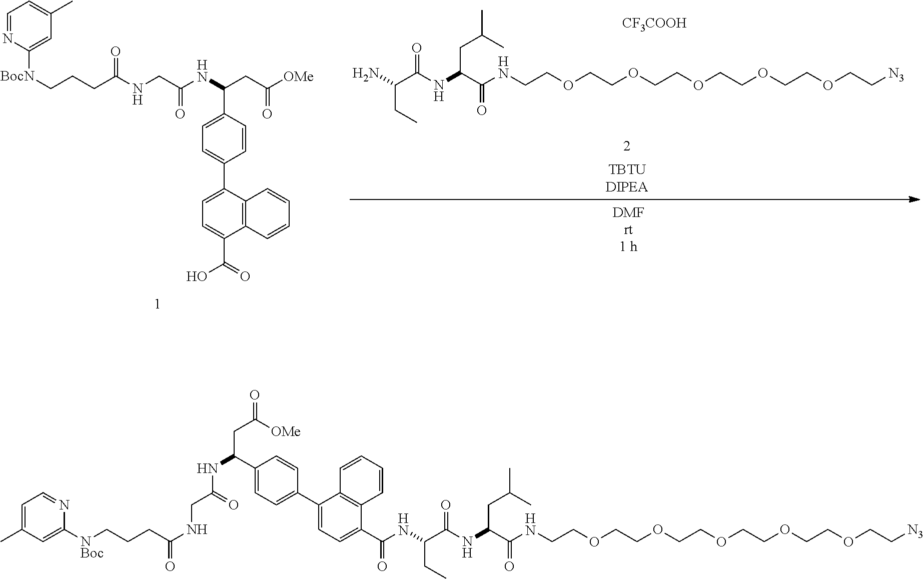 [SMILES: COC(=O)C[C@H](NC(=O)CNC(=O)CCCN(C(=O)OC(C)(C)C)c1cc(C)ccn1)c1ccc(-c2ccc(C(=O)O)c3ccccc23)cc1 ; CC[C@H](N)C(=O)N[C@@H](CC(C)C)C(=O)NCCOCCOCCOCCOCCOCCN=[N+]=[N-] ; "CF3COOH" ; CC[C@H](NC(=O)c1ccc(-c2ccc([C@H](CC(=O)OC)NC(=O)CNC(=O)CCCN(C(=O)OC(C)(C)C)c3cc(C)ccn3)cc2)c2ccccc12)C(=O)N[C@@H](CC(C)C)C(=O)NCCOCCOCCOCCOCCOCCN=[N+]=[N-]]

To a solution of compounds 1 (0.0440 g) and 2 (0.0399 g) in DMF was added TBTU (0.0248 g) and then DIPEA (0.034 mL) under ambient conditions. The reaction was stirred for 3 h until full conversion was observed by LC-MS. The reaction mixture was quenched with NaHCO₃ (8 mL), extracted with EtOAc (3×8 mL), and then washed with water (3×8 mL). The mixture was then dried over Na₂SO₄, filtered, and concentrated. The residue was purified by CombiFlash® using silica gel as the stationary phase with a gradient of DCM to 20% MeOH in DCM (0-50%), in which product eluted at 37% B. The product was concentrated under vacuum to provide a clear colorless oil. Yield: 0.0273 g (36.2%.) LC-MS: calculated [M+H]+ 1169.62 m/z, observed 1170.59 m/z.

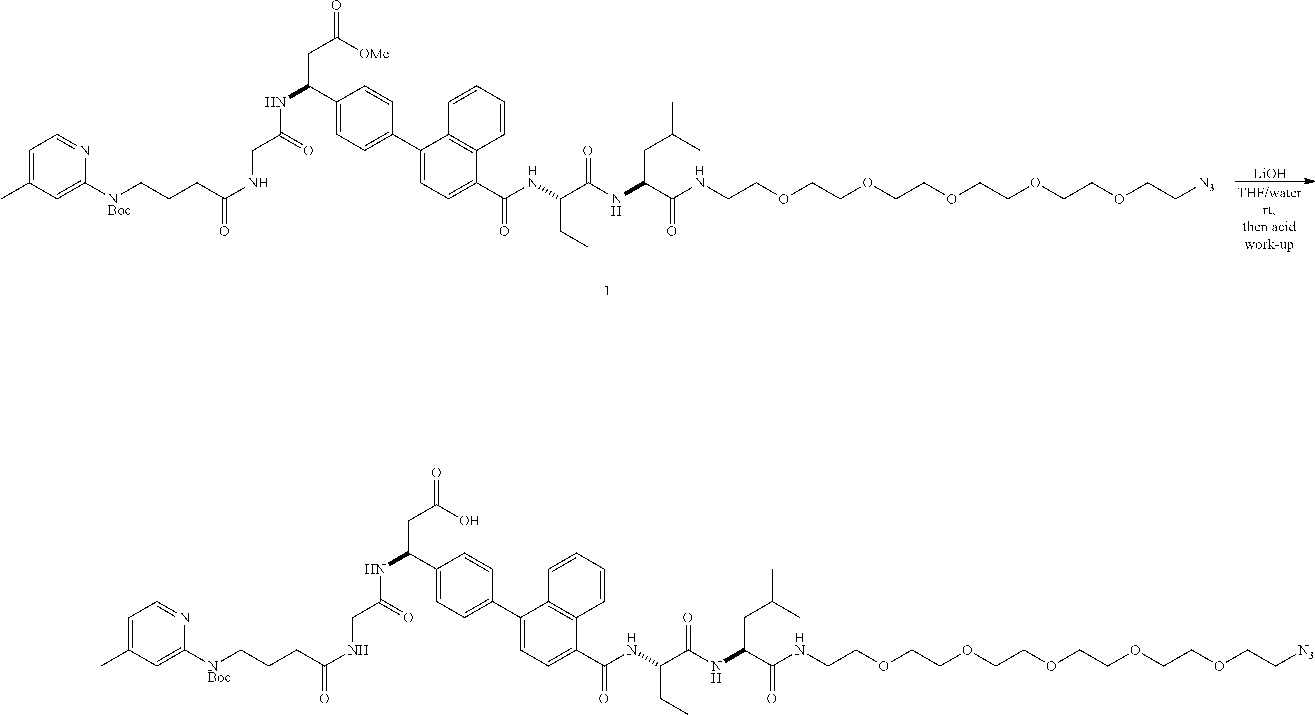

To a solution of compound 1 (0.0273 g) in 1:1 DMF/water was added LiOH (0.0017 g) at room temperature under normal atmosphere. The reaction was stirred at room temperature for 3 h until full conversion was observed by LC-MS. The reaction mixture was then acidifed with 6 N HCl to a pH of ˜4, extracted with EtOAc and then 20% CF₃CH₂OH/DCM (5×8 mL), and then washed with water and brine (3×8 mL). The product was then concentrated, providing a clear colorless oil. Yield: 0.0286 g (106%.) LC-MS: calculated [M+H]+ 1155.60 m/z, observed 1156.30 m/z.

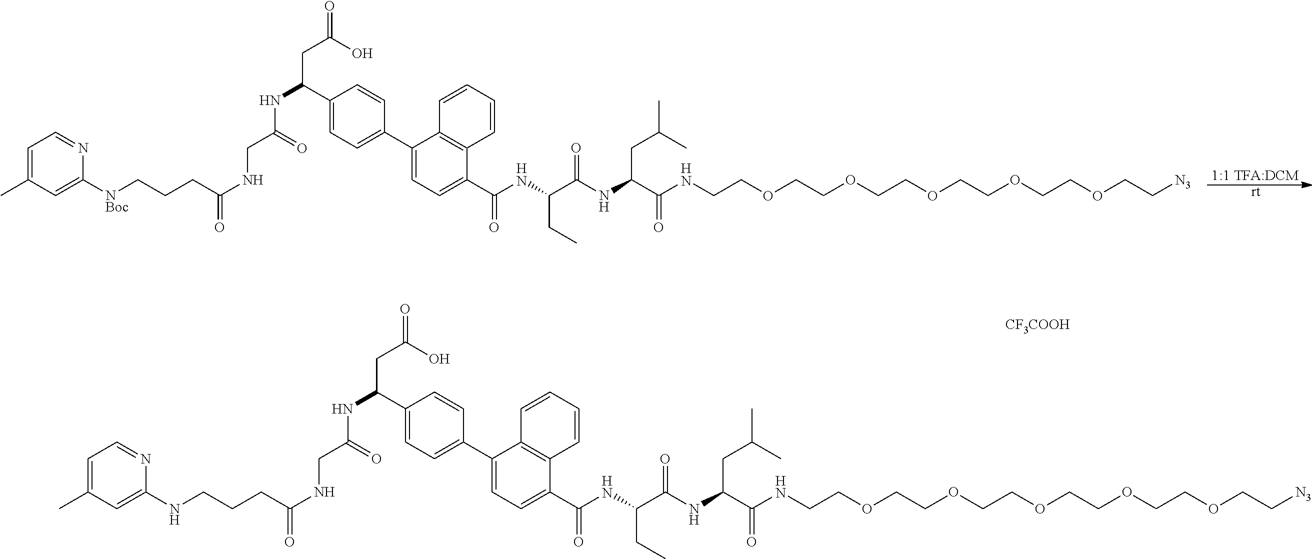 [SMILES: CC[C@H](NC(=O)c1ccc(-c2ccc([C@H](CC(=O)O)NC(=O)CNC(=O)CCCN(C(=O)OC(C)(C)C)c3cc(C)ccn3)cc2)c2ccccc12)C(=O)N[C@@H](CC(C)C)C(=O)NCCOCCOCCOCCOCCOCCN=[N+]=[N-] ; CC[C@H](NC(=O)c1ccc(-c2ccc([C@H](CC(=O)O)NC(=O)CNC(=O)CCCNc3cc(C)ccn3)cc2)c2ccccc12)C(=O)N[C@@H](CC(C)C)C(=O)NCCOCCOCCOCCOCCOCCN=[N+]=[N-] ; "CF3COOH"]

To a solution of compound 1 (0.0286 g) in DCM was added TFA (0.0847 g) at room temperature. The reaction was stirred under ambient conditions overnight. The following day, full conversion was confirmed via LC-MS. The reaction mixture was azeotroped with PhMe and concentrated under vacuum. No isolation was necessary. Concentration provided a light orange-yellow solid. Yield: 0.0384 g (133%.) LC-MS: calculated [M+H]+ 1055.55 m/z, observed 1056.08 m/z.

### Synthesis of Compound 54p, (S)-3-(4-(4-(((S)-1-azido-19-oxo-21-phenyl-3,6,9,12,15-pentaoxa-18-azahenicosan-20-yl)carbamoyl)naphthalen-1-yl)phenyl)-3-(2-(4-((4-methylpyridin-2-yl)amino)butanamido)acetamido)propanoic acid

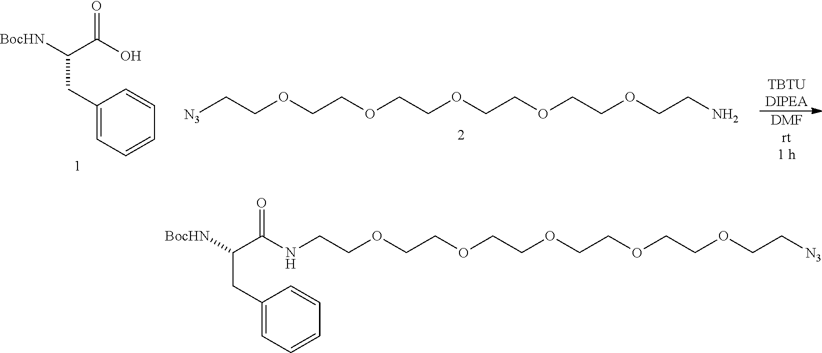

To a solution of compounds 1 (0.140 g) and 2 (0.170 g) in DMF was added TBTU (0.203 g) and then DIPEA (0.276 mL) under ambient conditions. Reaction was stirred for 1 h until full conversion was observed by LC-MS. The reaction mixture was then quenched with NaHCO₃ (10 mL). The product was extracted with EtOAc (3×15 mL) and then washed with water (3×10 mL). The combined organic phase was dried over Na₂SO₄, filtered, and concentrated. The residue was purified by CombiFlash® using silica gel as the stationary phase with a gradient of DCM to 20% of MeOH/DCM (0-40%), in which product eluted at 14% B. The product was concentrated under vacuum to provide a clear colorless oil. Yield: 0.261 g (89.5%.) LC-MS: calculated [M+H]+ 554.31 m/z, observed 554.76 m/z.

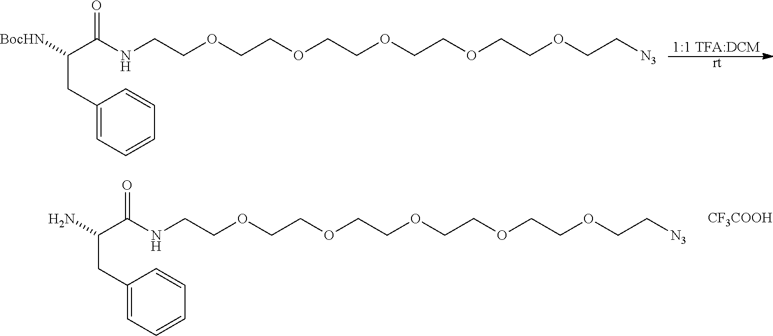

To a solution of compound 1 (0.261 g) in DCM was added TFA (1.08 mL) at room temperature. The reaction was stirred under ambient conditions. After 1 h, full conversion was confirmed via LC-MS. The reaction mixture was azeotroped with PhMe and concentrated under vacuum. No isolation was necessary. Concentration provided a yellow oil. Yield: 0.317 g (118%.) LC-MS: calculated [M+H]+ 454.26 m/z, observed 454.31 m/z.

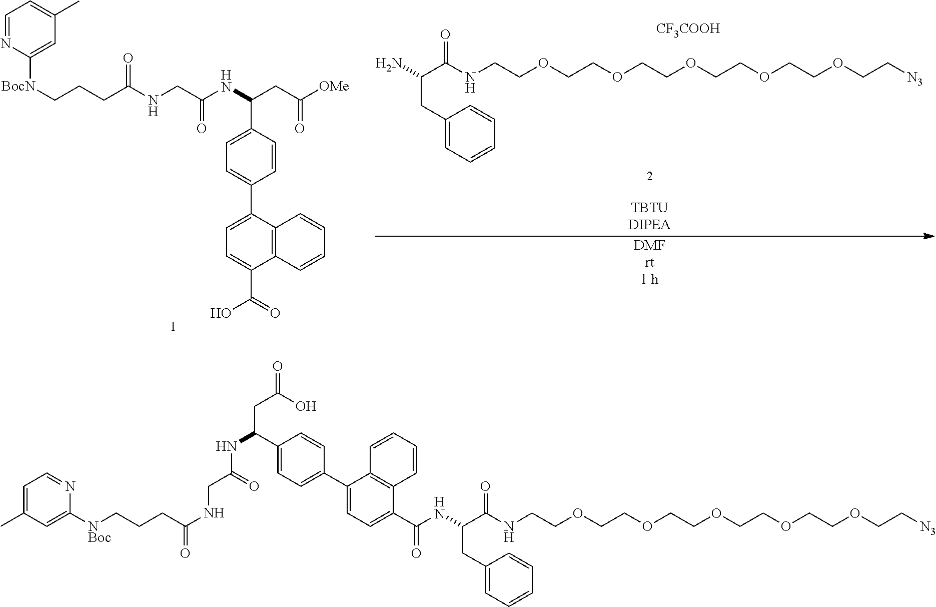 [SMILES: COC(=O)C[C@H](NC(=O)CNC(=O)CCCN(C(=O)OC(C)(C)C)c1cc(C)ccn1)c1ccc(-c2ccc(C(=O)O)c3ccccc23)cc1 ; [N-]=[N+]=NCCOCCOCCOCCOCCOCCNC(=O)[C@@H](N)Cc1ccccc1 ; "CF3COOH" ; Cc1ccnc(N(CCCC(=O)NCC(=O)N[C@@H](CC(=O)O)c2ccc(-c3ccc(C(=O)N[C@@H](Cc4ccccc4)C(=O)NCCOCCOCCOCCOCCOCCN=[N+]=[N-])c4ccccc34)cc2)C(=O)OC(C)(C)C)c1]

To a solution of compounds 1 (0.0400 g) and 2 (0.0333 g) in DMF was added TBTU (0.0226 g) and then DIPEA (0.031 mL) under ambient conditions. The reaction was stirred for 1 h until full conversion was observed by LC-MS. Reaction mixture was quenched with NaHCO₃ (8 mL), extracted with EtOAc and then 20% CF₃CH₂OH/DCM (3×8 mL), and then washed with water (3×8 mL). The mixture was then dried over Na₂SO₄, filtered, and concentrated. The residue was purified by CombiFlash using silica gel as the stationary phase with a gradient of DCM to 20% MeOH in DCM (0-50%), in which product eluted at 30% B. The product was concentrated under vacuum to provide a clear colorless oil. Yield: 0.0386 g (58.9%.) LC-MS: calculated [M+H]+ 1118.55 m/z, observed 1119.09 m/z.

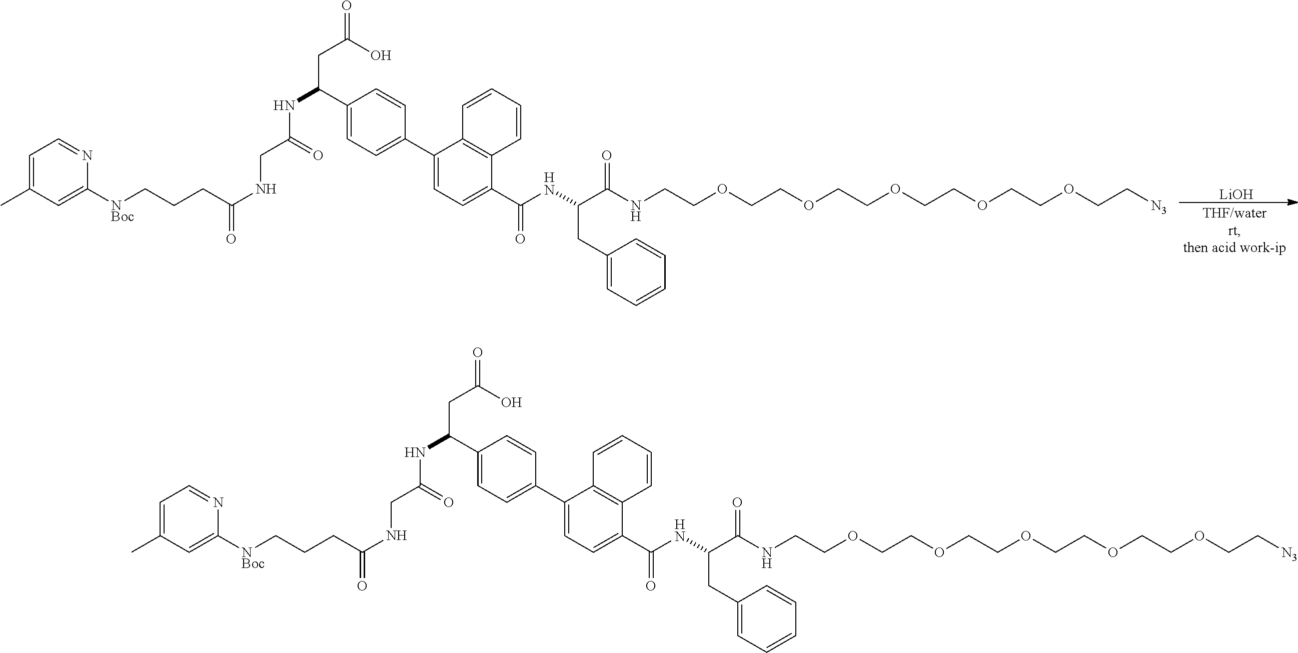 [SMILES: Cc1ccnc(N(CCCC(=O)NCC(=O)N[C@@H](CC(=O)O)c2ccc(-c3ccc(C(=O)N[C@@H](Cc4ccccc4)C(=O)NCCOCCOCCOCCOCCOCCN=[N+]=[N-])c4ccccc34)cc2)C(=O)OC(C)(C)C)c1 ; "LiOH" ; Cc1ccnc(N(CCCC(=O)NCC(=O)N[C@@H](CC(=O)O)c2ccc(-c3ccc(C(=O)N[C@@H](Cc4ccccc4)C(=O)NCCOCCOCCOCCOCCOCCN=[N+]=[N-])c4ccccc34)cc2)C(=O)OC(C)(C)C)c1]

To a solution of compound 1 (0.0386 g) in 1:1 DMF/water was added LiOH (0.0025 g) at rt under normal atmosphere. The reaction was stirred at room temperature for 3 h until full conversion was observed by LC-MS. The reaction mixture was then acidifed with 6 N HCl to a pH of ˜4, extracted with EtOAc and then 20% CF₃CH₂OH/DCM (5×8 mL), and then washed with water and brine (3×8 mL). The product was then concentrated, providing a white solid. Yield: 0.0665 g (174%.) LC-MS: calculated [M+H]+ 1104.53 m/z, observed 1105.05 m/z.

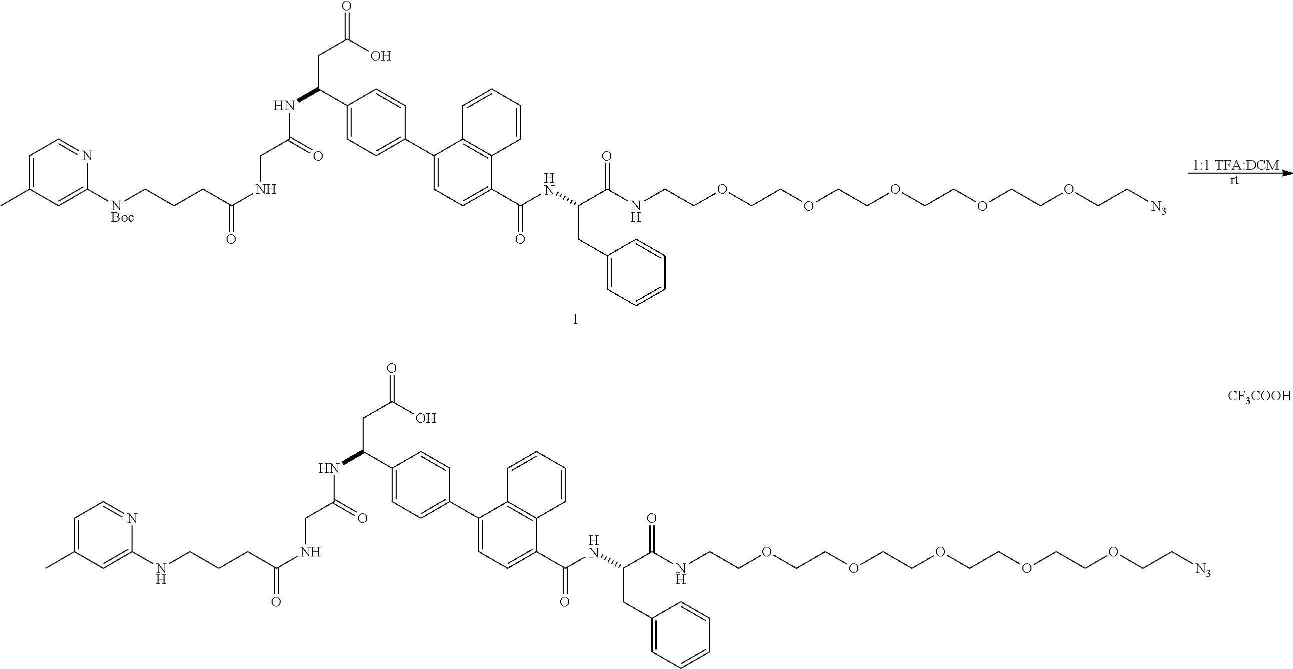 [SMILES: Cc1ccnc(N(CCCC(=O)NCC(=O)N[C@@H](CC(=O)O)c2ccc(-c3ccc(C(=O)N[C@@H](Cc4ccccc4)C(=O)NCCOCCOCCOCCOCCOCCN=[N+]=[N-])c4ccccc34)cc2)C(=O)OC(C)(C)C)c1 ; "1:1 TFA:DCM" ; Cc1ccnc(NCCCC(=O)NCC(=O)N[C@@H](CC(=O)O)c2ccc(-c3ccc(C(=O)N[C@@H](Cc4ccccc4)C(=O)NCCOCCOCCOCCOCCOCCN=[N+]=[N-])c4ccccc34)cc2)c1 ; "CF3COOH"]

To a solution of compound 1 (0.0665 g) in DCM was added TFA (0.138 mL) at room temperature. The reaction was stirred under ambient conditions for 3 h until full conversion was confirmed via LC-MS. The reaction mixture was azeotroped with PhMe and concentrated under vacuum. No isolation was necessary. Concentration provided an off-white solid. Yield: 0.0911 g (135%.) LC-MS: calculated [M+H]+ 1004.48 m/z, observed 1005.55 m/z.

### Synthesis of Compound 55p, (S)-3-(4-(4-((14-azido-3,6,9,12-tetraoxatetradecyl)oxy)naphthalen-1-yl)phenyl)-3-(2-(5-((4-methylpyrimidin-2-yl)amino)pentanamido)acetamido)propanoic acid

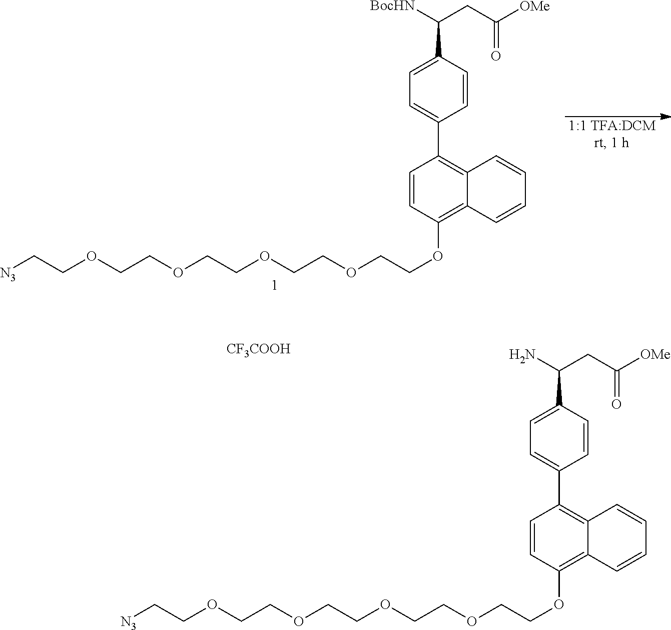 [SMILES: COC(=O)C[C@H](NC(=O)OC(C)(C)C)c1ccc(-c2ccc(OCCOCCOCCOCCOCCN=[N+]=[N-])c3ccccc23)cc1 ; COC(=O)C[C@H](N)c1ccc(-c2ccc(OCCOCCOCCOCCOCCN=[N+]=[N-])c3ccccc23)cc1 ; "CF3COOH"]

To a solution of compound 1 (0.126 g) in DCM was added TFA (0.433 mL) at room temperature. The reaction was stirred under ambient conditions. After 2 h, full conversion was confirmed via LC-MS. The reaction mixture was azeotroped with PhMe and concentrated under vacuum. No isolation was necessary. Concentration provided a yellow oil. Yield: 0.134 g (104%.) LC-MS: calculated [M+H]+ 567.27 m/z, observed 567.58 m/z.

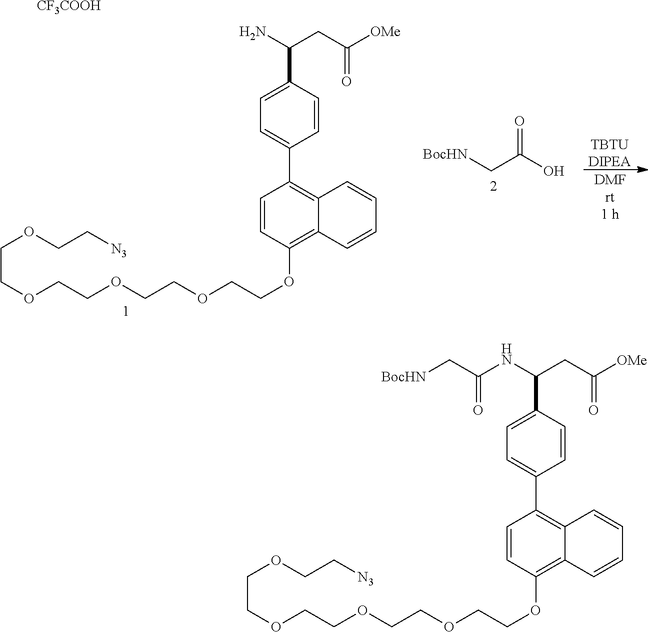 [SMILES: "CF3COOH" ; COC(=O)C[C@H](N)c1ccc(-c2ccc(OCCOCCOCCOCCOCCN=[N+]=[N-])c3ccccc23)cc1 ; CC(C)(C)OC(=O)NCC(=O)O ; COC(=O)C[C@H](NC(=O)CNC(=O)OC(C)(C)C)c1ccc(-c2ccc(OCCOCCOCCOCCOCCN=[N+]=[N-])c3ccccc23)cc1]

To a solution of compounds 1 (0.134 g) and 2 (0.0344 g) in DMF was added TBTU (0.0757 g) and then DIPEA (0.103 mL) under ambient conditions. The reaction was stirred for 3 h until full conversion was observed by TLC. The reaction mixture was then quenched with NaHCO₃ (8 mL). The product was extracted with EtOAc (3×8 mL) and then 20% CF₃CH₂OH/DCM (1×8 mL) and then washed with brine (1×8 mL) and then water (3×8 mL). The combined organic phase was dried over Na₂SO₄, filtered, and concentrated. The residue was purified by CombiFlash® using silica gel as the stationary phase with a gradient of DCM to 20% MeOH in DCM (0-40%), in which product eluted at 14% B. The product was concentrated under vacuum to provide a clear colorless residue. Yield: 0.0999 g (70.3%.) LC-MS: calculated [M+H]+ 724.35 m/z, observed 724.92 m/z.

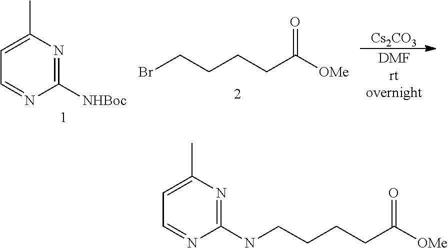

To a solution of compound 1 (0.100 g) in DMF was added Cs₂CO₃ (0.234 g) under ambient conditions. Compound 2 (0.068 mL) was then added slowly. The reaction was stirred overnight. Approx. 50% conversion to desired product by LC-MS was then confirmed. The reaction mixture was quenched with NaHCO₃ (10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of hex to EtOAc (0-70%), in which product eluted at 29% B. The product was concentrated under vacuum to provide a clear colorless oil. Yield: 0.0993 g (64.3%.) LC-MS: calculated [M+H]+ 324.18 m/z, observed 324.41 m/z.

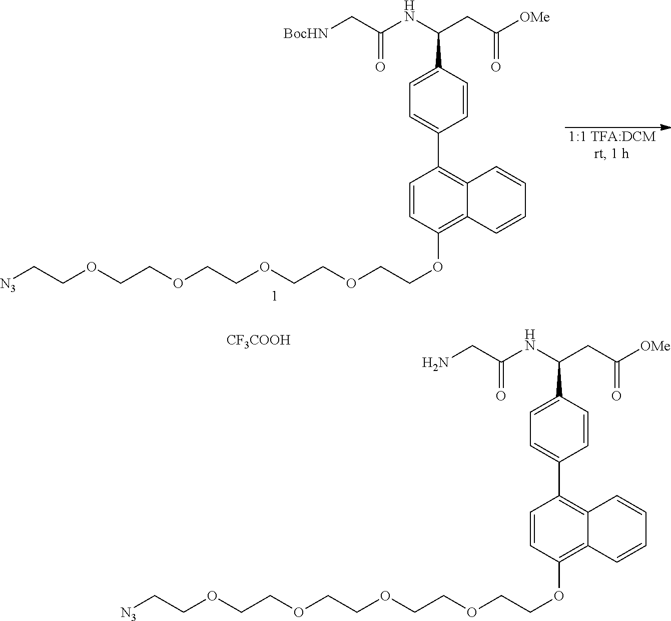 [SMILES: COC(=O)C[C@H](NC(=O)CNC(=O)OC(C)(C)C)c1ccc(-c2ccc(OCCOCCOCCOCCOCCN=[N+]=[N-])c3ccccc23)cc1 ; COC(=O)C[C@H](NC(=O)CN)c1ccc(-c2ccc(OCCOCCOCCOCCOCCN=[N+]=[N-])c3ccccc23)cc1 ; "CF3COOH"]

To a solution of compound 1 (0.0999 g) in DCM was added TFA (0.317 mL) at room temperature. The reaction was stirred under ambient conditions. After 5 h, full conversion was confirmed via TLC. The reaction mixture was azeotroped with PhMe and concentrated under vacuum. No isolation was necessary. Concentration provided a yellow oil. Yield: 0.1168 g (115%.) LC-MS: calculated [M+H]+ 624.30 m/z, observed 624.68 m/z.

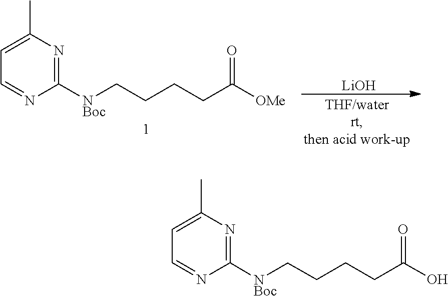 [SMILES: COC(=O)CCCCN(C(=O)OC(C)(C)C)c1nccc(C)n1 ; "LiOH" ; Cc1ccnc(N(CCCCC(=O)O)C(=O)OC(C)(C)C)n1]

To a solution of compound 1 (0.0993 g) in 1:1 THF/water was added LiOH (0.0221 g) at room temperature under normal atmosphere. The reaction was stirred at room temperature until full conversion was observed by TLC. After 4 h, the reaction mixture was acidifed with 6 N HCl to a pH of ˜3. The product was extracted with EtOAc (3×5 mL) and then 20% CF₃CH₂OH/DCM (3×5 mL). The combined organic phase was dried over Na₂SO₄, filtered, and concentrated, providing a clear, colorless oil. Yield: 0.0876 g (92.2%.) LC-MS: calculated [M+H]+ 310.17 m/z, observed 310.49 m/z.

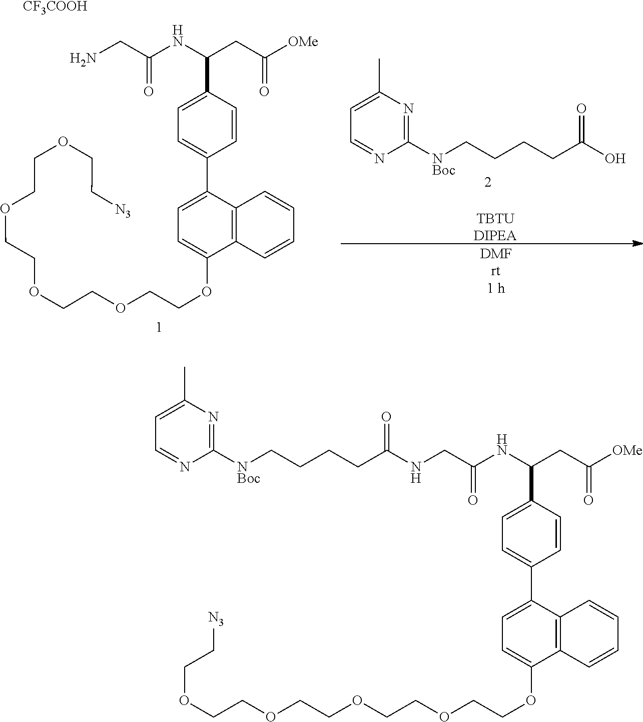 [SMILES: "CF3COOH" ; COC(=O)C[C@H](NC(=O)CN)c1ccc(-c2ccc(OCCOCCOCCOCCOCCN=[N+]=[N-])c3ccccc23)cc1 ; Cc1ccnc(N(CCCCC(=O)O)C(=O)OC(C)(C)C)n1 ; COC(=O)C[C@H](NC(=O)CNC(=O)CCCCN(C(=O)OC(C)(C)C)c1nccc(C)n1)c1ccc(-c2ccc(OCCOCCOCCOCCOCCN=[N+]=[N-])c3ccccc23)cc1]

To a solution of compounds 1 (0.113 g) and 2 (0.0472 g) in DMF was added DIPEA (0.080 mL) and then TBTU (0.0588 g) under ambient conditions. Reaction was stirred for 1 h until full conversion was observed by TLC. The reaction mixture was then quenched with NaHCO₃ (10 mL). The product was extracted with EtOAc (3×5 mL) and then 20% CF₃CH₂OH/DCM (3×8 mL) and then washed with water (3×10 mL). The combined organic phase was dried over Na₂SO₄, filtered, and concentrated. The residue was purified by CombiFlash using silica gel as the stationary phase with a gradient of DCM to 20% MeOH in DCM (0-70%), in which product eluted at 34% B. The product was concentrated under vacuum to provide a clear colorless oil. Yield: 0.0712 g (51.0%.) LC-MS: calculated [M+H]+ 915.45 m/z, observed 915.85 m/z.

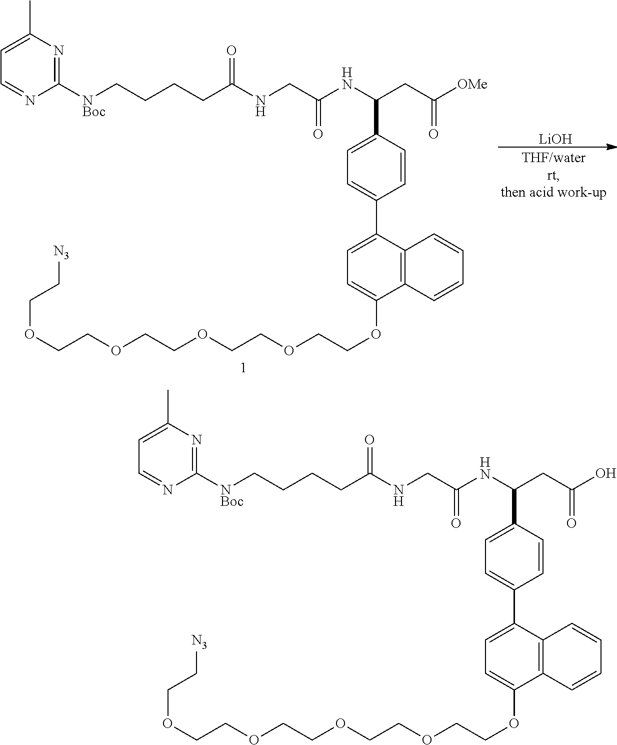

To a solution of compound 1 (0.0712 g) in 1:1 THF/water was added LiOH (0.0056 g) at rt under normal atmosphere. The reaction was stirred at room temperature until full conversion was observed by LC-MS. After 4 h, the reaction mixture was acidifed with 6 N HCl to a pH of ˜3. The product was extracted with EtOAc and then 20% CF₃CH₂OH/DCM (3×8 mL). The combined organic phase was dried over Na₂SO₄, filtered, and concentrated, providing a clear colorless oil. LC-MS: calculated [M+H]+ 901.44 m/z, observed 901.57 m/z.

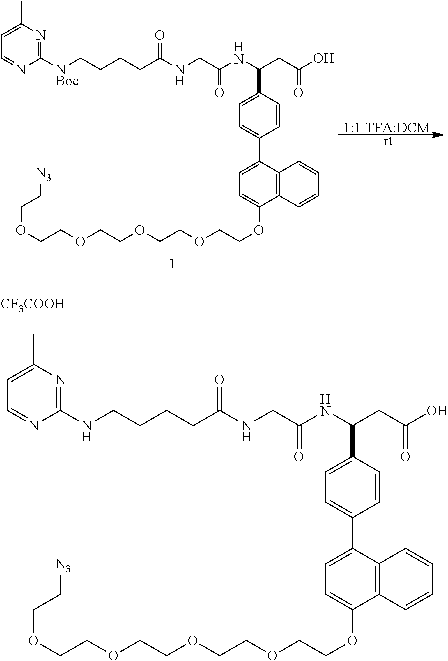 [SMILES: Cc1ccnc(N(CCCCC(=O)NCC(=O)N[C@@H](CC(=O)O)c2ccc(-c3ccc(OCCOCCOCCOCCOCCN=[N+]=[N-])c4ccccc34)cc2)C(=O)OC(C)(C)C)n1 ; "1:1 TFA:DCM" ; "CF3COOH" ; Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)n1]

To a solution of compound 1 (0.0640 g) in DCM was added TFA (0.163 mL) at room temperature. The reaction was stirred under ambient conditions overnight. The following day, desired product was observed via LC-MS. The reaction mixture was azeotroped with PhMe and concentrated under vacuum to provide a clear colorless oil. Yield: 0.0707 g (108%.) LC-MS: calculated [M+H]+ 801.39 m/z, observed 801.47 m/z.

### Synthesis of Compound 56p, (S)-3-(4-(4-((14-azido-3,6,9,12-tetraoxatetradecyl)oxy)naphthalen-1-yl)phenyl)-3-(2-(5-((6-methylpyridin-2-yl)amino)pentanamido)acetamido)propanoic acid

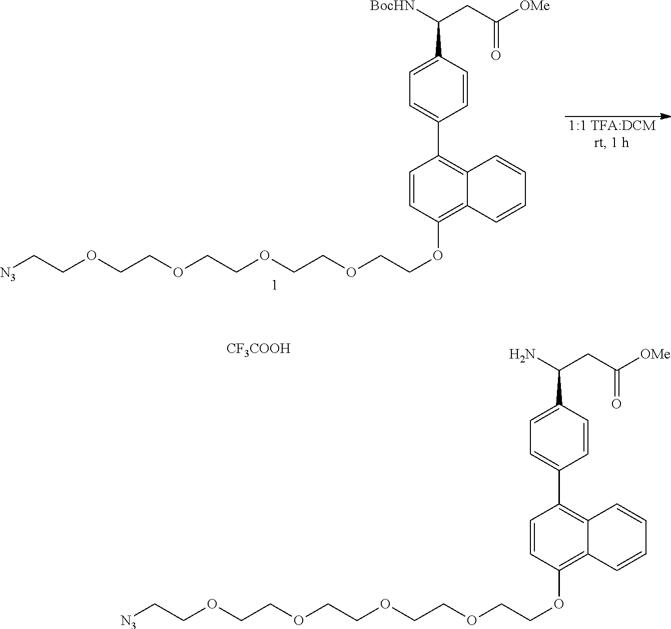 [SMILES: COC(=O)C[C@H](NC(=O)OC(C)(C)C)c1ccc(-c2ccc(OCCOCCOCCOCCOCCN=[N+]=[N-])c3ccccc23)cc1 ; COC(=O)C[C@H](N)c1ccc(-c2ccc(OCCOCCOCCOCCOCCN=[N+]=[N-])c3ccccc23)cc1 ; "CF3COOH"]

To a solution of compound 1 (0.356 g) in DCM was added TFA (1.227 mL) at room temperature. The reaction was stirred under ambient conditions. After 2 h, full conversion was confirmed via LC-MS. The reaction mixture was azeotroped with PhMe and concentrated under vacuum. No isolation was necessary. Concentration provided a deep honey-colored oil. Yield: 0.364 g (100%.) LC-MS: calculated [M+H]+ 567.27 m/z, observed 567.58 m/z.

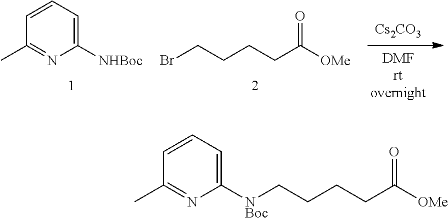

To a solution of compound 1 (0.0961 g) in DMF was added Cs₂CO₃ (0.226 g) under ambient conditions. Compound 2 (0.066 mL) was then added slowly. The reaction was stirred overnight. Approx. 50% conversion to desired product by LC-MS was then confirmed. The reaction mixture was quenched with NaHCO₃ (10 mL). The product was extracted with EtOAc (3×15 mL) and then washed with water (3×10 mL). The combined organic phase was dried over Na₂SO₄, filtered, and concentrated. The residue was purified by CombiFlash® using silica gel as the stationary phase with a gradient of hex to EtOAc (0-30%), in which product eluted at 19% B. The product was concentrated under vacuum to provide a clear colorless oil. Yield: 0.0354 g (23.8%.) LC-MS: calculated [M+H]+ 323.19 m/z, observed 323.10 m/z.

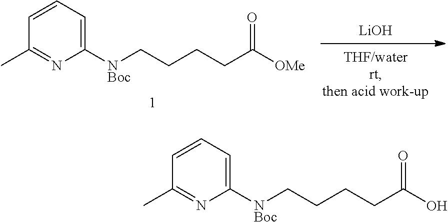 [SMILES: COC(=O)CCCCN(C(=O)OC(C)(C)C)c1cccc(C)n1 ; "LiOH" ; Cc1cccc(N(CCCCC(=O)O)C(=O)OC(C)(C)C)n1]

To a solution of compound 1 (0.0354 g) in 1:1 THF/water was added LiOH (0.0079 g) at room temperature under normal atmosphere. The reaction was stirred at room temperature until full conversion was observed by TLC. After 1 h, the reaction mixture was acidifed with 6 N HCl to a pH of ˜3. The product was extracted with EtOAc and then 20% CF₃CH₂OH/DCM (3×8 mL). The combined organic phase was dried over Na₂SO₄, filtered, and concentrated, providing a clear, colorless oil. Yield: 0.0625 g (184%.) LC-MS: calculated [M+H]+ 309.17 m/z, observed 309.42 m/z.

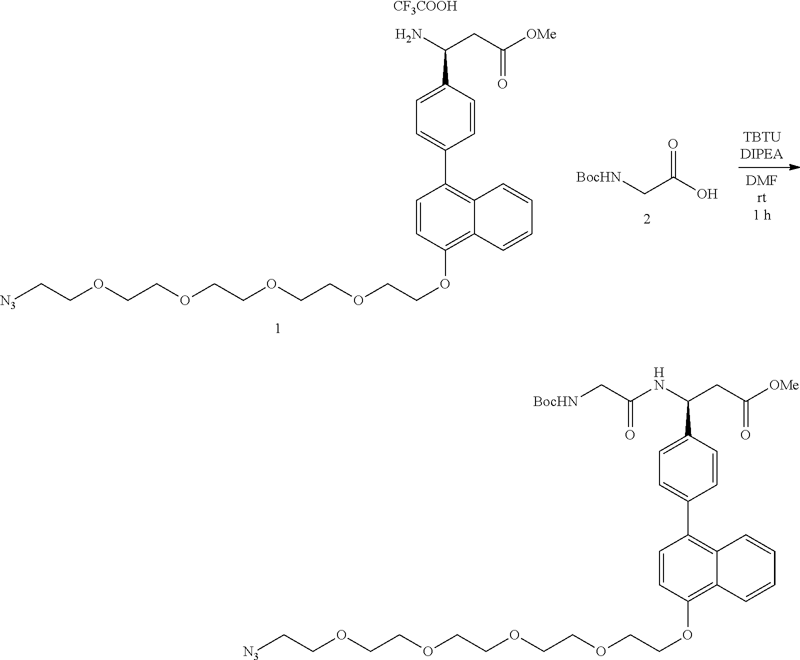 [SMILES: COC(=O)C[C@H](N)c1ccc(-c2ccc(OCCOCCOCCOCCOCCN=[N+]=[N-])c3ccccc23)cc1 ; "CF3COOH" ; CC(C)(C)OC(=O)NCC(=O)O ; COC(=O)C[C@H](NC(=O)CNC(=O)OC(C)(C)C)c1ccc(-c2ccc(OCCOCCOCCOCCOCCN=[N+]=[N-])c3ccccc23)cc1]

To a solution of compounds 1 (0.364 g) and 2 (0.0936 g) in DMF was added TBTU (0.206 g) and then DIPEA (0.279 mL) under ambient conditions. Reaction was stirred for 3 h. Then reaction mixture was quenched with NaHCO₃ (10 mL) and brine (15 mL). The product was extracted with EtOAc (2×5 mL) and then 20% CF₃CH₂OH/DCM (3×8 mL) and then washed with water (5×8 mL) and brine (1×5 mL). The combined organic phase was dried over Na₂SO₄, filtered, and concentrated. The residue was purified by CombiFlash using silica gel as the stationary phase with a gradient of DCM to 20% MeOH in DCM (0-25%), in which product eluted at 5% B to provide a clear colorless oil. Yield: 0.243 g (63.0%.) LC-MS: calculated [M+H]+ 724.35 m/z, observed 724.66 m/z.

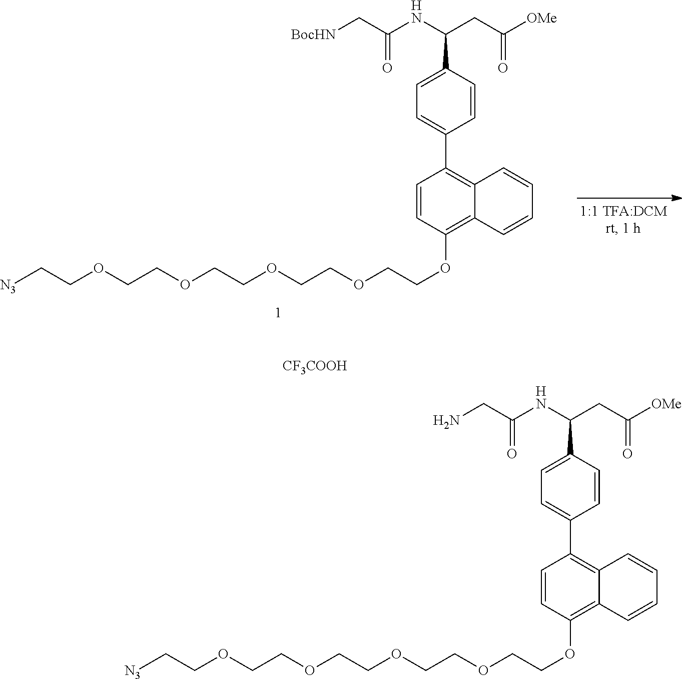 [SMILES: COC(=O)C[C@H](NC(=O)CNC(=O)OC(C)(C)C)c1ccc(-c2ccc(OCCOCCOCCOCCOCCN=[N+]=[N-])c3ccccc23)cc1 ; COC(=O)C[C@H](NC(=O)CN)c1ccc(-c2ccc(OCCOCCOCCOCCOCCN=[N+]=[N-])c3ccccc23)cc1 ; "CF3COOH"]

To a solution of compound 1 (0.244 g) in DCM was added TFA (0.774 mL) at room temperature. The reaction was stirred under ambient conditions. After 1 h, full conversion was confirmed via LC-MS. The reaction mixture was concentrated under vacuum. No isolation was necessary. Concentration provided a yellow oil. Yield: 0.281 g (113%.) LC-MS: calculated [M+H]+ 624.30 m/z, observed 624.56 m/z.

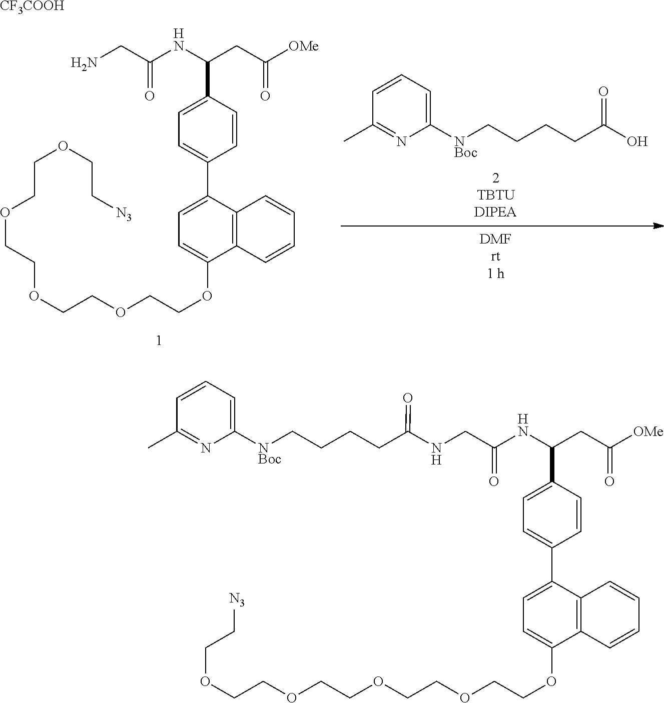

To a solution of compounds 1 (0.115 g) and 2 (0.0625 g) in DMF was added TBTU (0.0601 g) and then DIPEA (0.081 mL) under ambient conditions. The reaction was stirred for 3 h until full conversion was observed by LC-MS. The reaction mixture was then quenched with NaHCO₃ (8 mL). The product was extracted with EtOAc (2×5 mL) and then 20% CF₃CH₂OH/DCM (3×8 mL) and then washed with water (3×8 mL) and brine (8 mL). The combined organic phase was dried over Na₂SO₄, filtered, and concentrated. The residue was purified by CombiFlash® using silica gel as the stationary phase with a gradient of DCM to 20% MeOH in DCM (0-30%), in which product eluted at 20% B. The product was concentrated under vacuum to provide a clear colorless oil. Yield: 0.0450 g (31.6%.) LC-MS: calculated [M+H]+ 914.46 m/z, observed 914.79 m/z.

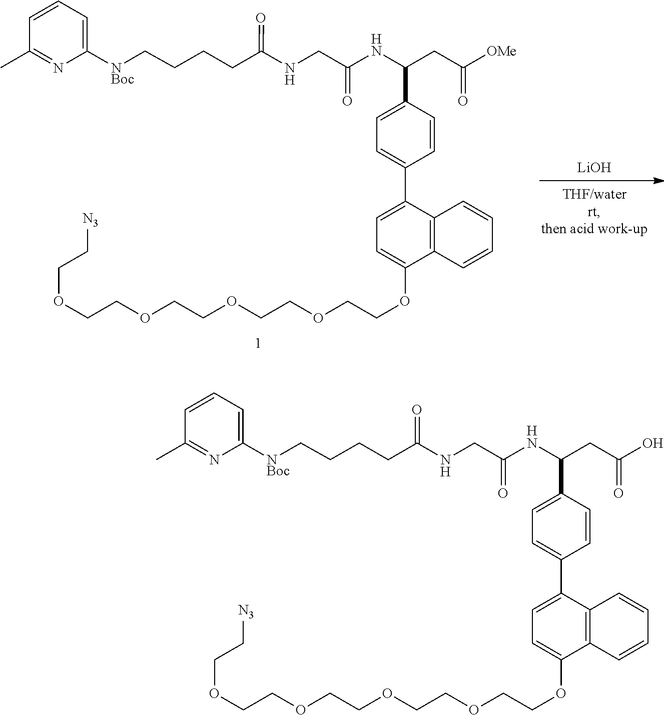

To a solution of compound 1 (0.450 g) in 1:1 THF/water was added LiOH (0.0035 g) at rt under normal atmosphere. The reaction was stirred at room temperature until full conversion was observed by LC-MS. After 2 h, the reaction mixture was acidifed with 6 N HCl to a pH of ˜3. The product was extracted with EtOAc and then 20% CF₃CH₂OH/DCM (3×8 mL). The combined organic phase was dried over Na₂SO₄, filtered, and concentrated, providing a clear colorless oil. Yield: 0.0425 g (95.9%.) LC-MS: calculated [M+H]+ 900.44 m/z, observed 900.74 m/z.

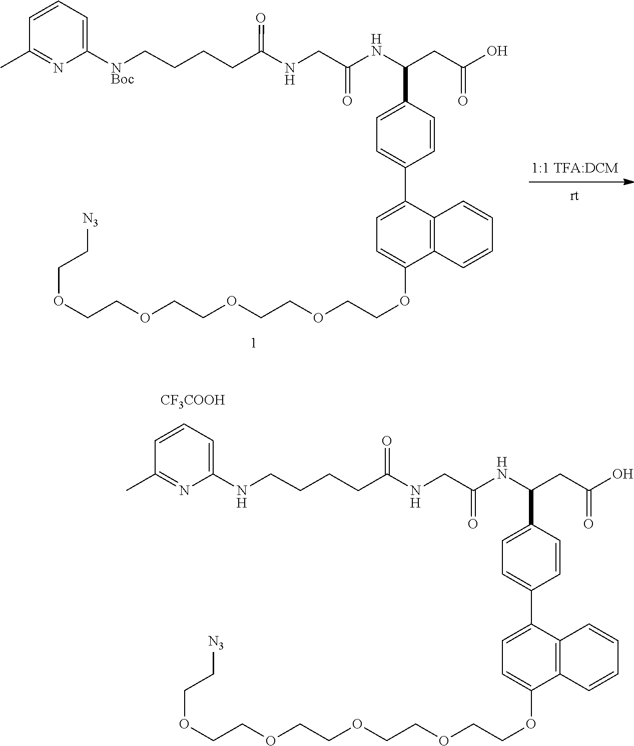

To a solution of compound 1 (0.0425 g) in DCM was added TFA (0.108 mL) at room temperature. The reaction was stirred overnight under ambient conditions until full conversion was observed by LC-MS. The reaction mixture was azeotroped with PhMe and concentrated under vacuum to provide a light yellow oil. Yield: 0.0468 g (108%.) LC-MS: calculated [M+H]+ 800.39 m/z, observed 800.73 m/z.

### Synthesis of Compound 57p, (S)-3-(4-(4-((14-azido-3,6,9,12-tetraoxatetradecyl)oxy)naphthalen-1-yl)phenyl)-3-(2-(5-((6-methoxypyridin-2-yl)amino)pentanamido)acetamido)propanoic acid

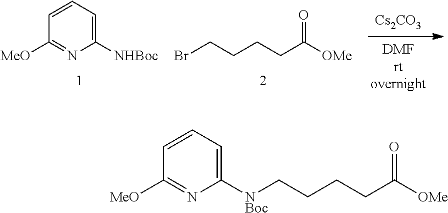

To a solution of compound 1 (0.1035 g) in DMF was added Cs₂CO₃ (0.226 g) under ambient conditions. Compound 2 (0.066 mL) was then added slowly. Reaction was stirred overnight. Approx. 50% conversion to desired product by LC-MS was then confirmed. The reaction mixture was quenched with NaHCO₃ (10 mL). The product was extracted with EtOAc (3×15 mL) and then washed with water (3×10 mL). The combined organic phase was dried over Na₂SO₄, filtered, and concentrated. The residue was purified by CombiFlash® using silica gel as the stationary phase with a gradient of hex to EtOAc (0-15%), in which product eluted at 6% B. The product was concentrated under vacuum to provide a clear colorless oil. Yield: 0.0438 g (28.0%.) LC-MS: calculated [M+H]+ 339.18 m/z, observed 339.48 m/z.

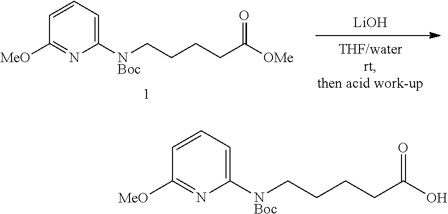 [SMILES: COC(=O)CCCCN(C(=O)OC(C)(C)C)c1cccc(OC)n1 ; "LiOH" ; COc1cccc(N(CCCCC(=O)O)C(=O)OC(C)(C)C)n1]

To a solution of compound 1 (0.0438 g) in 1:1 THF/water was added LiOH (0.0093 g) at room temperature under normal atmosphere. The reaction was stirred at room temperature until full conversion was observed by TLC. After 1 h, the reaction mixture was acidifed with 6 N HCl to a pH of ˜3. The product was extracted with EtOAc and then 20% CF₃CH₂OH/DCM (3×8 mL). The combined organic phase was dried over Na₂SO₄, filtered, and concentrated, providing a clear, colorless oil. Yield: 0.0485 g (115%.) LC-MS: calculated [M+H]+ 325.17 m/z, observed 325.35 m/z.

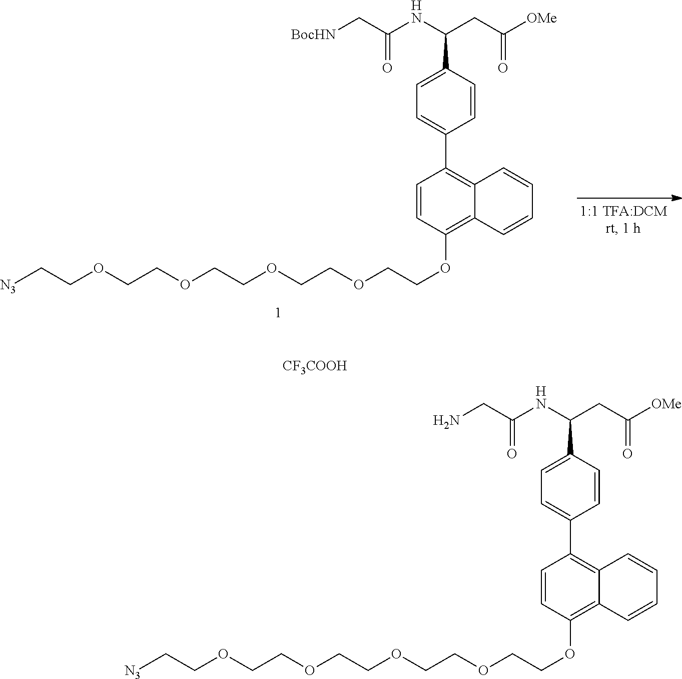 [SMILES: COC(=O)C[C@H](NC(=O)CNC(=O)OC(C)(C)C)c1ccc(-c2ccc(OCCOCCOCCOCCOCCN=[N+]=[N-])c3ccccc23)cc1 ; COC(=O)C[C@H](NC(=O)CN)c1ccc(-c2ccc(OCCOCCOCCOCCOCCN=[N+]=[N-])c3ccccc23)cc1 ; "CF3COOH"]

To a solution of compound 1 (0.244 g) in DCM was added TFA (0.774 mL) at room temperature. The reaction was stirred under ambient conditions. After 1 h, full conversion was confirmed via LC-MS. The reaction mixture was concentrated under vacuum. No isolation was necessary. Concentration provided a yellow oil. Yield: 0.281 g (113%.) LC-MS: calculated [M+H]+ 624.30 m/z, observed 624.56 m/z.

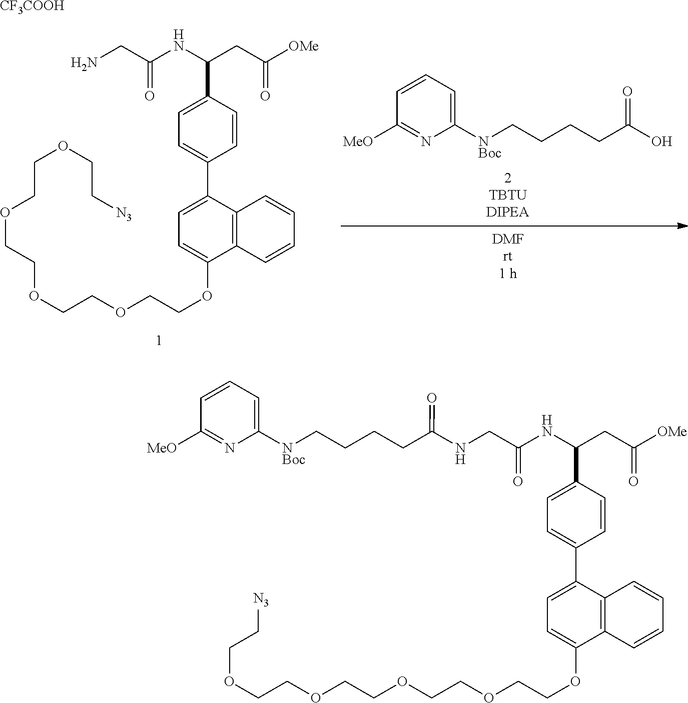

To a solution of compounds 1 (0.0850 g) and 2 (0.0486 g) in DMF was added TBTU (0.0444 g) and then DIPEA (0.060 mL) under ambient conditions. Reaction was stirred for 3 h until full conversion was observed by LC-MS. The reaction mixture was then quenched with NaHCO₃ (8 mL). The product was extracted with EtOAc (2×5 mL) and then 20% CF₃CH₂OH/DCM (3×8 mL) and then washed with water (3×8 mL) and brine (8 mL). The combined organic phase was dried over Na₂SO₄, filtered, and concentrated. The residue was purified by CombiFlash® using silica gel as the stationary phase with a gradient of DCM to 20% MeOH in DCM (0-30%), in which product eluted at 17% B. The product was concentrated under vacuum to provide a clear colorless oil. Yield: 0.0518 g (48.3%.) LC-MS: calculated [M+H]+ 930.45 m/z, observed 930.90 m/z.

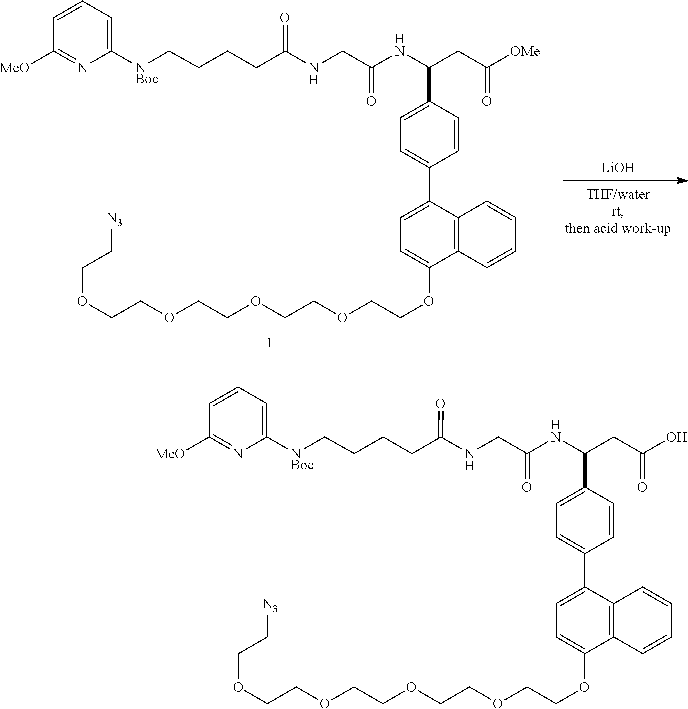

To a solution of compound 1 (0.0518 g) in 1:1 THF/water was added LiOH (0.0040 g) at room temperature under normal atmosphere. The reaction was stirred at rt until full conversion was observed by LC-MS. After 2 h, the reaction mixture was acidifed with 6 N HCl to a pH of ˜3. The product was extracted with EtOAc and then 20% CF₃CH₂OH/DCM (3×8 mL). The combined organic phase was dried over Na₂SO₄, filtered, and concentrated, providing a clear colorless oil. Yield: 0.0493 g (96.6%.) LC-MS: calculated [M+H]+ 916.44 m/z, observed 916.95 m/z.

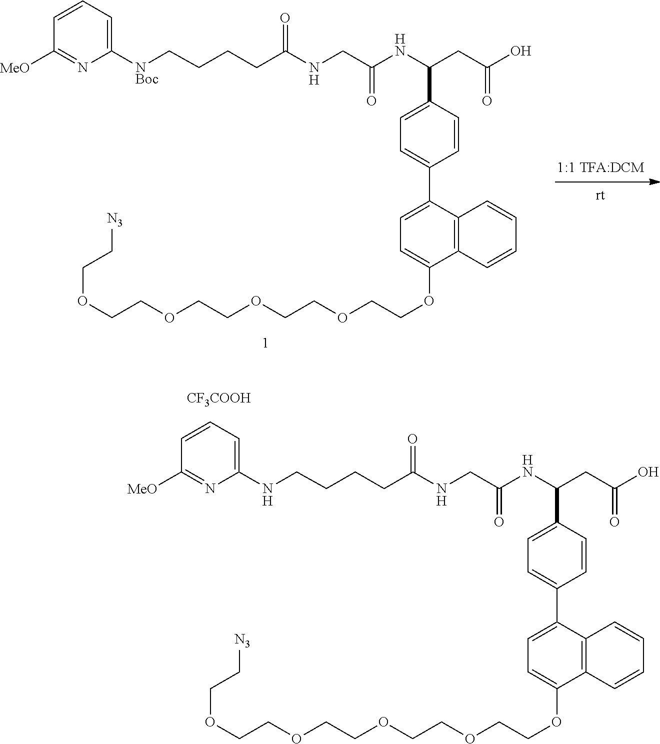

To a solution of compound 1 (0.0493 g) in DCM was added TFA (0.124 mL) at room temperature. The reaction was stirred overnight under ambient conditions until full conversion was observed by LC-MS. The reaction mixture was azeotroped with PhMe and concentrated under vacuum to provide a light yellow oil. Yield: 0.0531 g (Yield: 106%.) LC-MS: calculated [M+H]+ 816.39 m/z, observed 816.66 m/z.

### Synthesis of Compound 58p, (S)-3-(4-(4-((14-azido-3,6,9,12-tetraoxatetradecyl)oxy)naphthalen-1-yl)phenyl)-3-(2-(5-((4-chloropyridin-2-yl)amino)pentanamido)acetamido)propanoic acid

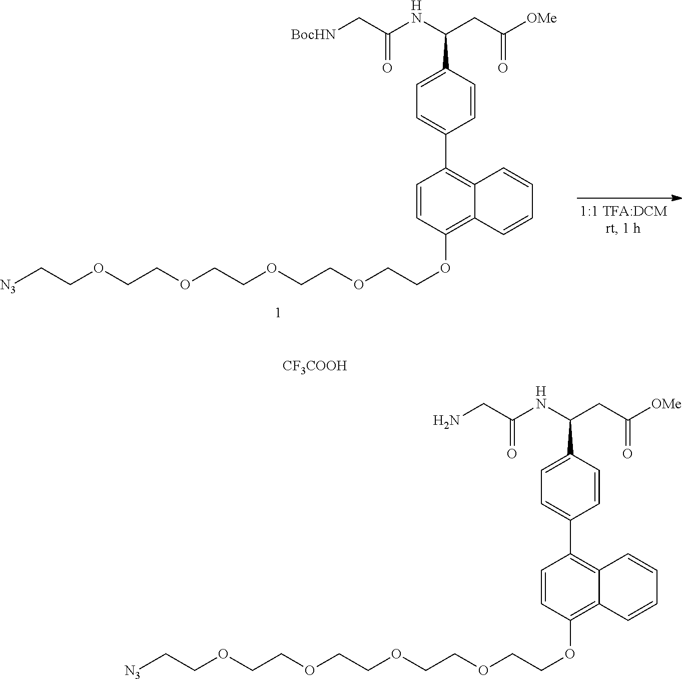 [SMILES: COC(=O)C[C@H](NC(=O)CNC(=O)OC(C)(C)C)c1ccc(-c2ccc(OCCOCCOCCOCCOCCN=[N+]=[N-])c3ccccc23)cc1 ; COC(=O)C[C@H](NC(=O)CN)c1ccc(-c2ccc(OCCOCCOCCOCCOCCN=[N+]=[N-])c3ccccc23)cc1 ; "CF3COOH"]

To a solution of compound 1 (0.244 g) in DCM was added TFA (1.15 g) at room temperature. The reaction was stirred under ambient conditions. After 1 h, full conversion was confirmed via LC-MS. The reaction mixture was concentrated under vacuum. No isolation was necessary. Concentration provided a yellow oil. Yield: 0.281 g (113%.) LC-MS: calculated [M+H]+ 624.30 m/z, observed 624.56 m/z.

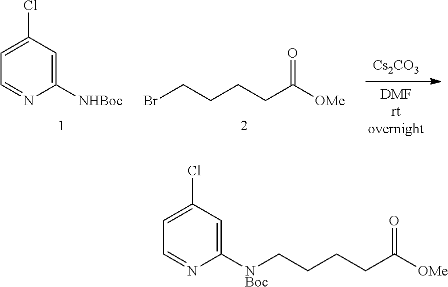

To a solution of compound 1 (0.300 g) in DMF was added Cs₂CO₃ (0.512 g) at room temperature. Compound 2 (0.269 g) was then added slowly dropwise. Reaction was stirred overnight. Approx. full conversion to desired product by LC-MS was then confirmed. The reaction mixture was quenched with NaHCO₃ (10 mL). The product was extracted with EtOAc (3×8 mL) and then washed with water (3×8 mL) and brine (8 mL). The combined organic phase was dried over Na₂SO₄, filtered, and concentrated. The residue was purified by CombiFlash® using silica gel as the stationary phase with a gradient of hex to EtOAc (0-60%), in which product eluted at 7.5% B. The product was concentrated under vacuum to provide a clear and colorless oil. Yield: 0.311 g (69.2%.) LC-MS: calculated [M+H]+ 343.13 m/z, observed 343.08 mz.

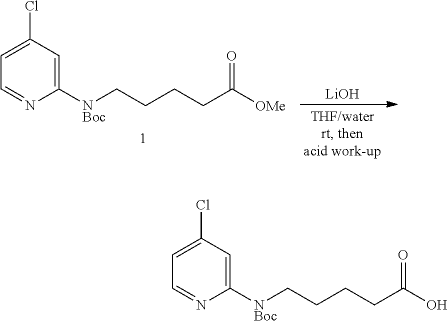 [SMILES: COC(=O)CCCCN(C(=O)OC(C)(C)C)c1cc(Cl)ccn1 ; "LiOH" ; "THF/waterrt, thenacid work-up" ; CC(C)(C)OC(=O)N(CCCCC(=O)O)c1cc(Cl)ccn1]

To a solution of compound 1 (0.311 g) in 1:1 THF/water was added LiOH (0.0652 g) at room temperature under normal atmosphere. The reaction was stirred at room temperature until full conversion was observed by LC-MS. After 1 h, the reaction mixture was acidifed with 6 N HCl to a pH of ˜3. The product was extracted with EtOAc (3×8 mL). The combined organic phase was dried over Na₂SO₄, filtered, and concentrated, providing a clear, colorless oil. Yield: 0.311 g (104%.) LC-MS: calculated [M+H]+ 329.12 m/z, observed 329.31 m/z.

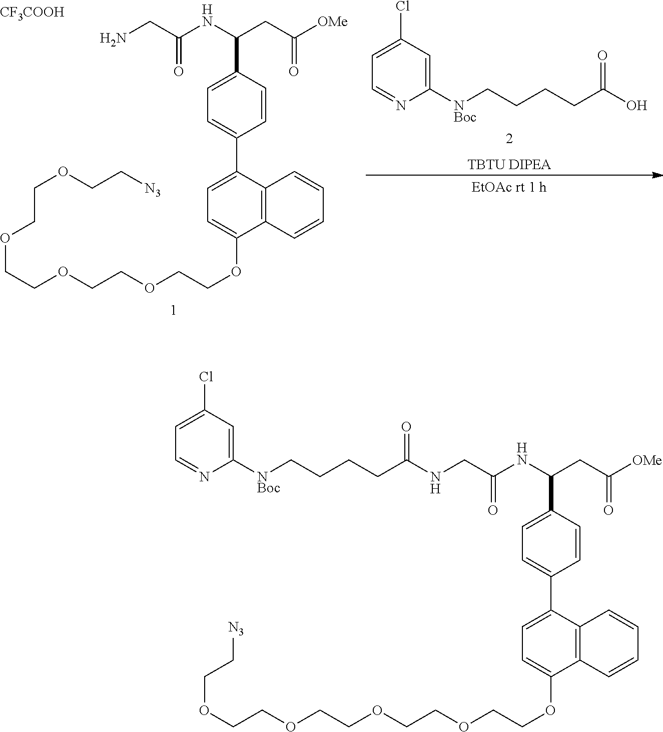 [SMILES: "CF3COOH" ; COC(=O)C[C@H](NC(=O)CN)c1ccc(-c2ccc(OCCOCCOCCOCCOCCN=[N+]=[N-])c3ccccc23)cc1 ; CC(C)(C)OC(=O)N(CCCCC(=O)O)c1cc(Cl)ccn1 ; "EtOAc rt 1 h" ; "TBTU DIPEA" ; COC(=O)C[C@H](NC(=O)CNC(=O)CCCCN(C(=O)OC(C)(C)C)c1cc(Cl)ccn1)c1ccc(-c2ccc(OCCOCCOCCOCCOCCN=[N+]=[N-])c3ccccc23)cc1]

To a solution of compounds 1 (0.0700 g) and 2 (0.0328 g) in EtOAc was added TBTU (0.0366 g) and then DIPEA (0.066 mL) under ambient conditions. The reaction was stirred for 1 h until full conversion was observed by LC-MS. The reaction mixture was then quenched with NaHCO₃ (8 mL). The product was extracted with EtOAc (3×5 mL) and 20% CF₃CH₂OH/DCM (3×5 mL) and then washed with water (3×5 mL) and brine (5 mL). The combined organic phase was dried over Na₂SO₄, filtered, and concentrated. The residue was purified by CombiFlash® using silica gel as the stationary phase with a gradient of DCM to 20% MeOH in DCM (0-100%), in which product eluted at 21% B. The product was concentrated under vacuum to provide a clear colorless oil. Yield: 0.0790 g (89.1%.) LC-MS: calculated [M+H]+ 934.40 m/z, observed 935.13 m/z.

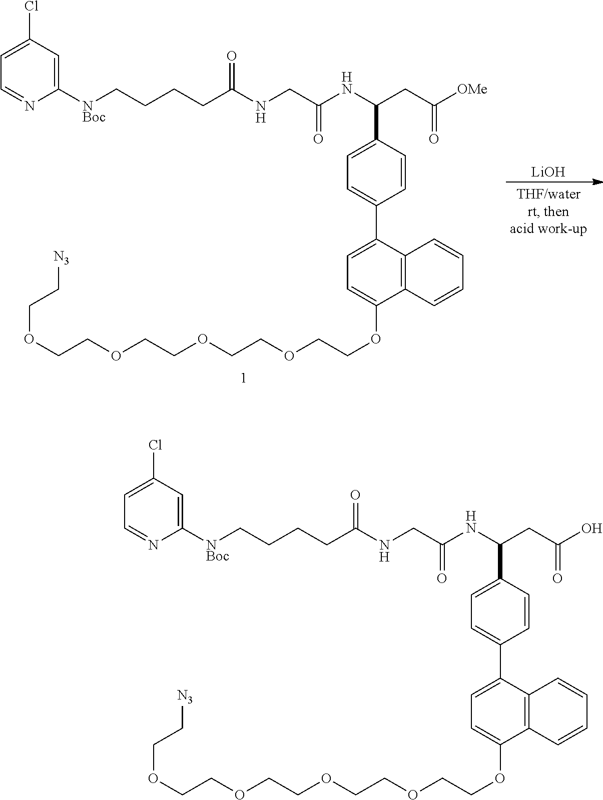 [SMILES: COC(=O)C[C@H](NC(=O)CNC(=O)CCCCN(C(=O)OC(C)(C)C)c1cc(Cl)ccn1)c1ccc(-c2ccc(OCCOCCOCCOCCOCCN=[N+]=[N-])c3ccccc23)cc1 ; "LiOH" ; "THF/waterrt, thenacid work-up" ; CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1cc(Cl)ccn1]

To a solution of compound 1 (0.0790 g) in 1:1 THF/water was added LiOH (0.0061 g) at room temperature under normal atmosphere. The reaction was stirred at room temperature until full conversion was observed by LC-MS. After 1 h, the reaction mixture was acidifed with 6 N HCl to a pH of ˜3-4. The product was extracted with 20% CF₃CH₂OH/DCM (3×8 mL). The combined organic phase was dried over Na₂SO₄, filtered, and concentrated, providing a clear and colorless oil. Yield: 0.0776 g (99.7%.) LC-MS: calculated [M+H]+ 920.39 m/z, observed 921.00 m/z.

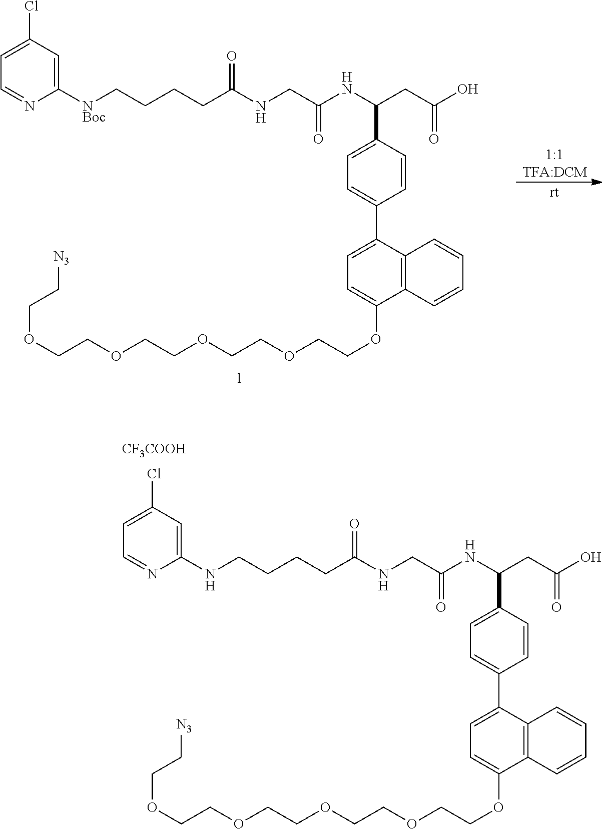 [SMILES: CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1cc(Cl)ccn1 ; "1:1TFA:DCM" ; "rt" ; "CF3COOH" ; [N-]=[N+]=NCCOCCOCCOCCOCCOc1ccc(-c2ccc([C@H](CC(=O)O)NC(=O)CNC(=O)CCCCNc3cc(Cl)ccn3)cc2)c2ccccc12]

To a solution of compound 1 (0.0776 g) in DCM was added TFA at rt (1:30 pm). The reaction was stirred under ambient conditions. Reaction was stirred overnight until full conversion was confirmed via LC-MS. The reaction mixture was azeotroped with PhMe and concentrated under vacuum. No isolation was necessary. Concentration provided a yellow oil. Yield: 0.0590 g (74.9%.) LC-MS: calculated [M+H]+ 820.34 m/z, observed 820.99 m/z.

### Synthesis of Compound 59p, (S)-3-(4-(4-((14-azido-3,6,9,12-tetraoxatetradecyl)oxy)naphthalen-1-yl)phenyl)-3-(2-(5-((4-fluoropyridin-2-yl)amino)pentanamido)acetamido)propanoic acid

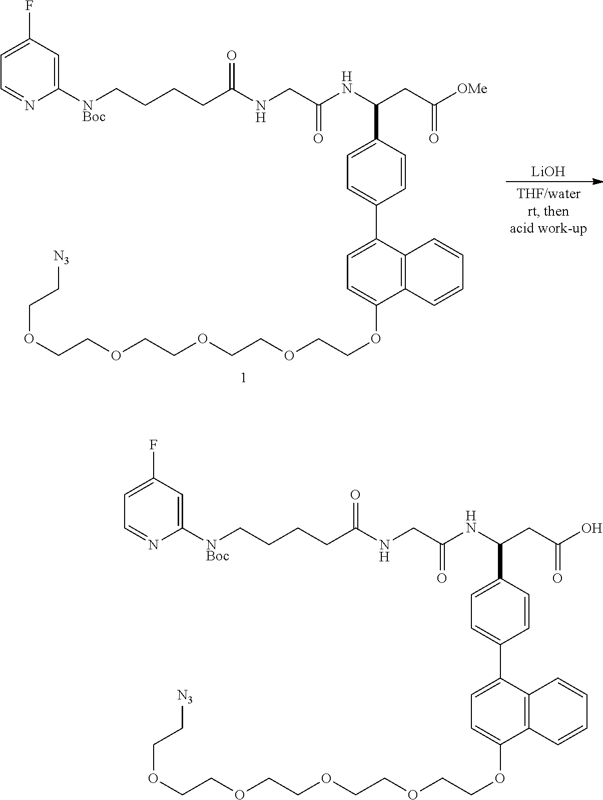 [SMILES: COC(=O)C[C@H](NC(=O)CNC(=O)CCCCN(C(=O)OC(C)(C)C)c1cc(F)ccn1)c1ccc(-c2ccc(OCCOCCOCCOCCOCCN=[N+]=[N-])c3ccccc23)cc1 ; "LiOH" ; "THF/waterrt, thenacid work-up" ; CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1cc(F)ccn1]

To a solution of compound 1 (0.121 g) in 1:1 THF/water was added LiOH (0.0095 g) at room temperature under normal atmosphere. The reaction was stirred at room temperature until full conversion was observed by LC-MS. After 1 h, the reaction mixture was acidifed with 6 N HCl to a pH of ˜3-4. The product was extracted with 20% CF₃CH₂OH/DCM (3×8 mL). The combined organic phase was dried over Na₂SO₄, filtered, and concentrated, providing a cream white solid. Yield: 0.0868 g (72.8%.) LC-MS: calculated [M+H]+ 904.42 m/z, observed 905.07 m/z.

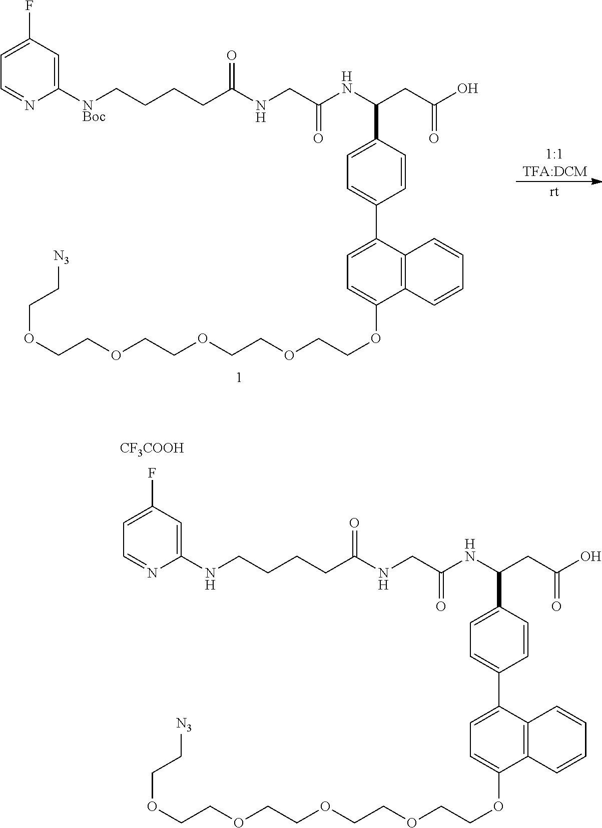 [SMILES: CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1cc(F)ccn1 ; "1:1TFA:DCM" ; "rt" ; "CF3COOH" ; [N-]=[N+]=NCCOCCOCCOCCOCCOc1ccc(-c2ccc([C@H](CC(=O)O)NC(=O)CNC(=O)CCCCNc3cc(F)ccn3)cc2)c2ccccc12]

To a solution of compound 1 (0.868 g) in DCM was added TFA (0.220 mL) at room temperature. The reaction was stirred under ambient conditions. The reaction was stirred overnight until full conversion was confirmed via LC-MS. The reaction mixture was azeotroped with PhMe and concentrated under vacuum. No isolation was necessary. Concentration provided a yellow oil. Yield: 0.0380 g (43.1%.) LC-MS: calculated [M+H]+ 804.37 m/z, observed 804.78 m/z.

### Synthesis of Compound 60p, (S)-3-(4-(4-((14-azido-3,6,9,12-tetraoxatetradecyl)oxy)naphthalen-1-yl)phenyl)-3-(2-(5-(pyridin-2-ylamino)pentanamido)acetamido)propanoic acid

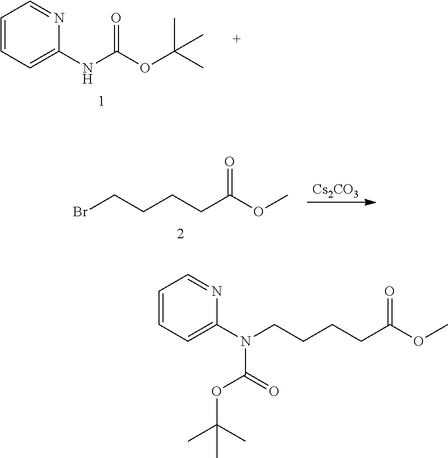

To a solution of compound 1 (211 mg, 1.086 mmol, 1.0 equiv.), and cesium carbonate (530 mg, 1.629 mmol, 1.5 equiv.) in anhydrous DMF (2 mL) was added compound 2 (0.187 mL, 1.303 mmol, 1.2 equiv.) at room temperature. The reaction was kept at room temperature for 72 hrs. The reaction was quenched with water (5 mL). The aqueous phase was extracted with ethyl acetate (3×5 mL). The organic phase was combined, dried over Na₂SO₄, and concentrated. The product was purified by CombiFlash® and eluted with 10-15% ethyl acetate in hexane. LC-MS: calculated [M+H]+ 309.17, found 309.42.

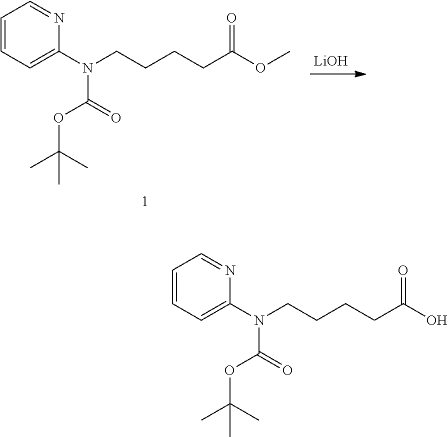

To a solution of compound 1 (348 mg, 1.128 mmol, 1.0 equiv.) in THF (5 mL) and water (5 mL) was added lithium hydroxide (81 mg, 3.385 mmol, 3.0 equiv.) at room temperature. The reaction was kept at room temperature for 1 hr. The reaction was quenched with HCl solution and the pH was adjusted to 3.0. The aqueous phase was extracted with ethyl acetate (3×10 mL). The organic phase was combined, dried over Na₂SO₄, and concentrated. The product was used directly without further purification. LC-MS: calculated [M+H]+ 295.16, found 295.38.

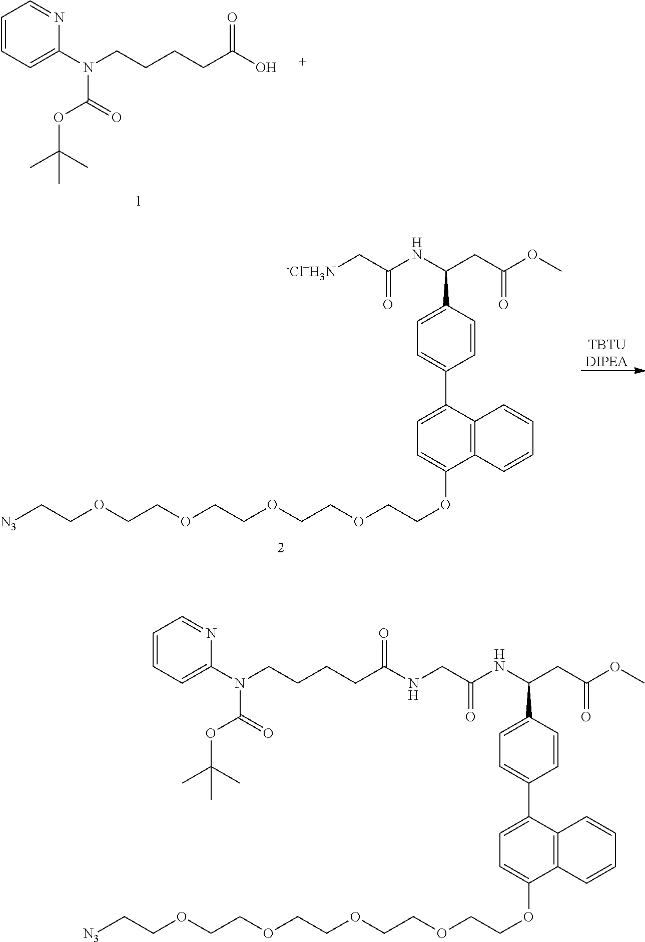

To a solution of compound 1 (44 mg, 0.149 mmol, 1.0 equiv.), compound 2 (108 mg, 0.164 mmol, 1.1 equiv.) and diisopropylethylamine (0.078 mL, 0.448 mmol, 3.0 equiv.) in anhydrous DMF (1 mL) was added TBTU (57 mg, 0.179 mmol, 1.2 equiv.) at room temperature. The reaction was kept at room temperature for 2 hrs. The reaction was quenched with saturated NaHCO₃ (5 mL), and the aqueous phase was extracted with ethyl acetate (3×5 mL). The organic phase was combined, dried over Na₂SO₄, and concentrated. The product was purified by CombiFlash® and eluted with 3-5% methanol in dichloromethane. LC-MS: calculated [M+H]+ 900.44, found 901.19.

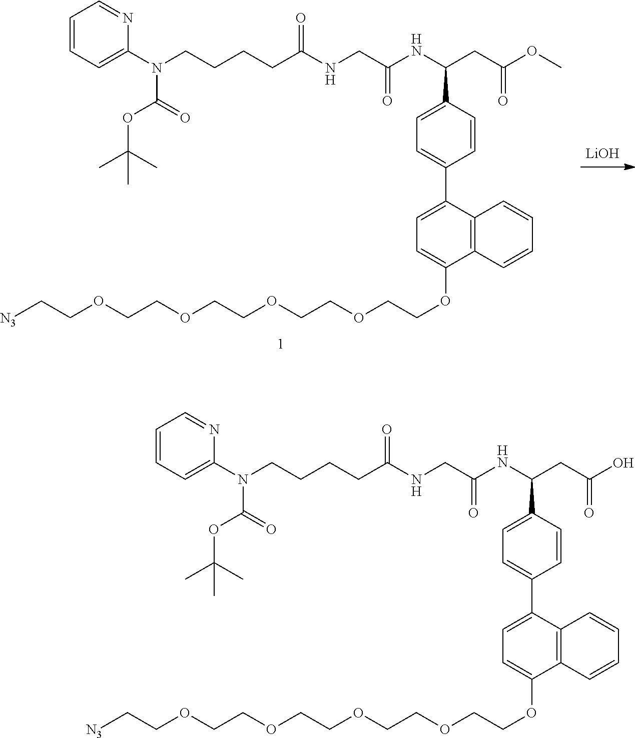 [SMILES: COC(=O)C[C@H](NC(=O)CNC(=O)CCCCN(C(=O)OC(C)(C)C)c1ccccn1)c1ccc(-c2ccc(OCCOCCOCCOCCOCCN=[N+]=[N-])c3ccccc23)cc1 ; "LiOH" ; CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1ccccn1]

To a solution of compound 1 (110 mg, 0.122 mmol, 1.0 equiv.) in THF (3 mL) and water (3 mL) was added lithium hydroxide (9 mg, 0.366 mmol, 3.0 equiv.) at room temperature. The reaction was kept at room temperature for 3 hrs. The reaction was quenched with HCl solution and the pH was adjusted to 3.0. The aqueous phase was extracted with ethyl acetate (3×5 mL). The organic phase was combined, dried over Na₂SO₄, and concentrated. The product was used directly without further purification. LC-MS: calculated [M+H]+886.43, found 886.97.

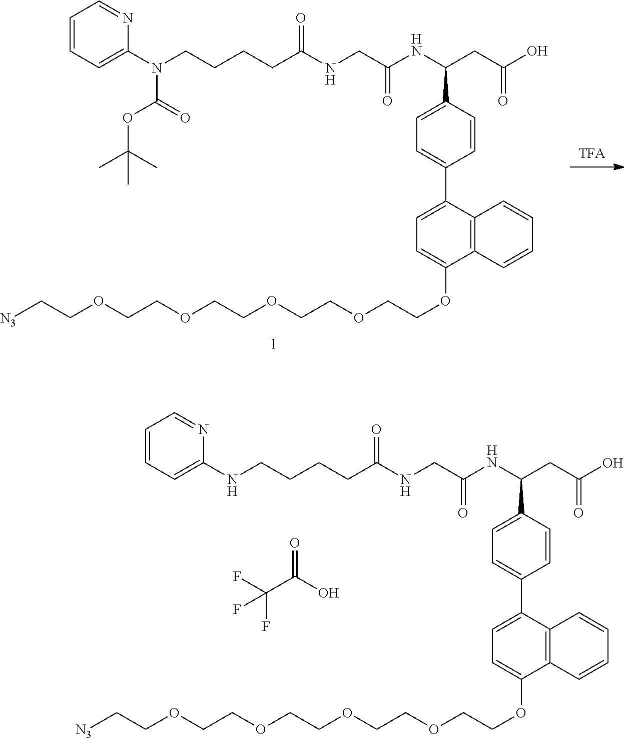 [SMILES: CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1ccccn1 ; [N-]=[N+]=NCCOCCOCCOCCOCCOc1ccc(-c2ccc([C@H](CC(=O)O)NC(=O)CNC(=O)CCCCNc3ccccn3)cc2)c2ccccc12 ; O=C(O)C(F)(F)F ; "TFA"]

To a solution of compound 1 (108 mg, 0.121 mmol, 1.0 equiv.) in dichloromethane (2 mL) was added trifluoroacetic acid (2 mL) at room temperature. The reaction was kept at room temperature for 2 hrs. The solvent was removed. The product was used directly without further purification. LC-MS: calculated [M+H]+ 786.37, found 787.05.

### Example 2. Syntheses of RNAi Agents and Conjugation Reactions

The αvβ6 integrin ligands can be conjugated to one or more RNAi agents useful for inhibiting the expression of one or more targeted genes. The αvβ6 integrin ligands facilitate the delivery of the RNAi agents to the targeted cells and/or tissues. Example 1, above, described the synthesis of certain αvβ6 integrin ligands disclosed herein. The following describes the general procedures for the syntheses of certain αvβ6 integrin ligand-RNAi agent conjugates that are illustrated in the non-limiting Examples set forth herein.

A. Synthesis ofRNAi Agents RNAi agents can be synthesized using methods generally known in the art. For the synthesis of the RNAi agents illustrated in the Examples set forth herein, the sense and antisense strands of the RNAi agents were synthesized according to phosphoramidite technology on solid phase used in oligonucleotide synthesis. Depending on the scale, a MerMade96E® (Bioautomation), a MerMade12@(Bioautomation), or an OP Pilot 100 (GE Healthcare) was used. Syntheses were performed on a solid support made of controlled pore glass (CPG, 500 Å or 600 Å, obtained from Prime Synthesis, Aston, PA, USA). All RNA and 2′-modified RNA phosphoramidites were purchased from Thermo Fisher Scientific (Milwaukee, WI, USA). Specifically, the following 2′-O-methyl phosphoramidites were used: (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropyl-amino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites carried the same protecting groups as the 2′-O-methyl RNA amidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes (Wilmington, MA, USA). The following UNA phosphoramidites were used: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diiso-propyl)]-phosphoramidite. TFA aminolink phosphoramidites were also commercially purchased (ThermoFisher).

In some examples, the αvβ6 integrin ligands disclosed herein are conjugated to the RNAi agents by linking the components to a scaffold that includes a tri-alkyne group. In some examples, the tri-alkyne group is added by using a tri-alkyne-containing phosphoramidite, which can be added at the 5′ terminal end of the sense strand of an RNAi agent. When used in connection with the RNAi agents presented in certain Examples herein, tri-alkyne-containing phosphoramidites were dissolved in anhydrous dichloromethane or anhydrous acetonitrile (50 mM), while all other amidites were dissolved in anhydrous acetonitrile (50 mM), and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 90 sec (2′ O-Me), and 60 sec (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

Alternatively, where the αvβ6 integrin ligands are conjugated to the RNAi agents via a tri-alkyne scaffold, instead of using a phosphoramidite approach, tri-alkyne-containing compounds can be introduced post-synthetically (see, for example, section E, below). When used in connection with the RNAi agents presented in certain Examples set forth herein, when attaching a tri-alkyne group post-synthetically to the 5′ end of the sense strand the 5′ terminal nucleotide of the sense strand was functionalized with a nucleotide that included a primary amine at the 5′ end to facilitate attachment to the tri-alkyne-containing scaffold. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 90 sec (2′ O-Me), and 60 sec (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

B. Cleavage and deprotection of support bound oligoiner. After finalization of the solid phase synthesis, the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).

C. Purification. Crude oligomers were purified by anionic exchange HPLC using a TSKgel SuperQ-5PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G-25 fine with a running buffer of 100 mM ammonium bicarbonate, pH 6.7 and 20% Acetonitrile or filtered water.

D. Annealing. Complementary strands were mixed by combining equimolar RNA solutions (sense and antisense) in 1× PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1× PBS. The solution absorbance at 260 nm was then multiplied by a conversion factor and the dilution factor to determine the duplex concentration. The conversion factor used was either 0.037 mg/(mL·cm), or, alternatively for some experiments, a conversion factor was calculated from an experimentally determined extinction coefficient.

E. Conjugation of Trigger-Ligand Linker. A DBCO linker having the formula:

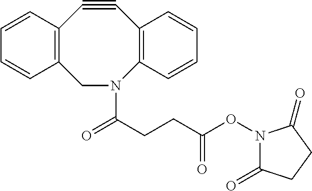 [SMILES: O=C(CCC(=O)N1Cc2ccccc2C#Cc2ccccc21)ON1C(=O)CCC1=O]

was used to conjugate αvβ6 ligands described herein for each of the Examples 4-7 below. An amidation reaction to link the free amine at the 5′ terminus of the sense strand, and a copper click reaction was used to conjugate the respective azide-containing ligands of formulas 40p-60p. Example conditions for the copper click reaction are provided in Example 2G below.

To conjugate an activated ester such as a DBCO linker to a 5′ amine or 3′ amine functionalized sense strand of an RNAi agent, the synthesized and annealed RNAi agent was first dissolved in DMSO and 10% water (v/v %) at 25 mg/mL. Then 50-100 equivalents of TEA and 3 equivalents of activated ester linker were added to the mixture. The solution was allowed to react for 1-2 hours, while monitored by RP-HPLC-MS (mobile phase A 100 mM HFIP, 14 mM TEA; mobile phase B: acetonitrile on an XBridge C18 column, Waters Corp.)

The product was then precipitated by adding 12 mL acetonitrile and 0.4 mL PBS and centrifuging the solid to a pellet. The pellet was then redissolved in 0.4 mL of 1×PBS and 12 mL of acetonitrile. The resulting pellet was dried on high vacuum for one hour.

F. Conjugation of αvβ6 Integrin Ligands.

i.Propargyl Linker. Either prior to or after annealing, the 5′ or 3′ tridentate alkyne functionalized sense strand is conjugated to the αvβ6 Integrin Ligands. The following example describes the conjugation of αvβ6 integrin ligands to the annealed duplex: Stock solutions of 0.5M Tris(3-hydroxypropyltriazolylmethyl)amine (THPTA), 0.5M of Cu(II) sulfate pentahydrate (Cu(II)SO₄·5 H₂O) and 2M solution of sodium ascorbate were prepared in deionized water. A 75 mg/mL solution in DMSO of αvβ6 integrin ligand was made. In a 1.5 mL centrifuge tube containing tri-alkyne functionalized duplex (3 mg, 75 μL, 40 mg/mL in deionized water, ˜15,000 g/mol), 25 μL of 1M Hepes pH 8.5 buffer is added. After vortexing, 35 μL of DMSO was added and the solution is vortexed. αvβ6 integrin ligand was added to the reaction (6 eq/duplex, 2 eq/alkyne, ˜15 μL) and the solution is vortexed. Using pH paper, pH was checked and confirmed to be pH ˜8. In a separate 1.5 mL centrifuge tube, 50 μL of 0.5M THPTA was mixed with 10 uL of 0.5M Cu(II)SO₄·5 H₂O, vortexed, and incubated at room temp for 5 min. After 5 min, THPTA/Cu solution (7.2 μL, 6 eq 5:1 THPTA:Cu) was added to the reaction vial, and vortexed. Immediately afterwards, 2M ascorbate (5 μL, 50 eq per duplex, 16.7 per alkyne) was added to the reaction vial and vortexed. Once the reaction was complete (typically complete in 0.5-1 h), the reaction was immediately purified by non-denaturing anion exchange chromatography.

ii. DBCO Linker. The pellet was dissolved in 50/50 DMSO/water at 50 mg/mL. Then 1.5 equivalents of ligand was added per DBCO linker. The reaction was allowed to proceed for 30-60 minutes. The reaction was monitored by RP-HPLC-MS (mobile phase A 100 mM HFIP, 14 mM TEA; mobile phase B: acetonitrile on an XBridge C18 column, Waters Corp.) The product was precipitated by adding 12 mL acetonitrile, 0.4 mL PBS and the solid was centrifuged to a pellet. The pellet was redissolved in 0.4 mL 1× PBS and then 12 mL of acetonitrile was added. The pellet was dried on high vacuum.

G. PK/PD Modulators

In some examples below, Pharmacokinetic and or Pharmacodynamic (PK/PD) modulators were attached to the RNAi agent in addition to the αvβ6 integrin receptor targeting ligands. Example PK/PD modulators as used in further examples are shown in the table below (PK/PD modulators were purchased from commercial suppliers where indicated):

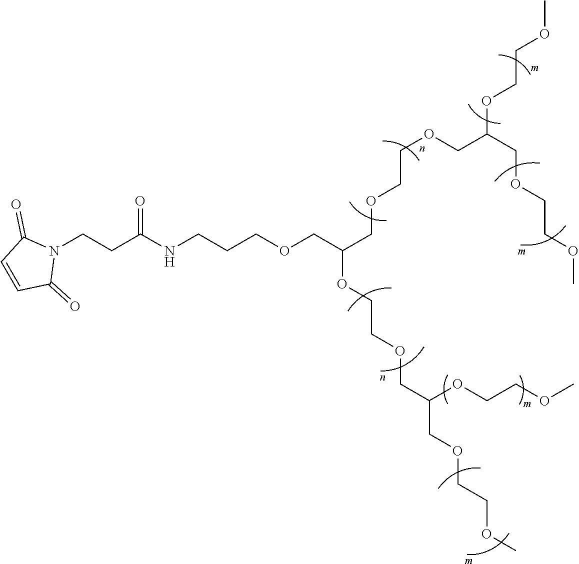

PEG40K (2x2-arm),
wherein n and m are integers, and the molecular weight of the PEG groups is about 40
kilodaltons
NOF, Sunbright ® GL4-400MA

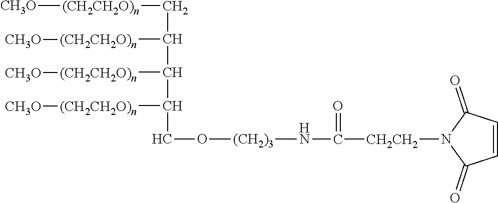

PEG40K (4-arm),
wherein n is an integer, and the molecular weight of the PEG groups is about 40 kilodaltons
NOF, Sunbright ® XY4-400MA

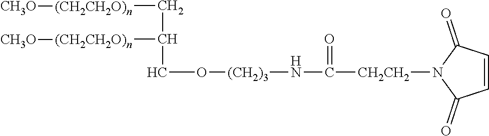

PEG40K (2-arm),
wherein n is an integer, and the molecular weight of the PEG groups is about 40 kilodaltons
NOF, Sunbright ® GL2-400MA

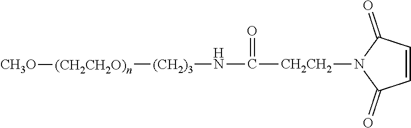

PEG40K,
wherein n is an integer, and the molecular weight of the PEG groups is about 40 kilodaltons
NOF, Sunbright ® ME-400MA

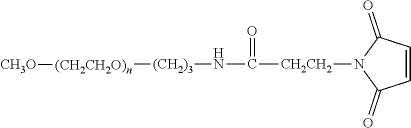

PEG10K,
wherein n is an integer, and the molecular weight of the PEG groups is about 10 kilodaltons
NOF, Sunbright ® ME-100MA

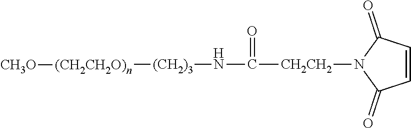

PEG5K,
wherein n is an integer, and the molecular weight of the PEG groups is about 5 kilodaltons
NOF, Sunbright ® ME-050MA

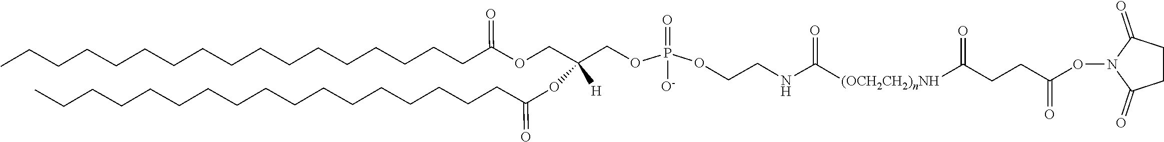

DSPE-PEG5K-NHS (Naonsoft Polymers ™ #SKU 1544)
(1,2-distearoyl-sn-glycero-3-phosphoethanolamine-N-[succinimidyl(polyethylene glycol)]),
wherein n is an integer and the molecular weight of the PEG groups is about 5 kilodaltons

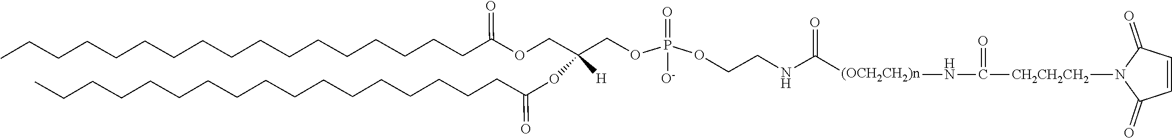

DSPE-PEG5K-MAL (Naonsoft Polymers ™ SKU #2049)
1,2-distearoyl-sn-glycero-3-phosphoethanolamine-N-[maleimide(polyethylene glycol)],
Wherein n is an integer and the molecular weight of the PEG groups is about 5 kilodaltons

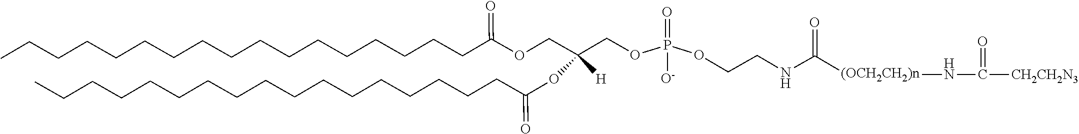

DSPE-PEG5K-N3 (Naonsoft Polymers ™ SKU #2274)
1,2-distearoyl-sn-glycero-3-phosphoethanolamine-N-[azido(polyethylene glycol)], wherein n
is an integer and the molecular weight of the PEG groups is about 5 kilodaltons

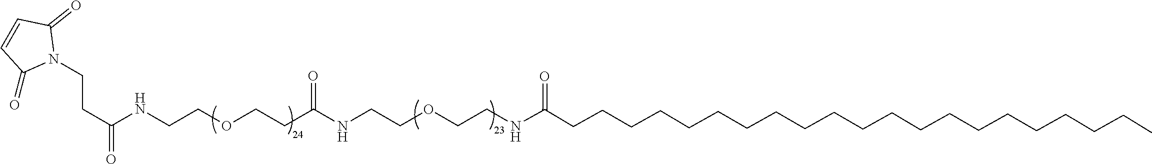

PEG47 + C22

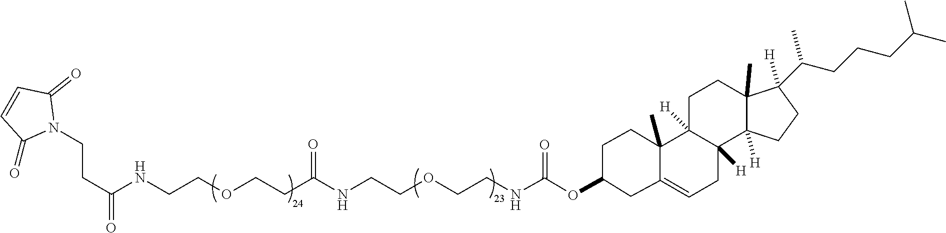

PEG47 + CLS (cholesterol)

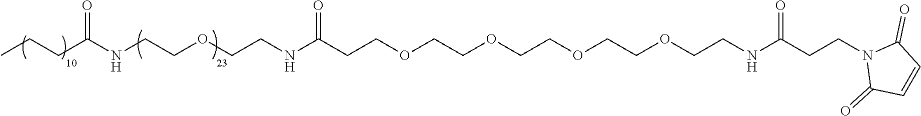

PEG23 + C22

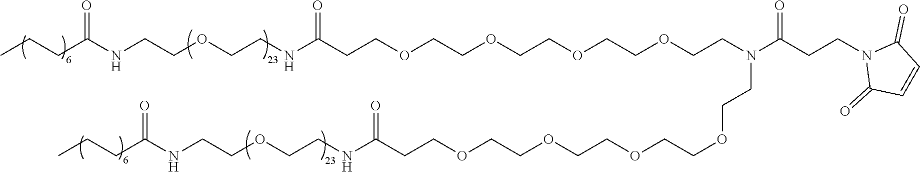

Bis(PEG23 + C14)

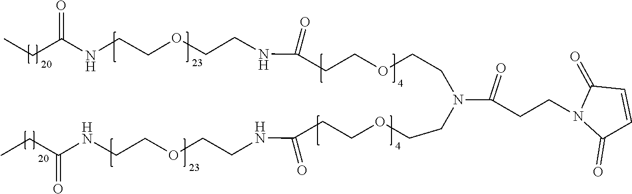

Bis(PEG23 + C22)

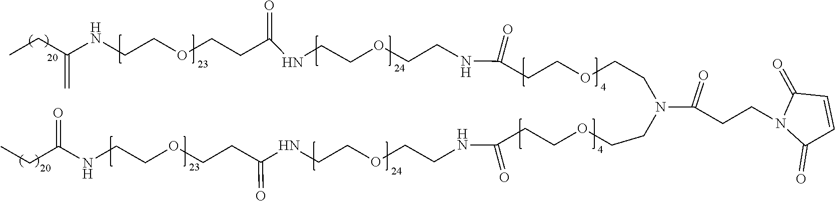

Bis(PEG47 + C22)

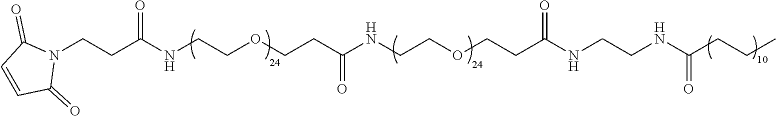

PEG48 + C22

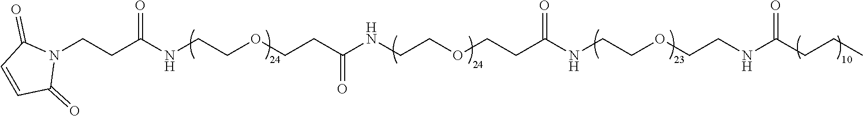

PEG71 + C22

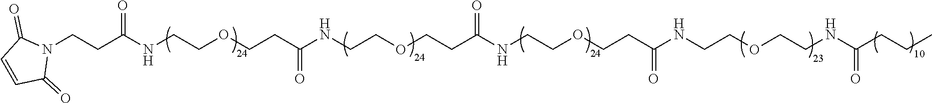

PEG95 + C22

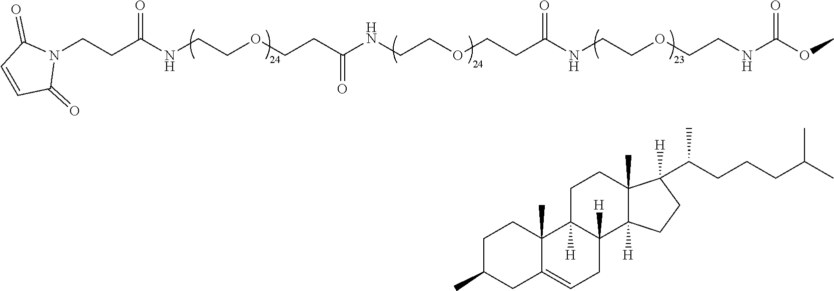

PEG71 + CLS

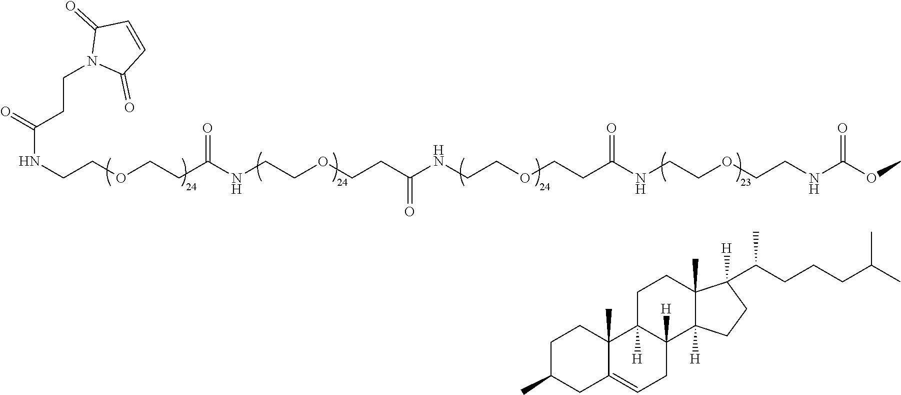

PEG95 + CLS

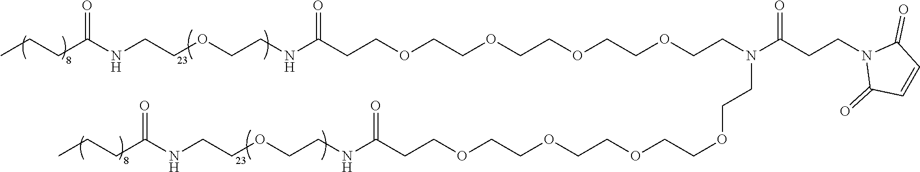

Bis(PEG23 + C18)

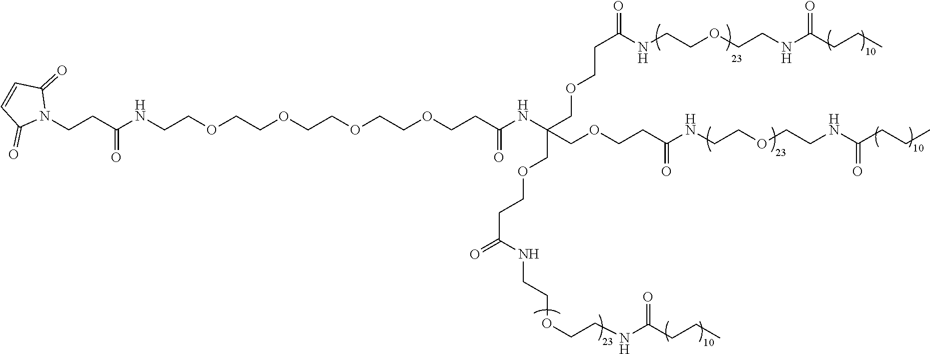

Tris(PEG23 + C22)

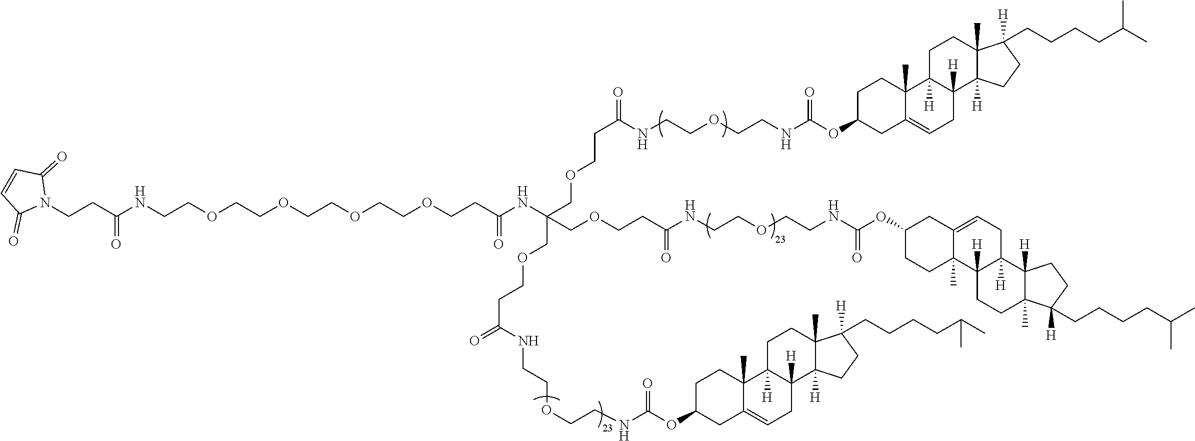

Tris(PEG23 + CLS)

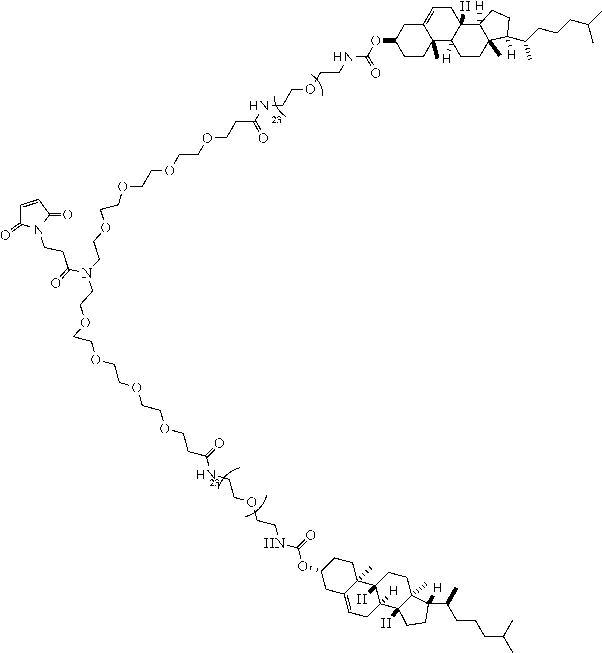

Bis(PEG23 + CLS)

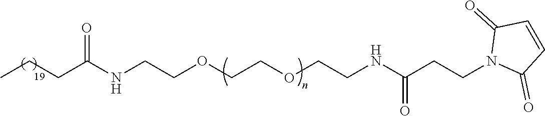

PEG5K + C22
wherein n is an integer and the molecular weight of the PEG units is about 5 kilodaltons

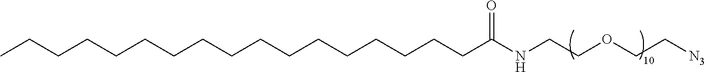

C18

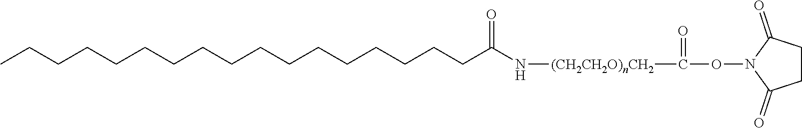

(NHS)-PEG1K + C18 (Naonsoft Polymers ™ SKU #10668-1000)
wherein n is an integer and the molecular weight of the PEG units is about 1 kilodalton

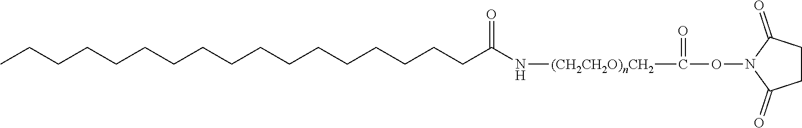

(NHS)-PEG2K + C18 (Naonsoft Polymers ™ SKU #10668-2000)
wherein n is an integer and the molecular weight of the PEG units is about 2 kilodaltons

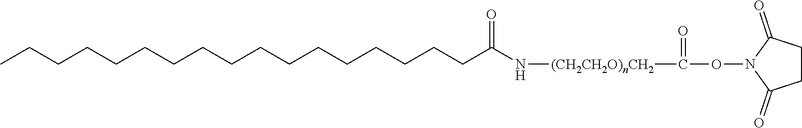

(NHS)-PEG5K + C18 (Naonsoft Polymers ™ SKU #10668-5000)
wherein n is an integer and the molecular weight of the PEG units is about 5 kilodaltons

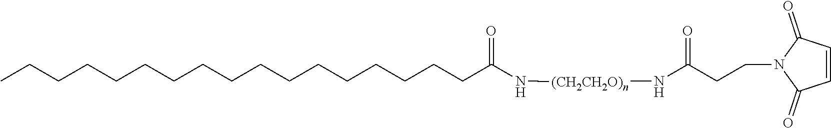

(MAL)-PEG5K + C18 (Naonsoft Polymers ™ SKU #10647)
wherein n is an integer and the molecular weight of the PEG units is about 5 kilodaltons

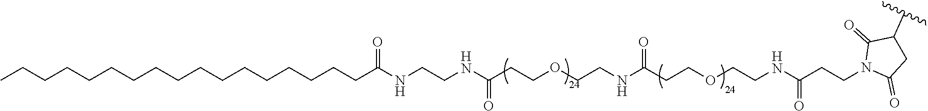

PEG48 + C18

### Synthesis of PEG95+C₂₂

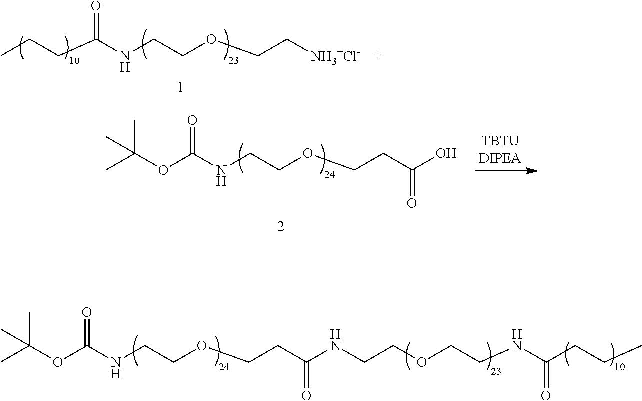

To a solution of compound 1 (60 mg, 0.0419 mmol, 1.0 equiv.), compound 2 (52 mg, 0.0419 mmol, 1.0 equiv.) and diisopropylethylamine (0.022 mL, 0.125 mmol, 3.0 equiv.) in anhydrous DMF (3 mL) was added TBTU (16 mg, 0.0503 mmol, 1.2 equiv.) at room temperature. The reaction was kept at room temperature for 2 hrs. The reaction mixture was concentrated. The product was purified by CombiFlash® and was eluted with 6-8% methanol in dichloromethane. LC-MS: calculated [M+4H]+/4 656.66, found 656.17. Yield: 0.063 g (57.3%.)

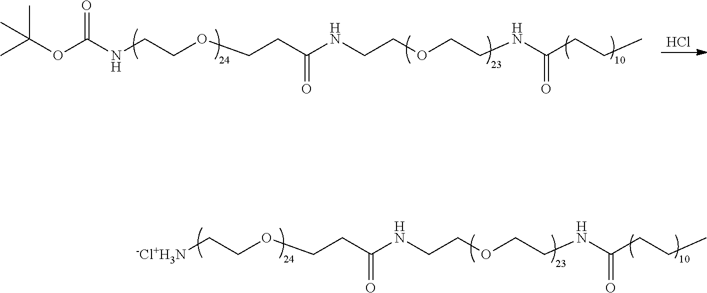

To a solution of compound 1 (60 mg, 0.0229 mmol, 1.0 equiv.) in anhydrous 1,4-dioxane (0.5 mL) was added HCl solution in dioxane (0.286 mL, 1.143 mmol, 50 equiv.) at room temperature. The reaction was kept at room temperature for 30 min and the solvent was concentrated. The product was used directly without further purification. LC-MS: calculated [M+3H]+/3 841.88, found 841.48, calculated [M+4H]+/4 631.66, found 632.41.

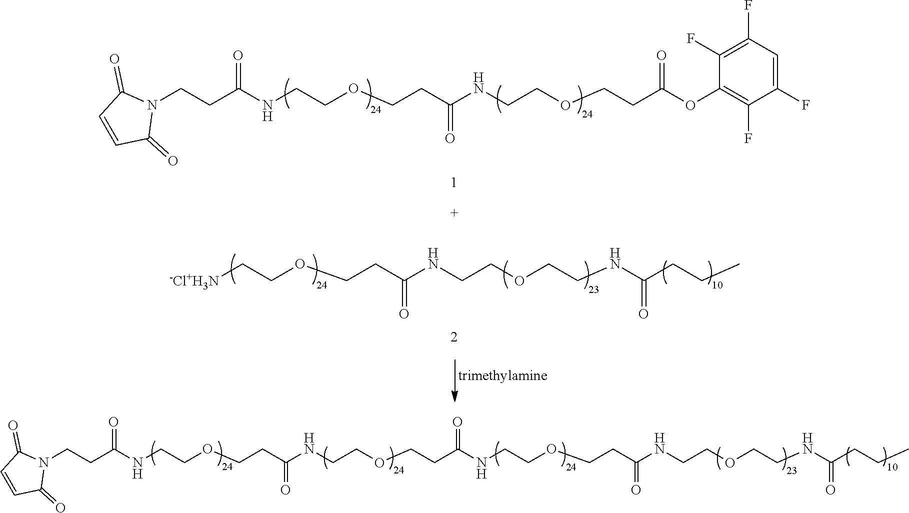

To a solution of compound 1 (55 mg, 0.0214 mmol, 1.0 equiv.) and compound 2 (54.7 mg, 0.0214 mmol, 1.0 equiv.) in anhydrous DMF (2 mL) was added triethylamine (0.009 mL, 0.0641 mmol, 3.0 equiv.) at room temperature. The reaction was kept at room temperature for 2 hrs and the solvent was concentrated. The product was separated by CombiFlash and was eluted with 15-20% methanol in dicholoromethane. LC-MS: calculated [M+5H]+/5 986.80, found 987.19, calculated [M+6H]+/6 822.50, found 822.64.

H. Conjugation ofPK/PD Modulators. Either prior to or after annealing and prior to or after conjugation of one or more targeting ligands, one or more PK enhancers can be linked to the an RNAi agent. The following describes the general conjugation process used to link PK enhancers to the constructs set forth in the Examples depicted herein. The following describes the general process used to link a maleimide-functionalized PK enhancer to the (C6-SS-C6) or (6-SS-6) functionalized sense strand of an RNAi agent by undertaking a dithiothreitol reduction of disulfide followed by a thiol-Michael Addition of the respective PK enhancer: In a vial containing functionalized sense strand was dissolved at 75 mg/mL in 0.1M Hepes pH 8.5 buffer, and 25 eq of dithiothreitol is added. Once the reaction was complete (typically complete in 0.5-1 h), the conjugate was precipitated three times in a solvent system of 1× phosphate buffered saline/acetonitrile (1:40 ratio), and dried. A 75 mg/mL solution of maleimide functionalized PK enhancer in DMSO was then made. The disulfide-reduced (i.e., 3′ C6-SH, 5′ HS-C6, or 3′ 6-SH functionalized) sense strand was dissolved 100 mg/mL in deionized water, and three equivalents of maleimide-functionalized PK enhancer was added. Once the reaction was complete (typically complete in 1 h-3 h), the conjugate was precipitated in a solvent system of 1× phosphate buffered saline/acetonitrile (1:40 ratio), and dried.

I. Synthesis of αvβ6 Peptide 1

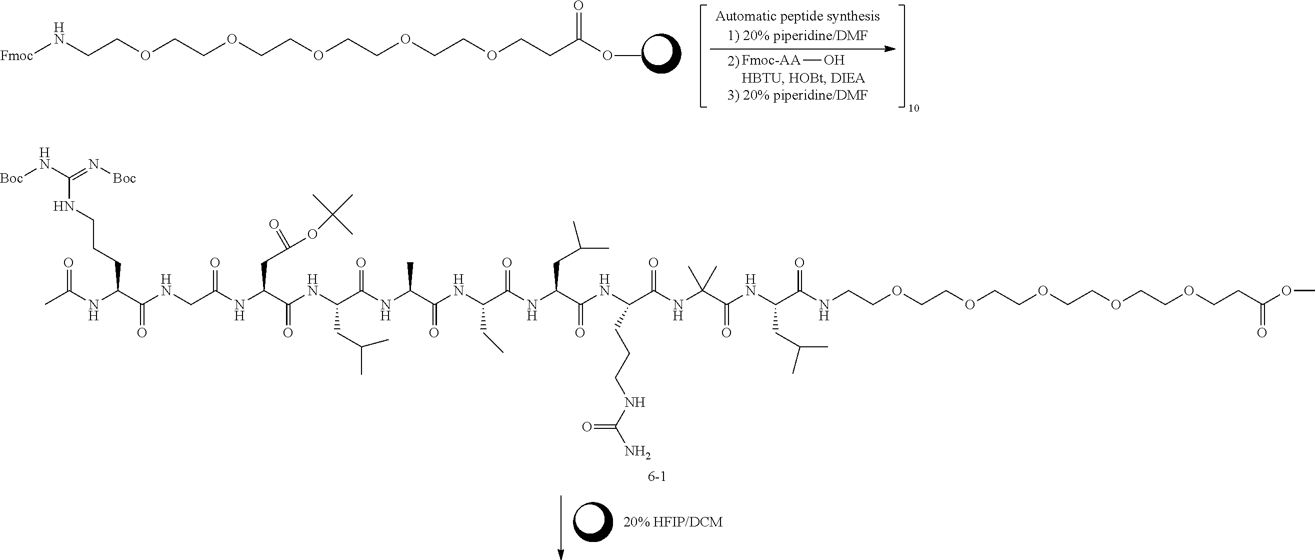

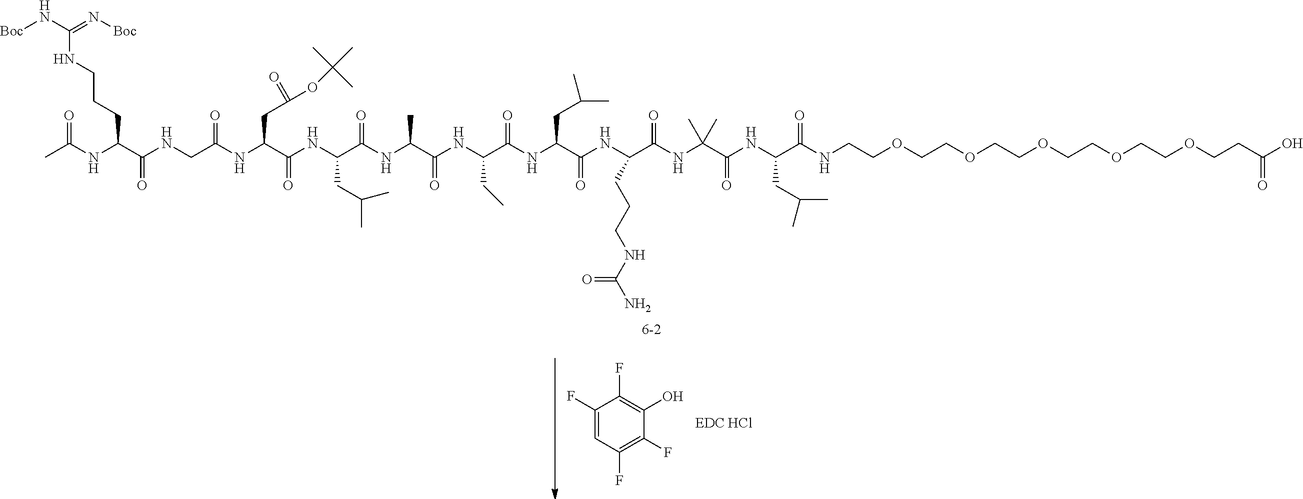 [SMILES: CC[C@H](NC(=O)[C@H](C)NC(=O)[C@H](CC(C)C)NC(=O)[C@H](CC(=O)OC(C)(C)C)NC(=O)CNC(=O)[C@H](CCCN/C(=N\C(=O)OC(C)(C)C)NC(=O)OC(C)(C)C)NC(C)=O)C(=O)N[C@@H](CC(C)C)C(=O)N[C@@H](CCCNC(N)=O)C(=O)NC(C)(C)C(=O)N[C@@H](CC(C)C)C(=O)NCCOCCOCCOCCOCCOCCC(=O)O ; Oc1c(F)c(F)cc(F)c1F]

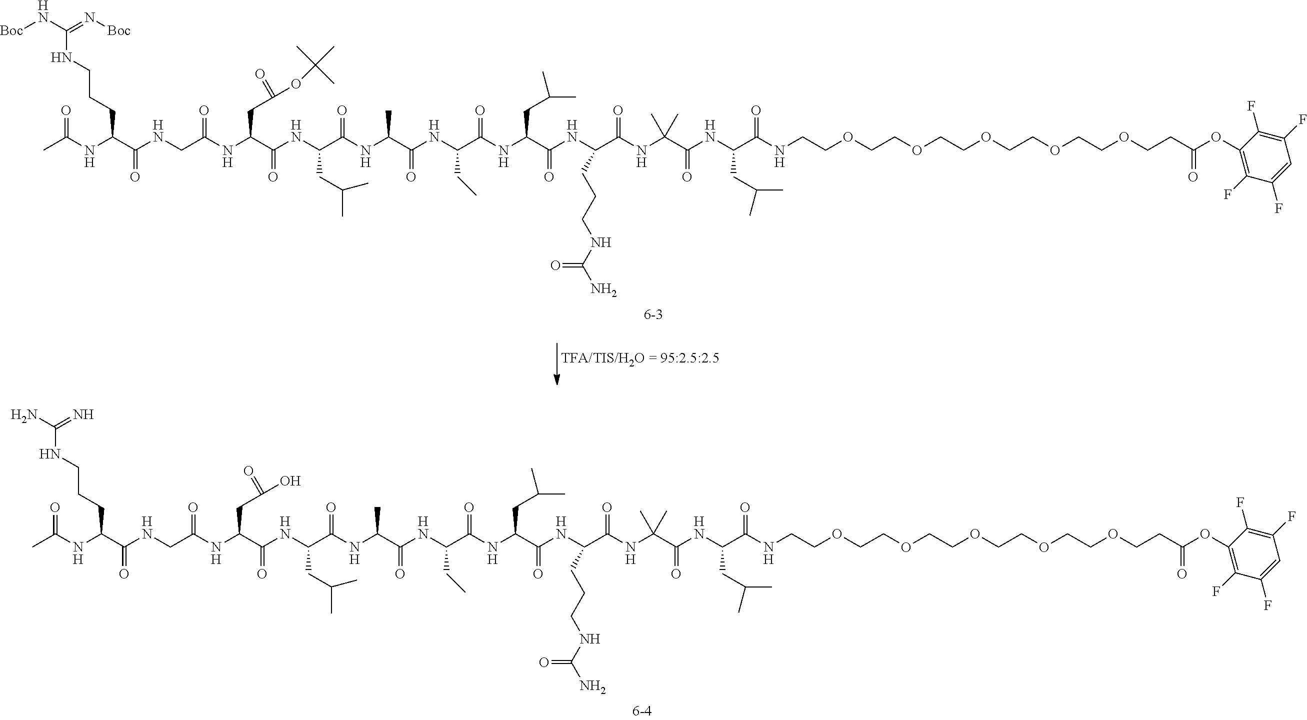 [SMILES: CC[C@H](NC(=O)[C@H](C)NC(=O)[C@H](CC(C)C)NC(=O)[C@H](CC(=O)OC(C)(C)C)NC(=O)CNC(=O)[C@H](CCCN/C(=N\C(=O)OC(C)(C)C)NC(=O)OC(C)(C)C)NC(C)=O)C(=O)N[C@@H](CC(C)C)C(=O)N[C@@H](CCCNC(N)=O)C(=O)NC(C)(C)C(=O)N[C@@H](CC(C)C)C(=O)NCCOCCOCCOCCOCCOCCC(=O)Oc1c(F)c(F)cc(F)c1F ; CC[C@H](NC(=O)[C@H](C)NC(=O)[C@H](CC(C)C)NC(=O)[C@H](CC(=O)O)NC(=O)CNC(=O)[C@H](CCCNC(=N)N)NC(C)=O)C(=O)N[C@@H](CC(C)C)C(=O)N[C@@H](CCCNC(N)=O)C(=O)NC(C)(C)C(=O)N[C@@H](CC(C)C)C(=O)NCCOCCOCCOCCOCCOCCC(=O)Oc1c(F)c(F)cc(F)c1F]

Peptide 1 was prepared by modification of Arg-Gly-Asp(tBu)-Leu-Ala-Abu-Leu-Cit-Aib-Leu-Peg₅-CO₂-2-Cl-Trt resin 1 that was obtained using general Fmoc peptide chemistry on CS Bio peptide synthesizer utilizing Fmoc-Peg₅-CO₂H preloaded 2-Cl-Trt resin on (0.79 mmol/g) at 4.1 mmol scale as described above. Following cleavage from resin the peptide 6-2 was converted into tetrafluorophenyl ester 6-3, and the crude product was used in the next step without purification.

Final deprotection was done by treatment of crude peptide 6-3 with deprotection cocktail TFA/TIS/H₂O=90:5:5 (80 mL) for 1.5 h. The reaction mixture was added dropwise to methyl tert-butyl ether (700 mL), and the resulting precipitate was collected by centrifugation. The pellets were washed with additional methyl tert-butyl ether (500 mL). The residue was purified by RP-HPLC (Phenomenex Gemini C18 250×50 mm, 10 micron, 60 mL/min, 30-45% ACN gradient in water containing 0.1% TFA, approx. 1 gram of crude per run), affording 4.25 g of pure peptide 6-4.

J. Conjugation of αvβ6 Peptide 1

The following procedure may be used to conjugate an activated ester-functionalized targeting ligand such as αvβ6 peptide 1 to an amine functionalized RNAi agent comprising an amine, such as C6-NH2, NH2-C6, or (NH2-C6)s, as shown in Table A, above.

An annealed, lyophilized RNAi agent was dissolved in DMSO and 10% water (v/v %) at 25 mg/mL. Then 50-100 equivalents TEA and three equivalents of activated ester targeting ligand were added to the mixture. The reaction was allowed to stir for 1-2 hours while monitored by RP-HPLC-MS (mobile phase A: 100 mM HFIP, 14 mM TEA; mobile phase B: Acetonitrile; column: XBridge C18). After the reaction was complete, 12 mL of acetonitrile was added followed by 0.4 mL of PBS and then the mixture was centrifuged. The solid pellet was collected and dissolved in 0.4 mL of 1× PBS and then 12 mL of acetonitrile was added. The resulting pellet was collected and dried on high vacuum for 1 hour.

### Example 3. In Vivo Intravenous Administration of RNAi Agents Targeting Myostatin Conjugated to αvβ6 Integrin Ligands in Mice

RNAi agents that included a sense strand and an antisense strand were synthesized according to phosphoramidite technology on solid phase in accordance with general procedures known in the art and commonly used in oligonucleotide synthesis as set forth in Example 2 herein. The RNAi agents included an antisense strand having a nucleobase sequence at least partially complementary to the myostatin gene. The myostatin RNAi agents were designed to be capable of degrading or inhibiting translation of messenger RNA (mRNA) transcripts of myostatin in a sequence specific manner, thereby inhibiting expression of the myostatin gene. The RNAi agent used in this Example (AD06326) was comprised of modified nucleotides and more than one non-phosphodiester linkage, and included the following nucleotide sequences: Sense strand sequence (5′→3′): (NH₂-C₆)s(invAb)sggccaugaUfCfUfugcuguaacas(invAb)(C6-SS-C6)dT (SEQ ID NO:1) Antisense strand sequence (5′→3′) usGfsusUfaCfagcaaGfaUfcAfuGfgCfsc (SEQ ID NO:2), wherein (invAb) represents an inverted (3′-3′ linked) abasic deoxyribonucleotide; s represents a phosphorothioate linkage; a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; (C6-SS-C6) represents a straight chain hexyl dithiol (see Table A) and (NH₂-C₆) represents a C₆ terminal amine to facilitate targeting ligand conjugation as desired (see, e.g., Table A).

As the person of ordinary skill in the art would clearly understand, the nucleotide monomers are linked by standard phosphodiester linkages except where inclusion of a phosphorothioate linkage, as shown in the modified nucleotide sequences disclosed herein, replaces the phosphodiester linkage typically present in an oligonucleotide.

In the following examples, various RNAi agents are used as cargo molecules to test the delivery of a cargo molecule via an αvβ6 integrin to a cell of interest.

On study day 1, female C₅₇BL6 mice were dosed via intravenous (“IV”) administration with 200 microliters, according to the following dosing Groups:

TABLE 1
Dosing Groups of mice in Example 3.

| | | Dosing |
|---|---|---|
| Group | RNAi Agent and Dose | Regimen |
| 1 | Isotonic saline (no RNAi agent) | Single IV |
| | | dose on day 1 |
| 2 | 3.0 mg/kg of myostatin double-stranded RNAi agent | Single IV |
| | (AD06326) conjugated to the αvβ6 integrin ligand of Peptide 1 | dose on day 1 |
| | and PEG40K (2 × 2 arm), formulated in isotonic saline. | |
| 3 | 3.0 mg/kg of myostatin double-stranded RNAi agent | Single IV |
| | (AD06326) conjugated to the αvβ6 integrin ligand of | dose on day 1 |
| | Compound 40b and PEG40K (2 × 2 arm), formulated in isotonic | |
| | saline. | |
| 4 | 3.0 mg/kg of myostatin double-stranded RNAi agent | Single IV |
| | (AD06326) conjugated to the αvβ6 integrin ligand of | dose on day 1 |
| | Compound 41b and PEG40K (2 × 2 arm), formulated in isotonic | |
| | saline. | |
| 5 | 3.0 mg/kg of myostatin double-stranded RNAi agent | Single IV |
| | (AD06326) conjugated to the αvβ6 integrin ligand of | dose on day 1 |
| | Compound 42b and PEG40K (2 × 2 arm), formulated in isotonic | |
| | saline. | |
| 6 | 3.0 mg/kg of myostatin double-stranded RNAi agent | Single IV |
| | (AD06326) conjugated to the αvβ6 integrin ligand of | dose on day 1 |
| | Compound 43b and PEG40K (2 × 2 arm), formulated in isotonic | |
| | saline. | |
| 7 | 3.0 mg/kg of myostatin double-stranded RNAi agent | Single IV |
| | (AD06326) conjugated to the αvβ6 integrin ligand of | dose on day 1 |
| | Compound 44b and PEG40K (2 × 2 arm), formulated in isotonic | |
| | saline. | |
| 8 | 3.0 mg/kg of myostatin double-stranded RNAi agent | Single IV |
| | (AD06326) conjugated to the αvβ6 integrin ligand of | dose on day 1 |
| | Compound 45b and PEG40K (2 × 2 arm), formulated in isotonic | |
| | saline. | |
| 9 | 3.0 mg/kg of myostatin double-stranded RNAi agent | Single IV |
| | (AD06326) conjugated to the αvβ6 integrin ligand of | dose on day 1 |
| | Compound 46b and PEG40K (2 × 2 arm), formulated in isotonic | |
| | saline. | |
| 10 | 3.0 mg/kg of myostatin double-stranded RNAi agent | Single IV |
| | (AD06326) conjugated to the αvβ6 integrin ligand of | dose on day 1 |
| | Compound 47b and PEG40K (2 × 2 arm), formulated in isotonic | |
| | saline. | |

The RNAi agents were synthesized having nucleotide sequences directed to target the myostatin gene, and included a functionalized amine reactive group (NH₂-C₆) at the 5′ terminal end of the sense strand to facilitate conjugation to a DBCO linker. The RNAi agents were also synthesized with a (C6-SS-C6)dT at the 3′ terminal end, which is used to conjugate a 40K PEG (2×2 arm) PK/PD modulator. The respective αvβ6 integrin ligands were then conjugated to the RNAi agents via a the DBCO click reaction, as described in Example 2G.ii., above. For the RNAi agent-av036 integrin ligand conjugates of Example 4, the RNAi agent as well as the linker structures, were consistent for each of the Groups 2-10. Thus, the only variable for Groups 2 through 10 was the specific av036 integrin ligand that was used.

Four mice were dosed in each Group (n=4). Mice were bled on days 1, 8, 15, and 22 prior to drug administration and the serum was isolated. An ELISA assay was performed on serum samples to determine the amount of mouse myostatin in serum. Average myostatin in serum samples is shown in Table 2 below.

TABLE 2
Average Relative myostatin mRNA Expression
on Days 8, 15, and 22 in Example 3.

| | Average | | Average | | Average | |
|---|---|---|---|---|---|---|
| | Relative | | Relative | | Relative | |
| | myostatin | | myostatin | | myostatin | |
| | mRNA | | mRNA | | mRNA | |
| | expression | Std | expression | Std | expression | Std |
| Group ID | Day 8 | Dev. | Day 15 | Dev. | Day 22 | Dev. |
| Group 1 (isotonic | 1.094 | 0.146 | 0.980 | 0.166 | 1.022 | 0.129 |
| saline) | | | | | | |
| Group 2 (avb6 peptide | 0.502 | 0.091 | 0.313 | 0.032 | 0.263 | 0.039 |
| 1-AD06326-PEG40K) | | | | | | |
| Group 3 (Compound | 0.747 | 0.066 | 0.534 | 0.070 | 0.556 | 0.064 |
| 40b-AD06326-PEG40K) | | | | | | |
| Group 4 (Compound | 0.774 | 0.023 | 0.589 | 0.066 | 0.711 | 0.066 |
| 41b-AD06326-PEG40K) | | | | | | |
| Group 5 (Compound | 0.989 | 0.073 | 0.821 | 0.059 | 0.886 | 0.028 |
| 42b-AD06326-PEG40K) | | | | | | |
| Group 6 (Compound | 0.961 | 0.151 | 0.755 | 0.109 | 0.874 | 0.106 |
| 43b-AD06326-PEG40K) | | | | | | |
| Group 7 (Compound | 1.306 | 0.109 | 1.023 | 0.075 | 1.199 | 0.083 |
| 44b-AD06326-PEG40K) | | | | | | |
| Group 8 (Compound | 0.648 | 0.147 | 0.373 | 0.078 | 0.359 | 0.109 |
| 45b-AD06326-PEG40K) | | | | | | |
| Group 9 (Compound | 0.612 | 0.106 | 0.380 | 0.059 | 0.330 | 0.068 |
| 46b-AD06326-PEG40K) | | | | | | |
| Group 10 (Compound | 0.997 | 0.074 | 0.891 | 0.080 | 0.994 | 0.040 |
| 47b-AD06326-PEG40K) | | | | | | |

As shown in Table 2 above, many of the myostatin RNAi agents showed a reduction in mRNA expression in mice compared to control.

### Example 4. In Vivo Intravenous Administration of RNAi Agents Targeting Myostatin Conjugated to αvβ6 Integrin Ligands in Mice

On study day 1, female C₅₇BL6 Mice were dosed via intravenous (“IV”) administration with 200 microliters, according to the following dosing Groups:

TABLE 3
Dosing Groups of mice in Example 4.

| | | Dosing |
|---|---|---|
| Group | RNAi Agent and Dose | Regimen |
| 1 | Isotonic saline (no RNAi agent) | Single IV |
| | | dose on day 1 |
| 2 | 3.0 mg/kg of myostatin double-stranded RNAi agent | Single IV |
| | (AD06326) conjugated to the αvβ6 integrin ligand of Peptide 1 | dose on day 1 |
| | and PEG40K (XY-4 arm), formulated in isotonic saline. | |
| 4 | 3.0 mg/kg of myostatin double-stranded RNAi agent | Single IV |
| | (AD06326) conjugated to the αvβ6 integrin ligand of | dose on day 1 |
| | Compound 49b and PEG40K (2 × 2 arm), formulated in isotonic | |
| | saline. | |
| 5 | 3.0 mg/kg of myostatin double-stranded RNAi agent | Single IV |
| | (AD06326) conjugated to the αvβ6 integrin ligand of | dose on day 1 |
| | Compound 50b and PEG40K (2 × 2 arm), formulated in isotonic | |
| | saline. | |
| 6 | 3.0 mg/kg of myostatin double-stranded RNAi agent | Single IV |
| | (AD06326) conjugated to the αvβ6 integrin ligand of | dose on day 1 |
| | Compound 51b and PEG40K (2 × 2 arm), formulated in isotonic | |
| | saline. | |
| 7 | 3.0 mg/kg of myostatin double-stranded RNAi agent | Single IV |
| | (AD06326) conjugated to the αvβ6 integrin ligand of | dose on day 1 |
| | Compound 52b and PEG40K (2 × 2 arm), formulated in isotonic | |
| | saline. | |
| 8 | 3.0 mg/kg of myostatin double-stranded RNAi agent | Single IV |
| | (AD06326) conjugated to the αvβ6 integrin ligand of | dose on day 1 |
| | Compound 53b and PEG40K (2 × 2 arm), formulated in isotonic | |
| | saline. | |
| 9 | 3.0 mg/kg of myostatin double-stranded RNAi agent | Single IV |
| | (AD06326) conjugated to the αvβ6 integrin ligand of | dose on day 1 |
| | Compound 54b and PEG40K (2 × 2 arm), formulated in isotonic | |
| | saline. | |
| 10 | 3.0 mg/kg of myostatin double-stranded RNAi agent | Single IV |
| | (AD06326) conjugated to the αvβ6 integrin ligand of | dose on day 1 |
| | Compound 47b and PEG40K (2 × 2 arm), formulated in isotonic | |
| | saline. | |

The RNAi agents were synthesized having nucleotide sequences directed to target the myostatin gene, and included a functionalized amine reactive group (NH₂-C₆) at the 5′ terminal end of the sense strand to facilitate conjugation to a DBCO linker. The RNAi agents were also synthesized with a (C6-SS-C6)dT at the 3′ terminal end, which is used to conjugate a 40K PEG (XY-4 arm) PK/PD modulator. The respective αvβ6 integrin ligands were then conjugated to the RNAi agents via a copper click reaction, as described in Example 2G. For the RNAi agent-αvβ6 integrin ligand conjugates of Example 4, the RNAi agent as well as the linker structures, were consistent for each of the Groups 2-10. Thus, the only variable for Groups 2 through 10 was the specific αvβ6 integrin ligand that was used.

Four mice were dosed in each Group (n=4). Mice were bled on days 1, 8, 15, and 22 prior to drug administration and the serum was isolated. An ELISA assay was performed on serum samples to determine the amount of mouse myostatin in serum. Average myostatin in serum samples is shown in Table 6 below.

TABLE 4
Average Relative myostatin mRNA Expression
on Days 8, 15, and 22 in Example 4.

| | Average | | Average | | Average | |
|---|---|---|---|---|---|---|
| | Relative | | Relative | | Relative | |
| | myostatin | | myostatin | | myostatin | |
| | mRNA | | mRNA | | mRNA | |
| | expression | Std | expression | Std | expression | Std |
| Group ID | Day 8 | Dev. | Day 15 | Dev. | Day 22 | Dev. |
| Group 1 (isotonic | 0.917 | 0.025 | 1.051 | 0.068 | 0.990 | 0.101 |
| saline) | | | | | | |
| Group 2 (avb6 peptide | 0.498 | 0.065 | 0.335 | 0.050 | 0.278 | 0.037 |
| 1-AD06326-PEG40K) | | | | | | |
| Group 4 (Compound | 0.790 | 0.011 | 0.803 | 0.038 | 0.760 | 0.025 |
| 49b-AD06326-PEG40K) | | | | | | |
| Group 5 (Compound | 0.783 | 0.017 | 0.729 | 0.077 | 0.758 | 0.063 |
| 50b-AD06326-PEG40K) | | | | | | |
| Group 6 (Compound | 0.573 | 0.047 | 0.462 | 0.057 | 0.450 | 0.061 |
| 51b-AD06326-PEG40K) | | | | | | |
| Group 7 (Compound | 0.622 | 0.021 | 0.466 | 0.034 | 0.483 | 0.069 |
| 52b-AD06326-PEG40K) | | | | | | |
| Group 8 (Compound | 0.695 | 0.083 | 0.521 | 0.043 | 0.518 | 0.052 |
| 53b-AD06326-PEG40K) | | | | | | |
| Group 9 (Compound | 0.794 | 0.030 | 0.697 | 0.053 | 0.667 | 0.065 |
| 54b-AD06326-PEG40K) | | | | | | |
| Group 10 (Compound | 1.088 | 0.033 | 1.009 | 0.079 | 1.172 | 0.084 |
| 55b-AD06326-PEG40K) | | | | | | |

As shown in Table 4 above, many of the myostatin RNAi agents showed a reduction in mRNA expression in mice compared to control.

### Example 5. In Vivo Intravenous Administration of RNAi Agents Targeting Myostatin Conjugated to αvβ6 Integrin Ligands in Mice

On study day 1, female C57BL6 Mice were dosed via intravenous (“IV”) administration with 200 microliters, according to the following dosing Groups:

TABLE 5
Dosing Groups of mice in Example 5.

| | | Dosing |
|---|---|---|
| Group | RNAi Agent and Dose | Regimen |
| 1 | Isotonic saline (no RNAi agent) | Single IV |
| | | dose on day 1 |
| 2 | 3.0 mg/kg of myostatin double-stranded RNAi agent | Single IV |
| | (AD06326) conjugated to the αvβ6 integrin ligand of Peptide 1 | dose on day 1 |
| | and PEG40K (PEG 40K 4 arm), formulated in isotonic saline. | |
| 3 | 3.0 mg/kg of myostatin double-stranded RNAi agent | Single IV |
| | (AD06326) conjugated to the αvβ6 integrin ligand of | dose on day 1 |
| | Compound 6.1b and PEG40K (PEG 40K 4 arm), formulated in | |
| | isotonic saline. | |
| 4 | 3.0 mg/kg of myostatin double-stranded RNAi agent | Single IV |
| | (AD06326) conjugated to the αvβ6 integrin ligand of | dose on day 1 |
| | Compound 45b and PEG40K (PEG 40K 4 arm), formulated in | |
| | isotonic saline. | |
| 5 | 3.0 mg/kg of myostatin double-stranded RNAi agent | Single IV |
| | (AD06326) conjugated to the αvβ6 integrin ligand of | dose on day 1 |
| | Compound 46b and PEG40K (PEG 40K 4 arm), formulated in | |
| | isotonic saline. | |
| 6 | 3.0 mg/kg of myostatin double-stranded RNAi agent | Single IV |
| | (AD06326) conjugated to the αvβ6 integrin ligand of | dose on day 1 |
| | Compound 51b and PEG40K (PEG 40K 4 arm), formulated in | |
| | isotonic saline. | |
| 7 | 3.0 mg/kg of myostatin double-stranded RNAi agent | Single IV |
| | (AD06326) conjugated to the αvβ6 integrin ligand of | dose on day 1 |
| | Compound 6.1b and PEG95 + C22, formulated in isotonic | |
| | saline. | |
| 8 | 3.0 mg/kg of myostatin double-stranded RNAi agent | Single IV |
| | (AD06326) conjugated to the αvβ6 integrin ligand of | dose on day 1 |
| | Compound 45b and PEG95 + C22, formulated in isotonic | |
| | saline. | |
| 9 | 3.0 mg/kg of myostatin double-stranded RNAi agent | Single IV |
| | (AD06326) conjugated to the αvβ6 integrin ligand of | dose on day 1 |
| | Compound 46b and PEG95 + C22, formulated in isotonic | |
| | saline. | |
| 10 | 3.0 mg/kg of myostatin double-stranded RNAi agent | Single IV |
| | (AD06326) conjugated to the αvβ6 integrin ligand of | dose on day 1 |
| | Compound 51b and PEG95 + C22, formulated in isotonic | |
| | saline. | |

The structure for compound 6.1 as used in group 3 is:

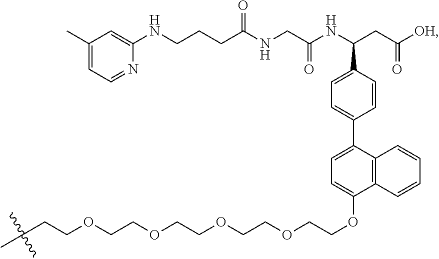 [SMILES: *CCOCCOCCOCCOCCOc1ccc(-c2ccc([C@H](CC(=O)O)NC(=O)CNC(=O)CCCNc3cc(C)ccn3)cc2)c2ccccc12]

which was conjugated to the RNAi agent using similar methods as described herein.

The RNAi agents were synthesized having nucleotide sequences directed to target the myostatin gene, and included a functionalized amine reactive group (NH₂-C₆) at the 5′ terminal end of the sense strand to facilitate conjugation to a DBCO linker. The RNAi agents were also synthesized with a (C6-SS-C6)dT at the 3′ terminal end, which is used to conjugate a 40K PEG (4-arm) PK/PD modulator or PEG95+C22 PK/PD modulator. The respective αvβ6 integrin ligands were then conjugated to the RNAi agents via a copper click reaction, as described in Example 2G.

Four mice were dosed in each Group (n=4). Mice were bled on days 1, 8, 15, and 22 prior to drug administration and the serum was isolated. An ELISA assay was performed on serum samples to determine the amount of mouse myostatin in serum. Average myostatin in serum samples is shown in Table 6 below.

TABLE 6
Average Relative myostatin mRNA Expression on Days 8, 15, and 22 in Example 5.

| | Average | | Average | | Average | |
|---|---|---|---|---|---|---|
| | Relative | | Relative | | Relative | |
| | myostatin | | myostatin | | myostatin | |
| | mRNA | | mRNA | | mRNA | |
| | expression | Std | expression | Std | expression | Std |
| Group ID | Day 8 | Dev. | Day 15 | Dev. | Day 22 | Dev. |
| Group 1 (isotonic | 0.917 | 0.099 | 0.905 | 0.074 | 0.995 | 0.092 |
| saline) | | | | | | |
| Group 2 (avb6 peptide | 0.532 | 0.032 | 0.236 | 0.009 | 0.261 | 0.014 |
| 1-AD06326-PEG40K) | | | | | | |
| Group 3(Compound | 0.640 | 0.078 | 0.379 | 0.076 | 0.393 | 0.063 |
| 6.1b-AD06326-PEG40K) | | | | | | |
| Group 4 (Compound | 0.598 | 0.030 | 0.354 | 0.054 | 0.354 | 0.075 |
| 45b-AD06326-PEG40K) | | | | | | |
| Group 5 (Compound | 0.580 | 0.058 | 0.317 | 0.025 | 0.307 | 0.041 |
| 46b-AD06326-PEG40K) | | | | | | |
| Group 6 (Compound | 0.558 | 0.038 | 0.346 | 0.036 | 0.367 | 0.041 |
| 51b-AD06326-PEG40K) | | | | | | |
| Group 7 (Compound | 0.551 | 0.021 | 0.331 | 0.020 | 0.332 | 0.048 |
| 6.1b-AD06326-PEG95 + C22) | | | | | | |
| Group 8 (Compound | 0.525 | 0.059 | 0.374 | 0.046 | 0.349 | 0.022 |
| 45b-AD06326-PEG95 + C22) | | | | | | |
| Group 9 (Compound | 0.658 | 0.054 | 0.435 | 0.067 | 0.414 | 0.035 |
| 46b-AD06326-PEG95 + C22) | | | | | | |
| Group 10 (Compound | 0.578 | 0.120 | 0.393 | 0.071 | 0.421 | 0.106 |
| 51b-AD06326-PEG95 + C22) | | | | | | |

As shown in Table 6 above, many of the myostatin RNAi agents showed a reduction in mRNA expression in mice compared to control.

### Example 6. In Vivo Intravenous Administration of RNAi Agents Targeting Myostatin Conjugated to αvβ6 Integrin Ligands in Mice

On study day 1, female C57BL6 mice were dosed via intravenous (“IV”) administration with 200 microliters, according to the following dosing Groups:

TABLE 7
Dosing Groups of mice in Example 6.

| | | Dosing |
|---|---|---|
| Group | RNAi Agent and Dose | Regimen |
| 1 | Isotonic saline (no RNAi agent) | Single IV |
| | | dose on day 1 |
| 2 | 3.0 mg/kg of myostatin double-stranded RNAi agent | Single IV |
| | (AD06326) conjugated to the αvβ6 integrin ligand of Peptide 1 | dose on day 1 |
| | and PEG40K (4 arm), formulated in isotonic saline. | |
| 6 | 3.0 mg/kg of myostatin double-stranded RNAi agent | Single IV |
| | (AD06326) conjugated to the αvβ6 integrin ligand of | dose on day 1 |
| | Compound 56b and PEG40K (4 arm), formulated in isotonic | |
| | saline. | |
| 7 | 3.0 mg/kg of myostatin double-stranded RNAi agent | Single IV |
| | (AD06326) conjugated to the αvβ6 integrin ligand of | dose on day 1 |
| | Compound 57b and PEG40K (4 arm), formulated in isotonic | |
| | saline. | |
| 8 | 3.0 mg/kg of myostatin double-stranded RNAi agent | Single IV |
| | (AD06326) conjugated to the αvβ6 integrin ligand of | dose on day 1 |
| | Compound 58b and PEG40K (4 arm), formulated in isotonic | |
| | saline. | |
| 9 | 3.0 mg/kg of myostatin double-stranded RNAi agent | Single IV |
| | (AD06326) conjugated to the αvβ6 integrin ligand of | dose on day 1 |
| | Compound 59b and PEG40K (4 arm), formulated in isotonic | |
| | saline. | |
| 10 | 3.0 mg/kg of myostatin double-stranded RNAi agent | Single IV |
| | (AD06326) conjugated to the αvβ6 integrin ligand of | dose on day 1 |
| | Compound 60b and PEG40K (4 arm), formulated in isotonic | |
| | saline. | |

The RNAi agents were synthesized having nucleotide sequences directed to target the myostatin gene, and included a functionalized amine reactive group (NH₂-C₆) at the 5′ terminal end of the sense strand to facilitate conjugation to a DBCO linker. The RNAi agents were also synthesized with a (C6-SS-C6)dT at the 3′ terminal end, which is used to conjugate a 40K PEG (4-arm) PK/PD modulator. The respective αvβ6 integrin ligands were then conjugated to the RNAi agents via a copper click reaction, as described in Example 2G. For the RNAi agent-αvβ6 integrin ligand conjugates of Example 4, the RNAi agent as well as the linker structures, were consistent for each of the Groups 2 and 6-10.

Four mice were dosed in each Group (n=4). Mice were bled on days 1, 8, 15, and 22 prior to drug administration and the serum was isolated. An ELISA assay was performed on serum samples to determine the amount of mouse myostatin in serum. Average myostatin in serum samples is shown in Table 8 below.

TABLE 8
Average Relative myostatin mRNA Expression
on Days 8, 15, and 22 in Example 6.

| | Average | | Average | | Average | |
|---|---|---|---|---|---|---|
| | Relative | | Relative | | Relative | |
| | myostatin | | myostatin | | myostatin | |
| | mRNA | | mRNA | | mRNA | |
| | expression | Std | expression | Std | expression | Std |
| Group ID | Day 8 | Dev. | Day 15 | Dev. | Day 22 | Dev. |
| Group 1 (isotonic | 1.003 | 0.056 | 1.046 | 0.083 | 0.816 | 0.058 |
| saline) | | | | | | |
| Group 2 (avb6 peptide | 0.509 | 0.045 | 0.315 | 0.049 | 0.195 | 0.013 |
| 1-AD06326-PEG40K) | | | | | | |
| Group 6 (Compound | 0.600 | 0.060 | 0.380 | 0.048 | 0.263 | 0.007 |
| 56b-AD06326-PEG40K) | | | | | | |
| Group 7 (Compound | 0.489 | 0.022 | 0.314 | 0.010 | 0.365 | 0.227 |
| 57b-AD06326-PEG40K) | | | | | | |
| Group 8 (Compound | 0.761 | 0.057 | 0.839 | 0.041 | 0.544 | 0.221 |
| 58b-AD06326-PEG40K) | | | | | | |
| Group 9 (Compound | 0.820 | 0.111 | 0.862 | 0.123 | 0.615 | 0.072 |
| 59b-AD06326-PEG40K) | | | | | | |
| Group 10 (Compound | 0.932 | 0.057 | 0.990 | 0.065 | 0.771 | 0.066 |
| 60b-AD06326-PEG40K) | | | | | | |

As shown in Table 8 above, many of the myostatin RNAi agents showed a reduction in mRNA expression in mice compared to control.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of Formula I: 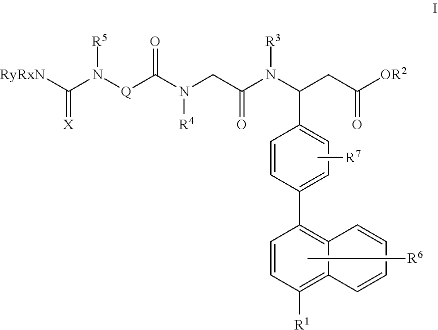
or a pharmaceutically acceptable salt thereof, wherein
R¹ is optionally substituted alkyl, optionally substituted alkoxy, or 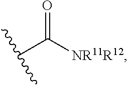
wherein R¹¹ and R¹² are each independently optionally substituted alkyl or a cargo molecule, or R¹ is a cargo molecule;
R² is H, optionally substituted alkyl, or a cargo molecule;
R³ is H or optionally substituted alkyl;
R⁴ is H or optionally substituted alkyl;
R⁵ is H or optionally substituted alkyl;
R⁶ is selected from the group consisting of H, optionally substituted alkyl, optionally substituted alkoxy, halo, optionally substituted amino, or a cargo molecule;
Q is optionally substituted aryl or optionally substituted alkylene;
X is O, CR⁸R⁹, NR⁸;
wherein R⁸ is selected from H, optionally substituted alkyl, or R⁸ is taken together with Rx or Ry to form a 4-, 5-, 6-, 7-, 8- or 9-membered ring, and R⁹ is H or optionally substituted alkyl;
Rx and Ry are each independently H, optionally substituted alkyl, a cargo molecule or Rx and Ry may be taken together to form a double bond with R¹⁰, wherein R¹⁰ is H, optionally substituted alkyl, or R¹⁰ may be taken together with X and the atoms to which it is attached to form a 4-, 5-, 6-, 7-, 8, or 9-membered ring;
wherein at least one of R¹, R², R⁶, R¹¹, R¹², Rx and Ry comprise a cargo molecule; and
wherein when Q is optionally substituted alkyl and the length of the optionally substituted alkyl chain represented by Q is 3 carbons, then R¹ is

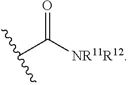

2. The compound of claim 1, wherein the compound is a compound of Formula Ia: 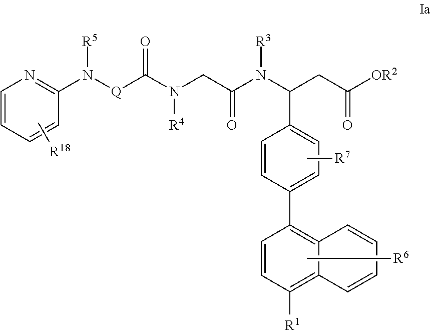
wherein R¹⁸ is selected from the group consisting of H, optionally substituted alkyl, optionally substituted alkoxy, halo, —NR¹⁹R²⁰, wherein R¹⁹ and R²⁰ are each independently H or optionally substituted alkyl.

3. The compound of claim 1, wherein the compound is a compound of Formula Ib:

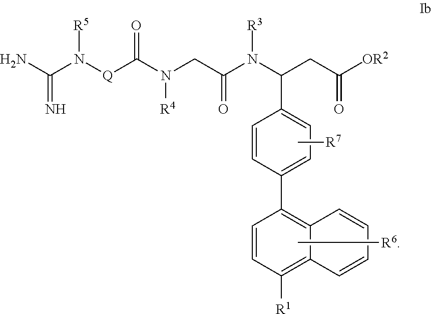

4. The compound of claim 1, wherein the compound is a compound of Formula Ic:

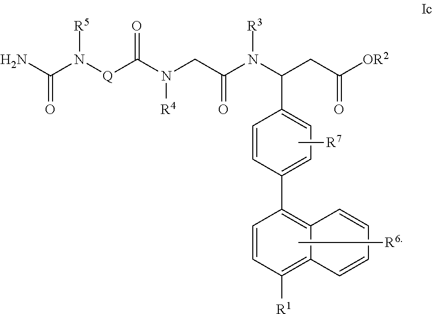

5. The compound of claim 1, wherein the compound is a compound of Formula Id: 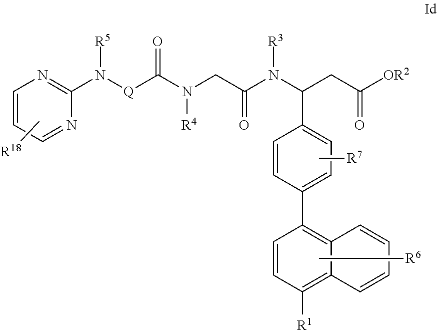
wherein R¹⁸ is selected from the group consisting of H, optionally substituted alkyl, optionally substituted alkoxy, halo, —NR¹⁹R²⁰, wherein R¹⁹ and R²⁰ are each independently H or optionally substituted alkyl.

6-7. (canceled)

8. The compound of claim 1, wherein Q is 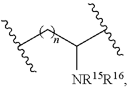
wherein R¹⁵ and R¹⁶ are each independently H, 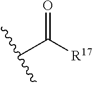
wherein R¹⁷ is optionally substituted alkyl, or optionally substituted alkyl; and n is an integer from 1 to 10.

9-15. (canceled)

16. The compound of claim 1, wherein R¹ comprises at least one polyethylene glycol (PEG) unit.

17-21. (canceled)

22. The compound of claim 1, wherein the compound has the formula.

| Compound | |
|---|---|
| Number | Formula |
| 41a | 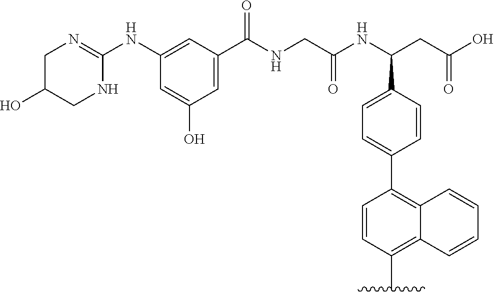 [SMILES: *c1ccc(-c2ccc([C@H](CC(=O)O)NC(=O)CNC(=O)c3cc(O)cc(NC4=NCC(O)CN4)c3)cc2)c2ccccc12] |
| 42a | 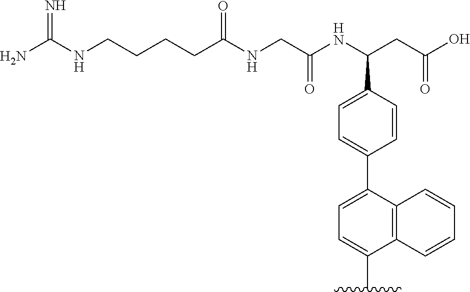 [SMILES: *c1ccc(-c2ccc([C@H](CC(=O)O)NC(=O)CNC(=O)CCCCNC(=N)N)cc2)c2ccccc12] |
| 43a | 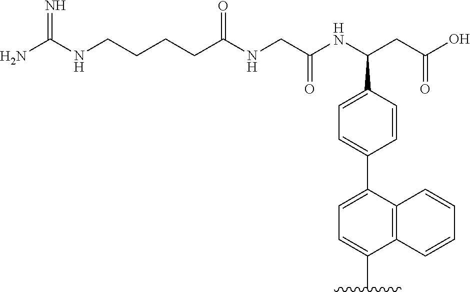 [SMILES: *c1ccc(-c2ccc([C@H](CC(=O)O)NC(=O)CNC(=O)CCCCNC(=N)N)cc2)c2ccccc12] |
| 44a | 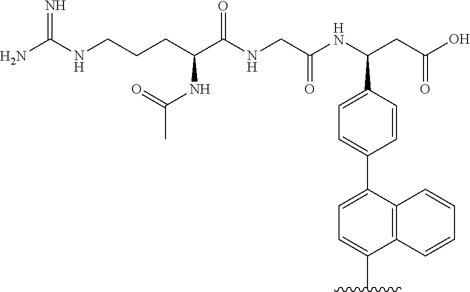 [SMILES: *c1ccc(-c2ccc([C@H](CC(=O)O)NC(=O)CNC(=O)[C@H](CCCNC(=N)N)NC(C)=O)cc2)c2ccccc12] |
| 45a | 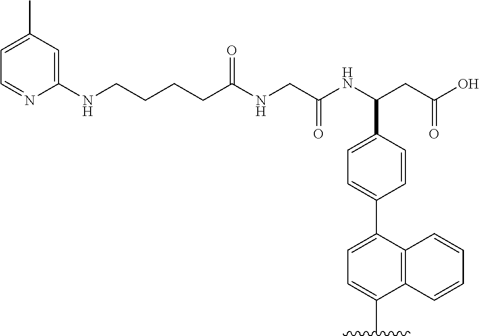 |
| 46a | 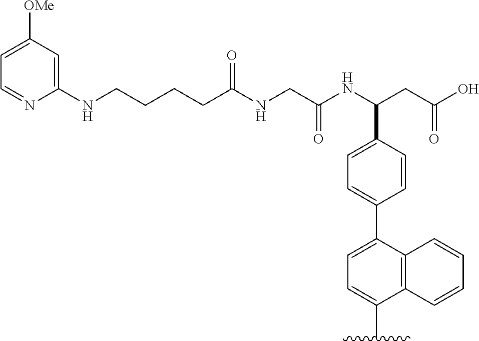 |
| 47a | 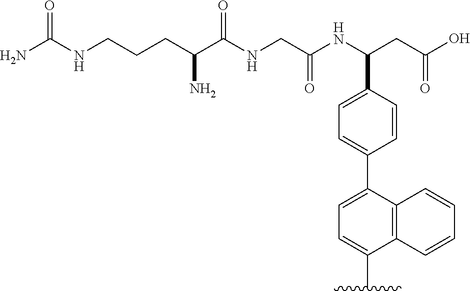 |
| 48a | 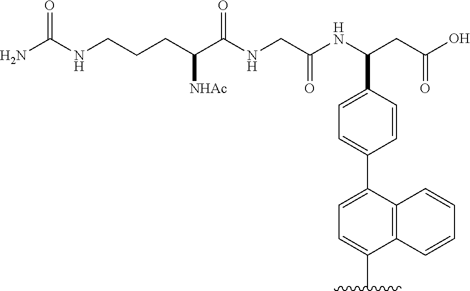 |
| 49a | 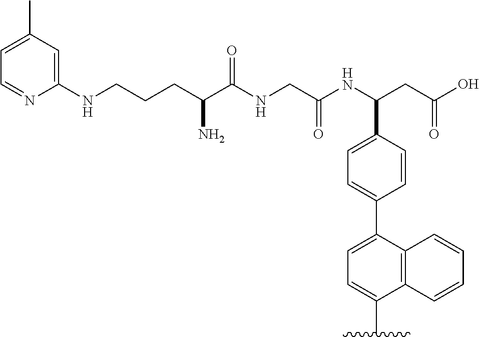 |
| 50a | 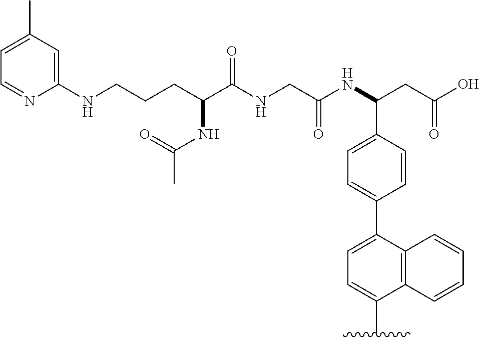 [SMILES: *c1ccc(-c2ccc([C@H](CC(=O)O)NC(=O)CNC(=O)[C@H](CCCNc3cc(C)ccn3)NC(C)=O)cc2)c2ccccc12] |
| 51a | 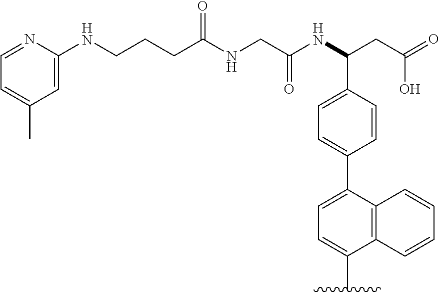 [SMILES: *c1ccc(-c2ccc([C@H](CC(=O)O)NC(=O)CNC(=O)CCCNc3cc(C)ccn3)cc2)c2ccccc12] |
| 52a | 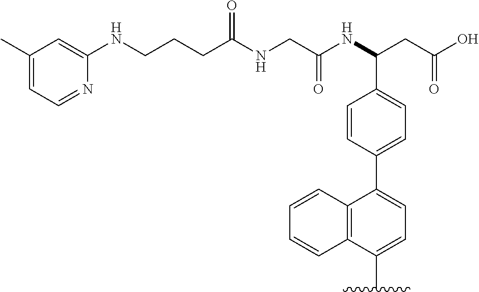 [SMILES: *c1ccc(-c2ccc([C@H](CC(=O)O)NC(=O)CNC(=O)CCCNc3cc(C)ccn3)cc2)c2ccccc12] |
| 53a | 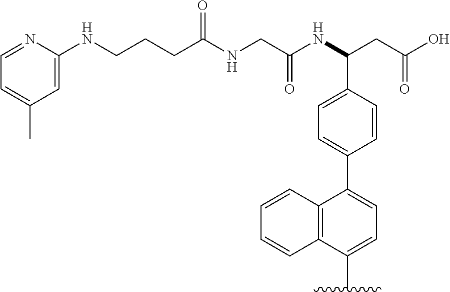 [SMILES: *c1ccc(-c2ccc([C@H](CC(=O)O)NC(=O)CNC(=O)CCCNc3cc(C)ccn3)cc2)c2ccccc12] |
| 54a | 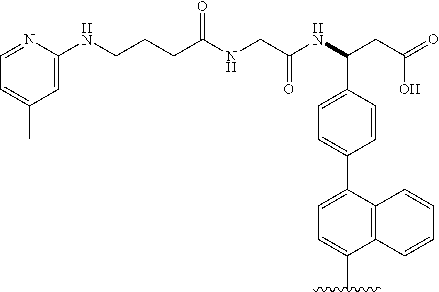 [SMILES: *c1ccc(-c2ccc([C@H](CC(=O)O)NC(=O)CNC(=O)CCCNc3cc(C)ccn3)cc2)c2ccccc12] |
| 55a | 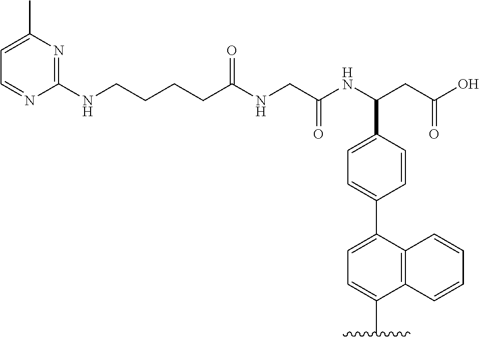 [SMILES: *c1ccc(-c2ccc([C@H](CC(=O)O)NC(=O)CNC(=O)CCCCNc3nccc(C)n3)cc2)c2ccccc12] |
| 56a | 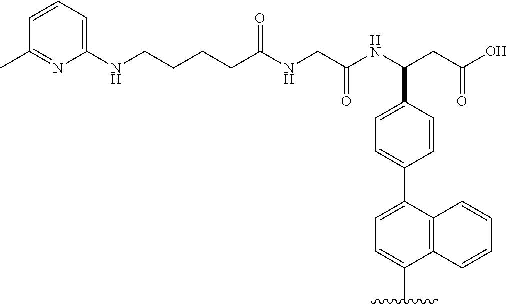 [SMILES: *c1ccc(-c2ccc([C@H](CC(=O)O)NC(=O)CNC(=O)CCCCNc3cccc(C)n3)cc2)c2ccccc12] |
| 57a | 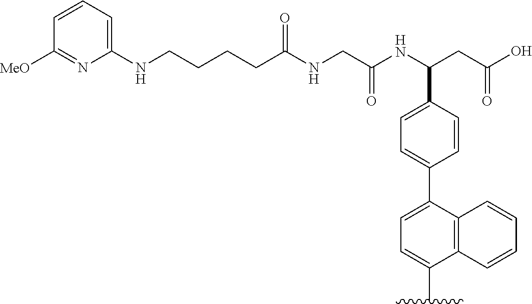 [SMILES: *c1ccc(-c2ccc([C@H](CC(=O)O)NC(=O)CNC(=O)CCCCNc3cccc(OC)n3)cc2)c2ccccc12] |
| 58a | 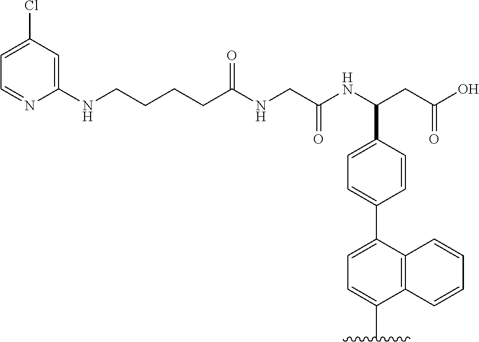 [SMILES: *c1ccc(-c2ccc([C@H](CC(=O)O)NC(=O)CNC(=O)CCCCNc3cc(Cl)ccn3)cc2)c2ccccc12] |
| 59a | 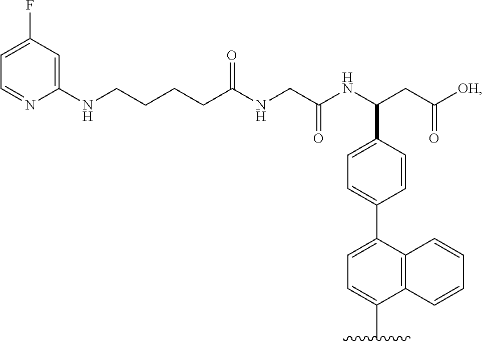 [SMILES: *c1ccc(-c2ccc([C@H](CC(=O)O)NC(=O)CNC(=O)CCCCNc3cc(F)ccn3)cc2)c2ccccc12] |
| | or |
| 60a | 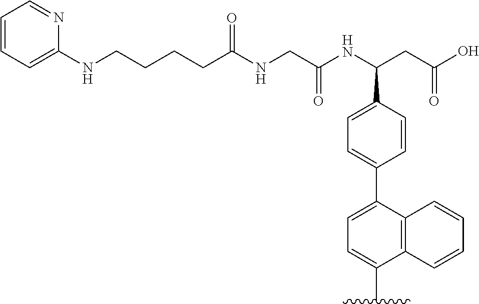 [SMILES: *c1ccc(-c2ccc([C@H](CC(=O)O)NC(=O)CNC(=O)CCCCNc3ccccn3)cc2)c2ccccc12] |

or a pharmaceutically acceptable salt thereof, and wherein [symbol] indicates the point of connection to a cargo molecule.

23. The compound of claim 1, wherein the compound has the formula:

| Compound | |
|---|---|
| Number | Formula |
| 41b | 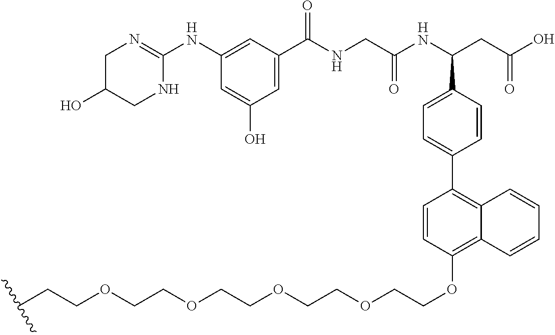 [SMILES: *CCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12] |
| 42b | 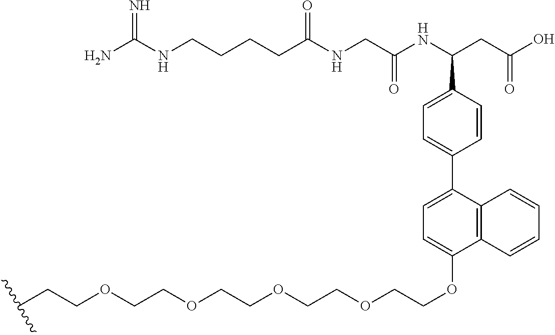 [SMILES: *CCOCCOCCOCCOCCOc1ccc(-c2ccc([C@H](CC(=O)O)NC(=O)CNC(=O)CCCCNC(=N)N)cc2)c2ccccc12] |
| 43b | 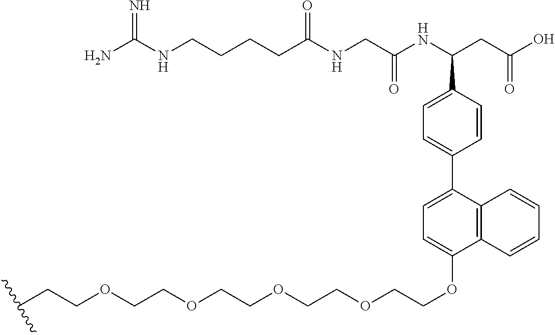 [SMILES: *CCOCCOCCOCCOCCOc1ccc(-c2ccc([C@H](CC(=O)O)NC(=O)CNC(=O)CCCCNC(=N)N)cc2)c2ccccc12] |
| 44b | 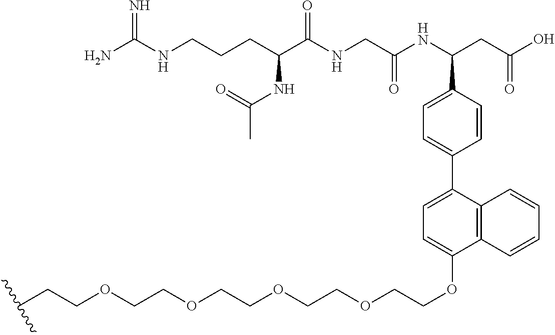 [SMILES: *CCOCCOCCOCCOCCOc1ccc(-c2ccc([C@H](CC(=O)O)NC(=O)CNC(=O)[C@H](CCCNC(=N)N)NC(C)=O)cc2)c2ccccc12] |
| 45b | 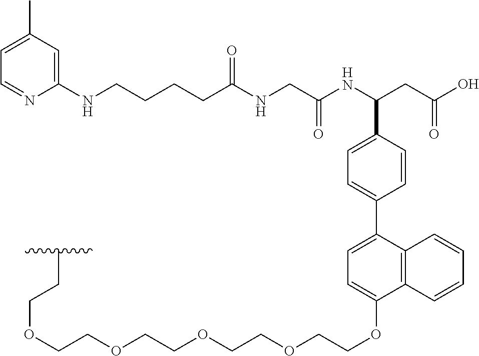 [SMILES: *CCOCCOCCOCCOCCOc1ccc(-c2ccc([C@H](CC(=O)O)NC(=O)CNC(=O)CCCCNc3cc(C)ccn3)cc2)c2ccccc12] |
| 46b | 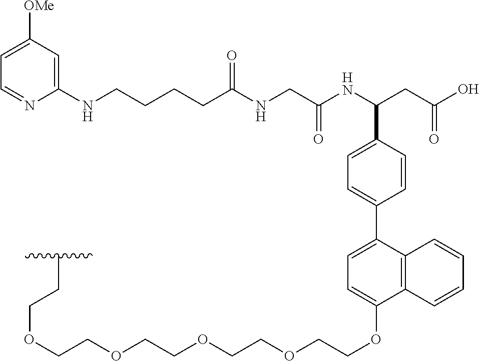 [SMILES: *CCOCCOCCOCCOCCOc1ccc(-c2ccc([C@H](CC(=O)O)NC(=O)CNC(=O)CCCCNc3cc(OC)ccn3)cc2)c2ccccc12] |
| 47b | 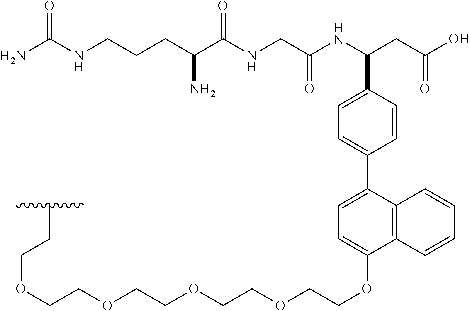 [SMILES: *CCOCCOCCOCCOCCOc1ccc(-c2ccc([C@H](CC(=O)O)NC(=O)CNC(=O)[C@@H](N)CCCNC(N)=O)cc2)c2ccccc12] |
| 48b | 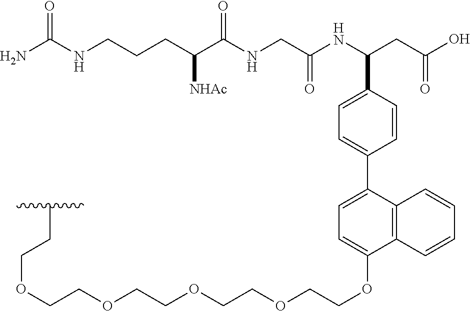 [SMILES: *CCOCCOCCOCCOCCOc1ccc(-c2ccc([C@H](CC(=O)O)NC(=O)CNC(=O)[C@H](CCCNC(N)=O)NC(C)=O)cc2)c2ccccc12] |
| 49b | 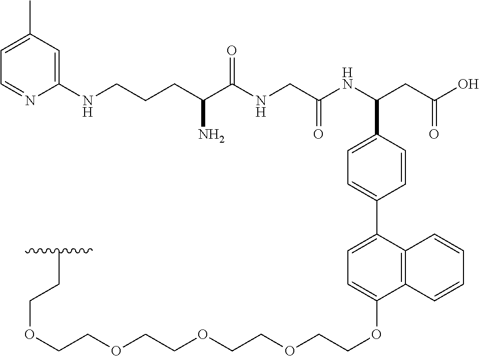 |
| 50b | 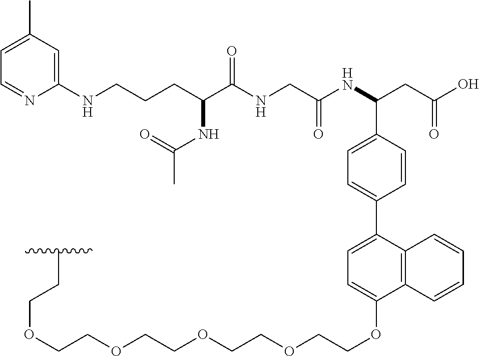 [SMILES: *CCOCCOCCOCCOCCOc1ccc(-c2ccc([C@H](CC(=O)O)NC(=O)CNC(=O)[C@H](CCCNc3cc(C)ccn3)NC(C)=O)cc2)c2ccccc12] |
| 51b | 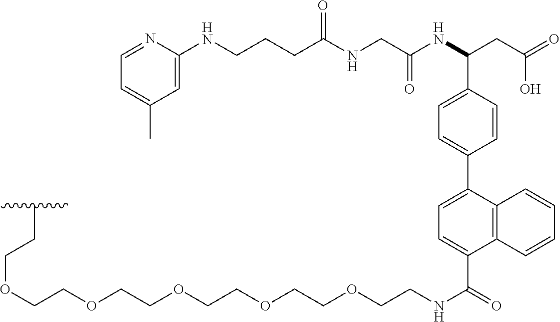 [SMILES: *CCOCCOCCOCCOCCOCCNC(=O)c1ccc(-c2ccc([C@H](CC(=O)O)NC(=O)CNC(=O)CCCNc3cc(C)ccn3)cc2)c2ccccc12] |
| 52b | 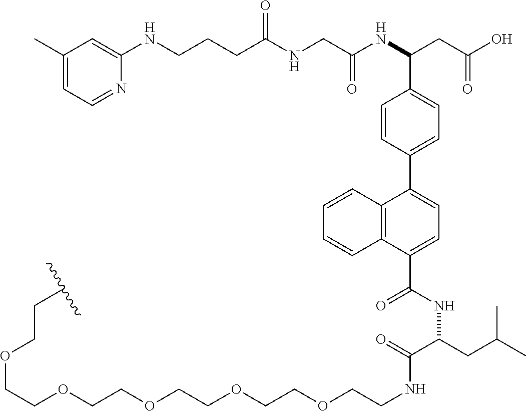 [SMILES: *CCOCCOCCOCCOCCOCCNC(=O)[C@@H](CC(C)C)NC(=O)c1ccc(-c2ccc([C@H](CC(=O)O)NC(=O)CNC(=O)CCCNc3cc(C)ccn3)cc2)c2ccccc12] |
| 53b | 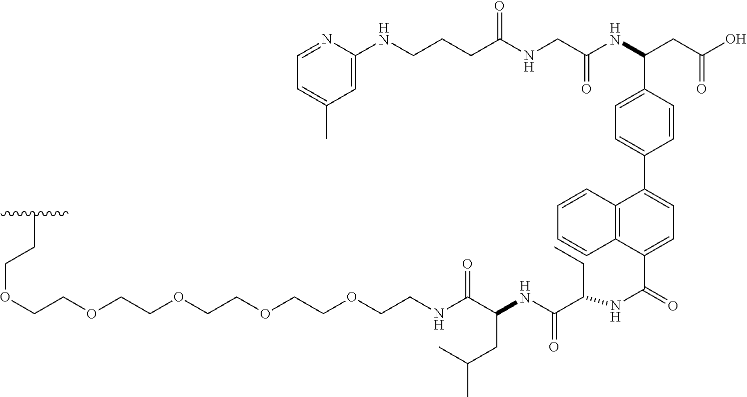 [SMILES: *CCOCCOCCOCCOCCOCCNC(=O)[C@H](CC(C)C)NC(=O)[C@H](CC)NC(=O)c1ccc(-c2ccc([C@H](CC(=O)O)NC(=O)CNC(=O)CCCNc3cc(C)ccn3)cc2)c2ccccc12] |
| 54b | 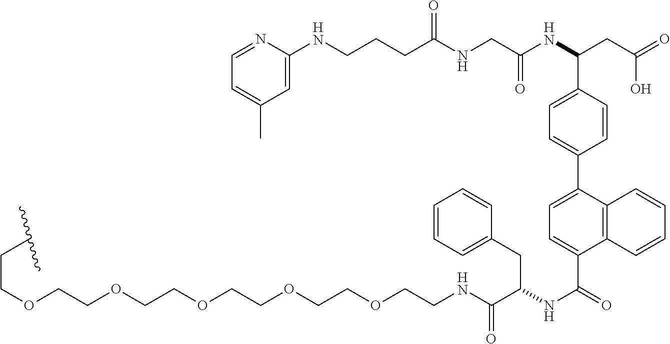 [SMILES: *CCOCCOCCOCCOCCOCCNC(=O)[C@H](Cc1ccccc1)NC(=O)c1ccc(-c2ccc([C@H](CC(=O)O)NC(=O)CNC(=O)CCCNc3cc(C)ccn3)cc2)c2ccccc12] |
| 55b | 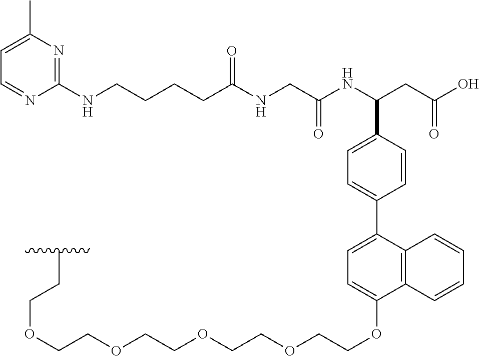 [SMILES: *CCOCCOCCOCCOCCOc1ccc(-c2ccc([C@H](CC(=O)O)NC(=O)CNC(=O)CCCCNc3nccc(C)n3)cc2)c2ccccc12] |
| 56b | 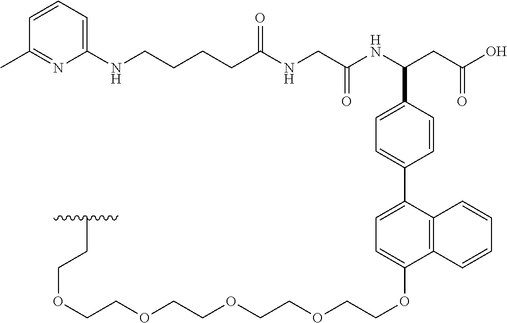 [SMILES: *CCOCCOCCOCCOCCOc1ccc(-c2ccc([C@H](CC(=O)O)NC(=O)CNC(=O)CCCCNc3cccc(C)n3)cc2)c2ccccc12] |
| 57b | 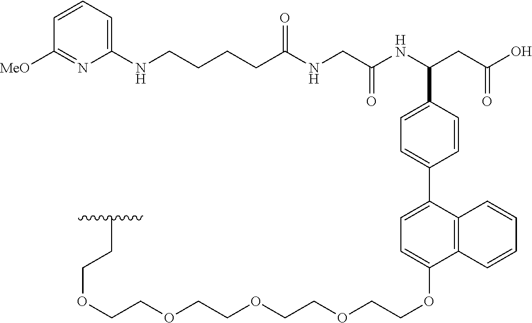 [SMILES: *CCOCCOCCOCCOCCOc1ccc(-c2ccc([C@H](CC(=O)O)NC(=O)CNC(=O)CCCCNc3cccc(OC)n3)cc2)c2ccccc12] |
| 58b | 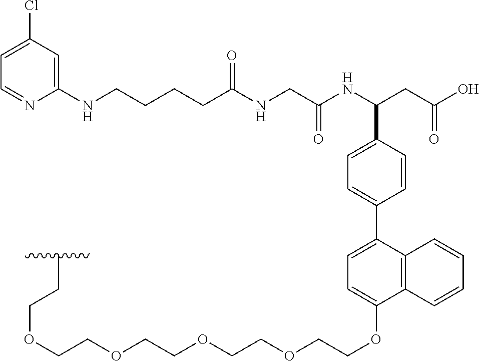 [SMILES: *CCOCCOCCOCCOCCOc1ccc(-c2ccc([C@H](CC(=O)O)NC(=O)CNC(=O)CCCCNc3cc(Cl)ccn3)cc2)c2ccccc12] |
| 59b | 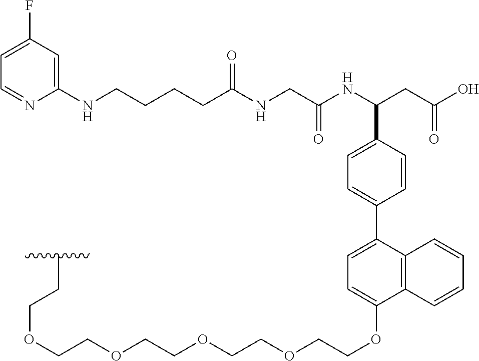 [SMILES: *CCOCCOCCOCCOCCOc1ccc(-c2ccc([C@H](CC(=O)O)NC(=O)CNC(=O)CCCCNc3cc(F)ccn3)cc2)c2ccccc12] |
| 60b | 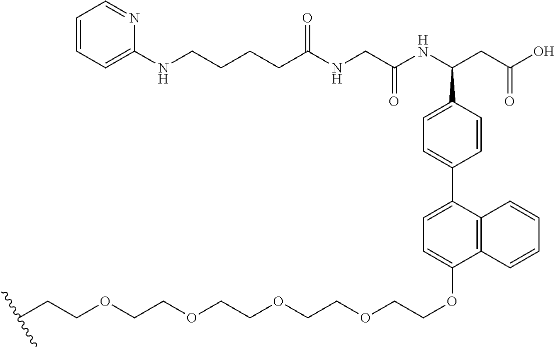 [SMILES: *CCOCCOCCOCCOCCOc1ccc(-c2ccc([C@H](CC(=O)O)NC(=O)CNC(=O)CCCCNc3ccccn3)cc2)c2ccccc12] |

or a pharmaceutically acceptable salt thereof, and wherein [symbol] indicates the point of connection to a cargo molecule.

24. The compound of claim 1, wherein the cargo molecule comprises an RNAi agent.

25. (canceled)

26. The compound of claim 24, wherein the compound is bound to the 5′ end of the sense strand of the RNAi agent.

27. A compound of Formula Ip: 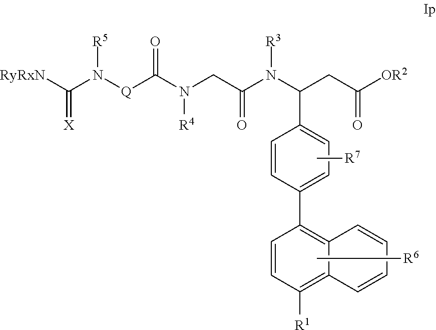
or a pharmaceutically acceptable salt thereof, wherein
R¹ is optionally substituted alkyl, optionally substituted alkoxy, or 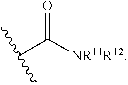
wherein R¹¹ and R¹² are each independently optionally substituted alkyl or a linking moiety, or R¹ is a linking moiety;
R² is H, optionally substituted alkyl, or a moiety;
R³ is H or optionally substituted alkyl;
R⁴ is H or optionally substituted alkyl;
R⁵ is H or optionally substituted alkyl;
R⁶ is selected from the group consisting of H, optionally substituted alkyl, optionally substituted alkoxy, halo, optionally substituted amino, or a linking moiety;
Q is optionally substituted aryl or optionally substituted alkylene;
X is O, CR⁸R⁹, NR⁸;
wherein R⁸ is selected from H, optionally substituted alkyl, or R⁸ is taken together with Rx or Ry to form a 4-, 5-, 6-, 7-, 8- or 9-membered ring, and R⁹ is H or optionally substituted alkyl;
Rx and Ry are each independently H, optionally substituted alkyl, a cargo molecule or Rx and Ry may be taken together to form a double bond with R¹⁰, wherein R¹⁰ is H, optionally substituted alkyl, or R¹⁰ may be taken together with X and the atoms to which it is attached to form a 4-, 5-, 6-, 7-, 8, or 9-membered ring;
wherein at least one of R¹, R², R⁶, R¹¹, R¹², Rx and Ry comprise a linking moiety; and
wherein when Q is optionally substituted alkyl and the length of the optionally substituted alkyl chain represented by Q is 3 carbons, then R¹ is

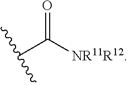

28. The compound of claim 27, wherein the linking moiety comprises a functional group selected from the group consisting of: azide, ester, carbamate, alkene, alcohol, amine, amide, carbonate, and alkyne.

29. (canceled)

30. The compound of claim 27, wherein the compound has the formula:

| Compound | |
|---|---|
| Number | Formula |
| 41p | 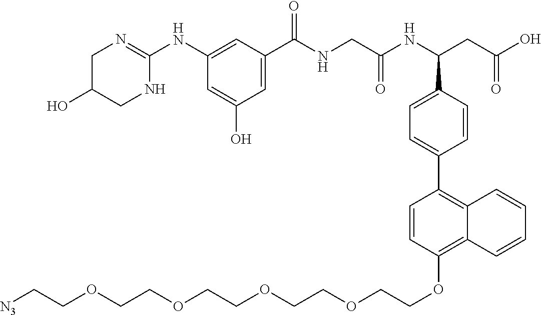 [SMILES: [N-]=[N+]=NCCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12] |
| 42p | 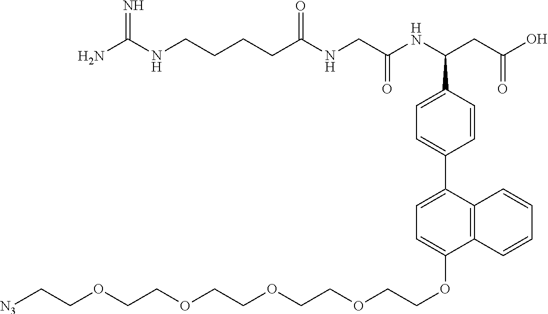 [SMILES: [N-]=[N+]=NCCOCCOCCOCCOCCOc1ccc(-c2ccc([C@H](CC(=O)O)NC(=O)CNC(=O)CCCCNC(=N)N)cc2)c2ccccc12] |
| 43p | 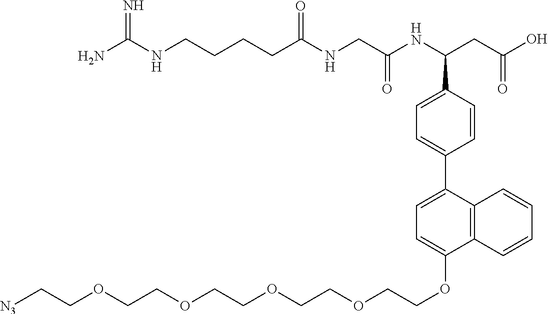 [SMILES: [N-]=[N+]=NCCOCCOCCOCCOCCOc1ccc(-c2ccc([C@H](CC(=O)O)NC(=O)CNC(=O)CCCCNC(=N)N)cc2)c2ccccc12] |
| 44p | 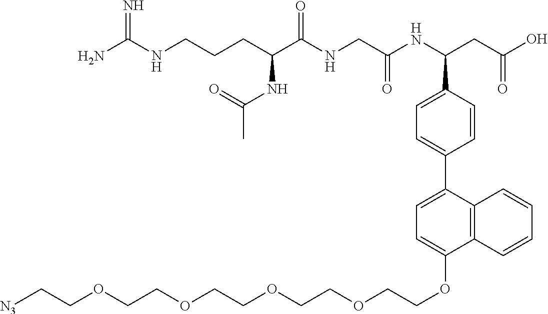 [SMILES: CC(=O)N[C@@H](CCCNC(=N)N)C(=O)NCC(=O)N[C@@H](CC(=O)O)c1ccc(-c2ccc(OCCOCCOCCOCCOCCN=[N+]=[N-])c3ccccc23)cc1] |
| 45p | 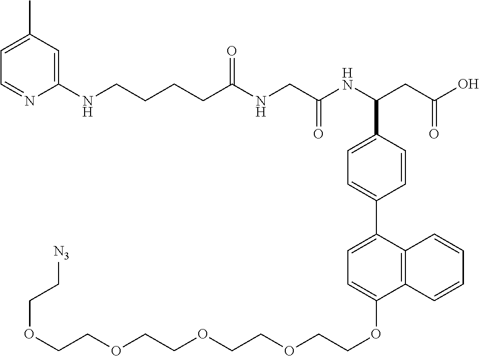 [SMILES: Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1] |
| 46p | 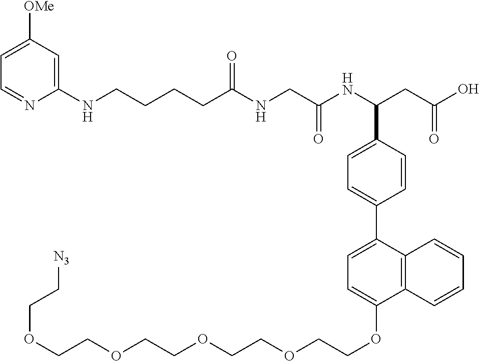 [SMILES: COc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1] |
| 47p | 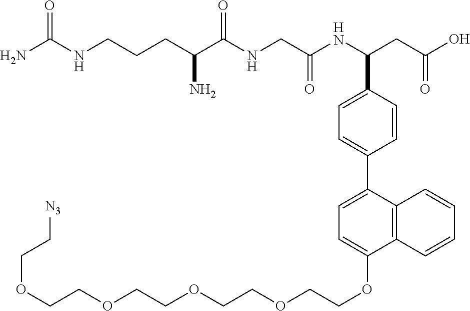 [SMILES: [N-]=[N+]=NCCOCCOCCOCCOCCOc1ccc(-c2ccc([C@H](CC(=O)O)NC(=O)CNC(=O)[C@@H](N)CCCNC(N)=O)cc2)c2ccccc12] |
| 48p | 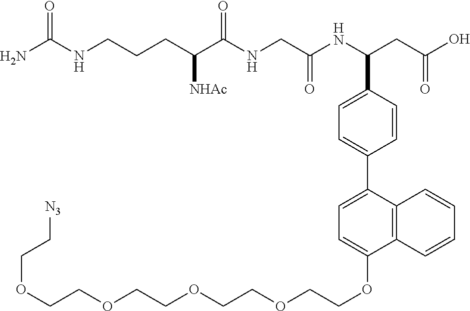 [SMILES: CC(=O)N[C@@H](CCCNC(N)=O)C(=O)NCC(=O)N[C@@H](CC(=O)O)c1ccc(-c2ccc(OCCOCCOCCOCCOCCN=[N+]=[N-])c3ccccc23)cc1] |
| 49p | 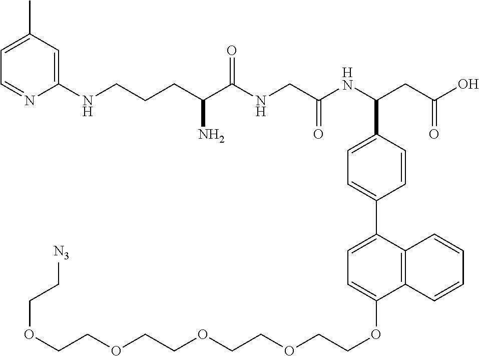 [SMILES: Cc1ccnc(NCCC[C@H](N)C(=O)NCC(=O)N[C@@H](CC(=O)O)c2ccc(-c3ccc(OCCOCCOCCOCCOCCN=[N+]=[N-])c4ccccc34)cc2)c1] |
| 50p | 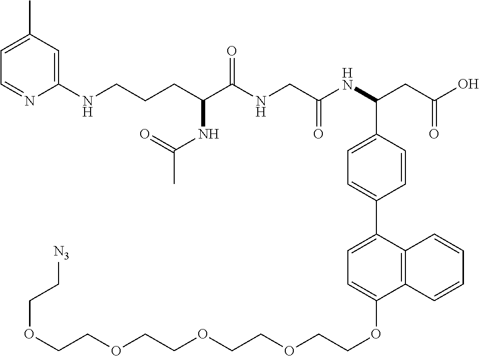 [SMILES: CC(=O)N[C@@H](CCCNc1cc(C)ccn1)C(=O)NCC(=O)N[C@@H](CC(=O)O)c1ccc(-c2ccc(OCCOCCOCCOCCOCCN=[N+]=[N-])c3ccccc23)cc1] |
| 51p | 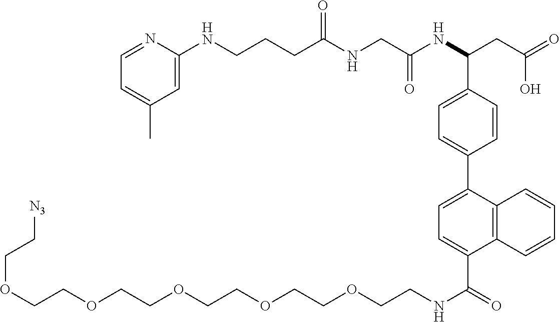 [SMILES: Cc1ccnc(NCCCC(=O)NCC(=O)N[C@@H](CC(=O)O)c2ccc(-c3ccc(C(=O)NCCOCCOCCOCCOCCOCCN=[N+]=[N-])c4ccccc34)cc2)c1] |
| 52p | 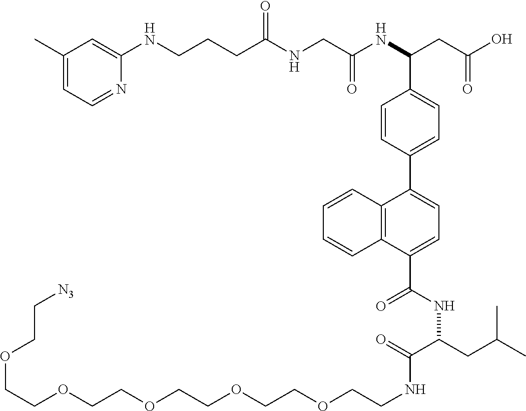 [SMILES: Cc1ccnc(NCCCC(=O)NCC(=O)N[C@@H](CC(=O)O)c2ccc(-c3ccc(C(=O)N[C@H](CC(C)C)C(=O)NCCOCCOCCOCCOCCOCCN=[N+]=[N-])c4ccccc34)cc2)c1] |
| 53p | 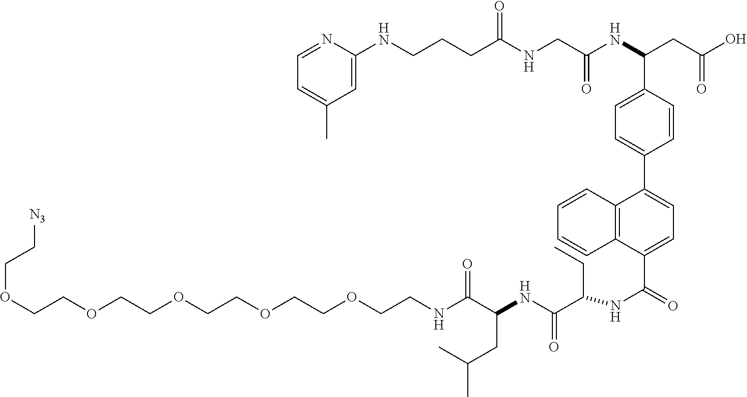 [SMILES: CC[C@H](NC(=O)c1ccc(-c2ccc([C@H](CC(=O)O)NC(=O)CNC(=O)CCCNc3cc(C)ccn3)cc2)c2ccccc12)C(=O)N[C@@H](CC(C)C)C(=O)NCCOCCOCCOCCOCCOCCN=[N+]=[N-]] |
| 54p | 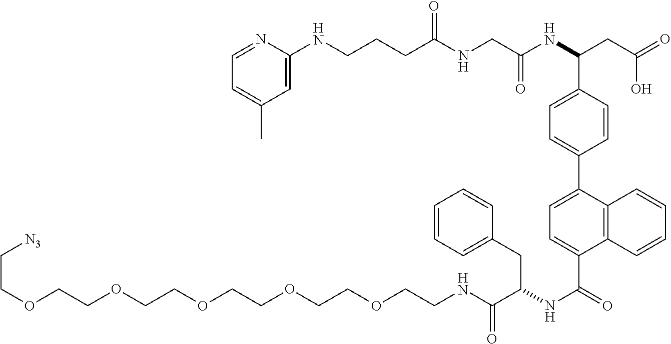 [SMILES: Cc1ccnc(NCCCC(=O)NCC(=O)N[C@@H](CC(=O)O)c2ccc(-c3ccc(C(=O)N[C@@H](Cc4ccccc4)C(=O)NCCOCCOCCOCCOCCOCCN=[N+]=[N-])c4ccccc34)cc2)c1] |
| 55p | 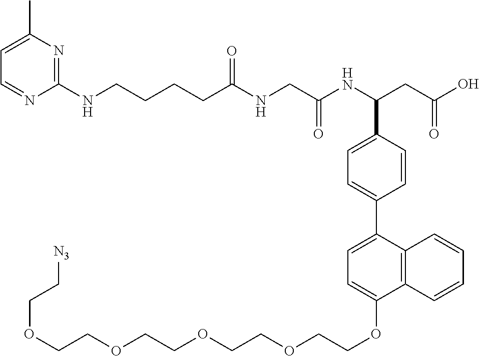 [SMILES: Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)n1] |
| 56p | 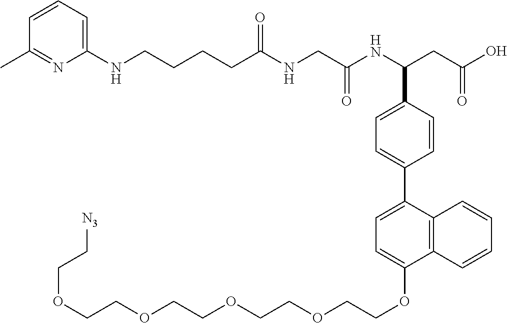 [SMILES: Cc1cccc(NCCCCC(=O)NCC(=O)N[C@@H](CC(=O)O)c2ccc(-c3ccc(OCCOCCOCCOCCOCCN=[N+]=[N-])c4ccccc34)cc2)n1] |
| 57p | 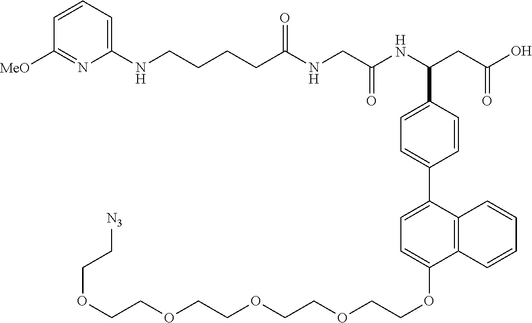 [SMILES: COc1cccc(NCCCCC(=O)NCC(=O)N[C@@H](CC(=O)O)c2ccc(-c3ccc(OCCOCCOCCOCCOCCN=[N+]=[N-])c4ccccc34)cc2)n1] |
| 58p | 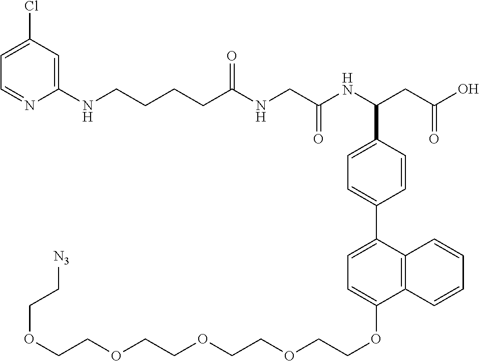 [SMILES: [N-]=[N+]=NCCOCCOCCOCCOCCOc1ccc(-c2ccc([C@H](CC(=O)O)NC(=O)CNC(=O)CCCCNc3cc(Cl)ccn3)cc2)c2ccccc12] |
| 59p | 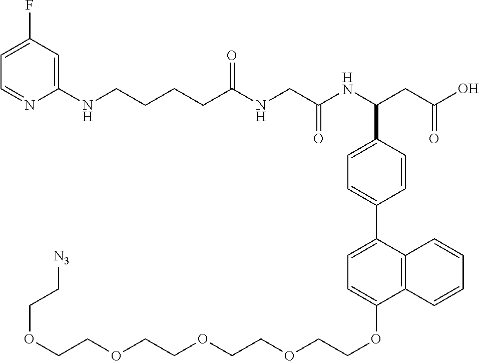 [SMILES: [N-]=[N+]=NCCOCCOCCOCCOCCOc1ccc(-c2ccc([C@H](CC(=O)O)NC(=O)CNC(=O)CCCCNc3cc(F)ccn3)cc2)c2ccccc12] |
| 60p | 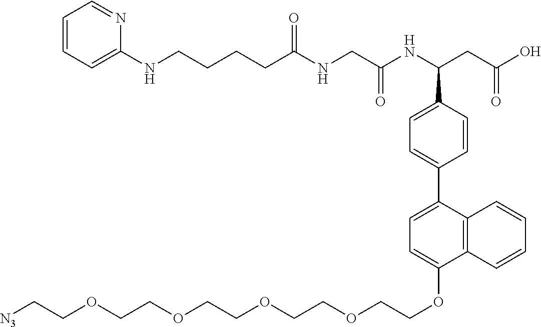 [SMILES: [N-]=[N+]=NCCOCCOCCOCCOCCOc1ccc(-c2ccc([C@H](CC(=O)O)NC(=O)CNC(=O)CCCCNc3ccccn3)cc2)c2ccccc12] |

or an acceptable salt thereof.

31. A method of making a compound of Formula I 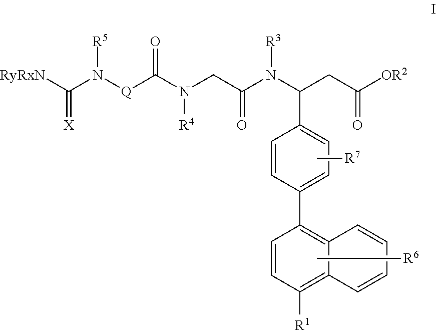
or a pharmaceutically acceptable salt thereof, wherein
R¹ is optionally substituted alkyl, optionally substituted alkoxy, or 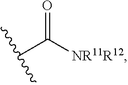
wherein R¹¹ and R¹² are each independently optionally substituted alkyl or a cargo molecule, or R¹ is a cargo molecule;
R² is H, optionally substituted alkyl, or a cargo molecule;
R³ is H or optionally substituted alkyl;
R⁴ is H or optionally substituted alkyl;
R⁵ is H or optionally substituted alkyl;
R⁶ is selected from the group consisting of H, optionally substituted alkyl, optionally substituted alkoxy, halo, optionally substituted amino, or a cargo molecule;
Q is optionally substituted aryl or optionally substituted alkylene;
X is O, CR⁸R⁹, NR⁸;
wherein R⁸ is selected from H, optionally substituted alkyl, or R⁸ is taken together with Rx or Ry to form a 4-, 5-, 6-, 7-, 8- or 9-membered ring, and R⁹ is H or optionally substituted alkyl;
Rx and Ry are each independently H, optionally substituted alkyl, a cargo molecule or Rx and Ry may be taken together to form a double bond with R¹⁰, wherein R¹⁰ is H, optionally substituted alkyl, or R¹⁰ may be taken together with X and the atoms to which it is attached to form a 4-, 5-, 6-, 7-, 8, or 9-membered ring;
wherein at least one of R¹, R², R⁶, R¹¹, R¹², Rx and Ry comprise a cargo molecule; and
wherein when Q is optionally substituted alkyl and the length of the optionally substituted alkyl chain represented by Q is 3 carbons, then R¹ is 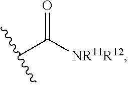
comprising reacting a compound of Formula Ip 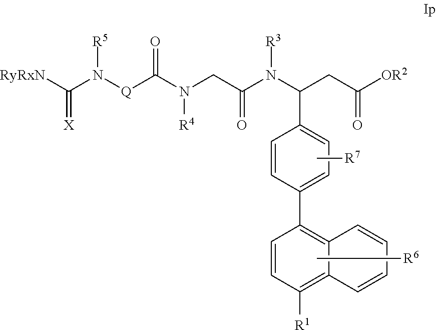
or a pharmaceutically acceptable salt thereof, wherein
R¹ is optionally substituted alkyl, optionally substituted alkoxy, or 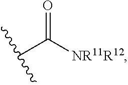
wherein R¹¹ and R¹² are each independently optionally substituted alkyl or a linking moiety, or R¹ is a linking moiety;
R² is H, optionally substituted alkyl, or a linking moiety;
R³ is H or optionally substituted alkyl;
R⁴ is H or optionally substituted alkyl;
R⁵ is H or optionally substituted alkyl;
R⁶ is selected from the group consisting of H, optionally substituted alkyl, optionally substituted alkoxy, halo, optionally substituted amino, or a linking moiety;
Q is optionally substituted aryl or optionally substituted alkylene;
X is O, CR⁸R⁹, NR⁸;
wherein R⁸ is selected from H, optionally substituted alkyl, or R⁸ is taken together with Rx or Ry to form a 4-, 5-, 6-, 7-, 8- or 9-membered ring, and R⁹ is H or optionally substituted alkyl;
Rx and Ry are each independently H, optionally substituted alkyl, a cargo molecule or Rx and Ry may be taken together to form a double bond with R¹⁰, wherein R¹⁰ is H, optionally substituted alkyl, or R¹⁰ may be taken together with X and the atoms to which it is attached to form a 4-, 5-, 6-, 7-, 8, or 9-membered ring;
wherein at least one of R¹, R², R⁶, R¹¹, R¹², Rx and Ry comprise a linking moiety; and
wherein when Q is optionally substituted alkyl and the length of the optionally substituted alkyl chain represented by Q is 3 carbons, then R¹ is 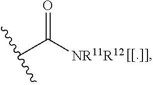
with a cargo molecule comprising a reactive moiety.

32. The method of claim 31, wherein the cargo molecule is an RNAi agent.

33. The method of claim 32, wherein the linking moiety comprises an azide and the reactive moiety is an alkyne.

34. A composition comprising the compound of claim 1, and a pharmaceutically acceptable excipient.

35. The composition of claim 34, wherein the cargo molecule is an RNAi agent targeting a gene located in a skeletal muscle cell.

36. (canceled)

37. A method of treating or preventing a disease or disorder that may be treated or ameliorated by knocking down gene expression in skeletal muscle cells comprising administering to a subject in need thereof a pharmaceutical composition comprising the composition of claim 34.

38. (canceled)
